SEQUENCE LISTING (18 sequences)

SEQ ID NO: 1 - DNA, 1080 bp, Homo sapiens
  CDS (1)..(1026)
atgcctttccttttgggtcttagacaggataaggaagcctgtgtgggtaccaacaatcaaagctacatctgtgacacaggacactgctgtggacagtctcagtgctgcaactactactatgaactctggtggttctggctggtgtggaccatcatcatcatcctgagctgctgctgtgtttgccaccaccgccgagccaagcaccgccttcaggcccagcagcggcaacatgaaatcaacctgatcgcttaccgagaagcccacaattactcagcgctgccattttatttcaggtttttgccaaactatttactacctccttatgaggaagtggtgaaccgacctccaactcctcccccaccatacagtgccttccagctacagcagcagcagctgctgcctccacagtgtggccctgcaggtggcagtcccccgggcatcgatcccaccaggggatcccagggggcacagagcagccccttgtctgagcccagcagaagcagcacaagacccccaagcatcgctgaccctgatccctctgacctaccagttgaccgagcagccaccaaagccccagggatggagcccagtggctctgtggctggcctgggggagctggacccgggggccttcctggacaaagatgcagaatgtagggaggagctgctgaaagatgacagctctgaacacggcgcacccgacagcaaagagaagacgcctgggagacatcgccgcttcacaggtgactcgggcattgaagtgtgtgtgtgcaaccggggccaccatgacgatgacctcaaagagttcaacacactcatcgatgatgctctggatgggcccctggacttctgcgacagctgccatgtgcggccccctggtgatgaggaggaaggcctctgtcagtcctctgaggagcaggctcgagagcctgggcacccgcacctgccacggccgcccgcatgcctgctgctgaacaccatcaacgagcaggactctcccaactcccagagcagcagctcccccagctagagcaggtcctgccagcacccagcaacttggcaaagcaaccagggtagggga

SEQ ID NO: 2 - protein, 342 aa, Homo sapiens
MPFLLGLRQDKEACVGTNNQSYICDTGHCCGQSQCCNYYYELWWFWLVWTIIIILSCCCVCHHRRAKHRLQAQQRQHEINLIAYREAHNYSALPFYFRFLPNYLLPPYEEVVNRPPTPPPPYSAFQLQQQQLLPPQCGPAGGSPPGIDPTRGSQGAQSSPLSEPSRSSTRPPSIADPDPSDLPVDRAATKAPGMEPSGSVAGLGELDPGAFLDKDAECREELLKDDSSEHGAPDSKEKTPGRHRRFTGDSGIEVCVCNRGHHDDDLKEFNTLIDDALDGPLDFCDSCHVRPPGDEEEGLCQSSEEQAREPGHPHLPRPPACLLLNTINEQDSPNSQSSSSPS

SEQ ID NO: 3 - DNA, 1140 bp, Homo sapiens
  CDS (1)..(1089)
atggagaggagaaggctcctgggtggcatggcgctcctgctcctccaggcgctgcccagccccttgtcagccagggctgaacccccgcaggataaggaagcctgtgtgggtaccaacaatcaaagctacatctgtgacacaggacactgctgtggacagtctcagtgctgcaactactactatgaactctggtggttctggctggtgtggaccatcatcatcatcctgagctgctgctgtgtttgccaccaccgccgagccaagcaccgccttcaggcccagcagcggcaacatgaaatcaacctgatcgcttaccgagaagcccacaattactcagcgctgccattttatttcaggtttttgccaaactatttactacctccttatgaggaagtggtgaaccgacctccaactcctcccccaccatacagtgccttccagctacagcagcagcagctgctgcctccacagtgtggccctgcaggtggcagtcccccgggcatcgatcccaccaggggatcccagggggcacagagcagccccttgtctgagcccagcagaagcagcacaagacccccaagcatcgctgaccctgatccctctgacctaccagttgaccgagcagccaccaaagccccagggatggagcccagtggctctgtggctggcctgggggagctggacccgggggccttcctggacaaagatgcagaatgtagggaggagctgctgaaagatgacagctctgaacacggcgcacccgacagcaaagagaagacgcctgggagacatcgccgcttcacaggtgactcgggcattgaagtgtgtgtgtgcaaccggggccaccatgacgatgacctcaaagagttcaacacactcatcgatgatgctctggatgggcccctggacttctgcgacagctgccatgtgcggccccctggtgatgaggaggaaggcctctgtcagtcctctgaggagcaggctcgagagcctgggcacccgcacctgccacggccgcccgcatgcctgctgctgaacaccatcaacgagcaggactctcccaactcccagagcagcagctcccccagctagagcaggtcctgccagcacccagcaacttggcaaagcaaccagggtagg

SEQ ID NO: 4 - protein, 363 aa, Homo sapiens
MERRRLLGGMALLLLQALPSPLSARAEPPQDKEACVGTNNQSYICDTGHCCGQSQCCNYYYELWWFWLVWTIIIILSCCCVCHHRRAKHRLQAQQRQHEINLIAYREAHNYSALPFYFRFLPNYLLPPYEEVVNRPPTPPPPYSAFQLQQQQLLPPQCGPAGGSPPGIDPTRGSQGAQSSPLSEPSRSSTRPPSIADPDPSDLPVDRAATKAPGMEPSGSVAGLGELDPGAFLDKDAECREELLKDDSSEHGAPDSKEKTPGRHRRFTGDSGIEVCVCNRGHHDDDLKEFNTLIDDALDGPLDFCDSCHVRPPGDEEEGLCQSSEEQAREPGHPHLPRPPACLLLNTINEQDSPNSQSSSSPS

SEQ ID NO: 5 - DNA, 22 bp, Artificial Sequence
  Description of Artificial Sequence Synthetic primer
ccaacgttagtgtggacgatgc

SEQ ID NO: 6 - DNA, 17 bp, Artificial Sequence
  Description of Artificial Sequence Synthetic primer
gcatggcgctcctgctc

SEQ ID NO: 7 - DNA, 24 bp, Artificial Sequence
  Description of Artificial Sequence Synthetic primer
gtagtagttgcagcactgagactg

SEQ ID NO: 8 - DNA, 31 bp, Artificial Sequence
  Description of Artificial Sequence Synthetic primer
ccacagcagtgtcctgtgtcacagatgtagc

SEQ ID NO: 9 - DNA, 24 bp, Artificial Sequence
  Description of Artificial Sequence Synthetic primer
cagtctcagtgctgcaactactac

SEQ ID NO: 10 - DNA, 21 bp, Artificial Sequence
  Description of Artificial Sequence Synthetic primer
ggcttctcggtaagcgatcag

SEQ ID NO: 11 - DNA, 31 bp, Artificial Sequence
  Description of Artificial Sequence Synthetic primer
ctcaggatgatgatgatggtccacaccagcc

SEQ ID NO: 12 - DNA, 21 bp, Artificial Sequence
  Description of Artificial Sequence Synthetic primer
ccaaggatgtgctgagtgtgg

SEQ ID NO: 13 - DNA, 22 bp, Artificial Sequence
  Description of Artificial Sequence Synthetic primer
cgtgttcagatagcctgtgtgg

SEQ ID NO: 14 - DNA, 20 bp, Artificial Sequence
  Description of Artificial Sequence Synthetic primer
ccaactggaccgtcaccaac

SEQ ID NO: 15 - DNA, 23 bp, Artificial Sequence
  Description of Artificial Sequence Synthetic primer
gaatggcaatctcatactctcgg

SEQ ID NO: 16 - DNA, 4122 bp, Homo sapiens
tgtttactttgtctgctttgctaaagaaggccggtgaaccaggaccaccgcacacacaggcccaccaggggcaatgctcattccaagaccttaacttttaagagccctttgttccaacgttagtgtggacgatgctcttgcaggatgcctttccttttgggtcttagacaggataaggaagcctgtgtgggtaccaacaatcaaagctacatctgtgacacaggacactgctgtggacagtctcagtgctgcaactactactatgaactctggtggttctggctggtgtggaccatcatcatcatcctgagctgctgctgtgtttgccaccaccgccgagccaagcaccgccttcaggcccagcagcggcaacatgaaatcaacctgatcgcttaccgagaagcccacaattactcagcgctgccattttatttcaggtttttgccaaactatttactacctccttatgaggaagtggtgaaccgacctccaactcctcccccaccatacagtgccttccagctacagcagcagcagctgctgcctccacagtgtggccctgcaggtggcagtcccccgggcatcgatcccaccaggggatcccagggggcacagagcagccccttgtctgagcccagcagaagcagcacaagacccccaagcatcgctgaccctgatccctctgacctaccagttgaccgagcagccaccaaagccccagggatggagcccagtggctctgtggctggcctgggggagctggacccgggggccttcctggacaaagatgcagaatgtagggaggagctgctgaaagatgacagctctgaacacggcgcacccgacagcaaagagaagacgcctgggagacatcgccgcttcacaggtgactcgggcattgaagtgtgtgtgtgcaaccggggccaccatgacgatgacctcaaagagttcaacacactcatcgatgatgctctggatgggcccctggacttctgcgacagctgccatgtgcggccccctggtgatgaggaggaaggcctctgtcagtcctctgaggagcaggctcgagagcctgggcacccgcacctgccacggccgcccgcatgcctgctgctgaacaccatcaacgagcaggactctcccaactcccagagcagcagctcccccagctagagcaggtcctgccagcacccagcaacttggcaaagcaaccagggtaggggagaaccacgagagaagcattaagtgactttcaaagactttcagagtacagccacttggttcctttttgtttgttttccttctcctctcctgcattttcctccatctccaggtacagttcggggtgtggatgcctcttcctccacaagggcacagtgttgtggagggctaagttggttctgtgactcattcctcataccctaactccatctcctttctttaaagtcaaatctcacctacctgtttgggtcagagagatgtgttttgaaagcccccaaggaaggaggctgggactgtgccctgacatgattcttggtgatggaataggtttgtgctctgattctagtttaagagaacgttgctgtatctcagtccaggagaggcagcccatcttggccctggatgaagaaggaaacccacagaggcccagggcttgtcattgggctgccagtgtctgccaagccagcattgagctaatcctgtgggaggatgagagctactgggccgttgtatgataggttggtaggggcttgttgatctgtcaaattccaggtgacaagatctatgcaccccatgcgtccttgaggggcctcttccccgcaggctctggctggccgcaggctggttctggtgtgaaaggttatactgccttttctttgtttgtttgtttttttctctaaaaacaaacagcaaaagacagctgaaaacaagaacttcaccggtgggcaggcaagaattctcttctggaaaatgacgtttgtggctctttcccaagttggccttcaaagagcctgcctgctgttgagccagaagatgtctcgtgtgaaggctggggtggcggctgtcttggaacctctgtgagcaggaggccctaagccgcagcagtggatagaggtgcagctctctgcctctctgccctttggtctgtgttcacaggtgacccgtgtcagcctgcatcgcaagcacacaccctgcgggccttcaagtctcactgttccgtatgaggaaacagacagcggactgaggaagcgatggccccagagaaagggcccctgtagcctggctctcacacagtattttatctttgattctgaataaatattttttgtggggttttttttttttttttggtggcagttgtttgttttaaactgaccacttggaagaaacaccttggttatctgtggttttcatgccttgtccctgcctctacccccaccccttttgagtcgggtgactcatttttctgtgtagagactcggtggcccaggcaggaggtgaaagcagccatccggaaggccctggggacccttgtgcctgttgctcgccttcaggtcaccagctgagctgcgataggaaaatctgaatggaggcagcaaacagccaaaacaaacattccccacccggccctgtgcatatgaagtctttcttcccccaactcttgaacgatgatgatattcagacgaagcattgatgttatggaagaaagaaagaaacaaacaaaaaatatatatatatgtccaaaaacagacaaatccaagggtgtgaggtaaacgagtgtctgcatttagattccacaaaaccaaaatccatgttgaacaaagttaagtccgtacacagtgactttttgggtgagccgtgtgtgtctgtctgttgtgtgtgtgcctcaagccctgttttcctgtgaagatactttgagtggcagccattctctccacgtgaaccacacgtctggagcacagacaggcctctcaaggtcattgatcttacgcatttactgtttaccgaacaaatgtctgactgtgtactcgggtgtactccgcagcattgtcgactgcagtcccctgtgtttgccagagatactgtgctcgaagtagaggttttactctactcatcactgcgatttgcacattgctccgtggacactcggaggcctgcgttctgttccctataaatggaagcgtgctctgagcctgtctgcctccctcggctgctgctggtcctcagtaccagcgcccgggggtgtccacaaccacttgggacagaagaaggtggaatttcagacagaagcttgactgggtcttcaatgacaggcttggactagctgtggcccagacatcggccctgcccagaattgccaggaggaggctttgcaggctctagaggagccgcagggcctgcctgcctctggtgagtccaacaggcacaagcaagctggcgtgtggccagaggtagccggagtgtgtcacagcccctcagatgcctttccttccacctttttttttattttttaagaatcccaaataactcactgaagtgtctcaaaggcgaacaagttttaccaaaatgaatcctttttcagttaacagatcaaatggatgagttctgagcctctcaagttcctttccccagttagagtggggaactgggcaagtgttaactgtgggactcactgcagcgtcctatcctaaaggcacgagaagacggaaatgcaacctgcggagctgggcttggttcccaggtcacagtttggcccccgctacaggatgctgccctgctcagagagagatttaatagggagctgaaggaatcgttagggggccagggagatgtgactgaggctggctttccacgtgaatgagacggggtcggtggagggtttggtgctacagccagtcagaagatttgcaaatgcgaacacattcctgtgtgaggcacgttaccctttgtcagttattgtgaatatgtgtattttaagcaataagattcagctggtcagacttttctgggcagtctcagtgacgcatttcctgtgctgtgattgttctgaagacagagtggctctaaccactgtgagaagcccaaataaaaattgatcccaaaaatgaaaaaaaaaaaaaa

SEQ ID NO: 17 - protein, 4 aa, Artificial Sequence
  Description of Artificial Sequence Synthetic peptide
DEAX

SEQ ID NO: 18 - DNA, 24 bp, Artificial Sequence
  Description of Artificial Sequence Synthetic primer
tttttttttttttttttttttttt

(19) United States
(12) Patent Application Publication
(10) Pub. No.: US 2006/0063156 A1
(43) Pub. Date: Mar. 23, 2006

(54) Outcome prediction and risk classification in childhood leukemia

(76) Inventors: Cheryl L. Willman, Albuquerque, NM (US); Paul Helman, Albuquerque, NM (US); Robert Veroff, Albuquerque, NM (US); Monica Mosquera-Caro, Albuquerque, NM (US); George S. Davidson, Albuquerque, NM (US); Shawn B. Martin, Albuquerque, NM (US); Susan R. Atlas, Albuquerque, NM (US); Erik Andries, Rio Rancho, NM (US); Huining Kang, Albuquerque, NM (US); Jonathan J. Shuster, Gainesville, FL (US); Xuefei Wang, Albuquerque, NM (US); Richard C. Harvey, Placitas, NM (US); David M. Haaland, Albuquerque, NM (US); Jeffrey W. Potter, Albuquerque, NM (US)

Correspondence Address: COLEMAN SUDOL SAPONE, P.C., 714 COLORADO AVENUE, BRIDGE PORT, CT, 06605-1601, US

(21) Appl. No.: 10/729,895

(22) Filed: Dec. 5, 2003

Related U.S. Application Data
(60) Provisional application No. 60/432,064, filed on Dec. 6, 2002.
(60) Provisional application No. 60/432,077, filed on Dec. 6, 2002.
(60) Provisional application No. 60/432,078, filed on Dec. 6, 2002.
(60) Provisional application No. 60/510,904, filed on Oct. 14, 2003.
(60) Provisional application No. 60/510,968, filed on Oct. 14, 2003.
(60) Provisional application No. 60/527,610, filed on Dec. 5, 2003.

(51) Int. Cl. C12Q 1/68; C07H 21/04; C12P 21/06; C12N 15/09; C07K 14/705

(52) U.S. Cl. 435/6; 435/69.1; 435/320.1; 435/325; 530/350; 536/23.2

(57) ABSTRACT

Genes and gene expression profiles useful for predicting outcome, risk classification, cytogenetics and/or etiology in pediatric acute lymphoblastic leukemia (ALL). OPAL1 is a novel gene associated with outcome and, along with other newly identified genes, represent a novel therapeutic targets.

This application claims the benefit of U.S. Provisional Application Ser. Nos. 60/432,064; 60/432,077; and 60/432,078; all of which were filed Dec. 6, 2002; and U.S. Provisional Application Ser. Nos. 60/510,904 and 60/510,968, both of which were filed Oct. 14, 2003; and a U.S. Provisional Application entitled “Outcome Prediction in Childhood Leukemia” filed on even date herewith. These provisional applications are incorporated herein by reference in their entireties.

### STATEMENT OF GOVERNMENT RIGHTS

This invention was made with government support under a grant from the National Institutes of Health (National Cancer Institute), Grant No. NIH NCI U01 CA88361; and under a contract from the Department of Energy, Contract No. DE-AC04-94AL85000. The U.S. Government has certain rights in this invention.

### BACKGROUND OF THE INVENTION

Leukemia is the most common childhood malignancy in the United States. Approximately 3,500 cases of acute leukemia are diagnosed each year in the U.S. in children less than 20 years of age. The large majority (>70%) of these cases are acute lymphoblastic leukemias (ALL) and the remainder acute myeloid leukemias (AML). The outcome for children with ALL has improved dramatically over the past three decades, but despite significant progress in treatment, 25% of children with ALL develop recurrent disease. Conversely, another 25% of children who now receive dose intensification are likely “over-treated” and may well be cured using less intensive regimens resulting in fewer toxicities and long term side effects. Thus, a major challenge for the treatment of children with ALL in the next decade is to improve and refine ALL diagnosis and risk classification schemes in order to precisely tailor therapeutic approaches to the biology of the tumor and the genotype of the host.

Leukemia in the first 12 months of life (referred to as infant leukemia) is extremely rare in the United States, with about 150 infants diagnosed each year. There are several clinical and genetic factors that distinguish infant leukemia from acute leukemias that occur in older children. First, while the percentage of acute lymphoblastic leukemia (ALL) cases is far more frequent (approximately five times) than acute myeloid leukemia in children from ages 1-15 years, the frequency of ALL and AML in infants less than one year of age is approximately equivalent. Secondly, in contrast to the extensive heterogeneity in cytogenetic abnormalities and chromosomal rearrangements in older children with ALL and AML, nearly 60% of acute leukemias in infants have chromosomal rerrangments involving the MLL gene (for Mixed Lineage Leukemia) on chromosome 11q23. MLL translocations characterize a subset of human acute leukemias with a decidedly unfavorable prognosis. Current estimates suggest that about 60% of infants with AML and about 80% of infants with ALL have a chromosomal rearrangment involving MLL abnormality in their leukemia cells. Whether hematopoietic cells in infants are more likely to undergo chromosomal rearrangements involving 11q13 or whether this 11q13 rearrangement reflects a unique environmental exposure or genetic susceptibliity remains to be determined.

The modern classification of acute leukemias in children and adults relies on morphologic and cytochemical features that may be useful in distinguishing AML from ALL, changes in the expression of cell surface antigens as a precursor cell differentiates, and the presence of specific recurrent cytogenetic or chromosomal rearrangements in leukemic cells. Using monoclonal antibodies, cell surface antigens (called clusters of differentiation (CD)) can be identified in cell populations; leukemias can be accurately classified by this means (immunophenotyping). By immunophenotyping, it is possible to classify ALL into the major categories of “common-CD10+B-cell precursor” (around 50%), “pre-B” (around 25%), “T” (around 15%), “null” (around 9%) and “B” cell ALL (around 1%). All forms other than T-ALL are considered to be derived from some stage of B-precursor cell, and “null” ALL is sometimes referred to as “early B-precursor” ALL.

Current risk classification schemes for ALL in children from 1-18 years of age use clinical and laboratory parameters such as patient age, initial white blood cell count, and the presence of specific ALL-associated cytogenetic abnormalities to stratify patients into “low,” “standard,” “high,” and “very high” risk categories. National Cancer Institute (NCI) risk criteria are first applied to all children with ALL, dividing them into “NCI standard risk” (age 1.00-9.99 years, WBC<50,000) and “NCI high risk” (age>10 years, WBC>50,000) based on age and initial white blood cell count (WBC) at disease presentation. In addition to these general NCI risk criteria, classic cytogenetic analysis and molecular genetic detection of frequently recurring cytogenetic abnormalities have been used to stratify ALL patients more precisely into “low,” “standard,” “high,” and “very high” risk categories. FIG. 1 shows the 4-year event free survival (EFS) projected for each of these groups.

These chromosomal aberrations primarily involve structural rearrangements (translocations) or numerical imbalances (hyperdiploidy—now assessed as specific chromosome trisomies, or hypodiploidy). Table 1 shows recurrent ALL genetic subtypes, their frequencies and their risk categorization.

TABLE 1
Recurrent Genetic Subtypes of B and T Cell ALL

| | Associated Genetic | | |
|---|---|---|---|
| Subtype | Abnormalities | Frequency in Children | Risk Category |
| B-Precursor ALL | Hyperdiploid DNA Content; | 25% of B Precursor Cases | Low |
| | Trisomies of Chromosomes 4, | | |
| | 10, 17 | | |
| | t(12; 21)(p13; q22): TEL/AML1 | 28% of B Precursor Cases | Low |
| | | 4% of B Precursor Cases; | |
| | | >80% of Infant ALL | |
| | 11q23/MLL Rearrangements; | 6% of B Precursor Cases | High |
| | particularly t(4; 11)(q21; q23) | | |
| | t(1; 19)9q23; p13) - E2A/PBX1 | 2% of B Precursor Cases | High |
| | t(9; 22)(q34; q11): BCR/ABL | Relatively Rare | Very High |
| | Hypodiploidy | | Very High |
| B-ALL | t(8; 14)(q24; q32) - IgH/MYC | 5% of all B lineage ALL | High |
| | | cases | |
| T-ALL | Numerous translocations | 7% of ALL cases | Not Clearly |
| | involving the TCR αβ (7q35) or | | Defined |
| | TCR γδ (14q11) loci | | |

The rate of disappearance of both B precursor and T ALL leukemic cells during induction chemotherapy (assessed morphologically or by other quantitative measures of residual disease) has also been used as an assessment of early therapeutic response and as a means of targeting children for therapeutic intensification (Gruhn et al., Leukemia 12:675-681, 1998; Foroni et al., Br. J. Haematol. 105:7-24, 1999; van Dongen et al., Lancet 352:1731-1738, 1998; Cavé et al., N. Engl. J. Med. 339:591-598, 1998; Coustan-Smith et al., Lancet 351:550-554, 1998; Chessells et al., Lancet 343:143-148, 1995; Nachman et al., N. Engl. J. Med. 338:1663-1671, 1998).

Children with “low risk” disease (22% of all B precursor ALL cases) are defined as having standard NCI risk criteria, the presence of low risk cytogenetic abnormalities (t(12;21)/TEL; AML1 or trisomies of chromosomes 4 and 10), and a rapid early clearance of bone marrow blasts during induction chemotherapy. Children with “standard risk” disease (50% of ALL cases) are NCI standard risk without “low risk” or unfavorable cytogenetic features, or, are children with low risk cytogenetic features who have NCI high risk criteria or slow clearance of blasts during induction. Although therapeutic intensification has yielded significant improvements in outcome in the low and standard risk groups of ALL, it is likely that a significant number of these children are currently “over-treated” and could be cured with less intensive regimens resulting in fewer toxicities and long term side effects. Conversely, a significant number of children even in these good risk categories still relapse and a precise means to prospectively identify them has remained elusive. Nearly 30% of children with ALL have “high” or “very high” risk disease, defined by NCI high risk criteria and the presence of specific cytogenetic abnormalities (such as t(1;19), t(9;22) or hypodiploidy) (Table 1); again, precise measures to distinguish children more prone to relapse in this heterogeneous group have not been established.

Despite these efforts, current diagnosis and risk classification schemes remain imprecise. Children with ALL more prone to relapse who require more intensive approaches and children with low risk disease who could be cured with less intensive therapies are not adequately predicted by current classification schemes and are distributed among all currently defined risk groups. Although pre-treatment clinical and tumor genetic stratification of patients has generally improved outcomes by optimizing therapy, variability in clinical course continues to exist among individuals within a single risk group and even among those with similar prognostic features. In fact, the most significant prognostic factors in childhood ALL explain no more than 4% of the variability in prognosis, suggesting that yet undiscovered molecular mechanisms dictate clinical behavior (Donadieu et al., Br J Haematol, 102:729-739, 1998). A precise means to prospectively identify such children has remained elusive.

### SUMMARY OF THE INVENTION

The present invention is directed to methods for outcome prediction and risk classification in childhood leukemia. In one embodiment, the invention provides a method for classifying leukemia in a patient that includes obtaining a biological sample from a patient; determining the expression level for a selected gene product to yield an observed gene expression level; and comparing the observed gene expression level for the selected gene product to a control gene expression level. The control gene expression level can the expression level observed for the gene product in a control sample, or a predetermined expression level for the gene product. An observed expression level that differs from the control gene expression level is indicative of a disease classification. In another aspect, the method can include determining a gene expression profile for selected gene products in the biological sample to yield an observed gene expression profile; and comparing the observed gene expression profile for the selected gene products to a control gene expression profile for the selected gene products that correlates with a disease classification; wherein a similarity between the observed gene expression profile and the control gene expression profile is indicative of the disease classification.

The disease classification can be, for example, a classification based on predicted outcome (remission vs therapeutic failure); a classification based on karyotype; a classification based on leukemia subtype; or a classification based on disease etiology. Where the classification is based on disease outcome, the observed gene product is preferably a gene such as OPAL1, G1, G2, FYN binding protein, PBK1 or any of the genes listed in Table 42.

A novel gene, referred to herein as OPAL1, has been found to be strongly predictive of outcome in childhood leukemia, and presents new opportunities for better diagnosis, risk classification and better therapeutic options. Thus, in another embodiment, the invention includes a polynucleotide that encodes OPAL1 and variations thereof, the putative protein gene product of OPAL1 and variations thereof, and an antibody that binds to OPAL1, as well as host cells and vectors that include OPAL1.

The invention further provides for a method for predicting therapeutic outcome in a leukemia patient that includes obtaining a biological sample from a patient; determining the expression level for a selected gene product associated with outcome to yield an observed gene expression level; and comparing the observed gene expression level for the selected gene product to a control gene expression level for the selected gene product. The control gene expression level for the selected gene product can include the gene expression level for the selected gene product observed in a control sample, or a predetermined gene expression level for the selected gene product; wherein an observed expression level that is different from the control gene expression level for the selected gene product is indicative of predicted remission. Preferably, the selected gene product is OPAL1. Optionally, the method further comprises determining the expression level for another gene product, such as G1 or G2, and comparing in a similar fashion the observed gene expression level for the second gene product with a control gene expression level for that gene product, wherein an observed expression level for the second gene product that is different from the control gene expression level for that gene product is further indicative of predicted remission.

The invention further includes a method for detecting an OPAL1 polynucleotide in a biological sample which includes contacting the sample with an OPAL1 polynucleotide, or its complement, under conditions in which the polynucleotide selectively hybridizes to an OPAL1 gene; detecting hybridization of the polynucleotide to the OPAL1 gene in the sample. Likewise, the invention provides a method for detecting the OPAL1 protein in a biological sample that includes contacting the sample with an OPAL1 antibody under conditions in which the antibody selectively binds to an OPAL1 protein; and detecting the binding of the antibody to the OPAL1 protein in the sample. Pharmaceutical compositions including an therapeutic agent that includes an OPAL1 polynucleotide, polypeptide or antibody, together with a pharmaceutically acceptable carrier, are also included.

The invention further includes a method for treating leukemia comprising administering to a leukemia patient a therapeutic agent that modulates the amount or activity of the polypeptide associated with outcome. Preferably, the therapeutic agent increases the amount or activity of OPAL1.

Also provided by the invention is an in vitro method for screening a compound useful for treating leukemia. The invention further provides an in vivo method for evaluating a compound for use in treating leukemia. The candidate compounds are evaluated for their effect on the expression level(s) of one or more gene products associated with outcome in leukemia patients. Preferably, the gene product whose expression level is evaluated is the product of an OPAL1, G1, G2, FYN binding protein or PBK1 gene, or any of the genes listed in Table 42. More preferably, the gene product is a product of the OPAL1 gene.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows the 4 year event free survival (EFS) projected for NCI risk categories.

FIG. 2 shows the nucleotide sequences and amino acid sequences for the coding regions of two distinct OPAL1/G0 splice forms. FIG. 2A shows nucleotide sequence (SEQ ID NO:1) and amino acid sequence (SEQ ID NO:2) for the OPAL1/G0 splice form incorporation exon 1; and FIG. 2B shows nucleotide sequence (SEQ ID NO:3) and amino acid sequence (SEQ ID NO:4) for the OPAL1/G0 splice form incorporation exon 1a. Exons 1 and 1a are highlighted by italicized bold print. Numbers to the right indicate nucleotide and amino acid positions. FIG. 2C shows the sequence (SEQ ID NO:16) for the full length cDNA of OPAL1. The first exon (exon 1 in this example) is underlined. The start and end positions for the exons in the cDNA and reference sequence (GenBank accession NT_{—}030059.11) are as follows: exon 1, bases 1 to 171 (23284530 to 23284700), exon 2, bases 172 to 274 (23306276 to 23306378), exon 3, bases 275 to 436 (23318176 to 23318337) and exon 4, bases 437 to 4008 (23320878 to 23324547). The polyadenylation signal (position 4086 to 4091) is show in bold and italics.

FIG. 3 shows a bootstrap statistical analysis of gene list stability.

FIG. 4 is a Bayesian tree associated with outcome in ALL.

FIG. 5 is schematic drawing of the structure of OPAL1/G0.

FIG. 6 is a topographic map produced using VxInsight showing 9 novel biologic clusters of ALL (2 distinct T ALL clusters (S1 and S2) and 7 distinct B precursor ALL clusters (A, B, C, X, Y, Z)) each with distinguishing gene expression profiles.

FIG. 7 shows a gene list comparison. Principal Component Analysis (PCA and the VxInsight clustering program (ANOVA) were employed to identify genes that determined T-cell leukemia cases. The gene lists are compared with those derived from the different feature selection methods used by Yeoh et al. (Cancer Cell, 1: 133-143, 2002) for T-cell classification. The yellow color represents overlap between the lists derived by PCA and the T-ALL characterizing gene lists; the cyan represents overlap between the ANOVA and the T-ALL characterizing gene lists. The green pattern represents genes that are shared by all the lists.

FIG. 8 shows a gene list comparison. Bayesian Networks were employed to identify genes that determined the gene expression patterns across the different translocations. The gene lists were compared with those derived using chi square analysis by Yeoh et al. (Cancer Cell, 1:133-143, 2002) for ALL classification. The colored cells represent overlap between the lists derived by Bayesian nets and the ALL characterizing gene lists from Yeoh et al. (Cancer Cell, 1:133-143, 2002).

FIG. 9 shows Principal Component Analysis of the infant gene expression data. Principal Component Analysis (PCA) projections are used to compare the ALL/AML partition, the MLL/Non-MLL partition, and the VxInsight partition of the infant gene expression data. The three by three grid of plots in this figure allows this comparison by using the same PCA projections with different colors for the different partitions. Each row of the grid shows a different partition and each column shows a different PCA projection. The ALL/AML partition is shown in the first row of the figure using light purple for ALL and dark purple for AML. The three plots in this row give two-dimensional projections of the data onto the first three principal components. Since there are three such projections there are three plots (from left to right): PC 1 vs. PC 2, PC 2 vs. PC 3, and PC 1 vs. PC 3. This scheme is repeated for the remaining two partitions. Specifically, the MLL/Non-MLL partition is shown using orange and dark green in the second row, and the VxInsight partition is shown using red, green, and blue in the last row. This grid enables both visualization of the data (by examining the rows) and comparison of the partitions (by examining the columns).

FIG. 10 shows results of the graphic directed algorithm applied to the infant dataset. The VxInsight program constructs a mountain terrain over the clusters such that the height of each mountain represents the number of elements in the cluster under the mountain. Top left: this force-directed clustering algorithm partitions the infant data into three clusters labeled A, B, and C. Top right: VxInsight terrain map showing the distribution of the leukemia types across the clusters. ALL cases are shown in white and AML are shown in green. Bottom left: VxInsight terrain map showing the distribution of MLL cases (shown in blue) across the clusters.

FIG. 11 shows hierarchical clustering of the 126 infant leukemia samples using the “cluster-characterizing” gene sets. The rows represent genes that distinguish between the VxInsight clusters from FIG. 2 (n=150). Genes were selected by ANOVA as being the 0.1% top discriminating between each one of the clusters and the rest of the cases. Each gene is normalized across all 126 cases and the relative expression is depicted in the heat map by color, as shown in the expression scale in the bottom of the figure. The patient-to-patient distance was computed using Pearson's correlation coefficient in the Genespring program (Silicon Genetics). The columns in the dendrogram represent patients as clustered by their gene expression. The correlation between these three resultant clusters and the VxInsight clusters is higher than 90%.

FIG. 12 shows gene expression for various hematopoietic stem cell antigens in the infant leukemia data set. FIG. 12A is a gene expression “heat map” of selected HOX genes and hematopoetic stem cell antigens. The columns represent genes, while the rows represent patients organized by their VxInsight cluster membership A, B or C (see FIG. 10). The gene expression signals of 31 genes from the 26 leukemia patients were normalized relative to the median signal for each gene. The color charcaterizes the relative expresssion from the median. Red represents expression greater than the median, black is equal to the median and green is less than the median. FIG. 12B shows HOX genes median expression across the VxInsight clusters of the infant leukemia data set. The red, blue and black bars represent the median of expression of each HOX family gene across all the cases in VxInsight clusters A, B and C, respectively.

FIG. 13 shows a VxInsight patient map showing the distribution of MLL cases across the clusters derived from gene expression similarities. Top left: Magnification of the cluster A (15 ALL/5 AML cases), characterized by a “stem cell-like” gene expression pattern. Top right: cluster B, mainly ALL (51 ALL/1 AML cases). Bottom left: cluster C, mainly AML (12 ALL/42 AML cases).

FIG. 14 shows Affymetrix gene expression signal for the FMS-related tyrosine kinase 3 (FLT3) gene across the different MLL translocations. The error bar represents the standard error of the mean. Other MLL translocations include t(7;11), t(X);11) and t(11;11).

FIG. 15 shows genes that characterize the t(4;11) translocation in A vs. B, derived from the VxInsight clustering program using ANOVA. The red color represents genes that have higher expression in the t(4;11) cases in VxInsight cluster A against the t(4;11) cases in VxInsight cluster B.

FIG. 16 shows genes that characterize each one of the MLL translocations (derived from Bayesian Networks Analysis). The highlighted genes represent possible therapeutic targets.

FIG. 17 shows genes that characterize each the t(4;11) translocation and the MLL translocations, derived from Bayesian Networks Analysis, Support Vector Machines (SVM), Fuzzy logics and Discriminant Analysis.

FIG. 18 shows genes that characterize the t(4;11) translocation (left column) and the MLL translocations (right column), derived from the VxInsight clustering program using ANOVA. The red color represents genes that have higher expression in the t(4;11) cases against the rest of the cases or the MLL cases against the rest.

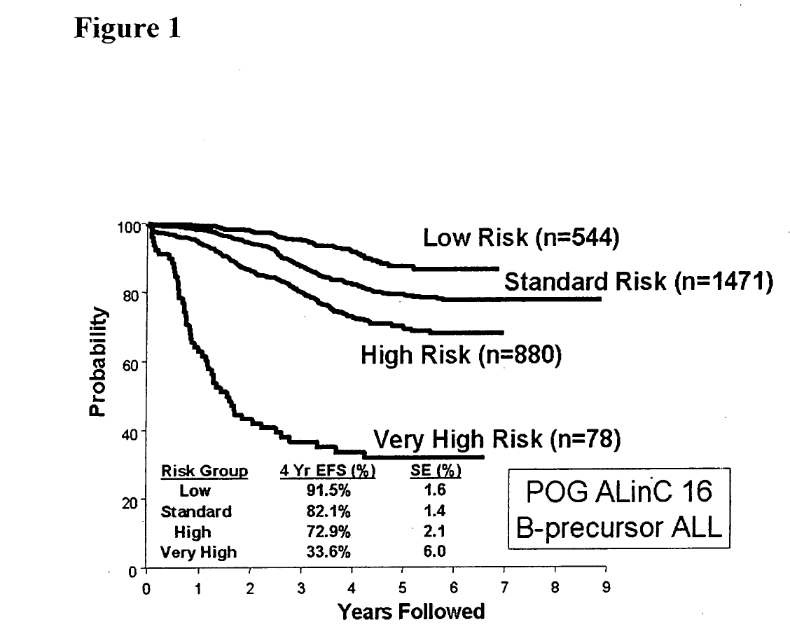

### DETAILED DESCRIPTION OF ILLUSTRATIVE EMBODIMENTS

Gene expression profiling can provide insights into disease etiology and genetic progression, and can also provide tools for more comprehensive molecular diagnosis and therapeutic targeting. The biologic clusters and associated gene profiles identified herein are useful for refined molecular classification of acute leukemias as well as improved risk assessment and classification. In addition, the invention has identified numerous genes, including but not limited to the novel gene OPAL1 (also referred to herein as “G0”), G protein β2, related sequence 1 (also referred to herein as “G1”); IL-10 Receptor alpha (also referred to herein as “G2”), FYN-binding protein and PBK1, and the genes listed in Table 42 that are, alone or in combination, strongly predictive of outcome in pediatric ALL. The genes identified herein, and the proteins they encode, can be used to refine risk classification and diagnostics, to make outcome predictions and improve prognostics, and to serve as therapeutic targets in infant leukemia and pediatric ALL.

“Gene expression” as the term is used herein refers to the production of a biological product encoded by a nucleic acid sequence, such as a gene sequence. This biological product, referred to herein as a “gene product,” may be a nucleic acid or a polypeptide. The nucleic acid is typically an RNA molecule which is produced as a transcript from the gene sequence. The RNA molecule can be any type of RNA molecule, whether either before (e.g., precursor RNA) or after (e.g., mRNA) post-transcriptional processing. cDNA prepared from the mRNA of a sample is also considered a gene product. The polypeptide gene product is a peptide or protein that is encoded by the coding region of the gene, and is produced during the process of translation of the mRNA.

The term “gene expression level” refers to a measure of a gene product(s) of the gene and typically refers to the relative or absolute amount or activity of the gene product.

The term “gene expression profile” as used herein is defined as the expression level of two or more genes. Typically a gene expression profile includes expression levels for the products of multiple genes in given sample, up to 13,000 in the experiments described herein, preferably determined using an oligonucleotide microarray.

Unless otherwise specified, “a,” “an,” “the,” and “at least one” are used interchangeably and mean one or more than one.

Diagnosis, Prognosis and Risk Classification

Current parameters used for diagnosis, prognosis and risk classification in pediatric ALL are related to clinical data, cytogenetics and response to treatment. They include age and white blood count, cytogenetics, the presence or absence of minimal residual disease (MRD), and a morphological assessment of early response (measured as slow or rapid early therapeutic response). As noted above however, these parameters are not always well correlated with outcome, nor are they precisely predictive at diagnosis.

The present invention provides an improved method for identifying and/or classifying acute leukemias. Expression levels are determined for one or more genes associated with outcome, risk assessment or classification, karyotpe (e.g., MLL translocation) or subtype (e.g., ALL vs. AML; pre-B ALL vs. T-ALL. Genes that are particularly relevant for diagnosis, prognosis and risk classification according to the invention include those described in the tables and figures herein. The gene expression levels for the gene(s) of interest in a biological sample from a patient diagnosed with or suspected of having an acute leukemia are compared to gene expression levels observed for a control sample, or with a predetermined gene expression level. Observed expression levels that are higher or lower than the expression levels observed for the gene(s) of interest in the control sample or that are higher or lower than the predetermined expression levels for the gene(s) of interest provide information about the acute leukemia that facilitates diagnosis, prognosis, and/or risk classification and can aid in treatment decisions. When the expression levels of multiple genes are assessed for a single biological sample, a gene expression profile is produced.

In one aspect, the invention provides genes and gene expression profiles that are correlated with outcome (i.e., complete continuous remission vs. therapeutic failure) in infant leukemia and/or in pediatric ALL. Assessment of one or more of these genes according to the invention can be integrated into revised risk classification schemes, therapeutic targeting and clinical trial design. In one embodiment, the expression levels of a particular gene are measured, and that measurement is used, either alone or with other parameters, to assign the patient to a particular risk category. The invention identifies several genes whose expression levels, either alone or in combination, are associated with outcome, including but not limited to OPAL1/G0, G1, G2, PBK1 (Affymetrix accession no. 39418_at, DKFZP564M182 protein; GenBank No. AJ007398); FYN-binding protein (Affymetrix accession no. 41819_at, FYB-120/130; GenBank No. AF001862; da Silva, Proc. Nat'l. Acad. Sci. USA 94(14):7493-7498 (1997)); and the genes listed in Table 42. Some of these genes (e.g., OPAL1/G0) exhibit a positive association between expression level and outcome. For these genes, expression levels above a predetermined threshold level (or higher than that exhibited by a control sample) is predictive of a positive outcome. Our data suggests that direct measurement of the expression level of OPAL1/G0, optionally in conjunction with G1 and/or G2, can be used in refining risk classification and outcome prediction in pediatric ALL. In particular, it is expected such measurements can be used to refine risk classification in children who are otherwise classified as having low risk ALL, as well as to precisely identify children with high risk ALL who could be cured with less intensive therapies.

OPAL1/G0, in particular, is a very strong predictor for outcome. Our data suggest that OPAL1/G0 (alone and/or together with G1 and/or G2) may prove to be the dominant predictor for outcome in infant leukemia or pediatric ALL, more powerful than the current risk stratification standards of age and white blood count. OPAL1/G0 tends to be expressed at lower frequencies and lower overall levels in ALL cases with cytogenetic abnormalities associated with a poorer prognosis (such as t(9;22) and t(4;11)). Indeed, regardless of risk classification, cytogenetics or biological group, roughly the same outcome statistics are seen based upon the expression level of OPAL1/G0.

We found that higher OPAL1 expression distinguished ALL cases with good (OPAL1 high: 87% long term remission) versus poor outcome (OPAL1 low: 32% long term remission) in a statistically designed, retrospective pediatric ALL case control study (detailed below). Low OPAL1 was associated with induction failure (p=0.0036) while high OPAL1 was associated with long term event free survival (p=0.02), particularly in males (p=0.0004). OPAL1 was more frequently expressed at higher levels in cases with t(12;21), normal karyotype, and hyperdiploidy (better prognosis karyotypes) compared to t(1;19) or t(9;22) (poorer prognosis karyotypes). 86% of ALL cases with t(12;21) and high OPAL1 achieved long term remission in contrast to only 35% of t(12;21) cases with low OPAL1, suggesting that OPAL1 may be useful in prospectively identifying children who might benefit from further intensification. In ALL cases classified as high risk by the NCI criteria, 87% of those that exhibited high OPAL1 levels actually achieved long term remission, compared an overall long term remission outcome of 44% in this cohort. OPAL1 was also highly predictive of a favorable outcome in T ALL (p=0.02) and a similar trend was observed in a distinct infant ALL data set (see below). Thus, high OPAL1 levels are expected to be associated with long term remissions on standard, less intensive therapies, and conversely low OPAL1 levels, even in otherwise low risk ALL patients defined by current risk classification schemes, can identify children who require therapeutic intensification for cure.

For genes such as PBK1 whose expression levels are inversely correlated with outcome, observed expression levels above a predetermined threshold level (or higher than those observed in a control sample) are useful for classifying a patient into a higher risk category due to the predicted unfavorable outcome. Expression levels for multiple genes can be measured. For example, if normalized expression levels for OPAL1/G0, G1 and G2 are all high, a favorable outcome can be predicted with greater certainty.

The expression levels of multiple (two or more) genes in one or more lists of genes associated with outcome can be measured, and those measurements are used, either alone or with other parameters, to assign the patient to a particular risk category. For example, gene expression levels of multiple genes can be measured for a patient (as by evaluating gene expression using an Affymetrix microarray chip) and compared to a list of genes whose expression levels (high or low) are associated with a positive (or negative) outcome. If the gene expression profile of the patient is similar to that of the list of genes associated with outcome, then the patient can be assigned to a low (or high, as the case may be) risk category. The correlation between gene expression profiles and class distinction can be determined using a variety of methods. Methods of defining classes and classifying samples are described, for example, in Golub et al, U.S. Patent Application Publication No. 2003/0017481 published Jan. 23, 2003, and Golub et al., U.S. Patent Application Publication No. 2003/0134300, published Jul. 17, 2003. The information provided by the present invention, alone or in conjunction with other test results, aids in sample classification and diagnosis of disease.

Computational analysis using the gene lists and other data, such as measures of statistical significance, as described herein is readily performed on a computer. The invention should therefore be understood to encompass machine readable media comprising any of the data, including gene lists, described herein. The invention further includes an apparatus that includes a computer comprising such data and an output device such as a monitor or printer for evaluating the results of computational analysis performed using such data.

In another aspect, the invention provides genes and gene expression profiles that are correlated with cytogenetics. This allows discrimination among the various karyotypes, such as MLL translocations or numerical imbalances such as hyperdiploidy or hypodiploidy, which are useful in risk assessment and outcome prediction.

In yet another aspect, the invention provides genes and gene expression profiles that are correlated with intrinsic disease biology and/or etiology. In other words, gene expression profiles that are common or shared among individual leukemia cases in different patents can be used to define intrinsically related groups (often referred to as clusters) of acute leukemia that cannot be appreciated or diagnosed using standard means such as morphology, immunophenotype, or cytogenetics. Mathematical modeling of the very sharp peak in ALL incidence seen in children 2-3 years old (>80 cases per million) has suggested that ALL may arise from two primary events, the first of which occurs in utero and the second after birth (Linet et al., Descriptive epidemiology of the leukemias, in Leukemias, 5^{th} Edition. E S Henderson et al. (eds). W B Saunders, Philadelphia. 1990). Interestingly, the detection of certain ALL-associated genetic abnormalities in cord blood samples taken at birth from children who are ultimately affected by disease supports this hypothesis (Gale et al., Proc. Natl. Acad. Sci. U.S.A., 94:13950-13954, 1997; Ford et al., Proc. Natl. Acad. Sci. U.S.A., 95:4584-4588, 1998).

Our results for both infant leukemia and pediatric ALL suggest that this disease is composed of novel intrinsic biologic clusters defined by shared gene expression profiles, and that these intrinsic subsets cannot be defined or predicted by traditional labels currently used for risk classification or by the presence or absence of specific cytogenetic abnormalities. We have identified 9 novel groups for pediatric ALL and 3 novel groups for infant leukemia using unsupervised learning methods for class discovery, and have used supervised learning methods for class prediction and outcome correlations that have identified candidate genes associated with classification and outcome. The gene expression profiles in the infant leukemia clusters provide some clues to novel and independent etiologies.

Some genes in these clusters are metabolically related, suggesting that a metabolic pathway that is associated with cancer initiation or progression. Other genes in these metabolic pathways, like the genes described herein but upstream or downstream from them in the metabolic pathway, thus can also serve as therapeutic targets.

In yet another aspect, the invention provides genes and gene expression profiles that discriminate acute myeloid leukemia (AML) from acute lymphoblastic leukemia (ALL) in infant leukemias by measuring the expression levels of a gene product correlated with ALL or AML.

Another aspect of the invention provides genes and gene expression profiles that discriminate pre-B lineage ALL from T ALL in pediatric leukemias by measuring expression levels of a gene product correlated with pre-B lineage ALL or T ALL.

It should be appreciated that while the present invention is described primarily in terms of human disease, it is useful for diagnostic and prognostic applications in other mammals as well, particularly in veterinary applications such as those related to the treatment of acute leukemia in cats, dogs, cows, pigs, horses and rabbits.

Further, the invention provides methods for computational and statistical methods for identifying genes, lists of genes and gene expression profiles associated with outcome, karyotype, disease subtype and the like as described herein.

Measurement of Gene Expression Levels

Gene expression levels are determined by measuring the amount or activity of a desired gene product (i.e., an RNA or a polypeptide encoded by the coding sequence of the gene) in a biological sample. Any biological sample can be analyzed. Preferably the biological sample is a bodily tissue or fluid, more preferably it is a bodily fluid such as blood, serum, plasma, urine, bone marrow, lymphatic fluid, and CNS or spinal fluid. Preferably, samples containing mononuclear bloods cells and/or bone marrow fluids and tissues are used. In embodiments of the method of the invention practiced in cell culture (such as methods for screening compounds to identify therapeutic agents), the biological sample can be whole or lysed cells from the cell culture or the cell supernatant.

Gene expression levels can be assayed qualitatively or quantitatively. The level of a gene product is measured or estimated in a sample either directly (e.g., by determining or estimating absolute level of the gene product) or relatively (e.g., by comparing the observed expression level to a gene expression level of another samples or set of samples). Measurements of gene expression levels may, but need not, include a normalization process.

Typically, mRNA levels (or cDNA prepared from such mRNA) are assayed to determine gene expression levels. Methods to detect gene expression levels include Northern blot analysis (e.g., Harada et al., Cell 63:303-312 (1990)), S1 nuclease mapping (e.g., Fujita et al., Cell 49:357-367 (1987)), polymerase chain reaction (PCR), reverse transcription in combination with the polymerase chain reaction (RT-PCR) (e.g., Example III; see also Makino et al., Technique 2:295-301 (1990)), and reverse transcription in combination with the ligase chain reaction (RT-LCR). Multiplexed methods that allow the measurement of expression levels for many genes simultaneously are preferred, particularly in embodiments involving methods based on gene expression profiles comprising multiple genes. In a preferred embodiment, gene expression is measured using an oligonucleotide microarray, such as a DNA microchip, as described in the examples below. DNA microchips contain oligonucleotide probes affixed to a solid substrate, and are useful for screening a large number of samples for gene expression.

Alternatively or in addition, polypeptide levels can be assayed. Immunological techniques that involve antibody binding, such as enzyme linked immunosorbent assay (ELISA) and radioimmunoassay (RIA), are typically employed. Where activity assays are available, the activity of a polypeptide of interest can be assayed directly.

The observed expression levels for the gene(s) of interest are evaluated to determine whether they provide diagnostic or prognostic information for the leukemia being analyzed. The evaluation typically involves a comparison between observed gene expression levels and either a predetermined gene expression level or threshold value, or a gene expression level that characterizes a control sample. The control sample can be a sample obtained from a normal (i.e., non-leukemic patient) or it can be a sample obtained from a patient with a known leukemia. For example, if a cytogenic classification is desired, the biological sample can be interrogated for the expression level of a gene correlated with the cytogenic abnormality, then compared with the expression level of the same gene in a patient known to have the cytogenetic abnormality (or an average expression level for the gene that characterizes that population).

Treatment of Infant Leukemia and Pediatric ALL

The genes identified herein that are associated with outcome and/or specific disease subtypes or karyotypes are likely to have a specific role in the disease condition, and hence represent novel therapeutic targets. Thus, another aspect of the invention involves treating infant leukemia and pediatric ALL patients by modulating the expression of one or more genes described herein.

In the case of OPAL1/G0, whose increased expression above threshold values is associated with a positive outcome, the treatment method of the invention involves enhancing OPAL1/G0 expression. For a number of the gene products identified herein increased expression is correlated with positive outcomes in leukemia patients. Thus, the invention includes a method for treating leukemia, such as infant leukemia and/or pediatric ALL, that involves administering to a patient a therapeutic agent that causes an increase in the amount or activity of OPAL1/G0 and/or other polypeptides of interest that have been identified herein to be positively correlated with outcome. Preferably the increase in amount or activity of the selected gene product is at least 10%, preferably 25%, most preferably 100% above the expression level observed in the patient prior to treatment.

The therapeutic agent can be a polypeptide having the biological activity of the polypeptide of interest (e.g., an OPAL1/G0 polypeptide) or a biologically active subunit or analog thereof. Alternatively, the therapeutic agent can be a ligand (e.g., a small non-peptide molecule, a peptide, a peptidomimetic compound, an antibody, or the like) that agonizes (i.e., increases) the activity of the polypeptide of interest. For example, in the case of OPAL1/G0, which is postulated to be a membrane-bound protein that may function as a receptor or signaling molecule, the invention encompasses the use of a proline-rich ligand of the WW-binding protein 1 to agonize OPAL1/G0 activity.

Gene therapies can also be used to increase the amount of a polypeptide of interest, such as OPAL1/G0 in a host cell of a patient. Polynucleotides operably encoding the polypeptide of interest can be delivered to a patient either as “naked DNA” or as part of an expression vector. The term vector includes, but is not limited to, plasmid vectors, cosmid vectors, artificial chromosome vectors, or, in some aspects of the invention, viral vectors. Examples of viral vectors include adenovirus, herpes simplex virus (HSV), alphavirus, simian virus 40, picornavirus, vaccinia virus, retrovirus, lentivirus, and adeno-associated virus. Preferably the vector is a plasmid. In some aspects of the invention, a vector is capable of replication in the cell to which it is introduced; in other aspects the vector is not capable of replication. In some preferred aspects of the present invention, the vector is unable to mediate the integration of the vector sequences into the genomic DNA of a cell. An example of a vector that can mediate the integration of the vector sequences into the genomic DNA of a cell is a retroviral vector, in which the integrase mediates integration of the retroviral vector sequences. A vector may also contain transposon sequences that facilitate integration of the coding region into the genomic DNA of a host cell.

Selection of a vector depends upon a variety of desired characteristics in the resulting construct, such as a selection marker, vector replication rate, and the like. An expression vector optionally includes expression control sequences operably linked to the coding sequence such that the coding region is expressed in the cell. The invention is not limited by the use of any particular promoter, and a wide variety is known. Promoters act as regulatory signals that bind RNA polymerase in a cell to initiate transcription of a downstream (3′ direction) operably linked coding sequence. The promoter used in the invention can be a constitutive or an inducible promoter. It can be, but need not be, heterologous with respect to the cell to which it is introduced.

Another option for increasing the expression of a gene like OPAL1/G0 wherein higher expression levels are predictive for outcome is to reduce the amount of methylation of the gene. Demethylation agents, therefore, can be used to re-activate expression of OPAL/G0 in cases where methylation of the gene is responsible for reduced gene expression in the patient.

For other genes identified herein as being correlated without outcome in infant leukemia or pediatric ALL, high expression of the gene is associated with a negative outcome rather than a positive outcome. An example of this type of gene is PBK1. These genes (and their associated gene products) accordingly represent novel therapeutic targets, and the invention provides a therapeutic method for reducing the amount and/or activity of these polypeptides of interest in a leukemia patient. Preferably the amount or activity of the selected gene product is reduced to at least 90%, more preferably at least 75%, most preferably at least 25% of the gene expression level observed in the patient prior to treatment A cell manufactures proteins by first transcribing the DNA of a gene for that protein to produce RNA (transcription). In eukaryotes, this transcript is an unprocessed RNA called precursor RNA that is subsequently processed (e.g. by the removal of introns, splicing, and the like) into messenger RNA (mRNA) and finally translated by ribosomes into the desired protein. This process may be interfered with or inhibited at any point, for example, during transcription, during RNA processing, or during translation. Reduced expression of the gene(s) leads to a decrease or reduction in the activity of the gene product.

The therapeutic method for inhibiting the activity of a gene whose expression is correlated with negative outcome involves the administration of a therapeutic agent to the patient. The therapeutic agent can be a nucleic acid, such as an antisense RNA or DNA, or a catalytic nucleic acid such as a ribozyme, that reduces activity of the gene product of interest by directly binding to a portion of the gene encoding the enzyme (for example, at the coding region, at a regulatory element, or the like) or an RNA transcript of the gene (for example, a precursor RNA or mRNA, at the coding region or at 5′ or 3′ untranslated regions) (see, e.g., Golub et al., U.S. Patent Application Publication No. 2003/0134300, published Jul. 17, 2003). Alternatively, the nucleic acid therapeutic agent can encode a transcript that binds to an endogenous RNA or DNA; or encode an inhibitor of the activity of the polypeptide of interest. It is sufficient that the introduction of the nucleic acid into the cell of the patient is or can be accompanied by a reduction in the amount and/or the activity of the polypeptide of interest. An RNA aptamer can also be used to inhibit gene expression. The therapeutic agent may also be protein inhibitor or antagonist, such as small non-peptide molecule such as a drug or a prodrug, a peptide, a peptidomimetic compound, an antibody, a protein or fusion protein, or the like that acts directly on the polypeptide of interest to reduce its activity.

The invention includes a pharmaceutical composition that includes an effective amount of a therapeutic agent as described herein as well as a pharmaceutically acceptable carrier. Therapeutic agents can be administered in any convenient manner including parenteral, subcutaneous, intravenous, intramuscular, intraperitoneal, intranasal, inhalation, transdermal, oral or buccal routes. The dosage administered will be dependent upon the nature of the agent; the age, health, and weight of the recipient; the kind of concurrent treatment, if any; frequency of treatment; and the effect desired. A therapeutic agent identified herein can be administered in combination with any other therapeutic agent(s) such as immunosuppressives, cytotoxic factors and/or cytokine to augment therapy, see Golub et al, Golub et al., U.S. Patent Application Publication No. 2003/0134300, published Jul. 17, 2003, for examples of suitable pharmaceutical formulations and methods, suitable dosages, treatment combinations and representative delivery vehicles.

The effect of a treatment regimen on an acute leukemia patient can be assessed by evaluating, before, during and/or after the treatment, the expression level of one or more genes as described herein. Preferably, the expression level of gene(s) associated with outcome, such as OPAL1/G0, G1 and/or G2 are monitored over the course of the treatment period. Optionally gene expression profiles showing the expression levels of multiple selected genes associated with outcome can be produced at different times during the course of treatment and compared to each other and/or to an expression profile correlated with outcome.

Screening for Therapeutic Agents

The invention further provides methods for screening to identify agents that modulate expression levels of the genes identified herein that are correlated with outcome, risk assessment or classification, cytogenetics or the like. Candidate compounds can be identified by screening chemical libraries according to methods well known to the art of drug discovery and development (see Golub et al., U.S. Patent Application Publication No. 2003/0134300, published Jul. 17, 2003, for a detailed description of a wide variety of screening methods). The screening method of the invention is preferably carried out in cell culture, for example using leukemic cell lines that express known levels of the therapeutic target, such as OPAL1/G0. The cells are contacted with the candidate compound and changes in gene expression of one or more genes relative to a control culture are measured. Alternatively, gene expression levels before and after contact with the candidate compound can be measured. Changes in gene expression indicate that the compound may have therapeutic utility. Structural libraries can be surveyed computationally after identification of a lead drug to achieve rational drug design of even more effective compounds.

The invention further relates to compounds thus identified according to the screening methods of the invention. Such compounds can be used to treat infant leukemia and/or pediatric ALL, as appropriate, and can be formulated for therapeutic use as described above.

OPAL1 Polynucleotide, Polypeptide and Antibody

The invention includes novel nucleotide sequences found to be strongly associated with outcome in pediatric ALL, as well as the novel polypeptides they encode. These sequences, which we originally called “G0” but now have named OPAL1 for Outcome Predictor in Acute Leukemia, appear to be associated with alternatively spliced products of a large and complex gene. Alternate 5′ exon usage likely causes the production of more than one distinct protein from the genomic sequence. We have now fully cloned both the genomic and cDNA sequences (SEQ ID NO:16) of OPAL1. Expression levels of OPAL1/G0 that are high in relation to a predetermined threshold or a control sample are indicative of good prognosis.

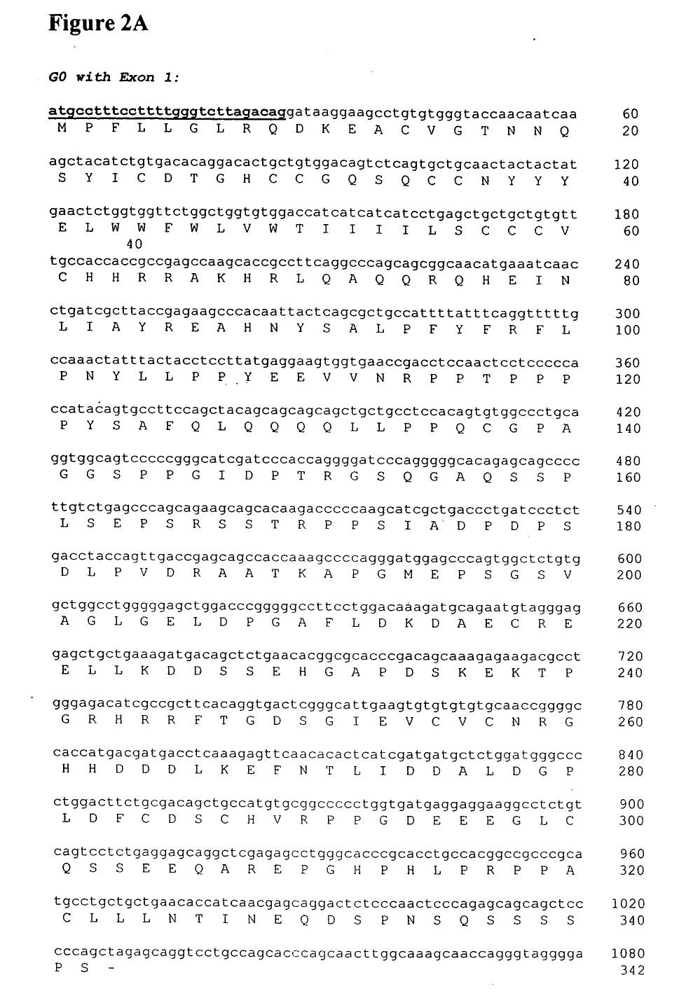

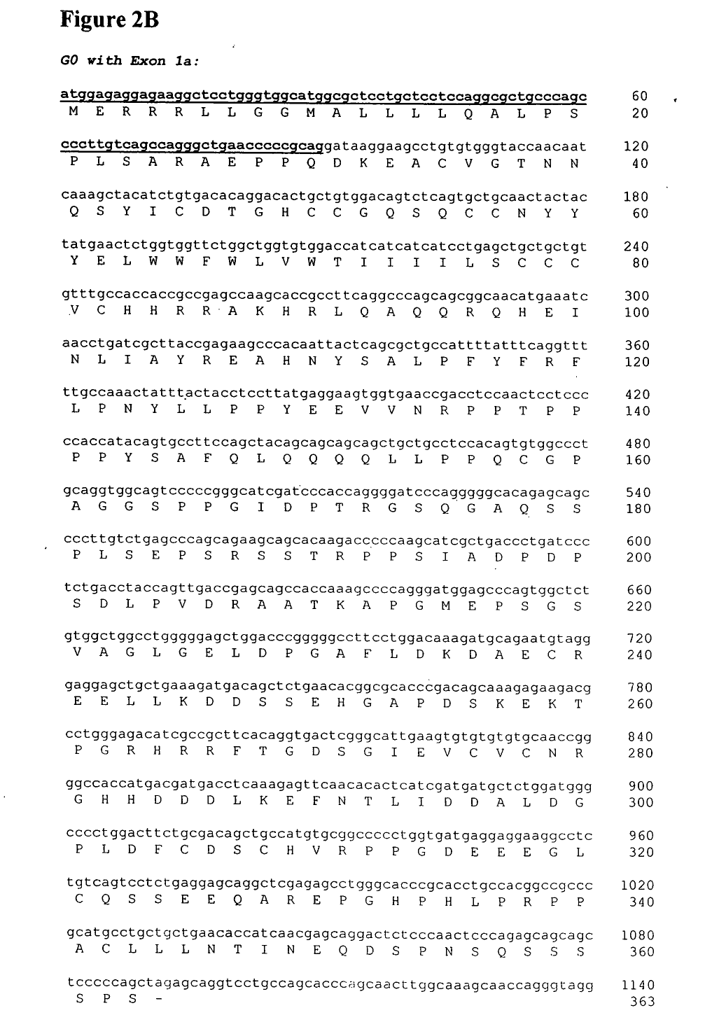

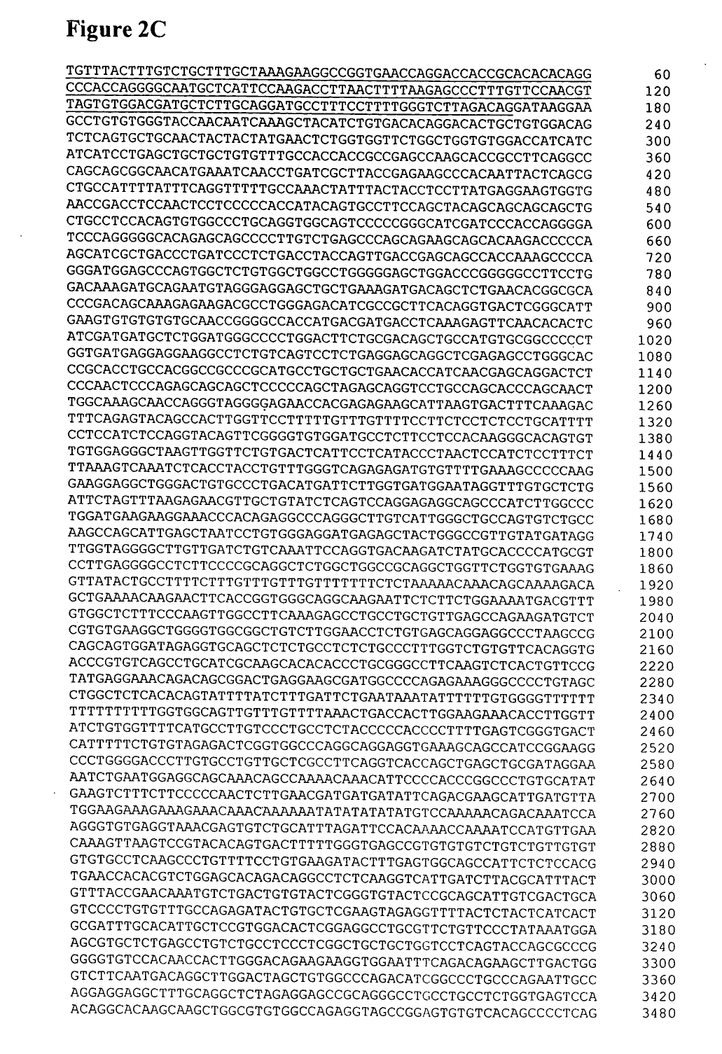

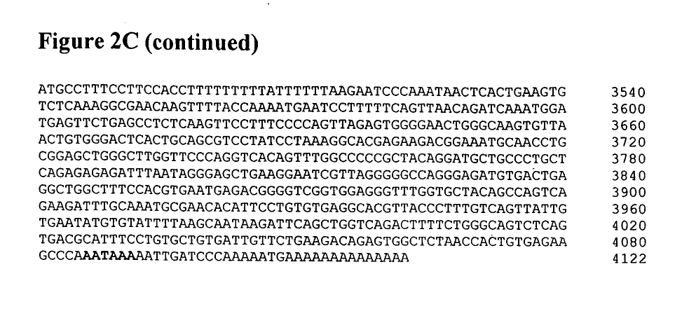

Nucleotide sequences (SEQ ID NOs:1 and 3) encoding two alternatively spliced forms of the polypeptide gene product, OPAL1/G0, are shown in FIG. 2. The putative amino acid sequences (SEQ ID NOs:2 and 4) of the two forms of protein OPAL1/G0 are also shown in FIG. 2. Analysis of the protein sequence suggests that OPAL1/G0 may be a transmembrane protein with a short (53 amino acid) extracellular domain and an intracellular domain. Both the short extracellular and longer intracellular domains have proline-rich regions that are homologous to proteins that bind WW domains such as the WBP-1 Domain-Binding Protein 1 located at human chromosome 2p12 (MIM #60691; WBP1 in HUGO; UniGene Hs. 7709). Like SH3 domans in proteins, WW domains interact with proline-rich transcription factors and cytoplasmic signaling molecules (such as OPAL1/G0) to mediate protein-protein interactions regulating gene expression and cell signaling. The data suggest that this novel coding sequence encodes a signaling protein having a WW-binding domain and it likely plays an important role in regulation of these cellular processes.

The present invention also includes polypeptides with an amino acid sequence having at least about 80% amino acid identity, at least about 90% amino acid identity, or about 95% amino acid identity with SEQ ID NO:2 or 4. Amino acid identity is defined in the context of a comparison between an amino acid sequence and SEQ ID NO:2 or 4, and is determined by aligning the residues of the two amino acid sequences (i.e., a candidate amino acid sequence and the amino acid sequence of SEQ ID NO:2 or 4) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence is the amino acid sequence being compared to an amino acid sequence present in SEQ ID NO:2 or 4. A candidate amino acid sequence can be isolated from a natural source, or can be produced using recombinant techniques, or chemically or enzymatically synthesized. Preferably, two amino acid sequences are compared using the Blastp program of the BLAST 2 search algorithm, as described by Tatusova et al. (FEMS Microbiol. Lett., 174:247-250, 1999, and available on the world wide web at ncbi.nlm.nih.gov/gorf/b12.html). Preferably, the default values for all BLAST 2 search parameters are used, including matrix=BLOSUM62; open gap penalty=11, extension gap penalty=1, gap×dropoff=50, expect=10, wordsize=3, and optionally, filter on. In the comparison of two amino acid sequences using the BLAST2 search algorithm, amino acid identity is referred to as “identities.” A polypeptide of the present invention that has at least about 80% identity with SEQ ID NO:2 or 4 also has the biological activity of OPAL1/G0.

The polypeptides of this aspect of the invention also include an active analog of SEQ ID NO:2 or 4. Active analogs of SEQ ID NO:2 or 4 include polypeptides having amino acid substitutions that do not eliminate the ability to perform the same biological function(s) as OPAL1/G0. Substitutes for an amino acid may be selected from other members of the class to which the amino acid belongs. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, aspartate, and glutamate. The positively charged (basic) amino acids include arginine, lysine, and histidine. The negatively charged (acidic) amino acids include aspartic acid and glutamic acid. Such substitutions are known to the art as conservative substitutions. Specific examples of conservative substitutions include Lys for Arg and vice versa to maintain a positive charge; Glu for Asp and vice versa to maintain a negative charge; Ser for Thr so that a free —OH is maintained; and Gln for Asn to maintain a free NH₂.

Active analogs, as that term is used herein, include modified polypeptides. Modifications of polypeptides of the invention include chemical and/or enzymatic derivatizations at one or more constituent amino acids, including side chain modifications, backbone modifications, and N- and C-terminal modifications including acetylation, hydroxylation, methylation, amidation, and the attachment of carbohydrate or lipid moieties, cofactors, and the like.

The present invention further includes polynucleotides encoding the amino acid sequence of SEQ ID NO:2 or 4. An example of the class of nucleotide sequences encoding the polypeptide having SEQ ID NO:2 is SEQ ID NO:1; and an example of the class of nucleotide sequences encoding the polypeptide having SEQ ID NO:4 is SEQ ID NO:3. The other nucleotide sequences encoding the polypeptides having SEQ ID NO:2 or 4 can be easily determined by taking advantage of the degeneracy of the three letter codons used to specify a particular amino acid. The degeneracy of the genetic code is well known to the art and is therefore considered to be part of this disclosure. The classes of nucleotide sequences that encode SEQ ID NO:2 and 4 are large but finite, and the nucleotide sequence of each member of the classes can be readily determined by one skilled in the art by reference to the standard genetic code.

The present invention also includes polynucleotides with a nucleotide sequence having at least about 90% nucleotide identity, at least about 95% nucleotide identity, or about 98% nucleotide identity with SEQ ID NO:1 or 3. Nucleotide identity is defined in the context of a comparison between an nucleotide sequence and SEQ ID NO:1 or 3, and is determined by aligning the residues of the two nucleotide sequences (i.e., a candidate nucleotide sequence and the nucleotide sequence of SEQ ID NO:1 or 3) to optimize the number of identical nucleotides along the lengths of their sequences; gaps in either or both sequences are permitted in making the alignment in order to optimize the number of identical nucleotides, although the nucleotides in each sequence must nonetheless remain in their proper order. A candidate nucleotide sequence is the nucleotide sequence being compared to an nucleotide sequence present in SEQ ID NO:2 or 4. A candidate nucleotide sequence can be isolated from a natural source, or can be produced using recombinant techniques, or chemically or enzymatically synthesized. Percent identity is determined by aligning two polynucleotides to optimize the number of identical nucleotides along the lengths of their sequences; gaps in either or both sequences are permitted in making the alignment in order to optimize the number of shared nucleotides, although the nucleotides in each sequence must nonetheless remain in their proper order. For example, the two nucleotide sequences are readily compared using the Blastn program of the BLAST 2 search algorithm, as described by Tatusova et al. (FEMS Microbiol. Lett., 174:247-250, 1999). Preferably, the default values for all BLAST 2 search parameters are used, including reward for match=1, penalty for mismatch=−2, open gap penalty=5, extension gap penalty=2, gap x_dropoff=50, expect=10, wordsize=11, and filter on.

Examples of polynucleotides encoding a polypeptide of the present invention also include those having a complement that hybridizes to the nucleotide sequence SEQ ID NO:1 or 3 under defined conditions. The term “complement” refers to the ability of two single stranded polynucleotides to base pair with each other, where an adenine on one polynucleotide will base pair to a thymine on a second polynucleotide and a cytosine on one polynucleotide will base pair to a guanine on a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary. As used herein, “hybridizes,” “hybridizing,” and “hybridization” means that a single stranded polynucleotide forms a noncovalent interaction with a complementary polynucleotide under certain conditions. Typically, one of the polynucleotides is immobilized on a membrane. Hybridization is carried out under conditions of stringency that regulate the degree of similarity required for a detectable probe to bind its target nucleic acid sequence. Preferably, at least about 20 nucleotides of the complement hybridize with SEQ ID NO:1 or 3, more preferably at least about 50 nucleotides, most preferably at least about 100 nucleotides.

Also provided by the invention is an OPAL1/G0 antibody, or antigen-binding portion thereof, that binds the novel protein OPAL1/G0. OPAL1/G0 antibodies can be used to detect OPAL1/G0 protein; they are also useful therapeutically to modulate expression of the OPAL1/G0 gene. An antibody may be polyclonal or monoclonal. Methods for making polyclonal and monoclonal antibodies are well known to the art. Monoclonal antibodies can be prepared, for example, using hybridoma techniques, recombinant, and phage display technologies, or a combination thereof. See Golub et al., U.S. Patent Application Publication No. 2003/0134300, published Jul. 17, 2003, for a detailed description of the preparation and use of antibodies as diagnostics and therapeutics.

Preferably the antibody is a human or humanized antibody, especially if it is to be used for therapeutic purposes. A human antibody is an antibody having the amino acid sequence of a human immunoglobulin and include antibodies produced by human B cells, or isolated from human sera, human immunoglobulin libraries or from animals transgenic for one or more human immunoglobulins and that do not express endogenous immunoglobulins, as described in U.S. Pat. No. 5,939,598 by Kucherlapati et al., for example. Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. U.S.A., 90:2551-2555 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

Antibodies generated in non-human species can be “humanized” for administration in humans in order to reduce their antigenicity. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Residues from a complementary determining region (CDR) of a human recipient antibody are replaced by residues from a CDR of a non-human species (donor antibody) such as mouse, rat or rabbit having the desired specificity. Optionally, Fv framework residues of the human immunoglobulin are replaced by corresponding non-human residues. See Jones et al., Nature, 321:522-525 (1986); Riechmann et al., Nature, 332:323-327 (1988); and Presta, Curr. Op. Struct. Biol., 2:593-596 (1992). Methods for humanizing non-human antibodies are well known in the art. See Jones et al., Nature, 321:522-525 (1986); Riechmann et al., Nature, 332:323-327 (1988); Verhoeyen et al., Science, 239:1534-1536 (1988); and (U.S. Pat. No. 4,816,567).

Laboratory Applications

The present invention further includes a microchip for use in clinical settings for detecting gene expression levels of one or more genes described herein as being associated with outcome, risk classification, cytogenics or subtype in infant leukemia and pediatric ALL. In a preferred embodiment, the microchip contains DNA probes specific for the target gene(s). Also provided by the invention is a kit that includes means for measuring expression levels for the polypeptide product(s) of one or more such genes, preferably OPAL/G0, G1, G2, FYN binding protein, PBK1, or any of the genes listed in Table 42. In a preferred embodiment, the kit is an immunoreagent kit and contains one or more antibodies specific for the polypeptide(s) of interest.

### EXAMPLES

The present invention is illustrated by the following examples. It is to be understood that the particular examples, materials, amounts, and procedures are to be interpreted broadly in accordance with the scope and spirit of the invention as set forth herein

### Example IA

### Laboratory Methods and Cohort Design

Leukemia Blast Purification, RNA Isolation, Amplification and Hybridization to Oligonucleotide Arrays

Laboratory techniques were developed to optimize sample handling and processing for high quality microarray studies for gene expression profiling in leukemia samples. Reproducible methods were developed for leukemia blast purification, RNA isolation, linear amplification, and hybridization to oligonucleotide arrays. Our optimized approach is a modification of a double amplification method originally developed by Ihor Lemischka and colleagues from Princeton University (Ivanova et al., Science 298(5593):601-604 (2002)).

Total RNA was isolated from leukemic blasts using Qiagen Rneasy. An average of 2×10⁷ cells were used for total RNA extraction with the Qiagen RNeasy mini kit (Valencia, Calif.). The yield and integrity of the purified total RNA were assessed with the RiboGreen assay (Molecular Probes, Eugene, Oreg.) and the RNA 6000 Nano Chip (Agilent Technologies, Palo Alto, Calif.), respectively.

Complementary RNA (cRNA) target was prepared from 2.5 μg total RNA using two rounds of Reverse Transcription (RT) and In Vitro Transcription (IVT). Following denaturation for 5 minutes at 70° C., the total RNA was mixed with 100 pmol T7-(dT)₂₄ oligonucleotide primer (Genset Oligos, La Jolla, Calif.) and allowed to anneal at 42° C. The mRNA was reverse transcribed with 200 units Superscript II (Invitrogen, Grand Island, N.Y.) for 1 hour at 42° C. After RT, 0.2 volume 5× second strand buffer, additional dNTP, 40 units DNA polymerase I, 10 units DNA ligase, 2 units RnaseH (Invitrogen) were added and second strand cDNA synthesis was performed for 2 hours at 16° C. After T4 DNA polymerase (10 units), the mix was incubated an additional 10 minutes at 16° C. An equal volume of phenol:chloroform:isoamyl alcohol (25:24:1)(Sigma, St. Louis, Mo.) was used for enzyme removal. The aqueous phase was transferred to a microconcentrator (Microcon 50. Millipore, Bedford, Mass.) and washed/concentrated with 0.5 ml DEPC water twice the sample was concentrated to 10-20 ul. The cDNA was then transcribed with T7 RNA polymerase (Megascript, Ambion, Austin, Tex.) for 4 hr at 37° C. Following IVT, the sample was phenol:chloroform:isoamyl alcohol extracted, washed and concentrated to 10-20 ul.

The first round product was used for a second round of amplification which utilized random hexamer and T7-(dT)₂₄ oligonucleotide primers, Superscript II, two RNase H additions, DNA polymerase I plus T4 DNA polymerase finally and a biotin-labeling high yield T7 RNA polymerase kit (Enzo Diagnostics, Farmingdale, N.Y.). The biotin-labeled cRNA was purified on Qiagen RNeasy mini kit columns, eluted with 50 ul of 45° C. RNase-free water and quantified using the RiboGreen assay.

Following RNA isolation and cRNA amplification using two rounds of poly dT primer-anchored Reverse Transcription and T7 RNA polymerase transcription, RNA and cRNA quality was assessed by capillary electrophoresis on Agilent RNA Lab-Chips. After the quality check on Agilent Nano 900 Chips, 15 ug cRNA were fragmented following the Affymetrix protocol (Affymetrix, Santa Clara, Calif.). The fragmented RNA was then hybridized for 20 hours at 45° C. to HG_U95Av2 probes. The hybridized probe arrays were washed and stained with the EukGE_WS2 fluidics protocol (Affymetrix), including streptavidin phycoerythrin conjugate (SAPE, Molecular Probes, Eugene, Oreg.) and an antibody amplification step (Anti-streptavidin, biotinylated, Vector Labs, Burlingame, Calif.). HG_U95Av2 chips were scanned at 488 nm, as recommended by Affymetrix. The expression value of each gene was calculated using Affymetrix Microarray Suite 5.0 software.

We routinely obtain 100-200 micrograms of amplified cRNA from 2.5 micrograms of leukemia cell-derived total RNA. Our detailed statistical analysis comparing various RNA inputs and single vs. double amplification methods have shown that this approach leads to an excellent representation of low as well as high abundance mRNAs and is highly reproducible. It has the added benefit of not losing the representation of low abundance genes frequently lost in methods that lack amplification or only perform single round amplifications. As only 15 micrograms of cRNA are required per Affymetrix chip, we are able to store residual cRNA in virtually all cases; this highly valuable cRNA can be used again in the future as array platforms and methods of analysis improve. Samples were studied using oligonucleotide microarrays containing 12,625 probes (Affymetrix U95Av2 array platform).

Statistical Design

We designed two retrospective cohorts of pediatric ALL patients registered to clinical trials previously coordinated by the Pediatric Oncology Group (POG): 1) a cohort 127 infant leukemias (the “infant” data set); and 2) a case control study of 254 pediatric B-precursor and T cell ALL cases (the “preB” dataset). These samples were obtained from patients with long term follow up who were registered to clinical trials completed by the Pediatric Oncology Group (POG). In the analysis of gene expression profiles for classification and particularly outcome prediction, it is essential to integrate gene expression data with laboratory parameters that impact the quality of the primary data, and to make sure that any derived cluster or gene list cannot be accounted for by variations in laboratory methodology. Thus we tracked and annotated our gene expression data set with all of the laboratory correlates shown below.

Laboratory Correlates

Vial Date=Sample Collection Date Value
Percent Leukemic Blasts in Sample=Integer
Sample Viability=Integer
RNA Method=Boolean
RNA Quality=Boolean
RNA Starting Amount=Amount Amplified (Floating Point)
Experimental Set=16/Arrays per Set (Integer)
Amplification Date=Date Value (Linked to Reagent Lot)
aRNA Quality=Quality of Amplified RNA
   Clinical, demographic, and outcome data are also essential for predictive profiling.
   Clinical/Patient Sample Correlates
COG_NO=Patient Identifier (Integer)
Study_NO=Treatment Study (Integer)
AGE_DAYS=Age at Initial Registration (Integer)
RAC=Patient Race (Strings)
SX=Patient Sex (String)
WBC_BLD=Presenting Blood Count (Floating Point)
DUR_CR=Duration of Complete Remission (Days)
REMISS=(CCR=Continuous Complete Remission)
FAIL=Failed Therapy; String but representing a Boolean)
ACH-CR=Achieved Initial CR (String, but Boolean)
DI=DNA Index (Leukemia Cell DNA Amount, Floating)
KARYOTYP=Cytogenetic Abnormality
   Blinded cohort studies were developed for the conduct of the array experiments. In this way, the individuals performing arrays were blinded to all clinical and outcome correlative variables.

For the retropective “infant” study, 142 retrospective cases from two POG infant trials (9407 for infant ALL; 9421 for infant AML) were initially chosen for analysis. Infants as defined were <365 days in age and had overall extremely poor survival rates (<25%). Of the 142 cases, 127 were ultimately retained in the study; 15 cases were excluded from the final analysis due to poor quality total RNA, cRNA amplification, or hybridization. Of the final 127 cases analyzed, 79 were considered traditional ALL by morphology and immunophenotyping and 48 were considered AML. 59/127 of these cases had rearrangements of the MLL gene.

The 254 member retrospective pre-B and T cell ALL case control study (the “preB” study) was selected from a number of pediatric POG clinical trials. A cohort design was developed that could compare and contrast gene expression profiles in distinct cytogenetic subgroups of ALL patients who either did or did not achieve a long term remission (for example comparing children with t(4;11) who failed vs. those who achieved long term remission). Such a design allowed us to compare and contrast the gene expression profiles associated with different outcomes within each genetic group and to compare profiles between different cytogenetic abnormalities. The design was constructed to look at a number of small independent case-control studies within B precursor ALL and T cell ALL. For the B cell ALL group, the representative recurrent translocations included t(4;11), t(9;22), t(1;19), monosomy 7, monosomy 21, Females, Males, African American, Hispanic, and AlinC15 arm A. Cases were selected from several completed POG trials, but the majority of cases came from the POG 9000 series, including 8602, 9406, 9005, and 9006 as long term follow up was available.

As standard cytogenetic analysis of the samples from patients registered to these older trials would not have usually detected the t(12;21), we performed RT-PCR studies on a large cohort of these cases to select ALL cases with t(12;21) who either failed (n=8) therapy or achieved long term remissions (n=22). Cases who “failed” had failed within 4 years while “controls” had achieved a complete continuous remission of 4 or more years. A case-control study of induction failures (cases) vs. complete remissions (CRs; controls) was also included in this cohort design as was a T cell cohort.

It is very important to recognize that the study was designed for efficiency, and maximum overlap, without adversely affecting the random sampling assumptions for the individual case-control studies. To design this cohort, the set of all patients (irrespective of study) who had inventory in the UNM POG/COG Tissue Repository and who had failed within 4 years of diagnosis (cases) were considered. Each such case was assigned a random number from zero to one. Cases were then sorted by this random number. The same process was applied to the totality of potential controls. For each case-control study, we then took the first N patients (requested in design) or all patients (whichever was smaller), meeting the entry requirements for the particular study. By maximizing the overlap in this fashion, a savings of over 20% compared to a design that required mutually exclusive entries was achieved. Yet for any given case-control study, the patients represent pure random samples of cases and controls. (For example if the first patient in the sort of the failure group were an African-American female with a t(1;19) translocation, she would participate in at least three case control studies). As for the infant leukemia cases, gene expression arrays were completed using 2.5 micrograms of RNA per case (all samples had >90% blasts) with double linear amplification. All amplified RNAs were hybridized to Affymetrix U95A.v2 chips.

### Example IB

### Computational Methods

The present invention makes use of a suite of high-end analytic tools for the analysis of gene expression data. Many of these represent novel implementations or significant extensions of advanced techniques from statistical and machine learning theory, or new data mining approaches for dealing with high-dimensional and sparse datasets. The approaches can be categorized into two major groups: knowledge discovery environments, and supervised classification methodologies.

Clustering, Visualization, and Text-Mining

1. VxInsight

VxInsight is a data mining tool (Davidson et al., J. Intellig. Inform. Sys. 11:259-285, 1998; Davidson et al., IEEE Information Visualization 2001, 23-30, 2001) originally developed to cluster and organize bibliographic databases, which has been extended and customized for the clustering and visualization of genomic data. It presents an intuitive way to cluster and view gene expression data collected from microarray experiments (Kim et al., Science 293:2087-92, 2001). It can be applied equally to the clustering of genes (e.g., in a time-series experiment) or to discover novel biologic clusters within a cohort of leukemia patient samples. Similar genes or patients are clustered together spatially and represented with a 3D terrain map, where the large mountains represent large clusters of similar genes/samples and smaller hills represent clusters with fewer genes/samples. The terrain metaphor is extremely intuitive, and allows the user to memorize the “landscape,” facilitating navigation through large datasets.

VxInsight's clustering engine, or ordination program, is based on a force-directed graph placement algorithm that utilizes all of the similarities between objects in the dataset. When applied to gene clustering, for example, the algorithm assigns genes into clusters such that the sum of two opposing forces is minimized. One of these forces is repulsive and pushes pairs of genes away from each other as a function of the density of genes in the local area. The other force pulls pairs of similar genes together based on their degree of similarity. The clustering algorithm terminates when these forces are in equilibrium. User-selected parameters determine the fineness of the clustering, and there is a tradeoff with respect to confidence in the reliability of the cluster versus further refinement into sub-clusters that may suggest biologically important hypotheses.

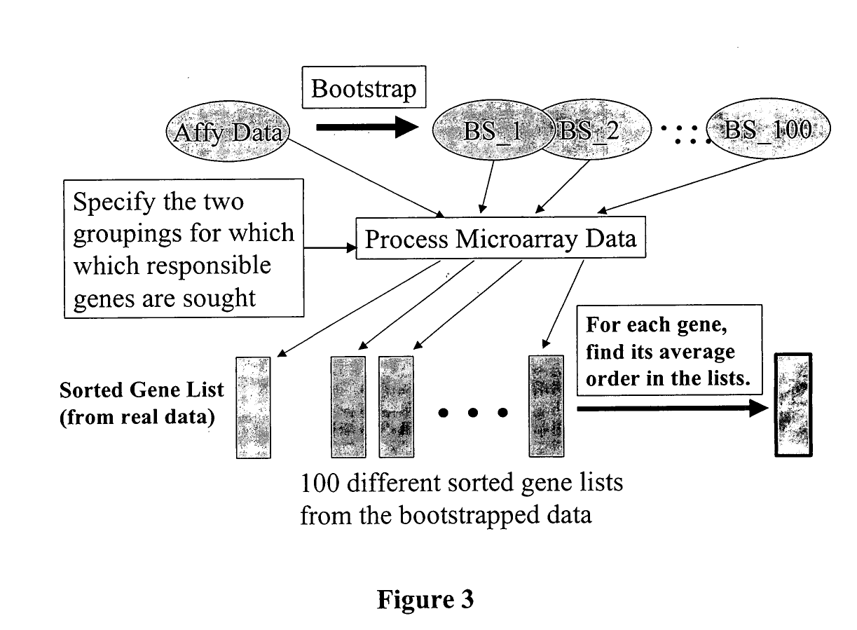

VxInsight was employed to identify clusters of infant leukemia patients with similar gene expression patterns, and to identify which genes strongly contributed to the separations. A suite of statistical analysis tools was developed for post-processing information gleaned from the VxInsight discovery process. Visual and clustering analyses generated gene lists, which when combined with public databases and research experience, suggest possible biological significance for those clusters. The array expression data were clustered by rows (similar genes clustered together), and by columns (patients with similar gene expression clustered together). In both cases Pearson's R was used to estimate the similarities. Analysis of variance (ANOVA) was used to determine which genes had the strongest differences between pairs of patient clusters. These gene lists were sorted into decreasing order based on the resulting F-scores, and were presented in an HTML format with links to the associated OMIM pages (Online Mendelian Inheritance in Man database, available on the world wide web through the National Center for Biotechnology Information), which were manually examined to hypothesize biological differences between the clusters. Gene list stability was investigated using statistical bootstraps (Efron, Ann. Statist. 7:1-26, 1979; Hjorth et al., Computer Intensive Statistical Methods, Validation Model Selection and Bootstrap. Chapman & Hall, London, 1994). For each pair of clusters 100 random bootstrap cases were constructed via resampling with replacement from the observed expressions (FIG. 3). Next, the resulting ordered lists of genes were determined, using the same ANOVA method as before. The average order in the set of bootstrapped gene lists was computed for all genes, and reported as an indication of rank order stability (the percentile from the bootstraps estimates a p-value for observing a gene at or above the list order observed using the original experimental values).

2. Principal Component Analysis

Principal component analysis (PCA) is a well-known and convenient method for performing unsupervised clustering of high-dimensional data. Closely related to the Singular Value Decomposition (SVD), PCA is an unsupervised data analysis technique whereby the most variance is captured in the least number of coordinates. It can serve to reduce the dimensionality of the data while also providing significant noise reduction. It is a standard technique in data analysis and has been widely applied to microarray data. Recently (Raychaudhuri et al., Pac. Symp. Biocomput., 5:455-466, 2002) PCA was used to analyze cell cycles in yeast (Chu et al., Science, 282:699-705, 1998; Spellman et al., Mol. Biol. Cell, 9:3273-97, 1998); PCA has also been applied to clustering (Hastie et al., Genome Biology 1:research0003, 2000; Holter et al., Proc. Natl. Acad. Sci., 97:8409-14, 2000); other applications of PCA to microarray data have been suggested (Wall et al., Bioinformatics 17, 566-568, 2001).

PCA works by providing a statistically significant projection of a dataset onto an orthonormal basis. This basis is computed so that a variety of quantities are optimized. In particular we have (Kirby, Geometric Data Analysis. John Wiley & Sons, New York, 2001):
maximization of the statistical variance,
minimization of mean square truncation error,
maximization of the mean squared projection,
minimization of entropy.
   Furthermore, the PCA basis optimizes these quantities by dimension. In other words, the first PCA basis vector provides the best one-dimensional projection of the data subject to the above conditions, the first and second PCA basis vectors provide the best two-dimensional projection, et cetera. The PCA basis is typically computed by solving an eigenvalue problem closely related to the SVD (Kirby, Geometric Data Analysis. John Wiley & Sons, New York, 2001; Trefethen et al., Numerical Linear Algebra. SIAM, Philadelphia, 1997). Consequently, the PCA basis vectors are often called eigenvectors; in the context of microarray data they are occasionally called eigen-genes, eigen-arrays, or eigen-patients. PCA is typically illustrated by finding the major and minor axes in a cloud of data filling an ellipse. The first eigenvector corresponds to the major axis of the ellipse while the second eigenvector corresponds to the minor axis. PCA is used to analyze the principal sources of error in microarray experiments, and to perform variance analysis of VxInsight-derived clusters.
   Supervised Learning Methods and Feature Selection for Class Prediction
   1. Bayesian Networks

The Bayesian network modeling and learning paradigm (Pearl, Probabilistic Reasoning for Intelligent Systems. Morgan Kaufmann, San Francisco, 1988; Heckerman et al., Machine Learning 20:197-243, 1995) has been studied extensively in the statistical machine learning literature. A Bayesian net is a graph-based model for representing probabilistic relationships between random variables. The random variables, which may, for example, represent gene expression levels, are modeled as graph nodes; probabilistic relationships are captured by directed edges between the nodes and conditional probability distributions associated with the nodes. In the context of genomic analysis, this framework is particularly attractive because it allows hypotheses of actor interactions (e.g., gene-gene, gene-protein, gene-polymorphism) to be generated and evaluated in a mathematically sound manner against existing evidence. Network reconstruction, pathway identification, diagnosis, and outcome prediction are among the many challenges of current interest that Bayesian networks can address. Introduction of new-network nodes (random variables) can model effects of previously hidden state variables, conditioning prediction on such factors as subject characteristics, disease subtype, polymorphic information, and treatment variables.

A Bayesian net asserts that each node (representing a gene or an outcome) is statistically independent of all its non-descendants, once the values of its parents (immediate ancestors) in the graph are known. Even with the focus on restricted subnetworks, the learning problem is enormously difficult, due to the large number of genes, the fact that the expression values of the genes are continuous, and the fact that expression data generally is rather noisy. Our approach to Bayesian network learning employs an initial gene selection algorithm to produce 20-30 genes, with a binary binning of each selected gene's expression value. The set of selected genes then is searched exhaustively for parent sets of size 5 or less, with the induced candidate networks being evaluated by the BD scoring metric (Heckerman et al., Machine Learning 20:197-243, 1995). This metric, along with our variance factor, is used to blend the predictions made by the 500 best scoring networks. Each of these 500 Bayesian networks can be viewed as a competing hypothesis for explaining the current evidence (i.e., training data and prior knowledge) for the corresponding classification task, and the gene interactions each suggests are potentially of independent interest as well.

Bayesian analysis allows the combining of disparate evidence in a principled way. Abstractly, the analysis synthesizes known or believed prior domain information with bodies of possibly diverse observational and experimental data (e.g., microarrays giving gene expression levels, polymorphism information, clinical data) to produce probabilistic hypotheses of interaction and prediction. Prior elicitation and representation quantifies the strength of beliefs in domain information, allowing this knowledge and observational and experimental data to be handled in uniform manner. Strong priors are akin to plentiful and reliable data; weaker priors are akin to sparse, noisy data. Similarly, observational and experimental data can be qualified by its reliability, accuracy, and variability, taking into account the different sources that produced the data and inherent differences in the natures of the data. Of course, observational and experimental data will eventually dominate the analysis if it is of sufficient size and quality.

In the context of outcome and disease subtype prediction, we applied a highly customized and extended Bayesian net methodology to high-dimensional sparse data sets with feature interaction characteristics such as those found in the genomics application. These customizations included the parent-set model for Bayesian net classifiers, the blending of competing parent sets into a single classifier, the pre-filtering of genes for information content, Helman-Veroff normalization to pre-process the data, methods for discretizing continuous data, the inclusion of a variance term in the BD metric, and the setting of priors. Our normalization algorithm is designed to address inter-sample differences in gene expression levels obtained from the microarray experiments It proceeds by scaling each sample's expression levels by a factor derived from the aggregate expression level of that sample. In this way, afer scaling, all samples have the same aggregate expession level.

A set of training data, labeled with outcome or disease subtype, was used to generate and evaluate hypotheses against the training data. A cross validation methodology was employed to learn parameter settings appropriate for the domain. Surviving hypotheses were blended in the Bayesian framework, yielding conditional outcome distributions. Hypotheses so learned are validated against an out-of-sample test set in order to assess generalization accuracy. This approach was successfully used to identify OPAL1/G0 as strong predictors of outcome in pediatric ALL as described in Example II.

2. Support Vector Machines.

Support vector machines (SVMs) are powerful tools for data classification (Cristianini et al., An Introduction to Support Vector Machines and Other Kernel-Based Learning Methods. Cambridge University Press, Cambridge, 2000; Vapnik, Statistical Learning Theory, John Wiley & Sons, New York, 1999). The original development of the SVM was motivated, in the simple case of two linearly separable classes, by the desire to choose an optimal linear classifier out of an infinite number of potential linear classifiers that could separate the data. This optimal classifier corresponds not only to a hyperplane that separates the classes but also to a hyperplane that attempts to be as far away as possible from all data points. If one imagines inserting the widest possible corridor between data points (with data points belonging to one class on one side of the corridor and data points belonging to the other class on the other side), then the optimal hyperplane would correspond to the imaginary line/plane/hyperplane running through the middle of this corridor.

The SVM has a number of characteristics that make it particularly appealing within the context of gene selection and the classification of gene expression data, namely: SVMs represent a multivariate classification algorithm that takes into account each gene simultaneously in a weighted fashion during training, and they scale quadratically with the number of training samples, N, rather than the number of features/genes, d. In order to be computationally feasible, other classification methods first have to reduce the number of dimensions (features/genes), and then classify the data in the reduced space. A univariate feature selection process or filter ranks genes according to how well each gene individually classifies the data. The overall classification is then heavily dependent upon how successful the univariate feature selection process is in pruning genes that have little class-distinction information content. In contrast, the SVM provides an effective mechanism for both classification and feature selection via the Recursive Feature Elimination algorithm (Guyon et al., Machine Learning 46, 389-422, 2002). This is a great advantage in gene expression problems where d is much greater than N, because the number of features does not have to be reduced a priori.

Recursive Feature Elimination (RFE) is an SVM-based iterative procedure that generates a nested sequence of gene subsets whereby the subset obtained at iteration k+1 is contained in the subset obtained at iteration k. The genes that are kept per iteration correspond to genes that have the largest weight magnitudes—the rationale being that genes with large weight magnitudes carry more information with respect to class discrimination than those genes with small weight magnitudes. We have implemented a version of SVM-RFE and obtained excellent results—comparable to Bayesian nets—for a range of infant leukemia classification tasks with blinded test sets.

3. Discriminant Analysis

Discriminant analysis is a widely used statistical analysis tool that can be applied to classification problems where a training set of samples, depending a set of p feature variables, is available (Duda et al., Pattern Classification (Second Edition). Wiley, New York, 2001). Each sample is regarded as a point in p-dimensional space R^{p}, and for a g-way classification problem, the training process yields a discriminant rule that partitions R^{p} into g disjoint regions, R₁ R₂, . . . , R_{g}. New samples with unknown class labels can then be classified based on the region Rᵢ to which the corresponding sample vector belongs. In many cases, determining the partitioning is equivalent to finding several linear or non-linear functions of the feature variables such that the value of the function differs significantly between different classes. This function is the so-called discriminant function. Discriminant rules fall into two categories: parametric and nonparametric. Parametric methods such as the maximum likelihood rule—including the special cases of linear discriminant analysis (LDA) and quadratic discriminant analysis (QDA) (Mardia et al., Multivariate Analysis. Academic Press, Inc., San Diego, 1979; Dudoit et al., J. Am. Stat. Ass'n. 97(457):77-87, 2002)—assume that there is an underlying probability distribution associated with each of the classes, and the training samples are used to estimate the distribution parameters. Non-parametric methods such as Fisher's linear discriminant and the k-nearest neighbor method (Duda et al., Pattern Classification (Second Edition). Wiley, New York, 2001) do not utilize parameter estimation of an underlying distribution in order to perform classifications based on a training set.

In applying discriminant analysis techniques to the gene expression classification problem, both categories of methods have been utilized, specifically LDA (binary classification) and Fisher's linear discriminant (multi-class problems). For the statistically designed infant leukemia dataset, LDA was applied successfully to the AML/ALL and t(4;11)/NOT class distinctions. Fisher's linear discriminant analysis was further used to identify three well-separated classes that clustered within the seven nominal MLL subclasses for which karyotype labels were available.

For both classes of methods, a major issue is the question of feature selection, either as an independent step prior to classification, or as part of the classifier training step. In addition to a simple ranking based on t-test score as used by other researchers (Dudoit et al., J. Am. Stat. Ass'n. 97(457):77-87, 2002), the use of stepwise discriminant analysis for determining optimal sets of distinguishing genes has been investigated. One challenge in the stepwise approach is the rapid increase of computational burden with the number of genes included in the initial set; the method is therefore being implemented on large-scale parallel computers. An alternative gene selection approach that is presently being explored is stepwise logistic regression (McCulloch et al., Generalized, Linear, and Mixed Models Wiley, New York, 2001; SAS Online Documentation for SAS System, Release 8.02, SAS Institute, Inc. 2001). Logistic regression is known to be well suited to binary classification problems involving mixed categorical and continuous data or to cases where the data are not normally distributed within the respective classes.

Various extensions of these techniques are expected to enable the incorporation of both categorical and continuous data in our classifiers. This enables the inclusion of known, discrete clinical labels (age, sex, genotype, white blood count, etc.) in conjunction with microrarray expression vectors, in order to perform more accurate classifications, particularly for outcome prediction. In addition to logistic regression as mentioned previously, one approach is to first quantify the categorical data (Hayashi, Ann. Inst. Statist. Math. 3:69-98, 1952), and then apply standard non-parameteric statistical classification techniques in the usual manner.

4. Fuzzy Inference

Traditional classification methods are based on the theory of crisp sets, where an element is either a member of a particular set or not. However many objects encountered in the real world do not fall into precisely defined membership criteria.

Fuzzy inference (also known as fuzzy logic) and adaptive neuro-fuzzy models are powerful learning methods for pattern recognition. Although researchers have previously investigated the use of fuzzy logic methods for reconstructing triplet relationships (activator/repressor/target) in gene regulatory networks (Woolf et al., Physiol. Genomics 3:9-15, 2000), these techniques have not been previously applied to the genomic classification problem. A significant advantage of fuzzy models is their ability to deal with problems where set membership is not binary (yes/no); rather, an element can reside in more than one set to varying degrees. For the classification problem, this results in a model that, like probabilistic methods such as Bayesian nets, can accommodate data sources that are incomplete, noisy, and may ultimately include non-numeric text-based expert knowledge derived from clinical data; polymorphisms or other forms of genomic data; or proteomic data that must be incorporated into the overall model in order to achieve a more accurate classification system in clinical contexts such as outcome prediction.

5. Genetic Algorithms

Fuzzy logic and other classification methods require the use of a gene selection method in order to reduce the size of the feature space to a numerically tractable size, and identify optimal sets of class-distinguishing genes for further analysis. We are exploring the use of genetic algorithms (GAs) for determining optimal feature sets during the training phase of a classification problem.

A GA is a simulation method that makes it possible to robustly search a very large space of possible solutions to an optimization problem, and find candidate solutions that are near optimal. Unlike traditional analytic approaches, GAs avoid “local minimum” traps, a classic problem arising in high-dimensional search spaces. Optimal feature selection for gene expression data where the sample size N is much smaller than the number of features d (for the Affymetrix leukemia data analyzed, d≈12,000 and N≈100-200) is a classic problem of this type. A genetic algorithm code has been developed by us to perform feature selection for the K-nearest neighbors classification method using the recently proposed GA/KNN approach (Li et al., Bioinformatics 17:1131-42, 2001); this method, which is compute-intensive, has been implemented on the parallel supercomputers. The approach has been applied recently to the statistically designed infant leukemia dataset, to evaluate biologic clusters discovered using unsupervised learning (VxInsight). The GA/KNN method was able to predict the hypothesized cluster labels (A,B,C) in one-vs.-all classification experiments.

### Example II

### Identification of a Gene Strongly Predictive of Outcome in Pediatric Acute Lymphoblastic Leukemia (ALL): OPAL1

Summary

To identify genes strongly predictive of outcome in pediatric ALL, we analyzed the retrospective case control study of 254 pediatric ALL samples described in Example IA. We divided the retrospective POG ALL case control cohort (n=254) into training (⅔ of cases, the “preB training set”) and test (⅓ of cases, the “preB test set”) sets, applied a Bayesian network approach, and performed statistical analyses. A particularly gene predictive of outcome in pediatric ALL was identified, corresponding to Affymetrix probe set 38652_at (“G0”: Hs. 10346; NM_Hypothetical Protein FLJ20154; partial sequences reported in GenBank Accession Number NM_{—}017787; NM_{—}017690; XM_{—}053688; NP_{—}060257). Two other genes, Affymetrix probe set 34610_at (“G1”: GNB2L1: G protein β2, related sequence 1; GenBank Accession Number NM_{—}006098;); and Affymetrix probe set 35659_at (“G2”: IL-10 Receptor alpha; GenBank Accession Number U00672), were identified as associated with outcome in conjunction with OPAL1/G0, but were substantially less significant. OPAL1/G0, which we have named OPAL1 for outcome predictor in acute leukemia, was a heretofore unknown human expressed sequence tag (EST), and had not been fully cloned until now. G1 (G protein β2, related sequence 1) encodes a novel RACK (receptor of activated protein kinase C) protein and is involved in signal transduction (Wang et al., Mol Biol Rep. 2003 March; 30(1):53-60) and G2 is the well-known IL-10 receptor alpha.

Importantly, we found that OPAL1/G0 was highly predictive of outcome (p=0.0014) in a completely different set of ALL cases assessed by gene expression profiling by another laboratory (the St. Jude set of ALL cases previously published by Yeoh et al. (Cancer Cell 1; 133-143, 2002)). We also observed a trend between high OPAL1/G0 and improved outcome in our retrospective cohort of infant ALL cases.

We have fully cloned the human homologue of OPAL1/G0 and characterized its genomic structure. OPAL1/G0 is highly conserved among eukaryotes, maps to human chromosome 10q24, and appears to be a novel transmembrane signaling protein with a short membrane insertion sequence and a potential transmembrane domain. This protein may be a protein inserted into the extracellular membrane (and function like a signaling receptor) or within an intracellular domain. We have also developed specific automated quantitative real time RT-PCR assays to precisely monitor the expression of OPAL1/G0 and other genes that we have found to be associated with outcome in ALL.

Bayesian Networks

We used Bayesian networks, a supervised learning algorithm as described in Example IB, to identify one or more genes that could be used to predict outcome as well as therapeutic resistance and treatment failure. To identify genes strongly predictive of outcome in pediatric ALL, we divided the retrospective POG ALL case control cohort (n=254) described above into training (⅔ of cases) and test (⅓ of cases) sets. Computational scientists were blinded to all clinical and biologic co-variables during training, except those necessary for the computational tasks. A large number of computational experiments were performed, in order to properly sample the space of Bayesian nets satisfying the constraints of the problem. In the context of high-dimensional gene expression data, the inclusion of more nets than is typical in the literature appears to yield better results. Our initial results using Bayesian nets showed classification rates in excess of 90-95%.

Identification of Genes Associated with Outcome

A particularly strong set of genes predictive of outcome was identified by applying a Bayesian network analysis to the preB training set. The three genes in the strongest predictive tree identified by Bayesian networks are provided in Table 2.

TABLE 2
Genes Strongly Predictive of Outcome in Pediatric ALL

| Gene | | | |
|---|---|---|---|
| Identifier: | Affymetrix | | Previously Known |
| Bayesian | Oligo | | Function/ |
| Network | Sequence | Gene/Protein Name | Comment |
| G0 | 38652_at | Hs. 10346; | Unknown human |
| | | NM_Hypothetical | EST, not previously |
| | | Protein FLJ20154 | fully cloned. |
| G1 | 34610_at | GNB2L1: G protein β2, | Signal |
| | | related sequence 1 | Transduction; |
| | | | Activator of Protein |
| | | | Kinase C |
| G2 | 35659_at | IL-10 Receptor alpha | IL-10 Receptor |
| | | | alpha |

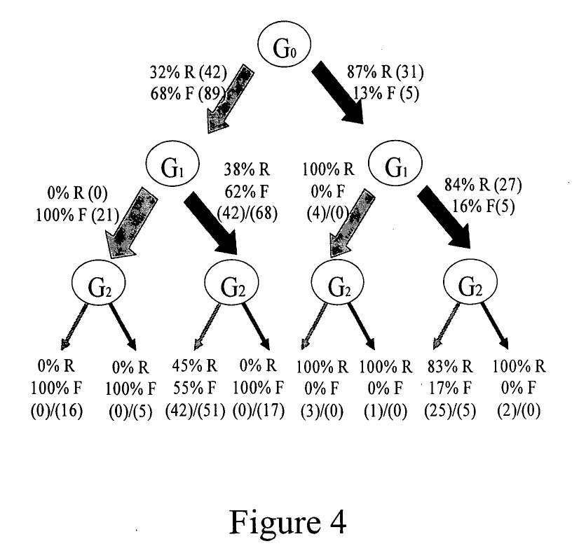

FIG. 4 shows a graphic representation of statistics that were extracted from the Bayesian net (Bayesian tree) that show association with outcome in ALL. The circles represent the key genes; the lighter arrows pointing toward the left denote low expression levels while the darker arrows pointing toward the right denote high expression of each gene. The percentage of patients achieving remission (R) or therapeutic failure (F) is shown for high or low expression of each gene, along with the number of patients in each group in parentheses.

Our analysis showed that pediatric ALL patients whose leukemic cells contain relatively high levels of expression of OPAL1/G0 have an extremely good outcome while low levels of expression of OPAL1/G0 is associated with treatment failure. At the top of the Bayesian network, OPAL1/G0 conferred the strongest predictive power; by assessing the level of OPAL1/G0 expression alone, ALL cases could be split into those with good outcomes (OPAL1/G0 high: 87% long term remissions) versus those with poor outcomes (OPAL1/G0 low: 32% long term remissions, 68% treatment failure). Detailed statistical analyses of the significance of OPAL1/G0 expression in the retrospective cohort revealed that low OPAL1/G0 expression was associated with induction failure (p=0.0036) while high OPAL1/G0 expression was associated with long term event free survival (p=0.02), particularly in males (p=0.0004). Higher levels of OPAL1/G0 expression were also associated with certain cytogenetic abnormalities (such as t(12;21)) and normal cytogenetics. Although the number of cases were limited in our initial retrospective cohort, low levels of OPAL1/G0 appeared to define those patients with low risk ALL who failed to achieve long term remission, suggesting that OPAL1/G0 may be useful in prospectively identifying children who would otherwise be classified as having low or standard risk disease, but who would benefit from further intensification.

The pre-B test set (containing the remaining 87 members of the pre-B cohort) was also analyzed. Unexpectedly, OPAL1/G0 when evaluated on the pre B test set showed a far less significant correlation with outcome. This is the only one of the four data sets (infant, pre-B training set, pre-B test set, and the Downing data set, below) in which no correlation was observed. One possible explanation is that, despite the fact that the preB data set was split into training and test sets by what should have been a random process, in retrospect, the composition of the test set differed very significantly from the training set. For example, the test set contains a disproportionately high fraction of studies involving high risk patients with poorer prognosis cytogenetic abnormalities which lack OPAL1/G0 expression; these children were also treated on highly different treatment regimens than the patients in the training set. Thus, there may not have been enough leukemia cases that expressed higher OPAL1/G0 levels (there were only sixteen patients with a high OPAL1/G0 expresion value in the test set) for us to reach statistcal significance. Finally, the p-value observed for the preB training set was so strong, as was the validation p-value for OPAL1/G0 outcome prediction in the independent data sets, that it would be virtually impossible that the observed correlation between OPAL1/G0 and outcome is an artifact.

In addition, PCR experiments recently completed in accordance with the methods outlined in Example III support the importance of OPAL1/G0 as a predictor of outcome. Although a large fraction (30%) of the 253 pre B cases could not be assessed by PCR due to sample availability, including 8 of the 36 cases from the pre B training set in which OPAL1/G0 was highly expressed, an initial analysis of the results on the 174 cases which could be assessed supports a clear statistical correlation between OPAL1/G0 and outcome (a p-value of about 0.005 on the PCR data alone, when the OPAL1/G0-high threshold is considered fixed). It should be noted that these PCR samples cut across the pre B training and test sets, and that the PCR results do not seem to reflect the same dichotomy in training and test set correlation as was seen in the microarray data. Furthermore, the RNA target for the PCR assays (directly amplified cDNA) and the Afffymetrix array experiments (linearly amplified twice cDNA) are quite different and it is satisfying that a moderately strong correlation (r=0.62) was observed between these two quite distinct methodologies to quantitate gene expression. Additionally, in a random re-sampling (bootstrap) procedure reported in herein, OPAL1/G0 does exhibit consistent significance.

As noted above, we evaluated expression levels of OPAL1/G0 in three entirely different and disjoint data sets. Two of the data sets, described above, were derived from retrospective cohorts of pediatric ALL patients registered to clinical trials previously coordinated by the Pediatric Oncology Group (POG): the statistically designed cohort of 127 infant leukemias (the “infant” data set); and the statistically designed case control study of 254 pediatric B-precursor and T cell ALL cases (the “pre-B” data set), specifically the 167 member “pre-B” training set. The third data set evaluated was a publicly available set of ALL cases previously published by Yeoh et al. (the “Downing” or “St. Jude” data set) (Cancer Cell 1; 133-143, 2002).

The following breakdown was conditioned on OPAL1/G0 expression level at its optimal threshold value, which in all data sets examined fell near the top quarter (22-25%) of the expression values. Low OPAL1/G0 expression was defined as having normalized OPAL1/G0 expression below this value, while high OPAL1/G0 expression was defined as having normalized OPAL1/G0 expression equal to or greater than this value.

Of the 167 members of the pre-B training set, 73 (44%) were classified as CCR (continuous complete remission) while 94 (56%) were classified as FAIL. Relative to the optimized threshold value, OPAL1/G0 expression was determined to be low in 131 samples and high in 36 samples. The following statistics were observed.

Low OPAL1/G0 Expression (131 Samples):
CCR: 42 32%
FAIL: 89 68%

High OPAL1/G0 Expression (36 Samples):
CCR: 31 86%
FAIL: δ 14%

The following p-values were observed for gene uncorrelated with outcome possessing any threshold point yielding our observations or better:
By Chi-squared: p-value ˜1.2*10ˆ(−7) (approximately 1 in ten million)
By TNoM: p-value ˜=5.7*10ˆ(−7) (approximately 1 in two million).
   where TNoM refers Threshold Number of Misclassifications=the number of misclassifications made by using a single-gene classifier with an optimally chosen threshold for separating the classes.

The significance of these p-values must be assessed in light of the fact that 12,000+ genes can be so considered (individually) against the training data. Even with 1.25×10⁴ candidate genes, under the null hypothesis of no associations, the expected number of genes that possess a threshold yielding our observation (or better) is still extremely small:
By Chi-squared: (1.2*10ˆ(−7))*(1.25*10ˆ4)=1.5*10ˆ(−3)
By TNoM: (5.7*10ˆ(−7))*(1.25*10ˆ4)=7.5*10ˆ(−3)
   Hence, one would expect to have to search approximately 667 independent data sets, each similar in composition to our pre-B training set (each consisting of 1.25*10ˆ4 candidate genes and 167 cases), in order to find even a single gene in one of these 667 data sets possessing a threshold yielding our observations or better as measured by Chi-squared, due to chance alone. (Using the p-value obtained from the TNoM statistic, we would expect to have to search 133 similar, independent data sets to find even a single gene possessing a threshold yielding a TNoM score at least as good as our observation.) These p-values are highly significant and support the conclusion that the observed statistical correlations are real, with high confidence.

Our analysis of the pre-B training set showed that pediatric ALL patients whose leukemic cells contain relatively high levels of expression of OPAL1/G0 have an extremely good outcome while low levels of expression of OPAL1/G0 is associated with treatment failure. In the entire pediatric ALL cohort under analysis, 44% of the patients were in long term remission for 4 or more years, while 56% of the patients had failed therapy within 4 years. At the top of the Bayesian network, OPAL1/G0 conferred the strongest predictive power; by assessing the level of OPAL1/G0 expression alone, ALL cases could be split into those with good outcomes (OPAL1/G0 high: 87% long term remission; 13% failures) versus those with poor outcomes (OPAL1/G0 low: 32% long term remissions, 68% treatment failure). Although the numbers are quite small as we continue down the Bayesian tree, outcome predictions can be somewhat refined by analyzing the expression levels of these G1 and G2.

We also investigated OPAL1/G0 expression level statistics across biological classifications typically utilized as predictive of outcome. The following represents a breakdown of OPAL1/G0 expression statistics within various subpopulations of the pre-B training set. The OPAL1/G0 threshold obtained by optimization in the original pre-B training set analysis (a value of 795) was used.

Normal Genotype (65 Members)

Outcome Statistics
26 CCR 40%
39 FAIL 60%

Low OPAL1/G0 Expression (51 Samples)
13 CCR 25%
38 FAIL 75%

High OPAL1/G0 Expression (14 Samples)
13 CCR 93%
1 FAIL 7%
   t(12:21) (Equivalent to TEL/AML1 in Downing Data Set, Below) (24 Members)

Outcome Statistics
18 CCR 75%
6 FAIL 25%

Low OPAL1/G0 Expression (Bottom 78%; 10 Samples)
6 CCR 60%
4 FAIL 40%

High OPAL1/G0 Expression (Top 22%; 14 Samples)
12 CCR 86%
2 FAIL 14%
   Hyperdiploid (17 Members)

Outcome Statistics
9 CCR 53%
8 FAIL 47%

Low OPAL1/G0 Expression (13 Samples)
5 CCR 38%
8 FAIL 62%

High OPAL1/G0 Expression (4 Samples)
4 CCR 100%
0 FAIL 0%
   t(4:11) and t(1:19) Combined (35 Members)

Outcome Statistics
13 CCR 37%
22 FAIL 63%

Low OPAL1/G0 Expression (34 Samples)
13 CCR 38%
21 FAIL 62%

High OPAL1/G0 Expression (1 Sample)
0 CCR 0%
1 FAIL 100%
   t(9:22) and Hypodiploid Combined (12 Members)

Outcome Statistics
2 CCR 17%
10 FAIL 83%

Low OPAL1/G0 Expression (12 Samples)
2 CCR 17%
10 FAIL 83%

High OPAL1/G0 Expression (0 Samples)
0 CCR --
0 FAIL --
   Low Age (<=10 Years) (109 Members)

Outcome Statistics
55 CCR 50%
54 FAIL 50%

Low OPAL1/G0 Expression (80 Samples)
30 CCR 38%
50 FAIL 62%

High OPAL1/G0 Expression (29 Samples)
25 CCR 86%
4 FAIL 14%
   High Age (>10 Years) (58 Members)

Outcome Statistics
18 CCR 31%
40 FAIL 69%

Low OPAL1/G0 Expression (51 Samples)
12 CCR 24%
39 FAIL 76%

High OPAL1/G0 Expression (7 Samples)
6 CCR 86%
1 FAIL 14%
   Low WBC (<=50,000) (79 Members)

Outcome Statistics
39 CCR 49%
40 FAIL 51%

Low OPAL1/G0 Expression (58 Samples)
21 CCR 36%
37 FAIL 64%

High OPAL1/G0 Expression (21 Samples)
18 CCR 86%
3 FAIL 14%
   High WBC (>50,000) (88 Members)

Outcome Statistics
34 CCR 39%
54 FAIL 61%

Low OPAL1/G0 Expression (73 Samples)
21 CCR 29%
52 FAIL 71%

High OPAL1/G0 Expression (15 Samples)
13 CCR 87%
2 FAIL 13%

The data evidence a number of interesting interactions between OPAL1/G0 and various parameters used for risk classification (karyotype and NCI risk criteria). Age and WBC (White Blood Count), in particular, are routinely used in the current risk stratification standards (age>10 years or WBC>50,000 are high risk), yet OPAL1/G0 appears to be the dominant predictor within both of these groups. Indeed, OPAL1/G0 appears to “trump” outcome prediction based on these biological classifications. In other words, regardless of biological classification, roughly the same OPAL1/G0 statistics are observed. For example, even though MLL translocation t(12:21) is generally associated with very good outcome, when OPAL1/G0 is low, the t(12:21) outcome is not nearly as good as when OPAL1/G0 is high. This association is also present in the Downing data set (see below), according to our analysis, although it was not recognized by Yeoh et al.

In our retrospective cohort balanced for remission/failure, OPAL1/G0 was more frequently expressed at higher levels in ALL cases with normal karyotype (14/65, 22%), t(12;21) (14/24, 58%) and hyperdiploidy (4/17, 24%%) compared to cases with t(1;19) (2%) and t(9;22) (0%). 86% of ALL cases with t(12;21) and high OPAL1/G0 achieved long term remission; while t(12;21) with low OPAL1/G0 had only a 40% remission rate. Interestingly, 100% of hyperdiploid cases and 93% of normal karyotype cases with high OPAL1/G0 attained remission, in contrast to an overall remission rate of 40% in each of these genetic groups.

Although our cases numbers were small and the cases highly selected, there appeared to be a correlation between low OPAL1/G0 and failure to achieve remission in children with low risk disease, suggesting that OPAL1/G0 may be useful in prospectively identifying children with low or standard risk disease who would benefit from further intensification. Interestingly, in children in the standard NCI risk group (age<10; WBC<50,000) and an overall remission rate of 50% in this case control study, children with high OPAL1/G0 had an 86% long term remission rate. Even children with NCI high risk criteria (age>10, WBC>50,000) and an overall remission rate of 31% in this selected cohort, children with high OPAL1/G0 had an 87% remission rate. Finally, OPAL1/G0 was also highly predictive of outcome in T ALL (p=0.02), as well as B precursor ALL.

Our statistical analyses of the significance of OPAL1/G0 expression in the retrospective cohort revealed that low OPAL1/G0 expression was associated with induction failure (p=0.0036) while high OPAL1/G0 expression was associated with long term event free survival (p=0.02), particularly in males (p=0.0004). Interestingly, actual quantitative levels of OPAL1/G0 appeared to be important and there was a clear expression threshold between remission and relapse.

To further validate the role of OPAL1/G0 in outcome prediction in ALL, we tested the usefulness of OPAL1/G0 on two additional independent set of ALL cases, the statistically designed infant ALL cohort described above, and the publicly available St. Jude ALL dataset (Yeoh et al., Cancer Cell 1; 133-143, 2002). In these two data sets, it should be noted that we explored OPAL1/G0's statistics specifically, and (in this context) did not test any other gene. Hence, the significance of the p-values computed for these two additional data sets should not be balanced against a large number of potential candidate genes. There was only one gene considered, and that was OPAL1/G0. Further, the threshold was fixed using the top 22% (17 samples) expressors as the threshold, not optimized as it was in the analysis of the pre-B training set.

Of the 76 members of the infant ALL data set (restricted to no-marginal ALLs), 29 (38%) were classified as CCR (continuous complete remission) while 47 (62%) were classified as FAIL. The following statistics were observed.

Low OPAL1/G0 Expression (Bottom 78%; 59 Samples)
CCR: 19 32%
FAIL: 40 68%

High OPAL1/G0 Expression (Top 22%; 17 Samples)
CCR: 10 59%
FAIL: 7 41%
By Chi-squared: p-value ˜=0.0465
By TNoM: p-value ˜=0.0453

For the Downing data set, “Heme Relapse” and “Other Relapse” were classified as FAIL and the 2nd AML was discarded as being of indeterminate outcome. Of the 232 members of the Downing data set, 201 (87%) were classified as CCR (continuous complete remission) while 31 (13%) were classified as FAIL. The following statistics were observed.

Low OPAL1/G0 Expression (Bottom 78%; 181 Samples)
CCR: 150 83%
FAIL: 31 17%

High OPAL1/G0 Expression (Top 22%; 51 Samples)
CCR: 51 100%
FAIL: 0 0%
By Chi-squared: p-value ˜=0.0014
TNoM is NA because same majority class in both groups
   An additional result against the Downing data set is that if the threshold is lowered slightly to include in the high group the top 25% of expressors (that is, 8 additional cases are above the OPAL1/G0 threshold), we obtained:

Low OPAL1/G0 Expression (Bottom 75%; 173 Samples)
CCR: 142 82%
FAIL: 31 18%

High OPAL1/G0 Expression (Top 25%; 59 Samples)
CCR: 59 100%
FAIL: 0 0%
By Chi-squared: p-value ˜=0.0004
   TNoM is NA because same majority class in both groups
      The more reflective p-value apparently lies closer to p=0.0004 than to 0.0014, since the threshold point is only a small distance from the predetermined 22% point and is characterized by a large gap in OPAL1/G0 expression values.

It should be noted that all three of these data sets are totally disjoint, and as a result the latter two studies represent independent validation of the statistics observed in the original “pre-B” training set evaluation. As previously discussed, Yeoh et al. were not able to identify or validate genes associated with outcome in the St. Jude dataset. The St. Jude data set was not balanced for remission versus failure; the overall long term remission rate in this series of cases was 87%. Additionally, Yeoh et al. employed SVMs which included many genes in the classification that masked the significance of OPAL1/G0. Our adapted BD metric controlled model complexity and allowed the significance of OPAL1/G0 to be realized in this data set. Indeed, we found that 100% of the cases in this St. Jude series with higher levels of OPAL1/G0, regardless of karyotype, achieved long term remissions (p=0.0014).

The following represents a breakdown of OPAL1/G0 expression statistics within various subpopulations of the Downing data set. The OPAL1/G0 threshold (25%) obtained by optimization in the original pre-B training set analysis was used. This yields 59 high OPAL/G0 cases in total, which are distributed among the various subgroups as follows:

TEL-AML1 (61 Members)

Outcome Statistics
57 CCR 93%
4 FAIL 7%

Low OPAL1/G0 Expression (7 Samples)
3 CCR 43%
4 FAIL 57%

High OPAL1/G0 Expression (54 Samples)
54 CCR 100%
0 FAIL 0%
   Hyperdiploid>50 (48 Samples)

Outcome Statistics
43 CCR 90%
5 FAIL 10%

Low OPAL1/G0 Expression (46 Samples)
41 CCR 89%
5 FAIL 11%

High OPAL1/G0 Expression
2 CCR 100%
0 FAIL 0%
   Hyperdiploid 47-50 (19 Members)

Outcome Statistics
19 CCR 100%
0 FAIL 0%

Low OPAL1/G0 Expression (18 Samples)
18 CCR 100%
0 FAIL 0%

High OPAL1/G0 Expression (1 Sample)
1 CCR 100%
0 FAIL 0%
   Pseudodiploid (21 Members)

Outcome Statistics
19 CCR 90%
2 FAIL 10%

Low OPAL1/G0 Expression (19 Samples)
17 CCR 89%
2 FAIL 11%

High OPAL1/G0 Expression (2 Samples)
2 CCR 100%
0 FAIL 0%
   As noted above, these data support the association of OPAL1/G0 with outcome across biological classifications, as noted above for the pre-B training set.
   Cloning and Characterization of OPAL1/G0

The human homologue of OPAL1/G0 was fully cloned and its genomic structure characterized. OPAL1/G0 is highly conserved among eukaryotes, maps to human chromosome 10q24, and appears to be a novel, potentially transmembrane signaling protein. To clone OPAL1/G0, RACE PCR was used to clone upstream sequences in the cDNA using lymphoid cell line RNAs. The genomic structure was derived from a comparison of OPAL1/G0 cDNAs to contiguous clones of germline DNA in GenBank. The total predicted mRNA length is approximately 4 kb (FIG. 2C; SEQ ID NO:16). We have developed very specific primers and probes to measure OPAL1/G0 (as well as G1 and G2) (see Example III) both qualitatively and quantitatively using PCR techniques.

Interestingly, preliminary studies reveal that the gene for OPAL1/G0 encodes two different RNAs (and potentially up to five different RNAs through alternative splicing of upstream exons) and presumably two different proteins based on alternative use of 5′ exons (1a and 1). These two different transcripts are differentially expressed in leukemia cell lines.

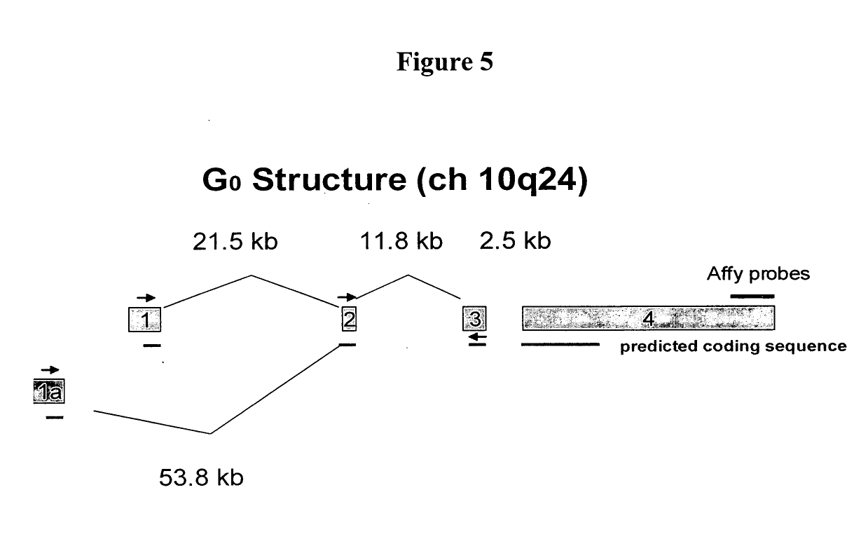

FIG. 5 is schematic drawing of the structure of OPAL1/G0. OPAL1/G0 is encoded by four different exons and was cloned using RACE PCR from the 3′ end of the gene using the Affymetrix oligonucleotide probe sequence (38652_at); interestingly the oligonucleotide (overlining labeled “Affy probes”) designed by Affymetrix from EST sequences turns out to be in the extreme 3′ untranslated region of this novel gene. The predicted coding region is shown as underlining for each exon. The location of primers we developed for use in quantitative detection of transcripts are shown as arrows above the exons.

Interestingly, OPAL1/G0 appears to encode at least two different proteins through alternative splicing of different 5′ exons (1 and 1a). FIG. 2A shows the nucleotide sequence (SEQ ID NO:1) and putative amino acid sequence (SEQ ID NO:2) of OPAL1/G0 (including exon 1), and FIG. 2B shows the nucleotide sequence (SEQ ID NO:3) and putative amino acid sequence (SEQ ID NO:4) of OPAL1/G0 (including exon 1a).

Table 3 shows the results of RT-PCR assays performed in accordance with Example III that confirm alternative exon use in OPAL1/G0. While all leukemia cell lines (REH, SUPB15) contained an OPAL1/G0 transcript with exons 2-3 and with exon 1a fused to exon 2; only ½ of the cell lines and the primary human ALL samples isolated to date express the alternative transcript (exon 1 fused to exon 2).

TABLE 3
RT-PCR assays of alternative exon use in OPAL1/G0.

| | G0 | | |
|---|---|---|---|
| Cell line | exon 1-2 | exon1a-2 | exon 2-3 |
| SUPB15 t(9; 22) e1a2 | − | + | + |
| REH t(12; 21) | + | + | + |
| K562 t(9; 22) b3a2 | + | + | + |
| BV173 t(9; 22) b2a2 | − | + | + |
| 697 t(1; 19) | + | + | + |
| NB-4 t(15; 17) | − | + | + |
| MV411 t(4; 11) | + | + | + |
| size | 154 | 158 | 166 |
| predicted | 148 | 155 | ˜168 |

100 ng equivalent RNA into each reaction

OPAL1/G0 appears to be rather ubiquitously expressed and it has a highly similar murine homologue. Preliminary examination of the translated coding sequence (FIG. 2) reveals a novel protein with a signal peptide, a short sequence (53 amino acids) which may be inserted in either the plasma membrane and be extracellular, or inserted within an intracellular membrane; a potential transmembrane domain; and an intracellular domain. Within the intracellular domain there are proline-rich regions that have strong homologies to proteins that bind WW domains and which are referred to as WW-binding protein 1 (WBP, see above). WW domains mediate interactions between proline-rich transcription factors and cytoplasmic signaling molecules. The data suggest that that this novel gene encodes a signaling protein, which may function as a receptor depending on its cellular location.
Characterization of G1 and G2

G1 encodes an interesting protein, a G protein β2 homologue that has been linked to activation of protein kinase C, to inhibition of invasion, and to chemosensitivity in solid tumors. It is also interesting that the Bayesian tree linked G2 (the IL-10 receptor a) to G6 and OPAL1/G0, as the interleukin IL-10 has been previously linked to improved outcome in pediatric ALL (Lauten et al., Leukemia 16:1437-1442, 2002; Wu et al., Blood Abstract, Blood Supplement 2002 (Abstract #3017).). IL-10 has been shown to be an autocrine factor for B cell proliferation and also to suppress T cell immune responses. ALL blasts that express a shortened, alternatively spliced form of IL-10 have been shown to have significantly better 5 year EFS (p=0.01) (Wu et al., Blood Abstract, Blood Supplement 2002 (Abstract #3017).). We have developed specific primers and probes to assess the direct expression of each of these genes in large ALL cohorts (Example III).

### Example III

### RT-PCR for Analysis of Expression Levels of OPAL1/G0, G1, G2 and Other Genes of Interest

We have developed direct RT-PCR assays to precisely measure the quantitative expression of these genes in an efficient two step approach. First, we perform a “qualitative” screen for positive cases using non-quantitative “end-point” RT-PCR assays with rapid and very inexpensive detection using the Agilent bioanalyzer. Positive cases detected with this simple, rapid, and highly sensitive methodology are then targeted for precise quantitative assessment of a particular gene using automated quantitative real time RT-PCR (Taqman technology).

Sequences for OPAL1/G0 (both splice forms) and pseudogenes identified from the other chromosomes were aligned, and OPAL1/G0 primers were designed to maximize the differences between the true OPAL1/G0 genes and the pseudogenes. The primers and probe sequences developed for specific quantitative assessment of the two alternatively spliced forms of OPAL1/G0 (assessed by quantifying mRNAs with exon 1 fused to exon 2 or alternatively exon 1a fused to exons 2) are:

For Exon 1 or 1a to 2 (the (+) Primers are Sense and the (−) are Antisense):

| | |
|---|---|
| Exon 1 (+) | |
| CCAACGTTAGTGTGGACGATGC | (SEQ ID NO:5) |
| Exon 1a (+) | |
| GCATGGCGCTCCTGCTC | (SEQ ID NO:6) |
| Exon 2 (−) | |
| GTAGTAGTTGCAGCACTGAGACTG | (SEQ ID NO:7) |
| Exon 2 probe (5′ FAM/3′ TAMRA) | |
| CCACAGCAGTGTCCTGTGTCACAGATGTAGC | (SEQ ID NO:8) |

For Exon 2 to 3:

| | |
|---|---|
| Exon 2 (+)a | |
| CAGTCTCAGTGCTGCAACTACTAC | (SEQ ID NO:9) |
| Exon 3 (−) | |
| GGCTTCTCGGTAAGCGATCAG | (SEQ ID NO:10) |
| Exon 3 probe (5′ FAM/3′ TAMRA) | |
| CTCAGGATGATGATGATGGTCCACACCAGCC | (SEQ ID NO:11) |

Using these primers and probes, we have developed highly sensitive and specific automated quantitative assays for OPAL1/G0 expression over a wide expression range. A standard curve was derived for the automated quantitative RT-PCR assays for the two alternatively spliced forms of OPAL1/G0. The assays were performed in cell lines shown in Table 3 and are highly linear over a large dynamic range.

The primers and probe sequences developed for specific quantitative assessment of G1 (G protein β2) and G2 (IL10Rα) are:

G1: Spans 2 introns (1.9 kb and 0.3 kb); from Exon 3 to Exon 5; 278 bp Amplicon

| | |
|---|---|
| G1e3 (+) | |
| CCAAGGATGTGCTGAGTGTGG | (SEQ ID NO:12) |
| G1e5 (−) | |
| CGTGTTCAGATAGCCTGTGTGG | (SEQ ID NO:13) |

G2: Spans 1 Intron of 3.6 kb; from Exon 3 to Exon 4; 189 bp Amplicon

| | |
|---|---|
| G2e3 (+) | |
| CCAACTGGACCGTCACCAAC | (SEQ ID NO:14) |
| G2e4 (−) | |
| GAATGGCAATCTCATACTCTCGG | (SEQ ID NO:15) |

Automated Quantitative RT-PCR

We routinely develop fluorogenic RT-PCR assays to detect the presence of leukemia-associated human genes, as well as viral genes, using an automated, closed analysis system (ABI 7700 Sequence Detector, PE-Applied Biosystems Inc., Foster City, Calif.). Accurate standards of cloned cDNAs containing the gene or sequence of interest are prepared in plasmid vectors (pCR 2.1, Invitrogen). These standard reagents are quantitated by fluorescence spectrometry and serially diluted over a six log range. Quantitative PCR is carried out in triplicate in the ABI 7700 instrument in a 96 well plate format, with optimized PCR conditions for each assay. The reverse transcriptase reaction employs 1 μg of RNA in a 20 μl volume consisting of 1× Perkin Elmer Buffer II, 7.5 mM MgCl₂, 5 μM random hexamers, 1 mM dNTP, 40 U RNasin and 100 U MMLV reverse transcriptase. The reaction is performed at 25° C. for 10 minutes, 48° C. for 60 min and 95° C. for 10 min. 4.5 μl of the resulting cDNA is used as template for the PCR. This is added to 1× Taqman Universal PCR Master Mix (PE Applied Biosystems, Foster City, Calif.), 100 nM fluorescently labeled Taqman probe and 100 nM of each primer in a 50 μl volume. The PCR is performed in the PRISM 7700 Sequence Detector as follows: “hot start” for 10 minutes at 95° C. (with AmpliTaq Gold, Perkin-Elmer) then 40 two step cycles of 95° C. for 15 seconds and 60° C. for 1 minute. This system detects the level of fluorescence from cleaved probe during each cycle of PCR and constructs the data into an amplification plot. This displays the threshold cycle (C_{T}) of detection for each reaction. The data collection and analysis are performed with Sequence Detection System v.1.6.3 software (PE Applied Biosystems, Foster City, Calif.). A standard concentration curve of C_{T} versus initial cDNA quantity is generated and analyzed with the ABI software to confirm the sensitivity range and reproducibility of the assay. To confirm RNA integrity, a segment of the ubiquitously expressed E2A gene is also amplified in all patient samples, along with a standard E2A or GAPDH cloned cDNA dilution series. This method can be utilized to quantitatively analyze expression levels for any gene of interest.

### Example IV

### Supervised Methods for Prediction of Outcome in Pediatric ALL Discretization

First the preB training set was discretized using a supervised method as well as an unsupervised discretization. Next p-values were computed by using the formula (nr/nh−er)/(er*(1−er)) then determine the likelihood of this value in a t-distribution. Here nr=number of remissions for gene high, nh=number of cases with gene high, and er=expected value of remission (44%). The results were ranked according to this p-value, and the preB training set was compared to entire preB data set. The results are shown in Tables 4-7. Tables 4 and 6 show two different lists based on the training set; Tables 5 and 7 show the entire preB data set for each of the two different approaches, respectively. Note that OPAL1/G0 is included on each of these lists as correlated with outcome, and there is substantial overlap between and among the lists. These lists thus identify potential additional genes that may be associated with OPAL1/G0 metabolically, might help determine the mechanism through which OPAL1/G0 acts, and might identify additional therapeutic or diagnostic genes.

Cumulative Distribution Functions (CDFS)

First the Helman-Veroff normalization scheme was applied to the preB training set data. Then CDFs were computed, followed by average and maximum difference between the CDFs. The distance between the two CDF curves reflects how different the two distributions are, hence the maximum distance and the average distance are measures of the way the two set differed. Finally, the genes were ranked by average and maximum differences for pre B training set and the entire preB data set. The results are shown in Tables 8-11.

The relative expression level for Affymetrix probe 39418_at (i.e., 0.5=half the median) was plotted across our pediatric ALL cases organized by outcome: FAIL (left panel) or REM (right panel), using Genespring (Silicon Genetics). The results showed that this gene's relative expression appears to be higher across failure cases and lower across remission cases.

Affymetrix probe 39418_at appears to be a probe from the consensus sequence of the cluster AJ007398, which includes Homo sapiens mRNA for the PBK1 protein (Huch et al., Placenta 19:557-567 (1998)). The sequence's approved gene symbol is DKFZP564M182, and the chromosomal location is 16p13.13. Originally, PBK1 was discovered through the identification of differentially expressed genes in human trophoblast cells by differential-display RT-PCR Functional annotations for the gene that this probe seems to represent are incomplete, however the sequence appears to have a protein domain similar to the ribosomal protein L1 (the largest protein from the large ribosomal subunit). PBK1 may prove to be a useful therapeutic target for treatment of pediatric ALL.

TABLE 4
Discretization/Training Set #1

| | Percent | | | | |
|---|---|---|---|---|---|
| Alpha | Remission | Number | Omim | | |
| (p-value) | High | Patients High | Link | Affy Id | Description |
| 0.000005 | 86.11 | 36 | | 38652_at | ****NM_017787 hypothetical protein FLJ20367 NM_017787 hypothetical protein |
| | | | | | FLJ20367 |
| 0.000463 | 68.75 | 48 | | 36012_at | NM_006346 analysis PIBF1 gene product |
| 0.000493 | 71.79 | 39 | 602731 | 41819_at | NM_001465 analysis FYN-binding protein FYB-120/130 |
| 0.000579 | 80 | 25 | 602982 | 38203_at | NM_002248 analysis potassium intermediate/small conductance calcium-activated |
| | | | | | channel subfamily N member 1 |
| 0.000611 | 73.53 | 34 | 603501 | 38270_at | NM_003631 analysis poly ADP-ribose glycohydrolase |
| 0.000637 | 65.52 | 58 | | 38838_at | NM_005033 analysis polymyositis/scleroderma autoantigen 1 75 kD |
| 0.000677 | 72.22 | 36 | | 32224_at | NM_014824 analysis KIAA0769 gene product |
| 0.000687 | 68.09 | 47 | 604076 | 36295_at | NM_003435 analysis zinc finger protein 134 clone pHZ-15 |
| 0.000744 | 71.05 | 38 | 605072 | 35756_at | NM_005716 analysis GLUT1 C-terminal binding protein |
| 0.000783 | 81.82 | 22 | | 39357_at | |
| 0.000785 | 66.67 | 51 | | 41559_at | |
| 0.000925 | 64.91 | 57 | 603026 | 38134_at | NM_002655 analysis pleiomorphic adenoma gene 1 |
| 0.001017 | 67.39 | 46 | 602600 | 32398_s_at | NM_004631 analysis low density lipoprotein receptor-related protein 8 |
| | | | | | apolipoprotein e receptor NM_017522 analysis apolipoprotein E receptor 2 |
| 0.001146 | 75 | 28 | | 39833_at | NM_015716 analysis Misshapen/NIK-related kinase |
| 0.001151 | 66 | 50 | | 41727_at | NM_016284 analysis KIAA1007 protein |
| 0.001389 | 78.26 | 23 | | 41192_at | NM_019610 analysis hypothetical protein 669 |
| 0.001408 | 67.44 | 43 | | 35669_at | |
| 0.001413 | 71.88 | 32 | 604463 | 33111_at | NM_007053 analysis natural killer cell receptor immunoglobulin superfamily member |
| 0.001441 | 87.5 | 16 | | 39768_at | |
| 0.001549 | 70.59 | 34 | | 36537_at | |
| 0.001681 | 65.31 | 49 | 603303 | 31473_s_at | NM_003747 analysis tankyrase TRF1-interacting ankyrin-related ADP-ribose |
| 0.001741 | 61.11 | 72 | | 32624_at | polymerase |
| 0.001741 | 61.11 | 72 | 147267 | 37343_at | NM_002224 analysis inositol 1 4 5-triphosphate receptor type 3 |
| 0.00182 | 68.42 | 38 | 137140 | 37062_at | NM_000807 analysis gamma-aminobutyric acid A receptor alpha 2 precursor |
| 0.00182 | 68.42 | 38 | 604092 | 572_at | NM_003318 analysis TTK protein kinase |
| 0.001929 | 63.64 | 55 | 152390 | 307_at | NM_000698 analysis arachidonate 5-lipoxygenase |
| 0.00226 | 86.67 | 15 | 251000 | 40105_at | NM_000255 analysis methylmalonyl Coenzyme A mutase precursor |
| 0.002336 | 69.7 | 33 | 136533 | 40570_at | NM_002015 analysis forkhead box O1A |
| 0.002381 | 60.87 | 69 | 300304 | 40141_at | NM_003588 analysis cullin 4B |
| 0.002419 | 75 | 24 | 107265 | 1116_at | NM_001770 analysis CD19 antigen |
| 0.002419 | 75 | 24 | 194550 | 40569_at | NM_003422 analysis zinc finger protein 42 myeloid-specific retinoic acid- responsive |
| 0.002447 | 64.58 | 48 | 602545 | 1488_at | NM_002844 analysis protein tyrosine phosphatase receptor type K |
| 0.002526 | 68.57 | 35 | | 38821_at | NM_006320 analysis progesterone membrane binding protein |
| 0.002694 | 73.08 | 26 | | 40177_at | |
| 0.002712 | 67.57 | 37 | 313650 | 112_g_at | NM_004606 analysis TATA box binding protein TBP associated factor RNA |
| | | | | | polymerase II A 250 kD |
| 0.002712 | 67.57 | 37 | | 1756_f_at | NM_000776 analysis cytochrome P450 subfamily IIIA niphedipine oxidase |
| | | | | | polypeptide 3 |
| 0.002712 | 67.57 | 37 | 600310 | 40161_at | NM_000095 analysis cartilage oligomeric matrix protein presursor |
| 0.002712 | 67.57 | 37 | 230000 | 41814_at | NM_000147 analysis fucosidase alpha-L- 1 tissue |
| 0.002776 | 57.73 | 97 | 191318 | 32557_at | NM_007279 analysis U2 small nuclear ribonucleoprotein auxiliary factor 65 kD |
| 0.002863 | 62.5 | 56 | 601958 | 34726_at | NM_000725 analysis calcium channel voltage-dependent beta 3 subunit |

TABLE 5
Discretization/Whole Set #1

| | Percent | Number | | | |
|---|---|---|---|---|---|
| Alpha | Remission | Patients | | | |
| (p-value) | High | High | Omim Link | Affy Id | Description |
| 0.000102 | 75.61 | 41 | 602982 | 38203_at | NM_002248 analysis potassium intermediate/small conductance calcium- |
| | | | | | activated channel subfamily N member 1 |
| 0.000118 | 71.15 | 52 | | 38652_at | ****NM_017787 hypothetical protein FLJ20154 NM_017787 hypothetical protein |
| | | | | | FLJ20154 |
| 0.000213 | 64.2 | 81 | 162096 | 577_at | NM_002391 analysis midkine neurite growth-promoting factor 2 |
| 0.000275 | 64.47 | 76 | 604076 | 36295_at | NM_003435 analysis zinc finger protein 134 clone pHZ-15 |
| 0.000369 | 59.83 | 117 | 147267 | 37343_at | NM_002224 analysis inositol 1 4 5-triphosphate receptor type 3 |
| 0.000379 | 61.96 | 92 | | 38838_at | NM_005033 analysis polymyositis/scleroderma autoantigen 1 75 kD |
| 0.000382 | 66.67 | 60 | | 35669_at | |
| 0.000391 | 64 | 75 | | 41727_at | NM_016284 analysis KIAA1007 protein |
| 0.000474 | 74.29 | 35 | | 38713_at | NM_019106 analysis septin 3 |
| 0.000584 | 60.61 | 99 | 602731 | 41819_at | NM_001465 analysis FYN-binding protein FYB-120/130 |
| 0.000588 | 65.57 | 61 | 604463 | 33111_at | NM_007053 analysis natural killer cell receptor immunoglobulin superfamily |
| | | | | | member |
| 0.000622 | 65.08 | 63 | 118820 | 41252_s_at | NM_020991 analysis chorionic somatomammotropin hormone 2 isoform 1 |
| | | | | | precursor NM_022644 analysis chorionic somatomammotropin hormone 2 |
| | | | | | isoform 2 precursor NM_022645 analysis chorionic somatomammotropin |
| | | | | | hormone 2 isoform 3 precursor NM_022646 analysis chori |
| 0.000651 | 70.73 | 41 | | 1756_f_at | NM_000776 analysis cytochrome P450 subfamily IIIA niphedipine oxidase |
| | | | | | polypeptide 3 |
| 0.000651 | 70.73 | 41 | | 40177_at | |
| 0.000667 | 61.9 | 84 | 602026 | 32724_at | NM_006214 analysis phytanoyl-CoA hydroxylase Refsum disease |
| 0.000709 | 66.67 | 54 | 145505 | 40617_at | NM_005622 analysis SA rat hypertension-associated homolog |
| 0.000753 | 63.38 | 71 | | 41559_at | |
| 0.000782 | 60.42 | 96 | 601798 | 34332_at | NM_005471 analysis glucosamine-6-phosphate isomerase |
| 0.000784 | 63.01 | 73 | | 36129_at | |
| 0.000873 | 62.03 | 79 | 603261 | 35741_at | NM_003559 analysis phosphatidylinositol-4-phosphate 5-kinase type II beta |
| 0.000892 | 64.52 | 62 | | 32224_at | NM_014824 analysis KIAA0769 gene product |
| 0.000892 | 64.52 | 62 | | 35066_g_at | NM_013303 analysis fetal hypothetical protein |
| 0.000928 | 61.45 | 83 | 603303 | 31473_s_at | NM_003747 analysis tankyrase TRF1-interacting ankyrin-related ADP-ribose |
| | | | | | polymerase |
| 0.000971 | 70 | 40 | 602793 | 34156_i_at | NM_003511 analysis H2A histone family member I |
| 0.00101 | 88.24 | 17 | 602015 | 41068_at | NM_002540 analysis outer dense fibre of sperm tails 2 |
| 0.001048 | 60.22 | 93 | | 36825_at | NM_006074 analysis stimulated trans-acting factor 50 kDa |
| 0.001063 | 62.86 | 70 | | 37814_g_at | |
| 0.001089 | 59.79 | 97 | 300248 | 36004_at | NM_003639 analysis inhibitor of kappa light polypeptide gene enhancer in B- |
| | | | | | cells kinase gamma |
| 0.001093 | 65.45 | 55 | 604092 | 572_at | NM_003318 analysis TTK protein kinase |
| 0.001104 | 62.5 | 72 | | 38926_at | |
| 0.001216 | 61.54 | 78 | | 41478_at | |
| 0.001225 | 58.26 | 115 | 122561 | 40650_r_at | NM_004382 analysis corticotropin releasing hormone receptor 1 |
| 0.001251 | 61.25 | 80 | 601958 | 34726_at | NM_000725 analysis calcium channel voltage-dependent beta 3 subunit |
| 0.001324 | 70.27 | 37 | 107265 | 1116_at | NM_001770 analysis CD19 antigen |
| 0.001333 | 63.49 | 63 | 602597 | 361_at | NM_004326 analysis B-cell CLL/lymphoma 9 |
| 0.001431 | 59.78 | 92 | 300059 | 34292_at | NM_003492 chromosome X open reading frame 12 |
| 0.001431 | 59.78 | 92 | 604518 | 38865_at | NM_004810 analysis GRB2-related adaptor protein 2 |
| 0.001444 | 62.69 | 67 | 602600 | 32398_s_at | NM_004631 analysis low density lipoprotein receptor-related protein 8 |
| | | | | | apolipoprotein e receptor NM_017522 analysis apolipoprotein E receptor 2 |
| 0.001455 | 59.57 | 94 | 123838 | 1923_at | NM_005190 analysis cyclin C |
| 0.001547 | 61.97 | 71 | 103270 | 40336_at | NM_004110 analysis ferredoxin reductase isoform 2 precursor NM_024417 |
| | | | | | ferredoxin reductase isoform 1 precursor |

TABLE 6
Discretization/Training Set #2

| | Percent | Number | | | |
|---|---|---|---|---|---|
| Alpha | Remission | Patients | | | |
| (p-value) | High | High | Omim Link | Affy Id | Description |
| 0.000326 | 72.5 | 40 | | 38652_at | ****NM_017787 hypothetical protein FLJ20154 NM_017787 hypothetical protein |
| | | | | | FLJ20154 |
| 0.000677 | 72.22 | 36 | 602731 | 41819_at | NM_001465 analysis FYN-binding protein FYB-120/130 |
| 0.001085 | 66.67 | 48 | 152390 | 307_at | NM_000698 analysis arachidonate 5-lipoxygenase |
| 0.001215 | 65.38 | 52 | | 41478_at | |
| 0.002082 | 66.67 | 42 | 137140 | 37062_at | NM_000807 analysis gamma-aminobutyric acid A receptor alpha 2 precursor |
| 0.002526 | 68.57 | 35 | | 32224_at | NM_014824 analysis KIAA0769 gene product |
| 0.002666 | 63.46 | 52 | | 39190_s_at | |
| 0.002768 | 62.96 | 54 | | 32624_at | |
| 0.003068 | 65.85 | 41 | 602600 | 32398_s_at | NM_004631 analysis low density lipoprotein receptor-related protein 8 apolipoprotein |
| | | | | | e receptor |
| | | | | | NM_017522 analysis apolipoprotein E receptor 2 |
| 0.003236 | 65.12 | 43 | 601798 | 34332_at | NM_005471 analysis glucosamine-6-phosphate isomerase |
| 0.003236 | 65.12 | 43 | 601974 | 587_at | NM_001400 analysis endothelial differentiation sphingolipid G-protein-coupled |
| | | | | | receptor 1 |
| 0.003547 | 63.83 | 47 | 300059 | 34292_at | NM_003492 chromosome X open reading frame 12 |
| 0.004271 | 65.79 | 38 | | 35669_at | |
| 0.004271 | 65.79 | 38 | | 36537_at | |
| 0.004502 | 65 | 40 | 600310 | 40161_at | NM_000095 analysis cartilage oligomeric matrix protein presursor |
| 0.004516 | 70.37 | 27 | 600703 | 32414_at | |
| 0.005118 | 63.04 | 46 | 605230 | 1711_at | NM_005657 analysis tumor protein p53-binding protein 1 |
| 0.005118 | 63.04 | 46 | 600735 | 625_at | |
| 0.005625 | 66.67 | 33 | 604090 | 40575_at | NM_004747 analysis discs large Drosophila homolog 5 |
| 0.005962 | 65.71 | 35 | | 35260_at | NM_014938 analysis KIAA0867 protein |
| 0.006102 | 60 | 60 | | 2091_at | |
| 0.006279 | 64.86 | 37 | 133171 | 1087_at | NM_000121 analysis erythropoietin receptor precursor |
| 0.006413 | 58.82 | 68 | | 31353_f_at | NM_012185 analysis forkhead box E2 |
| 0.007559 | 61.7 | 47 | 601920 | 35414_s_at | NM_000214 analysis jagged 1 precursor |
| 0.007559 | 61.7 | 47 | | 41559_at | |
| 0.007755 | 61.22 | 49 | 600074 | 266_s_at | NM_013230 CD24 antigen small cell lung carcinoma cluster 4 antigen |
| 0.007755 | 61.22 | 49 | | 33233_at | |
| 0.008091 | 60.38 | 53 | 309860 | 37628_at | NM_000898 analysis monoamine oxidase B |
| 0.008466 | 59.32 | 59 | | 39865_at | |
| 0.008781 | 64.71 | 34 | 600392 | 1043_s_at | NM_002879 analysis RAD52 S. cerevisiae homolog |
| 0.008781 | 64.71 | 34 | 130610 | 36733_at | NM_001961 analysis eukaryotic translation elongation factor 2 |
| 0.008781 | 64.71 | 34 | 162096 | 577_at | NM_002391 analysis midkine neurite growth-promoting factor 2 |
| 0.009185 | 63.89 | 36 | 601014 | 40246_at | NM_004087 analysis discs large Drosophila homolog 1 |
| 0.009556 | 63.16 | 38 | | 1756_f_at | NM_000776 analysis cytochrome P450 subfamily IIIA niphedipine oxidase |
| | | | | | polypeptide 3 |
| 0.009895 | 62.5 | 40 | 605179 | 33061_at | NM_001214 analysis chromosome 16 open reading frame 3 |
| 0.009895 | 62.5 | 40 | 312820 | 34068_f_at | NM_005635 analysis synovial sarcoma X breakpoint 1 |
| 0.009895 | 62.5 | 40 | | 34186_at | |
| 0.010201 | 61.9 | 42 | | 32233_at | |
| 0.010478 | 61.36 | 44 | | 32978_g_at | NM_015864 analysis PL48 |
| 0.010725 | 60.87 | 46 | 601632 | 35939_s_at | NM_006237 analysis POU domain class 4 transcription factor 1 |

TABLE 7
Discretization/Whole Set #2

| | | Number | | | |
|---|---|---|---|---|---|
| Alpha | Percent | Patients | Omim | | |
| (p-value) | Remission High | High | Link | Affy Id | Description |
| 0.000032 | 73.58 | 53 | 602731 | 41819_at | NM_001465 analysis FYN-binding protein FYB-120/130 |
| 0.000299 | 66.15 | 65 | 601798 | 34332_at | NM_005471 analysis glucosamine-6-phosphate isomerase |
| 0.000486 | 67.27 | 55 | 162096 | 577_at | NM_002391 analysis midkine neurite growth-promoting factor 2 |
| 0.001104 | 62.5 | 72 | 152390 | 307_at | NM_000698 analysis arachidonate 5-lipoxygenase |
| 0.001493 | 65.38 | 52 | 600392 | 1043_s_at | NM_002879 analysis RAD52 S. cerevisiae homolog |
| 0.001738 | 63.79 | 58 | 118820 | 41252_s_at | NM_020991 analysis chorionic somatomammotropin hormone 2 isoform 1 precursor |
| | | | | | NM_022644 analysis chorionic somatomammotropin hormone 2 isoform 2 precursor |
| | | | | | NM_022645 analysis chorionic somatomammotropin hormone 2 isoform 3 precursor |
| | | | | | NM_022646 analysis chori |
| 0.001927 | 65.96 | 47 | 162096 | 38124_at | NM_002391 analysis midkine neurite growth-promoting factor 2 |
| 0.002265 | 64.15 | 53 | 130610 | 36733_at | NM_001961 analysis eukaryotic translation elongation factor 2 |
| 0.002265 | 64.15 | 53 | | 39196_i_at | |
| 0.002431 | 60 | 80 | | 36331_at | |
| 0.002477 | 59.76 | 82 | 126420 | 34351_at | NM_003286 analysis topoisomerase DNA I |
| 0.002572 | 62.71 | 59 | | 41559_at | |
| 0.003001 | 60.87 | 69 | 601920 | 35414_s_at | NM_000214 analysis jagged 1 precursor |
| 0.003098 | 64 | 50 | | 32224_at | NM_014824 analysis KIAA0769 gene product |
| 0.003405 | 66.67 | 39 | | 35669_at | |
| 0.003739 | 56.88 | 109 | | 41727_at | NM_016284 analysis KIAA1007 protein |
| 0.004149 | 60.29 | 68 | | 41478_at | |
| 0.004387 | 59.46 | 74 | 603006 | 1483_at | NM_001794 analysis cadherin 4 type 1 R-cadherin retinal |
| 0.004387 | 59.46 | 74 | 124092 | 1548_s_at | NM_000572 analysis interleukin 10 |
| 0.004572 | 58.75 | 80 | | 39190_s_at | |
| 0.004613 | 62.75 | 51 | | 1756_f_at | NM_000776 analysis cytochrome P450 subfamily IIIA niphedipine oxidase |
| | | | | | polypeptide 3 |
| 0.004613 | 62.75 | 51 | 601013 | 33625_g_at | NM_000721 analysis calcium channel voltage-dependent alpha 1E subunit |
| 0.00478 | 57.78 | 90 | | 32058_at | NM_004854 analysis HNK-1 sulfotransferase |
| 0.005235 | 61.02 | 59 | 601184 | 33208_at | NM_006260 analysis DnaJ Hsp40 homolog subfamily C member 3 |
| 0.005282 | 65 | 40 | | 40177_at | |
| 0.005561 | 64.29 | 42 | 300097 | 35097_at | NM_002363 analysis melanoma antigen family B 1 |
| 0.005602 | 60 | 65 | 147267 | 37343_at | NM_002224 analysis inositol 1 4 5-triphosphate receptor type 3 |
| 0.005803 | 59.42 | 69 | 605230 | 1711_at | NM_005657 analysis tumor protein p53-binding protein 1 |
| 0.005803 | 59.42 | 69 | 300059 | 34292_at | NM_003492 chromosome X open reading frame 12 |
| 0.005826 | 63.64 | 44 | 604090 | 40575_at | NM_004747 analysis discs large Drosophila homolog 5 |
| 0.006398 | 56.19 | 105 | | 31353_f_at | NM_012185 analysis forkhead box E2 |
| 0.007277 | 60.34 | 58 | | 31653_at | |
| 0.007428 | 60 | 60 | | 38652_at | ****NM_017787 hypothetical protein FLJ20154 NM_017787 hypothetical protein |
| | | | | | FLJ20154 |
| 0.007566 | 59.68 | 62 | | 32707_at | NM_007044 analysis katanin p60 subunit A 1 |
| 0.007566 | 59.68 | 62 | | 35602_at | |
| 0.007692 | 59.38 | 64 | 605491 | 34873_at | NM_006393 analysis nebulette |
| 0.007806 | 59.09 | 66 | | 38530_at | |
| 0.007909 | 58.82 | 68 | 602149 | 37920_at | NM_002653 analysis paired-like homeodomain transcription factor 1 |
| 0.008012 | 63.41 | 41 | | 773_at | |
| 0.008081 | 58.33 | 72 | | 35066_g_at | NM_013303 analysis fetal hypothetical protein |

TABLE 8
Maximum Difference-Selected Genes (Training Set)

| | | | Omim | | |
|---|---|---|---|---|---|
| Index | Max Diff | Avg Diff | Link | Affy Id | Description |
| 6080 | 0.350189 | 0.133728 | | 38652_at | ****NM_017787 hypothetical protein FLJ20154 NM_017787 hypothetical protein |
| | | | | | FLJ20154 |
| 6031 | 0.342466 | 0.133158 | 142200 | 38585_at | NM_000559 analysis hemoglobin gamma A |
| 4022 | 0.339988 | 0.132256 | 140555 | 35965_at | NM_002155 analysis heat shock 70 kD protein 6 HSP70B |
| 6674 | 0.322064 | 0.130643 | | 39418_at | |
| 5053 | 0.307928 | 0.129113 | 147267 | 37343_at | NM_002224 analysis inositol 1 4 5-triphosphate receptor type 3 |
| 1662 | 0.306616 | 0.128926 | 191318 | 32557_at | NM_007279 analysis U2 small nuclear ribonucleoprotein auxiliary factor 65 kD |
| 7403 | 0.305159 | 0.125099 | 300151 | 40435_at | |
| 1717 | 0.304867 | 0.124241 | | 32624_at | |
| 2290 | 0.304722 | 0.120535 | 156491 | 33415_at | NM_002512 analysis non-metastatic cells 2 protein NM23B expressed in |
| 8278 | 0.303119 | 0.119869 | | 41559_at | |
| 5676 | 0.300495 | 0.118728 | 110750 | 38119_at | NM_002101 analysis glycophorin C isoform 1 NM_016815 analysis glycophorin |
| | | | | | C isoform 2 |
| 969 | 0.298892 | 0.11592 | | 31472_s_at | |
| 6169 | 0.297727 | 0.111653 | 600276 | 38750_at | NM_000435 analysis Notch Drosophila homolog 3 |
| 2429 | 0.297581 | 0.110325 | 300156 | 33637_g_at | NM_001327 analysis cancer/testis antigen |
| 740 | 0.295686 | 0.110118 | 156491 | 1980_s_at | NM_002512 analysis non-metastatic cells 2 protein NM23B expressed in |
| 1779 | 0.294521 | 0.107107 | 605031 | 32703_at | NM_014264 analysis serine/threonine kinase 18 |
| 297 | 0.291023 | 0.106625 | 187011 | 1403_s_at | NM_002985 analysis small inducible cytokine A5 RANTES |
| 831 | 0.289857 | 0.105829 | | 2091_at | |
| 4509 | 0.288254 | 0.104053 | 146691 | 36624_at | NM_000884 analysis IMP inosine monophosphate dehydrogenase 2 |
| 580 | 0.286797 | 0.103697 | 601645 | 176_at | NM_002719 analysis protein phosphatase 2 regulatory subunit B B56 gamma |
| | | | | | isoform |
| 6199 | 0.286797 | 0.103514 | 600673 | 38794_at | NM_014233 analysis upstream binding transcription factor RNA polymerase I |
| 93 | 0.286797 | 0.103116 | | 1126_s_at | |
| 5558 | 0.286651 | 0.100579 | 133171 | 37986_at | NM_000121 analysis erythropoietin receptor precursor |
| 4335 | 0.285194 | 0.10045 | 602524 | 36386_at | NM_002610 analysis pyruvate dehydrogenase kinase isoenzyme 1 |
| 6259 | 0.281988 | 0.100437 | 604518 | 38865_at | NM_004810 analysis GRB2-related adaptor protein 2 |
| 3749 | 0.281988 | 0.09987 | 142704 | 35606_at | NM_002112 analysis histidine decarboxylase |
| 813 | 0.280822 | 0.099596 | 602867 | 2062_at | NM_001553 analysis insulin-like growth factor binding protein 7 |
| 8219 | 0.27747 | 0.099577 | | 41478_at | |
| 5380 | 0.276159 | 0.098971 | | 37748_at | |
| 54 | 0.276013 | 0.097783 | 600210 | 106_at | NM_004350 analysis runt-related transcription factor 3 |
| 4892 | 0.275867 | 0.097033 | 604713 | 37147_at | NM_002975 analysis stem cell growth factor lymphocyte secreted C-type lectin |
| 8012 | 0.274847 | 0.09695 | | 41208_at | |
| 5668 | 0.274556 | 0.096929 | 118661 | 38111_at | NM_004385 analysis chondroitin sulfate proteoglycan 2 versican |
| 7036 | 0.27441 | 0.096861 | | 39932_at | |
| 8435 | 0.27441 | 0.096558 | 603413 | 41761_at | NM_003252 analysis TIA1 cytotoxic granule-associated RNA-binding protein-like |
| | | | | | 1 isoform 1 |
| | | | | | NM_022333 TIA1 cytotoxic granule-associated RNA-binding protein-like 1 |
| | | | | | isoform 2 |
| 4051 | 0.273244 | 0.09647 | | 36002_at | NM_014939 analysis KIAA1012 protein |
| 537 | 0.272952 | 0.096296 | 605230 | 1711_at | NM_005657 analysis tumor protein p53-binding protein 1 |
| 8601 | 0.271349 | 0.096014 | 600258 | 525_g_at | NM_000534 analysis postmeiotic segregation 1 |
| 3498 | 0.270329 | 0.096003 | 603083 | 35201_at | NM_001533 analysis heterogeneous nuclear ribonucleoprotein L |
| 1619 | 0.270184 | 0.095026 | | 324_f_at | |

TABLE 9
Average Difference-Selected Genes (Training Set)

| | | | Omim | | |
|---|---|---|---|---|---|
| Index | Max Diff | Avg Diff | Link | Affy Id | Description |
| 54 | 0.350189 | 0.133728 | 600210 | 106_at | NM_004350 analysis runt-related transcription factor 3 |
| 8702 | 0.342466 | 0.133158 | 182120 | 671_at | NM_003118 analysis secreted protein acidic cysteine-rich osteonectin |
| 5676 | 0.339988 | 0.132256 | 110750 | 38119_at | NM_002101 analysis glycophorin C isoform 1 NM_016815 analysis glycophorin C isoform 2 |
| 8219 | 0.322064 | 0.130643 | | 41478_at | |
| 3899 | 0.307928 | 0.129113 | | 35796_at | NM_007284 analysis protein tyrosine kinase 9-like A6-related protein |
| 6674 | 0.306616 | 0.128926 | | 39418_at | |
| 4801 | 0.305159 | 0.125099 | | 37006_at | NM_006425 analysis step II splicing factor SLU7 |
| 8799 | 0.304867 | 0.124241 | 605482 | 824_at | NM_004832 analysis glutathione-S-transferase like |
| 6327 | 0.304722 | 0.120535 | | 38971_r_at | NM_006058 analysis Nef-associated factor 1 |
| 6080 | 0.303119 | 0.119869 | | 38652_at | ****NM_017787 hypothetical protein FLJ20154 NM_017787 hypothetical protein FLJ20154 |
| 7348 | 0.300495 | 0.118728 | 139314 | 40365_at | NM_002068 analysis guanine nucleotide binding protein G protein alpha 15 Gq class |
| 8479 | 0.298892 | 0.11592 | 602731 | 41819_at | NM_001465 analysis FYN-binding protein FYB-120/130 |
| 4892 | 0.297727 | 0.111653 | 604713 | 37147_at | NM_002975 analysis stem cell growth factor lymphocyte secreted C-type lectin |
| 7693 | 0.297581 | 0.110325 | 601323 | 40817_at | NM_006184 analysis nucleobindin 1 |
| 2488 | 0.295686 | 0.110118 | 603593 | 33731_at | NM_003982 analysis solute carrier family 7 cationic amino acid transporter y system member 7 |
| 906 | 0.294521 | 0.107107 | 152390 | 307_at | NM_000698 analysis arachidonate 5-lipoxygenase |
| 6311 | 0.291023 | 0.106625 | 603109 | 38944_at | NM_005902 analysis MAD mothers against decapentaplegic Drosophila homolog 3 |
| 2097 | 0.289857 | 0.105829 | | 33188_at | NM_014337 analysis peptidylprolyl isomerase cydophilin like 2 |
| 1779 | 0.288254 | 0.104053 | 605031 | 32703_at | NM_014264 analysis serine/threonine kinase 18 |
| 1570 | 0.286797 | 0.103697 | 602600 | 32398_s_at | NM_004631 analysis low density lipoprotein receptor-related protein 8 apolipoprotein e receptor |
| | | | | | NM_017522 analysis apolipoprotein E receptor 2 |
| 6790 | 0.286797 | 0.103514 | | 39607_at | NM_015458 analysis DKFZP434K171 protein |
| 489 | 0.286797 | 0.103116 | 602130 | 1637_at | NM_004635 analysis mitogen-activated protein kinase-activated protein kinase 3 |
| 2989 | 0.286651 | 0.100579 | 602919 | 34433_at | NM_001381 analysis docking protein 1 |
| 8609 | 0.285194 | 0.10045 | 142230 | 538_at | NM_001773 analysis CD34 antigen |
| 4464 | 0.281988 | 0.100437 | | 36576_at | NM_004893 analysis H2A histone family member Y |
| 7403 | 0.281988 | 0.09987 | 300151 | 40435_at | |
| 5779 | 0.280822 | 0.099596 | 603501 | 38270_at | NM_003631 analysis poly ADP-ribose glycohydrolase |
| 8670 | 0.27747 | 0.099577 | 600735 | 625_at | |
| 4693 | 0.276159 | 0.098971 | 130410 | 36881_at | NM_001985 analysis electron-transfer-flavoprotein beta polypeptide |
| 7513 | 0.276013 | 0.097783 | 136533 | 40570_at | NM_002015 analysis forkhead box O1A |
| 1004 | 0.275867 | 0.097033 | 603624 | 31527_at | NM_002952 analysis ribosomal protein S2 |
| 316 | 0.274847 | 0.09695 | 603109 | 1433_g_at | NM_005902 analysis MAD mothers against decapentaplegic Drosophila homolog 3 |
| 5308 | 0.274556 | 0.096929 | 125290 | 37674_at | NM_000688 analysis aminolevulinate delta- synthase 1 |
| 1385 | 0.27441 | 0.096861 | 602362 | 32151_at | NM_002883 analysis Ran GTPase activating protein 1 |
| 7036 | 0.27441 | 0.096558 | | 39932_at | |
| 2132 | 0.273244 | 0.09647 | | 33233_at | |
| 4100 | 0.272952 | 0.096296 | 604857 | 36060_at | NM_003136 analysis signal recognition particle 54 kD |
| 528 | 0.271349 | 0.096014 | 602520 | 1698_g_at | NM_002757 analysis mitogen-activated protein kinase kinase 5 |
| 4643 | 0.270329 | 0.096003 | 604704 | 36812_at | NM_003567 analysis breast cancer antiestrogen resistance 3 |
| 4312 | 0.270184 | 0.095026 | 138322 | 36336_s_at | NM_002085 analysis glutathione peroxidase 4 |

TABLE 10
Maximum Difference-Selected Genes (Whole Set)

| | | | Omim | | |
|---|---|---|---|---|---|
| Index | Max Diff | Avg Diff | Link | Affy Id | Description |
| 4975 | 0.383929 | 0.133728 | 300051 | 37251_s_at | |
| 6031 | 0.357143 | 0.133158 | 142200 | 38585_at | NM_000559 analysis hemoglobin gamma A |
| 4022 | 0.305332 | 0.132256 | 140555 | 35965_at | NM_002155 analysis heat shock 70 kD protein 6 HSP70B |
| 6169 | 0.30508 | 0.130643 | 600276 | 38750_at | NM_000435 analysis Notch Drosophila homolog 3 |
| 5053 | 0.295397 | 0.129113 | 147267 | 37343_at | NM_002224 analysis inositol 1 4 5-triphosphate receptor type 3 |
| 6674 | 0.290241 | 0.128926 | | 39418_at | |
| 1662 | 0.288984 | 0.125099 | 191318 | 32557_at | NM_007279 analysis U2 small nuclear ribonucleoprotein auxiliary factor 65 kD |
| 5554 | 0.27578 | 0.124241 | 126660 | 37981_at | NM_004395 analysis drebrin 1 |
| 6530 | 0.26748 | 0.120535 | 186740 | 39226_at | NM_000073 analysis CD3G gamma precursor |
| 6199 | 0.263078 | 0.119869 | 600673 | 38794_at | NM_014233 analysis upstream binding transcription factor RNA polymerase I |
| 2429 | 0.262701 | 0.118728 | 300156 | 33637_g_at | NM_001327 analysis cancer/testis antigen |
| 8479 | 0.262575 | 0.11592 | 602731 | 41819_at | NM_001465 analysis FYN-binding protein FYB-120/130 |
| 1054 | 0.261318 | 0.111653 | 156350 | 31623_f_at | |
| 8635 | 0.259557 | 0.110325 | 162096 | 577_at | NM_002391 analysis midkine neurite growth-promoting factor 2 |
| 93 | 0.259306 | 0.110118 | | 1126_s_at | |
| 2290 | 0.2583 | 0.107107 | 156491 | 33415_at | NM_002512 analysis non-metastatic cells 2 protein NM23B expressed in |
| 4464 | 0.257671 | 0.106625 | | 36576_at | NM_004893 analysis H2A histone family member Y |
| 1312 | 0.25742 | 0.105829 | | 32058_at | NM_004854 analysis HNK-1 sulfotransferase |
| 6010 | 0.256288 | 0.104053 | | 38549_at | |
| 5600 | 0.251383 | 0.103697 | 600616 | 38038_at | NM_002345 analysis lumican |
| 5919 | 0.250377 | 0.103514 | | 38437_at | NM_007359 analysis MLN51 protein |
| 4308 | 0.247611 | 0.103116 | | 36331_at | |
| 4812 | 0.244341 | 0.100579 | 153430 | 37023_at | NM_002298 analysis L-plastin |
| 2907 | 0.243587 | 0.10045 | 601798 | 34332_at | NM_005471 analysis glucosamine-6-phosphate isomerase |
| 5315 | 0.241574 | 0.100437 | 604706 | 37681_i_at | NM_018834 analysis matrin 3 |
| 5458 | 0.241071 | 0.09987 | 147120 | 37864_s_at | |
| 5820 | 0.240568 | 0.099596 | 186790 | 38319_at | NM_000732 analysis CD3D antigen delta polypeptide TiT3 complex |
| 4053 | 0.240443 | 0.099577 | 300248 | 36004_at | NM_003639 analysis inhibitor of kappa light polypeptide gene enhancer in B-cells kinase |
| | | | | | gamma |
| 2590 | 0.239185 | 0.098971 | | 33857_at | NM_016143 analysis p47 |
| 1779 | 0.238179 | 0.097783 | 605031 | 32703_at | NM_014264 analysis serine/threonine kinase 18 |
| 3498 | 0.237425 | 0.097033 | 603083 | 35201_at | NM_001533 analysis heterogeneous nuclear ribonucleoprotein L |
| 3455 | 0.236796 | 0.09695 | 603039 | 35145_at | NM_020310 analysis MAX binding protein |
| 1861 | 0.236293 | 0.096929 | 186930 | 32794_g_at | |
| 5676 | 0.236293 | 0.096861 | 110750 | 38119_at | NM_002101 analysis glycophorin C isoform 1 NM_016815 analysis glycophorin C isoform 2 |
| 702 | 0.236167 | 0.096558 | 123838 | 1923_at | NM_005190 analysis cyclin C |
| 4360 | 0.235161 | 0.09647 | | 36434_r_at | |
| 2244 | 0.234406 | 0.096296 | | 33362_at | NM_006449 analysis Cdc42 effector protein 3 |
| 7206 | 0.234406 | 0.096014 | 601062 | 40150_at | NM_004175 analysis small nuclear ribonucleoprotein D3 polypeptide 18 kD |
| 813 | 0.234029 | 0.096003 | 602867 | 2062_at | NM_001553 analysis insulin-like growth factor binding protein 7 |
| 8485 | 0.233023 | 0.095026 | | 41825_at | |

TABLE 11
Average Difference-Selected Genes (Whole Set)

| | | | Omim | | |
|---|---|---|---|---|---|
| Index | Max Diff | Avg Diff | Link | Affy Id | Description |
| 54 | 0.383929 | 0.133728 | 600210 | 106_at | NM_004350 analysis runt-related transcription factor 3 |
| 8702 | 0.357143 | 0.133158 | 182120 | 671_at | NM_003118 analysis secreted protein acidic cysteine-rich osteonectin |
| 5676 | 0.305332 | 0.132256 | 110750 | 38119_at | NM_002101 analysis glycophorin C isoform 1 NM_016815 analysis glycophorin C isoform 2 |
| 8219 | 0.30508 | 0.130643 | | 41478_at | |
| 3899 | 0.295397 | 0.129113 | | 35796_at | NM_007284 analysis protein tyrosine kinase 9-like A6-related protein |
| 6674 | 0.290241 | 0.128926 | | 39418_at | |
| 4801 | 0.288984 | 0.125099 | | 37006_at | NM_006425 analysis step II splicing factor SLU7 |
| 8799 | 0.27578 | 0.124241 | 605482 | 824_at | NM_004832 analysis glutathione-S-transferase like |
| 6327 | 0.26748 | 0.120535 | | 38971_r_at | NM_006058 analysis Nef-associated factor 1 |
| 6080 | 0.263078 | 0.119869 | | 38652_at | ****NM_017787 hypothetical protein FLJ20154 NM_017787 hypothetical protein FLJ20154 |
| 7348 | 0.262701 | 0.118728 | 139314 | 40365_at | NM_002068 analysis guanine nucleotide binding protein G protein alpha 15 Gq class |
| 8479 | 0.262575 | 0.11592 | 602731 | 41819_at | NM_001465 analysis FYN-binding protein FYB-120/130 |
| 4892 | 0.261318 | 0.111653 | 604713 | 37147_at | NM_002975 analysis stem cell growth factor lymphocyte secreted C-type lectin |
| 7693 | 0.259557 | 0.110325 | 601323 | 40817_at | NM_006184 analysis nucleobindin 1 |
| 2488 | 0.259306 | 0.110118 | 603593 | 33731_at | NM_003982 analysis solute carrier family 7 cationic amino acid transporter y system member 7 |
| 906 | 0.2583 | 0.107107 | 152390 | 307_at | NM_000698 analysis arachidonate 5-lipoxygenase |
| 6311 | 0.257671 | 0.106625 | 603109 | 38944_at | NM_005902 analysis MAD mothers against decapentaplegic Drosophila homolog 3 |
| 2097 | 0.25742 | 0.105829 | | 33188_at | NM_014337 analysis peptidylprolyl isomerase cyclophilin like 2 |
| 1779 | 0.256288 | 0.104053 | 605031 | 32703_at | NM_014264 analysis serine/threonine kinase 18 |
| 1570 | 0.251383 | 0.103697 | 602600 | 32398_s_at | NM_004631 analysis low density lipoprotein receptor-related protein 8 apolipoprotein e receptor |
| | | | | | NM_017522 analysis apolipoprotein E receptor 2 |
| 6790 | 0.250377 | 0.103514 | | 39607_at | NM_015458 analysis DKFZP434K171 protein |
| 489 | 0.247611 | 0.103116 | 602130 | 1637_at | NM_004635 analysis mitogen-activated protein kinase-activated protein kinase 3 |
| 2989 | 0.244341 | 0.100579 | 602919 | 34433_at | NM_001381 analysis docking protein 1 |
| 8609 | 0.243587 | 0.10045 | 142230 | 538_at | NM_001773 analysis CD34 antigen |
| 4464 | 0.241574 | 0.100437 | | 36576_at | NM_004893 analysis H2A histone family member Y |
| 7403 | 0.241071 | 0.09987 | 300151 | 40435_at | |
| 5779 | 0.240568 | 0.099596 | 603501 | 38270_at | NM_003631 analysis poly ADP-ribose glycohydrolase |
| 8670 | 0.240443 | 0.099577 | 600735 | 625_at | |
| 4693 | 0.239185 | 0.098971 | 130410 | 36881_at | NM_001985 analysis electron-transfer-flavoprotein beta polypeptide |
| 7513 | 0.238179 | 0.097783 | 136533 | 40570_at | NM_002015 analysis forkhead box O1A |
| 1004 | 0.237425 | 0.097033 | 603624 | 31527_at | NM_002952 analysis ribosomal protein S2 |
| 316 | 0.236796 | 0.09695 | 603109 | 1433_g_at | NM_005902 analysis MAD mothers against decapentaplegic Drosophila homolog 3 |
| 5308 | 0.236293 | 0.096929 | 125290 | 37674_at | NM_000688 analysis aminolevulinate delta- synthase 1 |
| 1385 | 0.236293 | 0.096861 | 602362 | 32151_at | NM_002883 analysis Ran GTPase activating protein 1 |
| 7036 | 0.236167 | 0.096558 | | 39932_at | |
| 2132 | 0.235161 | 0.09647 | | 33233_at | |
| 4100 | 0.234406 | 0.096296 | 604857 | 36060_at | NM_003136 analysis signal recognition particle 54 kD |
| 528 | 0.234406 | 0.096014 | 602520 | 1698_g_at | NM_002757 analysis mitogen-activated protein kinase kinase 5 |
| 4643 | 0.234029 | 0.096003 | 604704 | 36812_at | NM_003567 analysis breast cancer antiestrogen resistance 3 |
| 4312 | 0.233023 | 0.095026 | 138322 | 36336_s_at | NM_002085 analysis glutathione peroxidase 4 |

### Example V

### SVM Analysis of Pre-B ALL Cohort Data to Discriminate Between Remission and Failure and Among Various Karyotypes

We applied linear SVM, SVM with recursive feature elimination (SVM-RFE), and nonlinear SVM methods (polynomial and gaussian) to the pre B training dataset o get a list of genes associated with CCR/Fail. Table 12 shows the top 40 genes for evaluating remission from failure (CCR vs. FAIL). However, CCR vs. FAIL was nonseparable using these methods.

We also used SVM-RFE to discriminate between members of the data set who have the certain MLL translocations from those who do not. Table 13 shows the top 40 genes found to discriminate t(12;21) from not t(12;21) (we excluded patients without t(12;21) data from this analysis). Table 14 shows the top 40 genes found to discriminate t(1;19) from not t(1;19). We did not see significant separation for t(9;22), t(4;11) or hyperdiploid karyotypes.

TABLE 12
CCR vs. Fail

| | |
|---|---|
| 38086_at | NM_001542 analysis immunoglobulin superfamily member 3 |
| 38652_at | NM_017787 hypothetical protein FLJ20154 NM_017787 hypothetical protein FLJ20154 |
| 31473_s_at | NM_003747 analysis tankyrase TRF1-interacting ankyrin-related ADP-ribose polymerase |
| 36144_at | |
| 40650_r_at | NM_004382 analysis corticotropin releasing hormone receptor 1 |
| 2009_at | NM_004103 analysis protein tyrosine kinase 2 beta |
| 33914_r_at | NM_000140 analysis ferrochelatase |
| 34612_at | NM_004057 analysis calbindin 3 |
| 32072_at | NM_005823 analysis megakaryocyte potentiating factor precursor NM_013404 |
| | analysis mesothelin isoform 2 precursor |
| 625_at | |
| 33316_at | NM_014729 analysis KIAA0808 gene product |
| 38838_at | NM_005033 analysis polymyositis/scleroderma autoantigen 1 75 kD |
| 38539_at | NM_004727 analysis solute carrier family 24 sodium/potassium/calcium exchanger member 1 |
| 32503_at | |
| 32930_f_at | NM_014893 analysis KIAA0951 protein |
| 40161_at | NM_000095 analysis cartilage oligomeric matrix protein presursor |
| 38840_s_at | NM_002628 analysis profilin 2 |
| 34045_at | |
| 34770_at | NM_005204 analysis mitogen-activated protein kinase kinase kinase 8 |
| 36154_at | |
| 38155_at | NM_002553 analysis origin recognition complex subunit 5 yeast homolog like |
| 35842_at | |
| 33946_at | |
| 39213_at | NM_012261 analysis similar to S68401 cattle glucose induced gene |
| 35872_at | NM_000922 analysis phosphodiesterase 3B cGMP-inhibited |
| 38768_at | NM_005327 analysis L-3-hydroxyacyl-Coenzyme A dehydrogenase short chain |
| 32035_at | |
| 36342_r_at | NM_005666 analysis H factor complement like 3 |
| 38700_at | NM_004078 analysis cysteine and glycine-rich protein 1 |
| 38025_r_at | NM_014961 analysis KIAA0871 protein |
| 36395_at | |
| 39001_at | NM_005918 analysis malate dehydrogenase 2 NAD mitochondrial |
| 33957_at | |
| 36927_at | NM_006820 analysis hypothetical protein expressed in osteoblast |
| 40387_at | NM_001401 analysis endothelial differentiation lysophosphatidic acid G-protein-coupled receptor 2 |
| 1368_at | NM_000877 analysis interleukin 1 receptor type I |
| 32551_at | NM_004105 analysis EGF-containing fibulin-like extracellular |
| | matrix protein 1 precursor isoform a precursor NM_018894 |
| | analysis EGF-containing fibulin-like extracellular matrix protein 1 isoform b |
| 32655_s_at | NM_006696 analysis thyroid hormone receptor coactivating protein |
| 36339_at | |
| 37946_at | NM_003161 analysis serine/threonine kinase 14 alpha |

TABLE 13
T (12; 21) vs. not T(12; 21)

| | |
|---|---|
| 40272_at | NM_001313 analysis collapsin response mediator protein 1 |
| 38267_at | NM_004170 analysis solute carrier family 1 neuronal/epithelial |
| | high affinity glutamate transporter system Xag member 1 |
| 38968_at | NM_004844 analysis SH3-domain binding protein 5 BTK-associated |
| 35019_at | NM_004876 analysis zinc finger protein 254 |
| 32227_at | NM_002727 analysis proteoglycan 1 secretory granule |
| 38925_at | NM_003296 analysis testis specific protein 1 probe H4-1 p3-1 |
| 41490_at | NM_002765 analysis phosphoribosyl pyrophosphate synthetase 2 |
| 35614_at | NM_006602 analysis transcription factor-like 5 basic helix-loop-helix |
| 1211_s_at | NM_003805 analysis CASP2 and RIPK1 domain containing adaptor with death domain |
| 1708_at | NM_002753 analysis mitogen-activated protein kinase 10 |
| 39696_at | |
| 40570_at | NM_002015 analysis forkhead box O1A |
| 32778_at | NM_002222 analysis inositol 1 4 5-triphosphate receptor type 1 |
| 339_at | NM_001233 analysis caveolin 2 |
| 32163_f_at | |
| 40367_at | NM_001200 analysis bone morphogenetic protein 2 precursor |
| 37816_at | NM_001735 analysis complement component 5 |
| 35362_at | NM_012334 analysis myosin X |
| 35712_at | |
| 32730_at | |
| 599_at | NM_021958 analysis H2.0 Drosophila like homeo box 1 |
| 39827_at | NM_019058 analysis hypothetical protein |
| 1077_at | NM_000448 analysis recombination activating gene 1 |
| 36524_at | NM_015320 analysis KIAA1112 protein |
| 39931_at | NM_003582 analysis dual-specificity tyrosine- Y phosphorylation regulated kinase 3 |
| 33686_at | |
| 39786_at | |
| 31883_at | NM_002454 analysis methionine synthase reductase isoform |
| | 1 NM_024010 methionine synthase reductase isoform 2 |
| 38938_at | NM_006593 analysis T-box brain 1 |
| 41442_at | NM_005187 analysis core-binding factor runt domain alpha subunit 2 translocated to 3 |
| 755_at | NM_002222 analysis inositol 1 4 5-triphosphate receptor type 1 |
| 35288_at | NM_015185 analysis Cdc42 guanine exchange factor GEF 9 |
| 38578_at | NM_001242 analysis CD27 antigen |
| 37198_r_at | |
| 32343_at | |
| 33910_at | |
| 1089_i_at | |
| 40166_at | NM_018639 analysis CS box-containing WD protein |
| 33494_at | NM_004453 analysis electron-transferring-flavoprotein dehydrogenase |
| 41446_f_at | NM_007372 analysis RNA helicase-related protein |

TABLE 14
T(1; 19) vs. not T(1; 19)

| | |
|---|---|
| 1788_s_at | NM_001394 analysis dual specificity phosphatase 4 |
| 37680_at | NM_005100 analysis A kinase PRKA anchor protein gravin 12 |
| 362_at | NM_002744 analysis protein kinase C zeta |
| 39878_at | NM_020403 analysis cadherin superfamily protein VR4-11 |
| 38748_at | NM_001112 analysis RNA-specific adenosine |
| | deaminase B1 isoform DRADA2a NM_015833 analysis RNA-specific adenosine |
| | deaminase B1 isoform DRABA2b NM_015834 analysis RNA-specific adenosine deaminase B1 isoform DRADA2c |
| 38010_at | NM_004052 analysis BCL2/adenovirus E1B 19 kD-interacting protein 3 |
| 39614_at | |
| 539_at | NM_002958 analysis RYK receptor-like tyrosine kinase precursor |
| 583_s_at | NM_001078 analysis vascular cell adhesion molecule 1 |
| 37967_at | NM_007161 analysis lymphocyte antigen 117 |
| 37132_at | NM_014425 analysis inversin |
| 38137_at | NM_003602 analysis FK506-binding protein 6 36 kD |
| 40155_at | NM_002313 analysis actin-binding LIM protein 1 isoform a |
| | NM_006719 analysis actin-binding LIM protein 1 isoform |
| | m NM_006720 analysis actin-binding LIM protein 1 isoform s |
| 38138_at | NM_005620 analysis S100 calcium-binding protein A11 |
| 37625_at | NM_002460 analysis interferon regulatory factor 4 |
| 35938_at | |
| 35927_r_at | NM_006669 analysis leukocyte immunoglobulin-like receptor subfamily B with TM and ITIM domains member 1 |
| 36305_at | NM_001044 analysis solute carrier family 6 neurotransmitter transporter dopamine member 3 |
| 36309_at | NM_005259 analysis growth differentiation factor 8 |
| 41317_at | NM_021033 analysis RAP2A member of RAS oncogene family |
| 36086_at | NM_001239 analysis cyclin H |
| 36889_at | NM_004106 analysis Fc fragment of IgE high affinity I receptor for gamma polypeptide precursor |
| 37493_at | NM_000395 analysis colony stimulating factor 2 receptor beta low-affinity granulocyte-macrophage |
| 33513_at | NM_003037 analysis signaling lymphocytic activation molecule |
| 40454_at | NM_005245 analysis cadherin family member 7 precursor |
| 38285_at | |
| 307_at | NM_000698 analysis arachidonate 5-lipoxygenase |
| 717_at | NM_021643 analysis GS3955 protein |
| 577_at | NM_002391 analysis midkine neurite growth-promoting factor 2 |
| 37536_at | NM_004233 analysis CD83 antigen activated B lymphocytes immunoglobulin superfamily |
| 38604_at | NM_000905 analysis neuropeptide Y |
| 951_at | NM_006814 analysis proteasome inhibitor |
| 854_at | NM_001715 analysis B lymphoid tyrosine kinase |
| 31811_r_at | NM_005038 analysis peptidylprolyl isomerase D cyclophilin D |
| 39829_at | NM_005737 analysis ADP-ribosylation factor-like 7 |
| 36343_at | NM_012465 tolloid-like 2 |
| 36491_at | NM_021992 analysis thymosin beta identified in neuroblastoma cells |
| 37306_at | |
| 33328_at | |
| 35926_s_at | NM_006669 analysis leukocyte immunoglobulin-like receptor subfamily B with TM and ITIM domains member 1 |

We then performed analyses to discriminate CCR vs. FAIL conditioned on various karyotypes (t(12;21), t(1;19), t(9/22), t(4,11) and hyperdiploid (Tables 15-19). Although the results are marginal, the associated gene lists may be useful in risk classification and/or the development of therapeutic strategies.

TABLE 15
CCR/Fail Conditioned on T(12; 21)

| | |
|---|---|
| 41093_at | NM_002545 analysis opioid-binding cell adhesion molecule precursor |
| 38092_at | NM_001430 analysis endothelial PAS domain protein 1 |
| 35535_f_at | |
| 32930_f_at | NM_014893 analysis KIAA0951 protein |
| 34142_at | |
| 995_g_at | NM_002845 analysis protein tyrosine phosphatase receptor type mu polypeptide |
| 37187_at | NM_002089 analysis GRO2 oncogene |
| 942_at | NM_004683 analysis regucalcin senescence marker protein-30 |
| 37864_s_at | |
| 38227_at | NM_000248 analysis microphthalmia-associated transcription factor |
| 281_s_at | NM_000944 analysis protein phosphatase 3 formerly 2B catalytic subunit alpha isoform calcineurin A alpha |
| 38355_at | NM_004660 analysis DEAD/H Asp-Glu-Ala-Asp/His box polypeptide Y chromosome |
| 37328_at | NM_002664 analysis pleckstrin |
| 33644_at | NM_002395 analysis cytosolic malic enzyme 1 |
| 1089_i_at | |
| 417_at | NM_005400 analysis protein kinase C epsilon |
| 39474_s_at | NM_013372 analysis cysteine knot superfamily 1 BMP antagonist 1 |
| 34052_at | NM_001980 analysis epimorphin |
| 36838_at | NM_002776 analysis kallikrein 10 |
| 961_at | NM_000267 analysis neurofibromin |
| 35405_at | NM_000353 analysis tyrosine aminotransferase |
| 326_i_at | |
| 36395_at | |
| 34824_at | NM_013444 analysis ubiquilin 2 |
| 1117_at | NM_001785 analysis cytidine deaminase |
| 40000_f_at | |
| 40727_at | NM_014885 analysis anaphase-promoting complex subunit 10 |
| 33400_r_at | NM_001010 analysis ribosomal protein S6 |
| 33120_at | NM_002925 analysis regulator of G-protein signaling 10 |
| 128_at | NM_000396 analysis cathepsin K pycnodysostosis |
| 39623_at | |
| 353_at | NM_012399 analysis phosphotidylinositol transfer protein beta |
| 38627_at | NM_002126 analysis hepatic leukemia factor |
| 31541_at | |
| 34852_g_at | NM_003600 analysis serine/threonine kinase 15 |
| 39627_at | NM_003566 analysis early endosome antigen 1 162 kD |
| 1002_f_at | |
| 38938_at | NM_006593 analysis T-box brain 1 |
| 33191_at | NM_018121 analysis hypothetical protein FLJ10512 |
| 33738_r_at | |

TABLE 16
CCR/Fail on T(1; 19)

| | |
|---|---|
| 32901_s_at | NM_001550 analysis interferon-related developmental regulator 1 |
| 32018_at | |
| 32746_at | NM_003879 analysis CASP8 and FADD-like apoptosis regulator |
| 1368_at | NM_000877 analysis interleukin 1 receptor type I |
| 31992_f_at | |
| 2083_at | NM_000731 analysis cholecystokinin B receptor |
| 33466_at | |
| 36400_at | |
| 34548_at | NM_000497 analysis cytochrome P450 subfamily XIB steroid 11-beta-hydroxylase polypeptide 1 |
| 41714_at | |
| 40303_at | NM_003222 analysis transcription factor AP-2 gamma activating enhancer-binding protein 2 gamma |
| 33730_at | |
| 1800_g_at | NM_005236 analysis excision repair cross-complementing rodent repair deficiency complementation group 4 |
| 1485_at | NM_004440 analysis EphA7 |
| 36873_at | |
| 41871_at | NM_006474 analysis lung type-I cell membrane-associated glycoprotein isoform 2 precursor NM_013317 |
| | analysis lung type-I cell membrane-associated glycoprotein isoform 1 |
| 607_s_at | NM_000552 analysis von Willebrand factor precursor |
| 41385_at | NM_012307 analysis erythrocyte membrane protein band 4.1-like 3 |
| 39102_at | NM_013296 analysis LGN protein |
| 32671_at | NM_014640 analysis KIAA0173 gene product |
| 34714_at | NM_015474 analysis DKFZP564A032 protein |
| 36419_at | |
| 36595_s_at | NM_001482 analysis glycine amidinotransferase L-arginine glycine amidinotransferase |
| 38552_f_at | NM_018844 analysis B-cell receptor-associated protein BAP29 |
| 40031_at | NM_000691 analysis aldehyde dehydrogenase 3 family member A1 |
| 32035_at | |
| 41266_at | NM_000210 analysis integrin alpha chain alpha 6 |
| 1986_at | NM_005611 analysis retinoblastoma-like 2 p130 |
| 32865_at | |
| 38223_at | NM_007063 analysis vascular Rab-GAP/TBC-containing |
| 40934_at | |
| 34056_g_at | NM_004302 analysis activin A type IB receptor precursor NM_020327 analysis activin A type IB receptor |
| | isoform b precursor NM_020328 analysis activin A type IB receptor isoform c precursor |
| 1745_at | |
| 31525_s_at | |
| 1484_at | NM_001796 analysis cadherin 8 type 2 |
| 36241_r_at | NM_000151 analysis glucose-6-phosphatase catalytic |
| 34120_r_at | |
| 33662_at | |
| 35284_f_at | NM_018199 analysis hypothetical protein FLJ10738 |
| 35919_at | NM_001062 analysis transcobalamin I vitamin B12 binding protein R binder family |

TABLE 17
CCR/Fail on T(9; 22)

| | |
|---|---|
| 38299_at | NM_000600 analysis interleukin 6 interferon beta 2 |
| 41214_at | NM_001008 analysis ribosomal protein S4 Y-linked |
| 37215_at | |
| 37187_at | NM_002089 analysis GRO2 oncogene |
| 37258_at | NM_003692 analysis transmembrane protein with EGF-like and two follistatin-like domains 1 |
| 33734_at | NM_006147 analysis interferon regulatory factor 6 |
| 34661_at | |
| 38198_at | |
| 33412_at | |
| 38322_at | NM_007003 analysis JM27 protein |
| 34263_s_at | NM_006729 analysis diaphanous 2 isoform 156 NM_007309 analysis diaphanous 2 isoform 12C |
| 32257_f_at | NM_003218 analysis telomeric repeat binding factor 1 isoform 2 NM_017489 |
| | analysis telomeric repeat binding factor 1 isoform 1 |
| 34615_at | NM_000223 analysis keratin 12 |
| 1147_at | |
| 40757_at | NM_006144 analysis granzyme A precursor |
| 2008_s_at | NM_002392 analysis mouse double minute 2 human homolog of full length protein isoform NM_006878 |
| | analysis mouse double minute 2 human homolog of protein isoform MDM2a NM_006879 analysis mouse |
| | double minute 2 human homolog of protein isoform MDM2b NM_006880 |
| 1304_at | |
| 200_at | |
| 40367_at | NM_001200 analysis bone morphogenetic protein 2 precursor |
| 37441_at | NM_015929 analysis lipoyltransferase |
| 41021_s_at | NM_000408 analysis glycerol-3-phosphate dehydrogenase 2 mitochondrial |
| 1369_s_at | NM_000584 analysis interleukin 8 |
| 1113_at | NM_001200 analysis bone morphogenetic protein 2 precursor |
| 802_at | NM_005644 analysis TATA box binding protein TBP associated factor RNA polymerase II J 20 kD |
| 35716_at | NM_001056 analysis sulfotransferase family cytosolic 1C member 1 |
| 38389_at | NM_002534 analysis 2 5 oligoadenylate synthetase 1 isoform E16 NM_016816 |
| | analysis 2 5 oligoadenylate synthetase 1 isoform E18 |
| 31862_at | NM_003392 analysis wingless-type MMTV integration site family member 5A |
| 35844_at | NM_002999 analysis syndecan 4 amphiglycan ryudocan |
| 39269_at | NM_002915 analysis replication factor C activator 1 3 38 kD |
| 1953_at | NM_003376 analysis vascular endothelial growth factor |
| 34324_at | NM_006493 analysis ceroid-lipofuscinosis neuronal 5 |
| 35658_at | NM_000021 analysis presenilin 1 isoform I-467 NM_007318 analysis |
| | presenilin 1 isoform I-463 NM_007319 analysis presenilin 1 isoform I-374 |
| 38220_at | NM_000110 analysis dihydropyrimidine dehydrogenase |
| 31359_at | |
| 658_at | NM_003247 analysis thrombospondin 2 |
| 40097_at | NM_004681 analysis eukaryotic translation initiation factor 1A Y chromosome |
| 41548_at | NM_003916 analysis adaptor-related protein complex 1 sigma 2 subunit |
| 38039_at | NM_000103 analysis cytochrome P450 subfamily XIX aromatization of androgens |
| 33538_at | NM_016132 analysis myelin gene expression factor 2 |
| 36674_at | NM_002984 analysis small inducible cytokine A4 homologous to mouse Mip-1b |

TABLE 18
CCR/Fail on T(9; 22)

| | |
|---|---|
| 38299_at | NM_000600 analysis interleukin 6 interferon beta 2 |
| 41214_at | NM_001008 analysis ribosomal protein S4 Y-linked |
| 37215_at | |
| 37187_at | NM_002089 analysis GRO2 oncogene |
| 37258_at | NM_003692 analysis transmembrane protein with EGF-like and two follistatin-like domains 1 |
| 33734_at | NM_006147 analysis interferon regulatory factor 6 |
| 34661_at | |
| 38198_at | |
| 33412_at | |
| 38322_at | NM_007003 analysis JM27 protein |
| 34263_s_at | NM_006729 analysis diaphanous 2 isoform 156 NM_007309 analysis diaphanous 2 isoform 12C |
| 32257_f_at | NM_003218 analysis telomeric repeat binding factor 1 isoform 2 NM_017489 |
| | analysis telomeric repeat binding factor 1 isoform 1 |
| 34615_at | NM_000223 analysis keratin 12 |
| 1147_at | |
| 40757_at | NM_006144 analysis granzyme A precursor |
| 2008_s_at | NM_002392 analysis mouse double minute 2 human homolog of full length protein isoform NM_006878 |
| | analysis mouse double minute 2 human homolog of protein isoform MDM2a NM_006879 analysis mouse |
| | double minute 2 human homolog of protein isoform MDM2b NM_006880 |
| 1304_at | |
| 200_at | |
| 40367_at | NM_001200 analysis bone morphogenetic protein 2 precursor |
| 37441_at | NM_015929 analysis lipoyltransferase |
| 41021_s_at | NM_000408 analysis glycerol-3-phosphate dehydrogenase 2 mitochondrial |
| 1369_s_at | NM_000584 analysis interleukin 8 |
| 1113_at | NM_001200 analysis bone morphogenetic protein 2 precursor |
| 802_at | NM_005644 analysis TATA box binding protein TBP associated factor RNA polymerase II J 20 kD |
| 35716_at | NM_001056 analysis sulfotransferase family cytosolic 1C member 1 |
| 38389_at | NM_002534 analysis 2 5 oligoadenylate synthetase 1 isoform E16 NM_016816 |
| | analysis 2 5 oligoadenylate synthetase 1 isoform E18 |
| 31862_at | NM_003392 analysis wingless-type MMTV integration site family member 5A |
| 35844_at | NM_002999 analysis syndecan 4 amphiglycan ryudocan |
| 39269_at | NM_002915 analysis replication factor C activator 1 3 38 kD |
| 1953_at | NM_003376 analysis vascular endothelial growth factor |
| 34324_at | NM_006493 analysis ceroid-lipofuscinosis neuronal 5 |
| 35658_at | NM_000021 analysis presenilin 1 isoform I-467 NM_007318 |
| | analysis presenilin 1 isoform I-463 NM_007319 analysis presenilin 1 isoform I-374 |
| 38220_at | NM_000110 analysis dihydropyrimidine dehydrogenase |
| 31359_at | |
| 658_at | NM_003247 analysis thrombospondin 2 |
| 40097_at | NM_004681 analysis eukaryotic translation initiation factor 1A Y chromosome |
| 41548_at | NM_003916 analysis adaptor-related protein complex 1 sigma 2 subunit |
| 38039_at | NM_000103 analysis cytochrome P450 subfamily XIX aromatization of androgens |
| 33538_at | NM_016132 analysis myelin gene expression factor 2 |
| 36674_at | NM_002984 analysis small inducible cytokine A4 homologous to mouse Mip-1b |

TABLE 19
CCR/Fail on Hyperdiploid

| | |
|---|---|
| 38940_at | NM_020675 analysis AD024 protein |
| 39572_at | NM_021956 analysis glutamate receptor ionotropic kainate 2 |
| 31616_r_at | |
| 931_at | NM_004951 analysis Epstein-Barr virus induced |
| | gene 2 lymphocyte-specific G protein-coupled receptor |
| 40231_at | NM_005585 analysis MAD mothers against decapentaplegic Drosophila homolog 6 |
| 40260_g_at | NM_014309 analysis RNA binding motif protein 9 |
| 32636_f_at | |
| 37941_at | NM_004533 analysis myosin-binding protein C fast-type |
| 34677_f_at | |
| 157_at | NM_006115 analysis preferentially expressed antigen of melanoma |
| 32985_at | NM_002968 analysis sal Drosophila like 1 |
| 37223_at | NM_000232 analysis sarcoglycan beta 43 kD dystrophin-associated glycoprotein |
| 40545_at | NM_007198 analysis proline synthetase co-transcribed bacterial homolog |
| 39990_at | NM_002202 analysis islet-1 |
| 1758_r_at | NM_000765 analysis cytochrome P450 subfamily IIIA polypeptide 7 |
| 38354_at | NM_005194 analysis CCAAT/enhancer binding protein C/EBP beta |
| 38155_at | NM_002553 analysis origin recognition complex subunit 5 yeast homolog like |
| 33585_at | |
| 33815_at | NM_000373 analysis uridine monophosphate |
| | synthetase orotate phosphoribosyl transferase and orotidine-5 decarboxylase |
| 38150_at | NM_002451 analysis 5 methylthioadenosine phosphorylase |
| 35472_at | NM_002243 analysis potassium inwardly-rectifying channel subfamily J member 15 |
| 764_s_at | |
| 31468_f_at | |
| 39780_at | NM_021132 analysis protein phosphatase 3 formerly 2B |
| | catalytic subunit beta isoform calcineurin A beta |
| 2044_s_at | NM_000321 analysis retinoblastoma 1 including osteosarcoma |
| 38652_at | NM_017787 hypothetical protein |
| | FLJ20154 NM_017787 hypothetical protein FLJ20154 |
| 537_f_at | NM_012165 analysis f-box and WD-40 domain protein 3 |
| 41145_at | NM_014883 analysis KIAA0914 gene product |
| 35669_at | |
| 33462_at | NM_014879 analysis KIAA0001 gene product putative |
| | G-protein-coupled receptor G protein coupled |
| | receptor for UDP-glucose |
| 1375_s_at | NM_003255 analysis tissue inhibitor of metalloproteinase 2 precursor |
| 40326_at | NM_004352 analysis cerebellin 1 precursor |
| 32368_at | NM_002590 analysis protocadherin 8 |
| 35014_at | |
| 38772_at | NM_001554 analysis cysteine-rich angiogenic inducer 61 |
| 32434_at | NM_002356 analysis myristoylated alanine-rich protein kinase C substrate |
| 1609_g_at | |
| 1648_at | NM_003999 analysis oncostatin M receptor |
| 35173_at | |
| 36693_at | NM_001990 analysis eyes absent Drosophila homolog 3 |

### Example VI

### Application of ANOVA to VxInsight Clusters to Identify Genes Associated with Outcome

To identify genes strongly predictive of outcome in pediatric ALL, we divided the retrospective POG ALL case control cohort (n=254) described above into training (⅔ of cases) and test (⅓ of cases) sets performed statistical analyses using VxInsight and ANOVA. Through this approach, we identified a limited set of novel genes that were predictive of outcome in pediatric ALL. Table 20 provides the list of the top 20 genes associated with remission vs. failure in the pre-B ALL cohort; several of these genes appear to reach statistical significance. These top 20 genes are ranked by ANOVA f statistics; we have also converted these f statistics to corresponding p values. Not surprisingly, overall p values for outcome prediction in VxInsight or with any other method are less than for prediction of genetic types or morphologic labels; we assume that this is due to the significant biologic heterogeneity of the outcome variable in our patient cohorts. A positive value in the “Contrast” column of Table 20 reveals that the gene identified is expressed at relatively higher levels in patients in long term remission; a negative value indicates that a particular gene is expressed at lower levels in patients in remission and at higher levels in patients who fail therapy.

TABLE 20
Genes Statistically Distinguishing R mission vs. Fail: VxInsight

| Order | ANOVA_F | nsiORF | Contrast | p | Description |
|---|---|---|---|---|---|
| 1 | 26.58 | 39418_at | −2279.06 | p <= 0.024 | DKFZP564M182 |
| | | | | | protein |
| 2 | 18.95 | 37981_at | 2461.77 | p <= 0.046 | drebrin 1 |
| 3 | 18.87 | 38971_r_at | −1874.42 | p <= 0.057 | Nef-associated factor 1 |
| 4 | 18.82 | 38119_at | −2515.9 | p <= 0.074 | glycophorin C isoform 2 |
| 5 | 17.18 | 671_at | −1340.48 | p <= 0.068 | secreted protein acidic |
| | | | | | cysteine-rich osteonectin |
| 6 | 16.74 | 577_at | 3653.53 | p <= 0.125 | midkine neurite growth- |
| | | | | | promoting factor 2 |
| 7 | 16.05 | 37343_at | 3009.04 | p <= 0.122 | inositol 1 4 5- |
| | | | | | triphosphate receptor |
| | | | | | type 3 |
| 8 | 14.37 | 1126_s_at | −2870.22 | p <= 0.177 | Human cell surface |
| | | | | | glycoprotein CD 44 gene, |
| | | | | | 3′ end of long tailed |
| | | | | | isoform |
| 9 | 14.33 | 32970_f_at | 1440.29 | p <= 0.127 | hyaluronan binding |
| | | | | | protein |
| 10 | 13.83 | 41185_f_at | 1446.05 | p <= 0.190 | SMT3 suppressor of mif |
| | | | | | two 3 yeast homolog 2 |
| 11 | 13.78 | 33362_at | −1537.08 | p <= 0.175 | Cdc42 effector protein 3 |
| 12 | 13.74 | 38652_at | 1811.99 | p <= 0.029 | NM_017787 hypothetical |
| | | | | | protein FLJ20154 |
| | | | | | NM_017787 hypothetical |
| | | | | | protein FLJ20154 |
| 13 | 13.31 | 824_at | −2173.7 | p <= 0.160 | glutathione-S- |
| | | | | | transferase like |
| 14 | 13.28 | 35796_at | −1815.29 | p <= 0.243 | protein tyrosine kinase |
| | | | | | 9-like A6-related protein |
| 15 | 13.06 | 40523_at | 1523.7 | P <= 0.178 | hepatocyte nuclear |
| | | | | | factor 3 beta |
| 16 | 13.06 | 37184_at | −2181.49 | p <= 0.151 | syntaxin 1A brain |
| 17 | 13.04 | 34890_at | −1087.46 | p <= 0.195 | ATPase H transporting |
| | | | | | lysosomal vacuolar |
| | | | | | proton pump alpha |
| | | | | | polypeptide 70 kD |
| | | | | | isoform 1 |
| 18 | 12.94 | 41257_at | −1030.55 | p <= 0.155 | calpastatin |
| 19 | 12.86 | 41819_at | 1020.59 | p <= 0.264 | FYN-binding protein |
| | | | | | FYB-120/130 |
| 20 | 12.71 | 32058_at | 1413.3 | p <= 0.214 | HNK-1 sulfotransferase |

Interestingly, OPAL1/G0 (38652_at; NM-Hypothetical protein FLJ20154); see Example II), at position 12 on the table, appeared on gene lists produced by four different supervised learning algorithms (Bayesian networks, SVM, Neurofuzzy logic) and was ranked extremely high (top 5 or 10 genes) or at the top (Bayesian) with each of these very distinct modeling approaches. The degree of overlap between outcome genes detected with these different modeling algorithms was quite striking.

The gene at the number 5 position on the table (Affy number 671_at, known as SPARC, secreted protein, acidic, cysteine-rich (osteonectin)) is interesting as a possible therapeutic target. Osteonectin is involved in development, remodeling, cell turnover and tissue repair. Because its principal functions in vitro seem to be involved in counteradhesion and antiproliferation (Yan et al., J. Histochem. Cytochemi. 47(12):1495-1505, 1999). These characteristics may be consistent with certain mechanisms of metastasis. Further, it appears to have a role in cell cycle regulation, which, again, may be important in cancer mechanisms. Furthermore, it should be noted that other significant (about p<0.10) genes on the list might also have mechanisms that, together, could be combined to suggest mechanisms consistent with the observed differences in CCR and FAILURE. The group of genes, or subsets of it, may have more explanatory power than any individual member alone.

### Example VII

### Genes that Distinguish Karyotype Identified by Bayesian Methods

In the context of disease karyotype subtype prediction, we applied Bayesian nets to the preB training set data in a supervised learning environment. A set of training data, labeled with disease karyotype subtype, is used to generate and evaluate hypotheses against the test data. The Bayesian net approach filters the space of all genes down to K (typically, K bewteen 20 and 50) genes selected by one of several evaluation criteria based on the genes' potential information content. For each classification task attempted, a cross validation methodology is employed to determine for what value of K, and for which of the candidate evaluation criteria, the best Bayesian net classification accuracy is observed in cross validation. Surviving hypotheses are blended in the Bayesian framework, yielding conditional outcome distributions. Hypotheses so learned are validated against an out-of-sample test set in order to assess generalization accuracy.

Approximately 30 genes from prediction of each karyotype were combined. The gene list in Table 21 can discriminate translocations of t(12;21), t(1;19), t(4;11), t(9;22) as well as hyperdiploid and hypodiploid karyotype from normal karyotype.

TABLE 21
Genes for karyotype distinction derived from Bayesian
Analysis of pediatric ALL microarray samples

| Affymetrix ID | Gene description |
|---|---|
| 35362_at | hg01449 cDNA clone for KIAA0799 has a 1204-bp insertion at position |
| | 373 of the sequence of KIAA0799. |
| 1325_at | Sma and Mad homolog |
| 1077_at | recombination activating protein |
| 34194_at | Source: Homo sapiens mRNA; cDNA DKFZp564B076 (from clone |
| | DKFZp564B076). |
| 32730_at | Source: Homo sapiens mRNA; cDNA DKFZp564H142 (from clone |
| | DKFZp564H142). |
| 34745_at | Source: Homo sapiens clone 24473 mRNA sequence. |
| 37986_at | Source: Human erythropoietin receptor mRNA, complete cds. |
| 40570_at | Source: Homo sapiens forkhead protein (FKHR) mRNA, complete cds. |
| 40272_at | Source: Homo sapiens mRNA for dihydropyrimidinase related protein- |
| | 1, complete cds. |
| 2036_s_at | Source: Human cell adhesion molecule (CD44) mRNA, complete cds. |
| 35940_at | Source: H. sapiens mRNA for RDC-1 POU domain containing protein. |
| 41097_at | telomeric protein |
| 39931_at | dual specificity protein kinase |
| 31472_s_at | hyaluronan-binding protein; soluble isoform CD44RC; alternatively |
| | spliced |
| 32227_at | hematopoetic proteoglycan core protein (AA 1-158) |
| 37280_at | Mad homolog |
| 36524_at | hj05505 cDNA clone for KIAA1112 has 983-bp and 352-bp insertions |
| | at the positions 820 and 1408 of the sequence of KIAA1112. |
| 39824_at | Source: tg16b02.x1 NCI_CGAP_CLL1 Homo sapiens cDNA clone |
| | IMAGE: 2108907 3′, mRNA sequence. |
| 35260_at | Source: Homo sapiens mRNA for KIAA0867 protein, complete cds. |
| 35614_at | Source: Homo sapiens TCFL5 mRNA for transcription factor-like 5, |
| | complete cds. |
| 37497_at | orphan homeobox gene |
| 41814_at | alpha-L-fucosidase precursor (EC 3.2.1.5) |
| 1980_s_at | Source: H. sapiens RNA for nm23-H2 gene. |
| 36008_at | potentially prenylated protein tyrosine phosphatase |
| 36638_at | Source: H. sapiens mRNA for connective tissue growth factor. |
| 40367_at | bone morphogenetic protein 2A |
| 32163_f_at | Source: zq95f07.s1 Stratagene NT2 neuronal precursor 937230 Homo |
| | sapiens cDNA clone IMAGE: 649765 3′ similar to contains LTR7.b3 |
| | LTR7 repetitive element;, mRNA sequence. |
| 755_at | Source: Human mRNA for type 1 inositol 1,4,5-trisphosphate receptor, |
| | complete cds. |
| 32724_at | Refsum disease gene |
| 39327_at | similar to D. melanogaster peroxidasin(U11052) |
| 39717_g_at | Source: tn15f08.x1 NCI_CGAP_Brn25 Homo sapiens cDNA clone |
| | IMAGE: 2167719 3′, mRNA sequence. |
| 33412_at | Source: vicpro2.D07.r conorm Homo sapiens cDNA 5′, mRNA |
| | sequence. |
| 40763_at | TALE homeobox protein |
| 31575_f_at | beta-galactoside-binding lectin |
| 1039_s_at | basic helix-loop-helix transcription factor |
| 36873_at | Source: Human gene for very low density lipoprotein receptor, exon |
| | 19. |
| 1914_at | Source: Human cyclin A1 mRNA, complete cds. |
| 32529_at | Source: H. sapiens p63 mRNA for transmembrane protein. |
| 32977_at | Source: Human placenta (Diff48) mRNA, complete cds. |
| 37724_at | c-myc oncogene |
| 39338_at | Source: qf71b11.x1 Soares_testis_NHT Homo sapiens cDNA clone |
| | IMAGE: 1755453 3′ similar to gb: M38591 CALPACTIN I LIGHT CHAIN |
| | (HUMAN);, mRNA sequence. |
| 1973_s_at | c-myc oncogene |
| 31444_s_at | Source: Human lipocortin (LIP) 2 pseudogene mRNA, complete cds- |
| | like region. |
| 36897_at | Source: Homo sapiens mRNA for KIAA0027 protein, partial cds. |
| 34210_at | Source: zb11b10.s1 Soares_fetal_lung_NbHL19W Homo sapiens |
| | cDNA clone IMAGE: 301723 3′ similar to gb: X62466 H. sapiens mRNA |
| | for CAMPATH-1 (HUMAN);, mRNA sequence. |
| 266_s_at | Source: Homo sapiens CD24 signal transducer mRNA, complete cds |
| | and 3′ region. |
| 769_s_at | Source: Homo sapiens mRNA for lipocortin II, complete cds. |
| 36536_at | Source: Homo sapiens clone 24732 unknown mRNA, partial cds. |
| 38413_at | Source: Human mRNA for DAD-1, complete cds. |
| 41170_at | Source: Homo sapiens mRNA for KIAA0663 protein, complete cds. |
| 37680_at | kinase scaffold protein |
| 38518_at | Source: Homo sapiens mRNA for SCML2 protein. |
| 36514_at | Source: Human cell growth regulator CGR19 mRNA, complete cds. |
| 40396_at | ionotropic ATP receptor |
| 40417_at | KIAA0098 is a human counterpart of mouse chaperonin containing |
| | TCP-1 gene. Start codon is not identified. ha01413 cDNA clone for |
| | KIAA0098 has a 2-bp insertion between 736-737 of the sequence of |
| | KIAA0098. |
| 486_at | prodomain of this protease is similar to the CED-3 prodomain; |
| | proMch6 is a new member of the aspartate-specific cysteine protease |
| | family |
| 32232_at | Source: Homo sapiens NADH-ubiquinone oxidoreductase subunit CI- |
| | SGDH mRNA, complete cds. |
| 33355_at | Source: Homo sapiens mRNA; cDNA DKFZp586J2118 (from clone |
| | DKFZp586J2118). |
| 36203_at | Source: Human gene for ornithine decarboxylase ODC (EC 4.1.1.17). |
| 37306_at | ha1025 is new |
| 1081_at | ornithine decarboxylase |
| 40454_at | Source: H. sapiens mRNA for hFat protein. |
| 1616_at | Source: Human mRNA for FGF-9, complete cds. |
| 36452_at | Source: Homo sapiens mRNA for KIAA1029 protein, complete cds. |
| 35727_at | Source: qj64d06.x1 NCI_CGAP_Kid3 Homo sapiens cDNA clone |
| | IMAGE: 1864235 3′ similar to WP: F19B6.1 CE05666 URIDINE KINASE;, |
| | mRNA sequence. |
| 753_at | Source: Homo sapiens mRNA for osteonidogen, complete cds. |
| 32063_at | Source: H. sapiens PBX1a and PBX1b mRNA, complete cds. |
| 1797_at | CDK inhibitor p19 |
| 362_at | Source: H. sapiens mRNA for protein kinase C zeta. |
| 39829_at | Source: Homo sapiens mRNA for ADP ribosylation factor-like protein, |
| | complete cds. |
| 717_at | Source: Homo sapiens mRNA for GS3955, complete cds. |
| 854_at | protein tyrosine kinase |
| 38285_at | Source: Homo sapiens mu-crystallin gene, exon 8 and complete cds. |
| 41138_at | Source: Human MIC2 mRNA, complete cds. |
| 40113_at | Source: Homo sapiens mRNA for GS3955, complete cds. |
| 36069_at | Source: Homo sapiens mRNA for KIAA0456 protein, partial cds. |
| 37579_at | inducible protein |
| 37225_at | similar to ankyrin of Chromatium vinosum. |
| 39614_at | hh01783 cDNA clone for KIAA0802 has a 152-bp insertion at position |
| | 2490 of the sequence of KIAA0802. |
| 38748_at | alternatively spliced |
| 33513_at | Source: Human signaling lymphocytic activation molecule (SLAM) |
| | mRNA, complete cds. |
| 39729_at | Source: Human natural killer cell enhancing factor (NKEFB) mRNA, |
| | complete cds. |
| 37493_at | Source: yj49e08.r1 Soares placenta Nb2HP Homo sapiens cDNA |
| | clone IMAGE: 152102 5′, mRNA sequence. |
| 1788_s_at | MAP kinase phosphatase |
| 39929_at | Source: Homo sapiens mRNA for KIAA0922 protein, partial cds. |
| 37701_at | also called RGS2 |
| 34335_at | Source: wi81c01.x1 NCI_CGAP_Kid12 Homo sapiens cDNA clone |
| | IMAGE: 2399712 3′, mRNA sequence. |
| 1636_g_at | ABL is the cellular homolog proto-oncogene of Abelson's murine |
| | leukemia virus and is associated with the t9: 22 chromosomal |
| | translocation with the BCR gene in chronic myelogenous and acute |
| | lymphoblastic leukemia; alternative splicing using exon 1a |
| 39730_at | p150 protein (AA 1-1130) |
| 37006_at | Source: wf23c07.x1 Soares_Dieckgraefe_colon_NHUC Homo sapiens |
| | cDNA clone IMAGE: 2351436 3′, mRNA sequence. |
| 33131_at | Source: H. sapiens mRNA for SOX-4 protein. |
| 36031_at | Source: Homo sapiens mRNA for p33, complete cds. |
| 38968_at | This protein preferentially associates with activated form of Btk(Sab). |
| 40202_at | three-times repeated zinc finger motif |
| 38119_at | Source: Human mRNA for erythrocyte membrane sialoglycoprotein |
| | beta (glycophorin C). |
| 36601_at | vinculin |
| 32260_at | Source: H. sapiens mRNA for major astrocytic phosphoprotein PEA-15. |
| 34550_at | Source: Human mRNA for D-1 dopamine receptor. |
| 37399_at | Source: Human mRNA for KIAA0119 gene, complete cds. |
| 38994_at | similar to product encoded by GenBank Accession Number AB004903 |
| 1583_at | Source: Human tumor necrosis factor receptor mRNA, complete cds. |
| 1461_at | Source: Homo sapiens MAD-3 mRNA encoding IkB-like activity, |
| | complete cds. |
| 33885_at | Source: Homo sapiens mRNA for KIAA0907 protein, complete cds. |
| 34889_at | Source: zk81f02.s1 Soares_pregnant_uterus_NbHPU Homo sapiens |
| | cDNA clone IMAGE: 489243 3′, mRNA sequence. |
| 40790_at | basic helix-loop-helix protein |
| 38276_at | Source: Human I kappa B epsilon (lkBe) mRNA, complete cds. |
| 36543_at | tissue factor versions 1 and 2 precursor |
| 36591_at | Source: Human HALPHA44 gene for alpha-tubulin, exons 1-3. |
| 37600_at | Source: Human extracellular matrix protein 1 mRNA, complete cds. |
| | 675_at interferon-inducible protein 9-27 |
| 1295_at | putative |
| 37732_at | Source: Homo sapiens mRNA; cDNA DKFZp564E1922 (from clone |
| | DKFZp564E1922). |
| 669_s_at | Source: Homo sapiens interferon regulatory factor 1 gene, complete |
| | cds. |
| 38313_at | Source: Homo sapiens mRNA for KIAA1062 protein, partial cds. |
| 35256_at | Source: Homo sapiens mRNA; cDNA DKFZp434F152 (from clone |
| | DKFZp434F152). |
| 35688_g_at | Source: H. sapiens MTCP1 gene, exons 2A to 7 (and joined mRNA). |
| 32139_at | Source: H. sapiens mRNA for ZNF185 gene. |
| 40296_at | match: proteins O43895 Q95333 Q07825 O15250 O54975 |
| 149_at | DEAD-box family member; contains DECD-box; similar to rat liver |
| | nuclear protein p47 (PIR Accession Number A42881) and D. |
| | melanogaster DEAD-box RNA helicase WM6 (PIR Accession Number |
| | S51601) |
| 32251_at | Source: zl25h05.s1 Soares_pregnant_uterus_NbHPU Homo sapiens |
| | cDNA clone IMAGE: 503001 3′, mRNA sequence. |
| 37014_at | p78 protein |
| 1272_at | Source: Human translation initiation factor elF-2 gamma subunit |
| | mRNA, complete cds. |
| 40771_at | match: proteins: Sw: P26038 Tr: O35763 Sw: P26041 Sw: P26042 |
| | Sw: P26044 Sw: P35241 Sw: P26043 Sw: P15311 Sw: P31976 |
| | Sw: P26040 Tr: Q26520 Tr: Q24788 Tr: Q24796 Tr: Q94815 |
| 32941_at | Source: Homo sapiens DNA-binding protein mRNA, complete cds. |
| 37001_at | Ca2-activated |
| 37421_f_at | Source: Human DNA sequence from clone RP3-377H14 on |
| | chromosome 6p21.32-22.1, complete sequence. |
| 39755_at | match: proteins: Sw: P17861 Tr: O35426 |
| 33936_at | Source: Homo sapiens DNA for galactocerebrosidase, exon 17 and |
| | complete cds. |
| 40370_f_at | Source: Human lymphocyte antigen (HLA-G1) mRNA, complete cds. |
| 32788_at | This giant protein comprises an amino-terminal 700-residue leucine- |
| | rich region, four RanBP1-homologous domains, eight zinc-finger motifs |
| | similar to those of NUP153 and a carboxy terminus with high homology |
| | to cyclophilin. |
| 34990_at | isolated by yeast two-hybrid screening |
| 36927_at | The submitters designated this product as GS3686 |
| 2031_s_at | Source: Human wild-type p53 activated fragment-1 (WAF1) mRNA, |
| | complete cds. |
| 40518_at | precursor polypeptide (AA −23 to 1120) |
| 38336_at | hj06791 cDNA clone for KIAA1013 has a 4-bp deletion at position |
| | between 1855 and 1860 of the sequence of KIAA1013. |
| 39059_at | D7SR |
| 547_s_at | NGF1-B/nur77 beta-type transcription factor homolog |
| 36048_at | Source: Homo sapiens HRIHFB2436 mRNA, partial cds. |
| 33061_at | Source: Homo sapiens C16orf3 large protein mRNA, complete cds. |
| 40712_at | CD156; ADAM8; MS2 |
| 39290_f_at | Source: 44c1 Human retina cDNA randomly primed sublibrary Homo |
| | sapiens cDNA, mRNA sequence. |
| 35408_i_at | Source: Human mRNA for zinc finger protein (clone 431). |
| 36103_at | Source: Homo sapiens gene for LD78 alpha precursor, complete cds. |

### Example VIII

### Disciminant Analysis of Pre-B ALL Cohort Data to Discriminate Between Remission and Failure and Among Various Karyotypes

Classification Tasks and the Class Labels

We used supervised learning methods to discriminate between positive and negative outcomes (Remission (CCR) vs. Failure) and to discriminate among various karyotypes. The outcome statistics for the 167 member “training set” derived from the 254 member pre-B ALL cohort are shown in Table 22.

TABLE 22
Class Labels for Outcome Prediction

| Label | Class Name | # of Samples in the Class |
|---|---|---|
| 1 | CCR | 73 |
| 2 | Failure | 94 |

To discriminate among the various karyotypes, we considered three different classifications of the karyotypes (Table 23).

TABLE 23
Class Labels for Karyotype Discrimination

| | | Class | # of Samples |
|---|---|---|---|
| No. | Karyotype | Labels | in the Class |
| 1 | T(12; 21) | 1 | 24 |
| 2 | T(4; 11) | 2 | 14 |
| 3 | T(1; 19) | 3 | 21 |
| 4 | T(9; 22) | 4 | 10 |
| 5 | Hyperdiploid | 5 | 17 |
| 6 | Hypodiploid | 4 | 2 |
| 7 | Normal | 6 | 65 |
| 8 | Unknown | 7 | 14 |

Data Preprocessing

The analysis was performed on the data set comprising the 167 training cases. We first eliminated the 54 of 67 control genes (those with accession ID starting with the AFFX prefix), and then eliminated those genes with all calls “Absent” for all 167 training cases. With these genes removed from the original 12625, we were left with 8582 genes. In addition, a natural log transformation was performed on 8582×167 matrix of the gene expression values prior to further analysis.

Ranking Genes

The 8582 genes are ranked by two methods based on ANOVA for each classification exercise. Method 1 ranks the genes in terms of the F-test statistic values. Method 2 assigns a rank to each gene in terms of the number of pairs of classes between which the gene's expression value differs significantly. Note that for binary classification problem (remission vs. failure), only Method 1 is applicable.

Discriminating Among the Classes

An optimal subset of prediction genes is further selected from top 200 genes of a given ranked gene list through the use of stepwise discriminant analysis. Then the classes are discriminated using the linear discriminant analysis. The classification error rate is estimated through the leave-one-out cross validation (LOOCV) procedure. A visualization of the class separation for each classification is produced with canonical discriminant analysis.

Discrimination Between Remission and Failure

The one way ANOVA (F-test, which is equivalent to two-sample t-test in this case) was performed for each of 8582 pre-selected genes and then the all these genes were ranked in terms of the p-value of F-test. The numbers of 0.05 and 0.01 significant discriminating genes are 493 and 108, respectively. The top 20 significant discriminating genes are tabulated in Table 24. An optimal subset of discriminating genes were selected from the top 200 genes using the stepwise discriminant analysis was also prepared. The number one significant prediction gene in both the ranked gene list and the optimal subset of prediction genes is 38652_at, hypothetical protein FLJ20154, corresponding to OPAL1/G0.

The optimal subset of discriminating genes was utilized with linear discriminant analysis to predict for Remission (CCR) vs. failure in the training set of 167 cases. The success rate of the predictor is estimated in three ways: Resubstitution, LOOCV with Fold Independent prediction genes, LOOCV with Fold dependent prediction genes, and the results are listed in Table 25.

TABLE 24
Top significant discriminating genes for Remission vs. Failure

| Rank | Stepwise | F | p-value | Probe Set | Probe Set Description |
|---|---|---|---|---|---|
| 1 | 1 | 22.8448 | 0.00000 | 38652_at | hypothetical protein FLJ20154 |
| 2 | 1 | 16.1718 | 0.00009 | 38119_at | glycophorin C (Gerbich blood group) |
| 3 | 0 | 14.9168 | 0.00016 | 39418_at | DKFZP564M182 protein |
| 4 | 0 | 14.5669 | 0.00019 | 671_at | secreted protein, acidic, cysteine-rich (osteonectin) |
| 5 | 0 | 13.8615 | 0.00027 | 41478_at | Homo sapiens cDNA FLJ30991 fis, clone HLUNG1000041 |
| 6 | 0 | 13.1511 | 0.00038 | 35796_at | protein tyrosine kinase 9-like (A6-related protein) |
| 7 | 0 | 12.8494 | 0.00044 | 38270_at | poly (ADP-ribose) glycohydrolase |
| 8 | 0 | 12.6702 | 0.00049 | 587_at | endothelial differentiation, sphingolipid G-protein-coupled receptor, 1 |
| 9 | 0 | 12.1639 | 0.00062 | 38971_r_at | Nef-associated factor 1 |
| 10 | 0 | 11.6172 | 0.00082 | 34760_at | KIAA0022 gene product |
| 11 | 0 | 11.3141 | 0.00096 | 31527_at | ribosomal protein S2 |
| 12 | 0 | 11.2706 | 0.00098 | 37674_at | Aminolevulinate, delta-, synthase 1 |
| 13 | 0 | 10.5358 | 0.00142 | 36144_at | KIAA0080 protein |
| 14 | 1 | 10.3798 | 0.00154 | 36154_at | KIAA0263 gene product |
| 15 | 0 | 10.3236 | 0.00158 | 1126_s_at | Homo sapiens CD44 isoform RC (CD44) mRNA, complete cds |
| 16 | 1 | 10.3063 | 0.00159 | 31695_g_at | regulatory solute carrier protein, family 1, member 1 |
| 17 | 0 | 10.1814 | 0.00170 | 36927_at | hypothetical protein, expressed in osteoblast |
| 18 | 0 | 10.1600 | 0.00172 | 34965_at | cystatin F (leukocystatin) |
| 19 | 0 | 10.1129 | 0.00176 | 32336_at | aldolase A, fructose-bisphosphate |
| 20 | 0 | 10.0426 | 0.00182 | 625_at | membrane protein of cholinergic synaptic vesicles |

Note:
stepwise = 1 means that the gene belongs to the optimal subset of prediction genes.

TABLE 25
Estimate for Prediction Success Rate

| | # of | Overall |
|---|---|---|
| Method | Misclassifications | Success Rate |
| Resubstitution | 3 | 0.9820 |
| LOOCV with fold | 8 | 0.9521 |
| independent prediction genes | | |
| LOOCV with fold dependent | 43 | 0.7425 |
| prediction genes | | |

Discrimination Among various Karyotypes

The one way ANOVA (F-test) and the pair-wise comparison t-test were performed for each of 8582 pre-selected genes for the karyotype classification problem. Next, all genes were ranked based on the two methods described for outcome discrimination. The top 20 genes in each of ranked gene lists are listed in Tables 26 and 27. The tables also list the values of the statistic F and the number of pairs of classes between which the gene expression value differs at confidence level α=0.10, which is labeled as SIG#. An optimal subset of discriminating genes for each of the classes was selected from the top 200 genes with the stepwise discriminant analysis.

Each optimal subset of discriminating genes was utilized with linear discriminant analysis to predict for the corresponding classes in the training set of 167 cases. The success rate of the predictor is estimated in the same way as described in above for outcome prediction and the results are listed in Table 28.

TABLE 26
Top significant discriminating genes for karyotype.
Genes selected by Method 1

| Rank | Stepwise | F | p-value | Sig # | Probe Set | Probe Set Description |
|---|---|---|---|---|---|---|
| 1 | 1 | 25.8207 | 0.00000 | 8 | 33355_at | Homo sapiens mRNA; cDNA |
| | | | | | | DKFZp586J2118 (from clone |
| | | | | | | DKFZp586J2118) |
| 2 | 1 | 22.6173 | 0.00000 | 6 | 36452_at | synaptopodin |
| 3 | 1 | 20.7497 | 0.00000 | 11 | 40272_at | collapsin response mediator |
| | | | | | | protein 1 |
| 4 | 1 | 20.5471 | 0.00000 | 13 | 34335_at | ephrin-B2 |
| 5 | 0 | 20.1257 | 0.00000 | 9 | 32063_at | pre-B-cell leukemia transcription |
| | | | | | | factor 1 |
| 6 | 0 | 18.1686 | 0.00000 | 10 | 38285_at | crystallin, mu |
| 7 | 0 | 17.4124 | 0.00000 | 14 | 1325_at | MAD (mothers against |
| | | | | | | decapentaplegic, Drosophila) |
| | | | | | | homolog 1 |
| 8 | 0 | 16.4965 | 0.00000 | 9 | 41097_at | telomeric repeat binding factor 2 |
| 9 | 0 | 16.1843 | 0.00000 | 15 | 37280_at | MAD (mothers against |
| | | | | | | decapentaplegic, Drosophila) |
| | | | | | | homolog 1 |
| 10 | 0 | 15.8108 | 0.00000 | 6 | 35362_at | myosin X |
| 11 | 1 | 15.7074 | 0.00000 | 15 | 33412_at | lectin, galactoside-binding, |
| | | | | | | soluble, 1 (galectin 1) |
| 12 | 0 | 15.4828 | 0.00000 | 14 | 35940_at | POU domain, class 4, |
| | | | | | | transcription factor 1 |
| 13 | 1 | 15.0498 | 0.00000 | 11 | 1081_at | ornithine decarboxylase 1 |
| 14 | 0 | 14.3251 | 0.00000 | 12 | 717_at | GS3955 protein |
| 15 | 1 | 14.2303 | 0.00000 | 16 | 40570_at | forkhead box O1A |
| | | | | | | (rhabdomyosarcoma) |
| 16 | 0 | 14.0783 | 0.00000 | 14 | 32977_at | chromosome 6 open reading |
| | | | | | | frame 32 |
| 17 | 0 | 14.0752 | 0.00000 | 15 | 37680_at | A kinase (PRKA) anchor protein |
| | | | | | | (gravin) 12 |
| 18 | 0 | 13.9742 | 0.00000 | 12 | 854_at | B lymphoid tyrosine kinase |
| 19 | 0 | 13.8677 | 0.00000 | 6 | 1077_at | recombination activating gene 1 |
| 20 | 0 | 13.7766 | 0.00000 | 17 | 37343_at | inositol 1,4,5-triphosphate |
| | | | | | | receptor, type 3 |

TABLE 27
Top significant discriminating genes karyotype
Genes selected by Method 2

| | Step- | | | | | |
|---|---|---|---|---|---|---|
| Rank | wise | F | p-value | Sig # | Probe Set | Probe Set Description |
| 1 | 0 | 13.7766 | 0.00000 | 17 | 37343_at | inositol 1,4,5-triphosphate |
| | | | | | | receptor, type 3 |
| 2 | 0 | 13.4313 | 0.00000 | 17 | 182_at | inositol 1,4,5-triphosphate |
| | | | | | | receptor, type 3 |
| 3 | 1 | 13.0765 | 0.00000 | 17 | 37539_at | RalGDS-like gene |
| 4 | 0 | 14.2303 | 0.00000 | 16 | 40570_at | forkhead box O1A |
| | | | | | | (rhabdomyosarcoma) |
| 5 | 1 | 13.0270 | 0.00000 | 16 | 307_at | arachidonate 5-lipoxygenase |
| 6 | 0 | 12.9726 | 0.00000 | 16 | 38340_at | huntingtin interacting protein- |
| | | | | | | 1-related |
| 7 | 0 | 12.7724 | 0.00000 | 16 | 32827_at | related RAS viral (r-ras) |
| | | | | | | oncogene homolog 2 |
| 8 | 0 | 11.6961 | 0.00000 | 16 | 36536_at | schwannomin-interacting |
| | | | | | | protein 1 |
| 9 | 0 | 11.4521 | 0.00000 | 16 | 32554_s_at | transducin (beta)-like 1 |
| 10 | 0 | 10.1963 | 0.00000 | 16 | 36650_at | cyclin D2 |
| 11 | 0 | 10.1845 | 0.00000 | 16 | 38968_at | SH3-domain binding protein 5 |
| | | | | | | (BTK-associated) |
| 12 | 0 | 10.0070 | 0.00000 | 16 | 38518_at | sex comb on midleg |
| | | | | | | (Drosophila)-like 2 |
| 13 | 0 | 8.6339 | 0.00000 | 16 | 37981_at | drebrin 1 |
| 14 | 0 | 7.6949 | 0.00000 | 16 | 35794_at | KIAA0942 protein |
| 15 | 0 | 16.1843 | 0.00000 | 15 | 37280_at | MAD (mothers against |
| | | | | | | decapentaplegic, Drosophila) |
| | | | | | | homolog 1 |
| 16 | 1 | 15.7074 | 0.00000 | 15 | 33412_at | lectin, galactoside-binding, |
| | | | | | | soluble, 1 (galectin 1) |
| 17 | 0 | 14.0752 | 0.00000 | 15 | 37680_at | A kinase (PRKA) anchor |
| | | | | | | protein (gravin) 12 |
| 18 | 0 | 12.8180 | 0.00000 | 15 | 675_at | interferon induced |
| | | | | | | transmembrane protein 1 (9-27) |
| 19 | 0 | 11.9668 | 0.00000 | 15 | 39929_at | KIAA0922 protein |
| 20 | 1 | 11.4160 | 0.00000 | 15 | 38748_at | adenosine deaminase, RNA- |
| | | | | | | specific, B1 (homolog of rat |
| | | | | | | RED1) |

TABLE 28
Estimates of Prediction Success Rates for
Karyotype Discrimination

| | Estimation | Number of | Overall Success |
|---|---|---|---|
| Task | method | misclassifications | Rate |
| Gene selection | Resubstitution | 9 | 0.9461 |
| method 1 | FIPG LOOCV | 28 | 0.8323 |
| | FDPG LOOCV | 58 | 0.6527 |
| Gene selection | Resubstitution | 10 | 0.9401 |
| method 2 | FIPG LOOCV | 30 | 0.8204 |
| | FDPG LOOCV | 55 | 0.6707 |

### Example IX

Uniformly Significant Genes that Are Correlated with CCR vs. Failure

The three data sets derived from the retrospective statistically designed 254 member Pre-B data set were analyzed for their association with outcome: the 167 member training set, the 87 member test set and overall 254 member data set. Three measures were used: ROC accuracy A, F-test statistic and TNoM. Table 29 shows a list of genes correlated with outcome with the ranks determined by these different measures with the different data sets.

Two genes were consistently significant in both training and test sets and they are number one and number two significant genes in the overall data set. The two genes are 39418_at, DKFZP564M182 protein (PBK1) and 41819_at, FYN-binding protein (FYB-120/130). FYN is a tyrosine kinast found in fibroblasts and T lymphocytes (Popescu et al., Oncogene 1(4):449-451 (1987)).

Unexpectedly, although OPAL1/G0 was the most significant gene in the training data set, it was a much less significant gene in the test data set. Indeed, most of the significant genes in training set, like OPAL1/G0, became less significant in test set. The fact that most genes that did well in the training set did poorly in the test set lends support to our hypothesis that the test set's composition differed significantly from that of the training set. We therefore sought to increase the robustness of this statistical analysis.

Re-Sampling Training and Test Data Sets

Our goal was to identify genes that are significant irrespective of the data set. One way to get a stable (robust) list of genes that are highly correlated with the distinction of CCR vs. Failure is through the use of a random re-sampling (bootstrap) procedure. We randomly divided the overall data set into training and test sets 172 times. The numbers of CCRs and Failures in the training set was fixed to agree with the original training set, (i.e. 73 CCR s and 94 Failures). Each time the genes are ranked in the same way as in Table 1. That is, we produced 172 tables like Table 29 for the 172 different training and test sets.

We found that the gene ranking in the two data sets (training and test randomly resampled in each time) are typically quite different. However, in most runs, the two genes 39418_at (PBK1) and 41819_at (FYN-binding protein) were consistently significant in both the random training and test sets. We called these two genes the uniformly most significant genes. OPAL1/G0 (38652_at) also consistently shows significance.

Generation of a Robust Gene List (a List of Uniformly Significant Genes)

The following rule was used to assign a quantitative value to each gene to evaluate the extent that the gene is uniformly significant: in each training and test set, the genes are ranked by three measures. After 172 resamplings, each gene has 172 ranks on the three measures in each of two data sets. We calculate the average or mean of the 172 ranks of each gene. We then sorted the genes on the mean ranks. In this way we get a robust gene list corresponding to each of three measures in each of the two data sets.

The top 100 genes in the robust gene list are presented in Table 30 with the robust ranks determined by the three different measures. We found that the ranks in training set and test set closely agree with each other and with the rank determined by the overall data set. The two most uniformly significant genes (39418_at and 41819_at) were ranked first and second. OPAL1/G0 survives in this analysis and had good average ranks on the three measures, but was only about 10^{th} best overall.

TABLE 29
Ranks of significant Genes Generated in Original Training, Test and
Overall Data Sets

| In Training | | | | | | | | | | |
|---|---|---|---|---|---|---|---|---|---|---|
| Data Set | | | In Test Data Set | | | In Overall Data Set | | | | |
| A | F | TNoM | A | F | TNoM | A | F | TNoM | | |
| Rank | Rank | Rank | Rank | Rank | Rank | Rank | Rank | Rank | Accession # | Gene Description |
| 1 | 1 | 1 | 7695 | 7493 | 7251 | 10 | 7 | 6 | 38652_at | hypothetical |
| | | | | | | | | | | protein |
| | | | | | | | | | | FLJ20154 |
| 2 | 2 | 54 | 60 | 122 | 94 | 1 | 1 | 7 | 39418_at | DKFZP564M182 |
| | | | | | | | | | | protein |
| 3 | 5 | 22 | 3757 | 3530 | 4708 | 14 | 17 | 32 | 41478_at | Homo sapiens |
| | | | | | | | | | | cDNA FLJ30991 |
| | | | | | | | | | | fis, clone |
| | | | | | | | | | | HLUNG1000041 |
| 4 | 14 | 32 | 8337 | 8425 | 1894 | 132 | 253 | 266 | 37674_at | aminolevulinate, |
| | | | | | | | | | | delta-, synthase 1 |
| 5 | 6 | 10 | 4353 | 4210 | 5827 | 31 | 23 | 83 | 38270_at | poly (ADP- |
| | | | | | | | | | | ribose) |
| | | | | | | | | | | glycohydrolase |
| 6 | 3 | 49 | 2354 | 818 | 2966 | 12 | 2 | 81 | 38119_at | glycophorin C |
| | | | | | | | | | | (Gerbich blood |
| | | | | | | | | | | group) |
| 7 | 4 | 35 | 1026 | 945 | 2202 | 6 | 3 | 65 | 671_at | secreted protein, |
| | | | | | | | | | | acidic, cysteine- |
| | | | | | | | | | | rich (osteonectin) |
| 8 | 20 | 12 | 1702 | 933 | 1418 | 8 | 12 | 66 | 1126_s_at | Homo sapiens |
| | | | | | | | | | | CD44 isoform |
| | | | | | | | | | | RC (CD44) |
| | | | | | | | | | | mRNA, complete |
| | | | | | | | | | | cds |
| 9 | 7 | 38 | 3684 | 7525 | 5011 | 25 | 78 | 143 | 31527_at | ribosomal |
| | | | | | | | | | | protein S2 |
| 10 | 9 | 61 | 7679 | 6989 | 7628 | 150 | 166 | 286 | 587_at | endothelial |
| | | | | | | | | | | differentiation, |
| | | | | | | | | | | sphingolipid G- |
| | | | | | | | | | | protein-coupled |
| | | | | | | | | | | receptor, 1 |
| 11 | 26 | 45 | 3263 | 4366 | 6960 | 30 | 86 | 168 | 36144_at | KIAA0080 |
| | | | | | | | | | | protein |
| 12 | 22 | 63 | 6526 | 6224 | 7633 | 97 | 125 | 204 | 625_at | membrane |
| | | | | | | | | | | protein of |
| | | | | | | | | | | cholinergic |
| | | | | | | | | | | synaptic vesicles |
| 13 | 10 | 212 | 6098 | 6724 | 5394 | 75 | 93 | 335 | 34760_at | KIAA0022 gene |
| | | | | | | | | | | product |
| 14 | 18 | 143 | 2541 | 1713 | 7043 | 20 | 21 | 359 | 36927_at | hypothetical |
| | | | | | | | | | | protein, |
| | | | | | | | | | | expressed in |
| | | | | | | | | | | osteoblast |
| 15 | 8 | 17 | 5147 | 5142 | 7971 | 72 | 34 | 162 | 35796_at | protein tyrosine |
| | | | | | | | | | | kinase 9-like |
| | | | | | | | | | | (A6-related |
| | | | | | | | | | | protein) |
| 16 | 35 | 14 | 7445 | 8457 | 7792 | 175 | 205 | 460 | 32336_at | aldolase A, |
| | | | | | | | | | | fructose- |
| | | | | | | | | | | bisphosphate |
| 17 | 161 | 74 | 6925 | 5891 | 6648 | 138 | 374 | 318 | 33188_at | peptidylprolyl |
| | | | | | | | | | | isomerase |
| | | | | | | | | | | (cyclophilin)-like 2 |
| 18 | 109 | 11 | 38 | 63 | 104 | 2 | 8 | 2 | 41819_at | FYN-binding |
| | | | | | | | | | | protein (FYB- |
| | | | | | | | | | | 120/130) |
| 19 | 56 | 36 | 3000 | 4192 | 4982 | 45 | 161 | 139 | 2062_at | insulin-like |
| | | | | | | | | | | growth factor |
| | | | | | | | | | | binding protein 7 |
| 20 | 43 | 124 | 6998 | 5801 | 6770 | 333 | 514 | 1373 | 34349_at | SEC63 protein |
| 21 | 25 | 184 | 7476 | 7310 | 8582 | 168 | 175 | 1219 | 932_i_at | zinc finger |
| | | | | | | | | | | protein 91 |
| | | | | | | | | | | (HPF7, HTF10) |
| 22 | 198 | 149 | 2380 | 3049 | 2927 | 36 | 238 | 80 | 37748_at | KIAA0232 gene |
| | | | | | | | | | | product |
| 23 | 12 | 83 | 3966 | 8153 | 4329 | 115 | 231 | 175 | 38440_s_at | hypothetical |
| | | | | | | | | | | protein |
| 24 | 33 | 96 | 6080 | 6141 | 6364 | 144 | 119 | 856 | 106_at | runt-related |
| | | | | | | | | | | transcription |
| | | | | | | | | | | factor 3 |
| 25 | 54 | 20 | 80 | 90 | 177 | 4 | 6 | 3 | 37343_at | inositol 1,4,5- |
| | | | | | | | | | | triphosphate |
| | | | | | | | | | | receptor, type 3 |
| 26 | 59 | 199 | 3436 | 3294 | 6609 | 78 | 123 | 316 | 32703_at | serine/threonine |
| | | | | | | | | | | kinase 18 |
| 27 | 31 | 18 | 1805 | 2464 | 4031 | 35 | 36 | 121 | 36154_at | KIAA0263 gene |
| | | | | | | | | | | product |
| 28 | 50 | 48 | 1479 | 1275 | 1931 | 1520 | 2214 | 3445 | 38111_at | chondroitin |
| | | | | | | | | | | sulfate |
| | | | | | | | | | | proteoglycan 2 |
| | | | | | | | | | | (versican) |
| 29 | 36 | 5 | 4225 | 4623 | 4966 | 68 | 111 | 19 | 1980_s_at | non-metastatic |
| | | | | | | | | | | cells 2, protein |
| | | | | | | | | | | (NM23B) |
| | | | | | | | | | | expressed in |
| 30 | 21 | 214 | 4722 | 4614 | 6831 | 87 | 58 | 693 | 34965_at | cystatin F |
| | | | | | | | | | | (leukocystatin) |
| 31 | 39 | 118 | 410 | 385 | 297 | 9 | 10 | 11 | 33412_at | lectin, |
| | | | | | | | | | | galactoside- |
| | | | | | | | | | | binding, soluble, |
| | | | | | | | | | | 1 (galectin 1) |
| 32 | 48 | 159 | 4699 | 3446 | 7359 | 667 | 1045 | 2761 | 39607_at | myotubularin |
| | | | | | | | | | | related protein 8 |
| 33 | 87 | 677 | 4246 | 4880 | 4929 | 908 | 1194 | 4856 | 1698_g_at | mitogen- |
| | | | | | | | | | | activated protein |
| | | | | | | | | | | kinase kinase 5 |
| 34 | 41 | 42 | 7549 | 7856 | 7947 | 195 | 212 | 119 | 35322_at | Kelch-like ECH- |
| | | | | | | | | | | associated |
| | | | | | | | | | | protein 1 |
| 35 | 200 | 75 | 2290 | 4897 | 5290 | 53 | 484 | 155 | 33866_at | tropomyosin 4 |
| 36 | 23 | 728 | 1700 | 2677 | 1584 | 37 | 54 | 149 | 32623_at | gamma- |
| | | | | | | | | | | aminobutyric |
| | | | | | | | | | | acid (GABA) B |
| | | | | | | | | | | receptor, 1 |
| 37 | 38 | 348 | 2662 | 3937 | 4001 | 57 | 67 | 1022 | 35939_s_at | POU domain, |
| | | | | | | | | | | class 4, |
| | | | | | | | | | | transcription |
| | | | | | | | | | | factor 1 |
| 38 | 24 | 132 | 6369 | 8517 | 6890 | 629 | 371 | 346 | 35614_at | transcription |
| | | | | | | | | | | factor-like 5 |
| | | | | | | | | | | (basic helix- |
| | | | | | | | | | | loop-helix) |
| 39 | 15 | 422 | 3450 | 2407 | 4730 | 91 | 25 | 417 | 41656_at | N- |
| | | | | | | | | | | myristoyltransferase 2 |
| 40 | 82 | 299 | 5587 | 5878 | 5033 | 215 | 354 | 454 | 31830_s_at | smoothelin |
| 41 | 28 | 297 | 4620 | 2982 | 5023 | 140 | 51 | 892 | 31695_g_at | regulatory solute |
| | | | | | | | | | | carrier protein, |
| | | | | | | | | | | family 1, |
| | | | | | | | | | | member 1 |
| 42 | 27 | 210 | 2295 | 3602 | 1699 | 67 | 68 | 112 | 34433_at | docking protein |
| | | | | | | | | | | 1, 62 kD |
| | | | | | | | | | | (downstream of |
| | | | | | | | | | | tyrosine kinase |
| | | | | | | | | | | 1) |
| 43 | 67 | 432 | 656 | 367 | 3375 | 16 | 13 | 205 | 824_at | glutathione-S- |
| | | | | | | | | | | transferase like; |
| | | | | | | | | | | glutathione |
| | | | | | | | | | | transferase |
| | | | | | | | | | | omega |
| 44 | 53 | 631 | 5724 | 6981 | 6154 | 712 | 587 | 2164 | 40817_at | nucleobindin 1 |
| 45 | 37 | 87 | 3277 | 3624 | 6098 | 88 | 81 | 400 | 40365_at | guanine |
| | | | | | | | | | | nucleotide |
| | | | | | | | | | | binding protein |
| | | | | | | | | | | (G protein), |
| | | | | | | | | | | alpha 15 (Gq |
| | | | | | | | | | | class) |
| 46 | 321 | 183 | 4355 | 2425 | 4813 | 1178 | 4723 | 2240 | 843_at | protein tyrosine |
| | | | | | | | | | | phosphatase type |
| | | | | | | | | | | IVA, member 1 |
| 47 | 29 | 170 | 7282 | 6865 | 6155 | 523 | 402 | 583 | 40821_at | S- |
| | | | | | | | | | | adenosylhomocy |
| | | | | | | | | | | steine hydrolase |
| 48 | 81 | 101 | 8352 | 6490 | 3444 | 308 | 737 | 623 | 1452_at | LIM domain |
| | | | | | | | | | | only 4 |
| 49 | 11 | 2 | 2576 | 5715 | 3725 | 54 | 101 | 5 | 33415_at | non-metastatic |
| | | | | | | | | | | cells 2, protein |
| | | | | | | | | | | (NM23B) |
| | | | | | | | | | | expressed in |
| 50 | 72 | 311 | 1693 | 2506 | 930 | 41 | 79 | 313 | 32629_f_at | butyrophilin, |
| | | | | | | | | | | subfamily 3, |
| | | | | | | | | | | member A1 |
| 51 | 30 | 19 | 5994 | 5551 | 4154 | 846 | 652 | 1057 | 37147_at | stem cell growth |
| | | | | | | | | | | factor; |
| | | | | | | | | | | lymphocyte |
| | | | | | | | | | | secreted C-type |
| | | | | | | | | | | lectin |
| 52 | 57 | 162 | 6231 | 6377 | 8551 | 232 | 225 | 1144 | 39932_at | Homo sapiens |
| | | | | | | | | | | mRNA; cDNA |
| | | | | | | | | | | DKFZp586F2224 |
| | | | | | | | | | | (from clone |
| | | | | | | | | | | DKFZp586F2224) |
| 53 | 74 | 26 | 1585 | 1098 | 2297 | 47 | 35 | 17 | 1711_at | tumor protein |
| | | | | | | | | | | p53-binding |
| | | | | | | | | | | protein, 1 |
| 54 | 274 | 21 | 3295 | 2921 | 3154 | 74 | 278 | 43 | 40141_at | cullin 4B |
| 55 | 16 | 46 | 3687 | 5454 | 1826 | 1278 | 442 | 252 | 36537_at | Rho-specific |
| | | | | | | | | | | guanine |
| | | | | | | | | | | nucleotide |
| | | | | | | | | | | exchange factor |
| | | | | | | | | | | p114 |
| 56 | 62 | 33 | 5966 | 5635 | 7169 | 220 | 214 | 173 | 37986_at | erythropoietin |
| | | | | | | | | | | receptor |
| 57 | 55 | 24 | 1793 | 2145 | 4887 | 44 | 50 | 95 | 1403_s_at | small inducible |
| | | | | | | | | | | cytokine A5 |
| | | | | | | | | | | (RANTES) |
| 58 | 185 | 201 | 5797 | 4517 | 2477 | 159 | 331 | 151 | 32843_s_at | fibrillarin |
| 59 | 88 | 265 | 5254 | 3724 | 4435 | 202 | 170 | 565 | 39302_at | desmocollin 2 |
| 60 | 13 | 606 | 2770 | 1145 | 5922 | 82 | 11 | 771 | 38971_r_at | Nef-associated |
| | | | | | | | | | | factor 1 |
| 61 | 40 | 40 | 5525 | 6158 | 6715 | 245 | 211 | 482 | 33757_f_at | pregnancy |
| | | | | | | | | | | specific beta-1- |
| | | | | | | | | | | glycoprotein 11 |
| 62 | 286 | 28 | 2620 | 2264 | 5008 | 83 | 236 | 142 | 31472_s_at | Homo sapiens |
| | | | | | | | | | | CD44 isoform |
| | | | | | | | | | | RC (CD44) |
| | | | | | | | | | | mRNA, complete |
| | | | | | | | | | | cds |
| 63 | 305 | 318 | 1023 | 2872 | 307 | 26 | 310 | 154 | 33637_g_at | cancer/testis |
| | | | | | | | | | | antigen |
| 64 | 184 | 190 | 4452 | 3255 | 3517 | 223 | 241 | 445 | 207_at | stress-induced- |
| | | | | | | | | | | phosphoprotein 1 |
| | | | | | | | | | | (Hsp70/Hsp90- |
| | | | | | | | | | | organizing |
| | | | | | | | | | | protein) |
| 65 | 101 | 399 | 5221 | 4264 | 7422 | 249 | 206 | 798 | 40183_at | coactivator- |
| | | | | | | | | | | associated |
| | | | | | | | | | | arginine |
| | | | | | | | | | | methyltransferase-1 |
| 66 | 91 | 56 | 2163 | 3116 | 3162 | 1969 | 1848 | 2792 | 40246_at | discs, large |
| | | | | | | | | | | (Drosophila) |
| | | | | | | | | | | homolog 1 |
| 67 | 19 | 370 | 2898 | 1532 | 2878 | 107 | 20 | 260 | 37280_at | MAD (mothers |
| | | | | | | | | | | against |
| | | | | | | | | | | decapentaplegic, |
| | | | | | | | | | | Drosophila) |
| | | | | | | | | | | homolog 1 |
| 68 | 71 | 911 | 2538 | 3388 | 5963 | 1680 | 1549 | 7785 | 39221_at | leukocyte |
| | | | | | | | | | | immunoglobulin- |
| | | | | | | | | | | like receptor, |
| | | | | | | | | | | subfamily B |
| | | | | | | | | | | (with TM and |
| | | | | | | | | | | ITIM domains), |
| | | | | | | | | | | member 2 |
| 69 | 203 | 7 | 437 | 440 | 929 | 3017 | 4275 | 466 | 32624_at | DKFZp566D133 |
| | | | | | | | | | | protein |
| 70 | 60 | 94 | 6844 | 6653 | 6358 | 785 | 640 | 425 | *** | NO_.SIF_seq |
| 71 | 76 | 817 | 4663 | 4498 | 5550 | 1073 | 1187 | 2548 | 36060_at | signal |
| | | | | | | | | | | recognition |
| | | | | | | | | | | particle 54 kD |
| 72 | 44 | 627 | 2530 | 2272 | 6120 | 113 | 52 | 402 | 40507_at | solute carrier |
| | | | | | | | | | | family 2 |
| | | | | | | | | | | (facilitated |
| | | | | | | | | | | glucose |
| | | | | | | | | | | transporter), |
| | | | | | | | | | | member 1 |
| 73 | 58 | 307 | 4991 | 4702 | 5083 | 254 | 171 | 225 | 32211_at | proteasome |
| | | | | | | | | | | (prosome, |
| | | | | | | | | | | macropain) 26S |
| | | | | | | | | | | subunit, non- |
| | | | | | | | | | | ATPase, 13 |
| 74 | 46 | 825 | 3943 | 2954 | 8016 | 191 | 70 | 2586 | 36500_at | NAD(P) |
| | | | | | | | | | | dependent |
| | | | | | | | | | | steroid |
| | | | | | | | | | | dehydrogenase- |
| | | | | | | | | | | like; H105e3 |
| 75 | 264 | 397 | 5397 | 4257 | 7394 | 224 | 362 | 572 | 39865_at | Homo sapiens |
| | | | | | | | | | | cDNA FLJ30639 |
| | | | | | | | | | | fis, clone |
| | | | | | | | | | | CTONG2002803 |
| 76 | 77 | 104 | 4288 | 5778 | 2331 | 1055 | 679 | 444 | 2035_s_at | enolase 1, |
| | | | | | | | | | | (alpha) |
| 77 | 97 | 373 | 2644 | 2657 | 5748 | 94 | 117 | 738 | 37572_at | cholecystokinin |
| 78 | 45 | 111 | 5526 | 6106 | 3614 | 197 | 201 | 226 | 32254_at | vesicle- |
| | | | | | | | | | | associated |
| | | | | | | | | | | membrane |
| | | | | | | | | | | protein 2 |
| | | | | | | | | | | (synaptobrevin |
| | | | | | | | | | | 2) |
| 79 | 291 | 92 | 4357 | 7049 | 4748 | 188 | 790 | 202 | 41761_at | TIA1 cytotoxic |
| | | | | | | | | | | granule- |
| | | | | | | | | | | associated RNA- |
| | | | | | | | | | | binding protein- |
| | | | | | | | | | | like 1 |
| 80 | 242 | 233 | 8287 | 8066 | 7012 | 478 | 956 | 1963 | 36624_at | IMP (inosine |
| | | | | | | | | | | monophosphate) |
| | | | | | | | | | | dehydrogenase 2 |
| 81 | 133 | 240 | 1388 | 1748 | 1871 | 2911 | 2910 | 2622 | 37263_at | gamma-glutamyl |
| | | | | | | | | | | hydrolase |
| | | | | | | | | | | (conjugase, |
| | | | | | | | | | | folylpolygamma |
| | | | | | | | | | | glutamyl |
| | | | | | | | | | | hydrolase) |
| 82 | 103 | 175 | 2570 | 3861 | 4671 | 112 | 158 | 88 | 41224_at | KIAA0788 |
| | | | | | | | | | | protein |
| 83 | 64 | 250 | 917 | 955 | 1183 | 38 | 26 | 371 | 38087_s_at | S100 calcium- |
| | | | | | | | | | | binding protein |
| | | | | | | | | | | A4 (calcium |
| | | | | | | | | | | protein, |
| | | | | | | | | | | calvasculin, |
| | | | | | | | | | | metastasin, |
| | | | | | | | | | | murine placental |
| | | | | | | | | | | homolog) |
| 84 | 129 | 31 | 6589 | 4786 | 1770 | 417 | 305 | 13 | 35669_at | KIAA0633 |
| | | | | | | | | | | protein |
| 85 | 212 | 119 | 1435 | 3718 | 3729 | 2286 | 2573 | 2422 | 33433_at | DKFZP564F052 |
| | | | | | | | | | | 2 protein |
| 86 | 183 | 244 | 5029 | 5157 | 5729 | 241 | 394 | 261 | 37441_at | lipoyltransferase |
| 87 | 83 | 228 | 7786 | 7738 | 8485 | 451 | 283 | 1025 | 36002_at | KIAA1012 |
| | | | | | | | | | | protein |
| 88 | 120 | 548 | 7750 | 7722 | 7015 | 515 | 548 | 1968 | 36678_at | transgelin 2 |
| 89 | 42 | 139 | 1062 | 926 | 163 | 32 | 18 | 15 | 36129_at | KIAA0397 gene |
| | | | | | | | | | | product |
| 90 | 34 | 200 | 259 | 1166 | 25 | 15 | 19 | 10 | 32724_at | phytanoyl-CoA |
| | | | | | | | | | | hydroxylase |
| | | | | | | | | | | (Refsum disease) |
| 91 | 65 | 57 | 4461 | 4427 | 4570 | 176 | 159 | 809 | 40435_at | solute carrier |
| | | | | | | | | | | family 25 |
| | | | | | | | | | | (mitochondrial |
| | | | | | | | | | | carrier; adenine |
| | | | | | | | | | | nucleotide |
| | | | | | | | | | | translocator), |
| | | | | | | | | | | member 6 |
| 92 | 132 | 68 | 2452 | 3105 | 1473 | 95 | 163 | 18 | 1923_at | cyclin C |
| 93 | 70 | 142 | 6343 | 7528 | 7031 | 860 | 689 | 719 | 36835_at | protein kinase C- |
| | | | | | | | | | | like 2 |
| 94 | 157 | 103 | 7459 | 4945 | 3449 | 738 | 1513 | 1241 | 1473_s_at | v-myb avian |
| | | | | | | | | | | myeloblastosis |
| | | | | | | | | | | viral oncogene |
| | | | | | | | | | | homolog |
| 95 | 158 | 410 | 585 | 1147 | 217 | 3710 | 3944 | 2837 | 41060_at | cyclin E1 |
| 96 | 240 | 277 | 6070 | 4715 | 4629 | 279 | 419 | 820 | 40859_at | Homo sapiens |
| | | | | | | | | | | mRNA; cDNA |
| | | | | | | | | | | DKFZp762G207 |
| | | | | | | | | | | (from clone |
| | | | | | | | | | | DKFZp762G207) |
| 97 | 190 | 9 | 8035 | 6314 | 5815 | 574 | 560 | 542 | 38134_at | pleiomorphic |
| | | | | | | | | | | adenoma gene 1 |
| 98 | 32 | 235 | 2988 | 3846 | 4106 | 145 | 55 | 515 | 36783_f_at | Krueppel-related |
| | | | | | | | | | | zinc finger |
| | | | | | | | | | | protein |
| 99 | 259 | 437 | 5264 | 5003 | 4852 | 274 | 443 | 1646 | 1062_g_at | interleukin 10 |
| | | | | | | | | | | receptor, alpha |
| 100 | 227 | 823 | 2199 | 1173 | 4045 | 111 | 122 | 1035 | 36207_at | SEC14 (S. cerevisiae)- |
| | | | | | | | | | | like 1 |

*** = AFFX-HUMGAPDH/M33197_M_at

TABLE 30
Lists of Most Uniformly Significant Genes
(Generated from 172 resampled Training and Test Data sets)

| In Training | | | | | | | | | | |
|---|---|---|---|---|---|---|---|---|---|---|
| Data Set | | | In Test Data Set | | | In Overall Data Set | | | | |
| A | F | TNoM | A | F | TnoM | A | F | TNoM | | Gene |
| Rank | Rank | Rank | Rank | Rank | Rank | Rank | Rank | Rank | Accession # | Description |
| 1 | 1 | 6 | 1 | 1 | 2 | 1 | 1 | 7 | 39418_at | DKFZP564M1 |
| | | | | | | | | | | 82 protein |
| 2 | 8 | 2 | 3 | 8 | 1 | 2 | 8 | 2 | 41819_at | FYN-binding |
| | | | | | | | | | | protein (FYB- |
| | | | | | | | | | | 120/130) |
| 3 | 4 | 53 | 2 | 3 | 20 | 3 | 5 | 42 | 37981_at | drebrin 1 |
| 4 | 2 | 1 | 4 | 5 | 3 | 5 | 4 | 1 | 577_at | midkine |
| | | | | | | | | | | (neurite |
| | | | | | | | | | | growth- |
| | | | | | | | | | | promoting |
| | | | | | | | | | | factor 2) |
| 5 | 5 | 5 | 5 | 9 | 5 | 4 | 6 | 3 | 37343_at | inositol 1,4,5- |
| | | | | | | | | | | triphosphate |
| | | | | | | | | | | receptor, type 3 |
| 6 | 9 | 44 | 7 | 6 | 23 | 7 | 9 | 71 | 32058_at | HNK-1 |
| | | | | | | | | | | sulfotransferase |
| 7 | 10 | 10 | 10 | 12 | 12 | 9 | 10 | 11 | 33412_at | lectin, |
| | | | | | | | | | | galactoside- |
| | | | | | | | | | | binding, |
| | | | | | | | | | | soluble, 1 |
| | | | | | | | | | | (galectin 1) |
| 8 | 12 | 31 | 14 | 20 | 13 | 8 | 12 | 66 | 1126_s_at | Homo sapiens |
| | | | | | | | | | | CD44 isoform |
| | | | | | | | | | | RC (CD44) |
| | | | | | | | | | | mRNA, |
| | | | | | | | | | | complete cds |
| 9 | 6 | 52 | 6 | 4 | 46 | 6 | 3 | 65 | 671_at | secreted |
| | | | | | | | | | | protein, acidic, |
| | | | | | | | | | | cysteine-rich |
| | | | | | | | | | | (osteonectin) |
| 10 | 13 | 23 | 9 | 14 | 15 | 11 | 14 | 35 | 32970_f_at | intracellular |
| | | | | | | | | | | hyaluronan- |
| | | | | | | | | | | binding protein |
| 11 | 11 | 116 | 18 | 19 | 317 | 16 | 13 | 205 | 824_at | glutathione-S- |
| | | | | | | | | | | transferase |
| | | | | | | | | | | like; |
| | | | | | | | | | | glutathione |
| | | | | | | | | | | transferase |
| | | | | | | | | | | omega |
| 12 | 17 | 9 | 19 | 30 | 10 | 15 | 19 | 10 | 32724_at | phytanoyl- |
| | | | | | | | | | | CoA |
| | | | | | | | | | | hydroxylase |
| | | | | | | | | | | (Refsum |
| | | | | | | | | | | disease) |
| 13 | 7 | 8 | 13 | 7 | 18 | 10 | 7 | 6 | 38652_at | hypothetical |
| | | | | | | | | | | protein |
| | | | | | | | | | | FLJ20154 |
| 14 | 22 | 41 | 15 | 27 | 39 | 13 | 24 | 40 | 36331_at | Homo sapiens |
| | | | | | | | | | | mRNA; cDNA |
| | | | | | | | | | | DKFZp586C0 |
| | | | | | | | | | | 91 (from clone |
| | | | | | | | | | | DKFZp586C0 |
| | | | | | | | | | | 91) |
| 15 | 19 | 30 | 8 | 13 | 24 | 14 | 17 | 32 | 41478_at | Homo sapiens |
| | | | | | | | | | | cDNA |
| | | | | | | | | | | FLJ30991 fis, |
| | | | | | | | | | | clone |
| | | | | | | | | | | HLUNG10000 |
| | | | | | | | | | | 41 |
| 16 | 3 | 117 | 11 | 2 | 128 | 12 | 2 | 81 | 38119_at | glycophorin C |
| | | | | | | | | | | (Gerbich blood |
| | | | | | | | | | | group) |
| 17 | 24 | 417 | 34 | 28 | 401 | 20 | 21 | 359 | 36927_at | hypothetical |
| | | | | | | | | | | protein, |
| | | | | | | | | | | expressed in |
| | | | | | | | | | | osteoblast |
| 18 | 38 | 81 | 27 | 49 | 71 | 18 | 33 | 53 | 35145_at | MAX binding |
| | | | | | | | | | | protein |
| 19 | 248 | 122 | 52 | 414 | 91 | 26 | 310 | 154 | 33637_g_at | cancer/testis |
| | | | | | | | | | | antigen |
| 20 | 15 | 186 | 92 | 71 | 558 | 38 | 26 | 371 | 38087_s_at | S100 calcium- |
| | | | | | | | | | | binding protein |
| | | | | | | | | | | A4 (calcium |
| | | | | | | | | | | protein, |
| | | | | | | | | | | calvasculin, |
| | | | | | | | | | | metastasin, |
| | | | | | | | | | | murine |
| | | | | | | | | | | placental |
| | | | | | | | | | | homolog) |
| 21 | 104 | 643 | 23 | 118 | 275 | 28 | 120 | 1044 | 36576_at | H2A histone |
| | | | | | | | | | | family, |
| | | | | | | | | | | member Y |
| 22 | 31 | 64 | 20 | 18 | 75 | 24 | 31 | 62 | 40523_at | hepatocyte |
| | | | | | | | | | | nuclear factor |
| | | | | | | | | | | 3, beta |
| 23 | 40 | 12 | 12 | 21 | 7 | 17 | 29 | 12 | 34332_at | glucosamine- |
| | | | | | | | | | | 6-phosphate |
| | | | | | | | | | | isomerase |
| 24 | 60 | 180 | 16 | 46 | 134 | 21 | 59 | 314 | 32650_at | neuronal |
| | | | | | | | | | | protein |
| 25 | 960 | 21 | 31 | 599 | 9 | 19 | 767 | 9 | 41727_at | KIAA1007 |
| | | | | | | | | | | protein |
| 26 | 79 | 230 | 47 | 141 | 145 | 25 | 78 | 143 | 31527_at | ribosomal |
| | | | | | | | | | | protein S2 |
| 27 | 83 | 60 | 36 | 105 | 55 | 22 | 62 | 27 | 38437_at | MLN51 |
| | | | | | | | | | | protein |
| 28 | 20 | 118 | 22 | 15 | 90 | 23 | 16 | 122 | 36524_at | Rho guanine |
| | | | | | | | | | | nucleotide |
| | | | | | | | | | | exchange |
| | | | | | | | | | | factor (GEF) 4 |
| 29 | 56 | 70 | 49 | 90 | 116 | 43 | 77 | 165 | 36081_s_at | chromosome |
| | | | | | | | | | | 21 open |
| | | | | | | | | | | reading frame |
| | | | | | | | | | | 18 |
| 30 | 47 | 191 | 37 | 38 | 106 | 33 | 41 | 294 | 160030_at | growth |
| | | | | | | | | | | hormone |
| | | | | | | | | | | receptor |
| 31 | 102 | 146 | 42 | 111 | 113 | 30 | 86 | 168 | 36144_at | KIAA0080 |
| | | | | | | | | | | protein |
| 32 | 244 | 108 | 87 | 341 | 239 | 36 | 238 | 80 | 37748_at | KIAA0232 |
| | | | | | | | | | | gene product |
| 33 | 26 | 90 | 32 | 17 | 141 | 31 | 23 | 83 | 38270_at | poly (ADP- |
| | | | | | | | | | | ribose) |
| | | | | | | | | | | glycohydrolase |
| 34 | 63 | 132 | 35 | 41 | 97 | 37 | 54 | 149 | 32623_at | gamma- |
| | | | | | | | | | | aminobutyric |
| | | | | | | | | | | acid (GABA) |
| | | | | | | | | | | B receptor, 1 |
| 35 | 57 | 158 | 30 | 67 | 61 | 50 | 69 | 296 | 1676_s_at | eukaryotic |
| | | | | | | | | | | translation |
| | | | | | | | | | | elongation |
| | | | | | | | | | | factor 1 |
| | | | | | | | | | | gamma |
| 36 | 165 | 61 | 21 | 121 | 50 | 34 | 149 | 28 | 38865_at | GRB2-related |
| | | | | | | | | | | adaptor protein 2 |
| 37 | 28 | 157 | 74 | 63 | 171 | 76 | 43 | 310 | 324_f_at | NO_.SIF_seq |
| 38 | 84 | 3 | 59 | 119 | 4 | 54 | 101 | 5 | 33415_at | non-metastatic |
| | | | | | | | | | | cells 2, protein |
| | | | | | | | | | | (NM23B) |
| | | | | | | | | | | expressed in |
| 39 | 134 | 136 | 28 | 80 | 64 | 27 | 71 | 156 | 34171_at | hypothetical |
| | | | | | | | | | | protein from |
| | | | | | | | | | | EUROIMAGE |
| | | | | | | | | | | 2021883 |
| 40 | 21 | 24 | 44 | 23 | 34 | 32 | 18 | 15 | 36129_at | KIAA0397 |
| | | | | | | | | | | gene product |
| 41 | 106 | 29 | 40 | 82 | 33 | 56 | 135 | 14 | 36004_at | Homo sapiens |
| | | | | | | | | | | cDNA |
| | | | | | | | | | | FLJ20586 fis, |
| | | | | | | | | | | clone |
| | | | | | | | | | | KAT09466, |
| | | | | | | | | | | highly similar |
| | | | | | | | | | | to AF091453 |
| | | | | | | | | | | Homo sapiens |
| | | | | | | | | | | NEMO protein |
| 42 | 39 | 66 | 64 | 68 | 74 | 42 | 37 | 94 | 1189_at | cyclin- |
| | | | | | | | | | | dependent |
| | | | | | | | | | | kinase 8 |
| 43 | 48 | 154 | 50 | 51 | 92 | 44 | 50 | 95 | 1403_s_at | small |
| | | | | | | | | | | inducible |
| | | | | | | | | | | cytokine A5 |
| | | | | | | | | | | (RANTES) |
| 44 | 54 | 779 | 56 | 64 | 557 | 57 | 67 | 1022 | 35939_s_at | POU domain, |
| | | | | | | | | | | class 4, |
| | | | | | | | | | | transcription |
| | | | | | | | | | | factor 1 |
| 45 | 30 | 379 | 67 | 47 | 429 | 60 | 38 | 246 | 35675_at | vinexin beta |
| | | | | | | | | | | (SH3- |
| | | | | | | | | | | containing |
| | | | | | | | | | | adaptor |
| | | | | | | | | | | molecule-1) |
| 46 | 33 | 26 | 103 | 72 | 84 | 77 | 44 | 25 | 35856_r_at | glutamate |
| | | | | | | | | | | receptor, |
| | | | | | | | | | | ionotropic, |
| | | | | | | | | | | kainate 1 |
| 47 | 37 | 516 | 55 | 43 | 265 | 49 | 40 | 442 | 1818_at | NO_.SIF_seq |
| 48 | 197 | 56 | 17 | 65 | 19 | 29 | 142 | 37 | 35059_at | Homo sapiens |
| | | | | | | | | | | clone FBA1 |
| | | | | | | | | | | Cri-du-chat |
| | | | | | | | | | | region mRNA |
| 49 | 65 | 37 | 71 | 92 | 45 | 39 | 53 | 78 | 36069_at | KIAA0456 |
| | | | | | | | | | | protein |
| 50 | 94 | 11 | 78 | 156 | 11 | 68 | 111 | 19 | 1980_s_at | non-metastatic |
| | | | | | | | | | | cells 2, protein |
| | | | | | | | | | | (NM23B) |
| | | | | | | | | | | expressed in |
| 51 | 81 | 147 | 45 | 79 | 63 | 46 | 75 | 150 | 32739_at | N- |
| | | | | | | | | | | acetylglucosamine- |
| | | | | | | | | | | phosphate |
| | | | | | | | | | | mutase |
| 52 | 115 | 85 | 51 | 112 | 144 | 51 | 114 | 57 | 361_at | B-cell |
| | | | | | | | | | | CLL/lymphoma 9 |
| 53 | 100 | 256 | 39 | 96 | 112 | 41 | 79 | 313 | 32629_f_at | butyrophilin, |
| | | | | | | | | | | subfamily 3, |
| | | | | | | | | | | member A1 |
| 54 | 189 | 181 | 33 | 115 | 76 | 45 | 161 | 139 | 2062_at | insulin-like |
| | | | | | | | | | | growth factor |
| | | | | | | | | | | binding protein 7 |
| 55 | 55 | 106 | 29 | 34 | 60 | 35 | 36 | 121 | 36154_at | KIAA0263 |
| | | | | | | | | | | gene product |
| 56 | 88 | 566 | 48 | 99 | 291 | 52 | 84 | 663 | 32878_f_at | Homo sapiens |
| | | | | | | | | | | cDNA |
| | | | | | | | | | | FLJ32819 fis, |
| | | | | | | | | | | clone |
| | | | | | | | | | | TESTI2002937, |
| | | | | | | | | | | weakly |
| | | | | | | | | | | similar to |
| | | | | | | | | | | HISTONE |
| | | | | | | | | | | H3.2 |
| 57 | 27 | 196 | 97 | 50 | 400 | 72 | 34 | 162 | 35796_at | protein |
| | | | | | | | | | | tyrosine kinase |
| | | | | | | | | | | 9-like (A6- |
| | | | | | | | | | | related protein) |
| 58 | 41 | 315 | 25 | 22 | 198 | 40 | 32 | 273 | 39518_at | Homo sapiens, |
| | | | | | | | | | | clone |
| | | | | | | | | | | MGC: 9628 |
| | | | | | | | | | | IMAGE: 3913311, |
| | | | | | | | | | | mRNA, |
| | | | | | | | | | | complete cds |
| 59 | 92 | 33 | 65 | 107 | 30 | 58 | 90 | 39 | 35425_at | BarH-like |
| | | | | | | | | | | homeobox 2 |
| 60 | 32 | 264 | 114 | 76 | 216 | 73 | 42 | 622 | 143_s_at | TAF5 RNA |
| | | | | | | | | | | polymerase II, |
| | | | | | | | | | | TATA box |
| | | | | | | | | | | binding protein |
| | | | | | | | | | | (TBP)- |
| | | | | | | | | | | associated |
| | | | | | | | | | | factor, 100 kD |
| 61 | 91 | 59 | 26 | 52 | 28 | 55 | 85 | 52 | 34238_at | immunoglobulin |
| | | | | | | | | | | superfamily, |
| | | | | | | | | | | member 1 |
| 62 | 525 | 194 | 63 | 480 | 179 | 53 | 484 | 155 | 33866_at | tropomyosin 4 |
| 63 | 80 | 513 | 75 | 120 | 579 | 94 | 117 | 738 | 37572_at | cholecystokinin |
| 64 | 34 | 459 | 70 | 53 | 336 | 80 | 49 | 1089 | 37961_at | phosphoinositide- |
| | | | | | | | | | | 3-kinase, |
| | | | | | | | | | | regulatory |
| | | | | | | | | | | subunit, |
| | | | | | | | | | | polypeptide 3 |
| | | | | | | | | | | (p55, gamma) |
| 65 | 67 | 1046 | 94 | 97 | 610 | 92 | 95 | 1403 | 35201_at | heterogeneous |
| | | | | | | | | | | nuclear |
| | | | | | | | | | | ribonucleoprotein L |
| 66 | 49 | 140 | 126 | 124 | 99 | 93 | 83 | 135 | 1255_g_at | guanylate |
| | | | | | | | | | | cyclase |
| | | | | | | | | | | activator 1A |
| | | | | | | | | | | (retina) |
| 67 | 62 | 67 | 95 | 62 | 88 | 63 | 56 | 54 | 35368_at | zinc finger |
| | | | | | | | | | | protein 207 |
| 68 | 259 | 25 | 122 | 345 | 48 | 74 | 278 | 43 | 40141_at | cullin 4B |
| 69 | 29 | 45 | 98 | 56 | 100 | 59 | 27 | 82 | 38124_at | midkine |
| | | | | | | | | | | (neurite |
| | | | | | | | | | | growth- |
| | | | | | | | | | | promoting |
| | | | | | | | | | | factor 2) |
| 70 | 16 | 43 | 61 | 11 | 115 | 70 | 15 | 44 | 40617_at | hypothetical |
| | | | | | | | | | | protein |
| | | | | | | | | | | FLJ20274 |
| 71 | 35 | 1074 | 62 | 33 | 703 | 61 | 30 | 1527 | 38970_s_at | Nef-associated |
| | | | | | | | | | | factor 1 |
| 72 | 42 | 84 | 41 | 25 | 65 | 48 | 28 | 84 | 38684_at | ATPase, Ca++ |
| | | | | | | | | | | transporting, |
| | | | | | | | | | | type 2C, |
| | | | | | | | | | | member 1 |
| 73 | 50 | 207 | 68 | 37 | 180 | 66 | 47 | 283 | 41535_at | CDK2- |
| | | | | | | | | | | associated |
| | | | | | | | | | | protein 1 |
| 74 | 103 | 240 | 171 | 226 | 228 | 78 | 123 | 316 | 32703_at | serine/threonine |
| | | | | | | | | | | kinase 18 |
| 75 | 46 | 4 | 83 | 32 | 8 | 62 | 39 | 4 | 36295_at | zinc finger |
| | | | | | | | | | | protein 134 |
| | | | | | | | | | | (clone pHZ-15) |
| 76 | 123 | 988 | 79 | 171 | 757 | 64 | 115 | 1181 | 41208_at | S164 protein |
| 77 | 93 | 394 | 167 | 242 | 242 | 103 | 138 | 481 | 33595_r_at | recombination |
| | | | | | | | | | | activating gene 2 |
| 78 | 53 | 22 | 121 | 91 | 27 | 86 | 61 | 38 | 35414_s_at | jagged 1 |
| | | | | | | | | | | (Alagille |
| | | | | | | | | | | syndrome) |
| 79 | 132 | 203 | 91 | 131 | 168 | 108 | 154 | 215 | 31353_f_at | forkhead box |
| | | | | | | | | | | E2 |
| 80 | 161 | 16 | 43 | 93 | 17 | 69 | 151 | 23 | 35066_g_{—} | fetal |
| | | | | | | | | | at | hypothetical |
| | | | | | | | | | | protein |
| 81 | 374 | 231 | 86 | 428 | 201 | 71 | 369 | 247 | 35784_at | vesicle- |
| | | | | | | | | | | associated |
| | | | | | | | | | | membrane |
| | | | | | | | | | | protein 3 |
| | | | | | | | | | | (cellubrevin) |
| 82 | 240 | 174 | 138 | 356 | 129 | 83 | 236 | 142 | 31472_s_at | Homo sapiens |
| | | | | | | | | | | CD44 isoform |
| | | | | | | | | | | RC (CD44) |
| | | | | | | | | | | mRNA, |
| | | | | | | | | | | complete cds |
| 83 | 86 | 82 | 84 | 100 | 138 | 67 | 68 | 112 | 34433_at | docking protein |
| | | | | | | | | | | 1, 62 kD |
| | | | | | | | | | | (downstream of |
| | | | | | | | | | | tyrosine kinase |
| | | | | | | | | | | 1) |
| 84 | 126 | 151 | 142 | 147 | 348 | 104 | 134 | 268 | 38105_at | hypothetical |
| | | | | | | | | | | protein |
| | | | | | | | | | | FLJ11021 |
| | | | | | | | | | | similar to |
| | | | | | | | | | | splicing factor, |
| | | | | | | | | | | arginine/serine- |
| | | | | | | | | | | rich 4 |
| 85 | 76 | 76 | 107 | 117 | 157 | 129 | 128 | 103 | 31722_at | ribosomal |
| | | | | | | | | | | protein L3 |
| 86 | 52 | 77 | 38 | 31 | 41 | 65 | 45 | 51 | 34104_i_at | immunoglobulin |
| | | | | | | | | | | heavy |
| | | | | | | | | | | constant |
| | | | | | | | | | | gamma 3 (G3m |
| | | | | | | | | | | marker) |
| 87 | 69 | 511 | 110 | 110 | 475 | 121 | 103 | 603 | 41825_at | PTEN induced |
| | | | | | | | | | | putative kinase 1 |
| 88 | 25 | 261 | 93 | 29 | 276 | 91 | 25 | 417 | 41656_at | N- |
| | | | | | | | | | | myristoyltransferase 2 |
| 89 | 36 | 696 | 184 | 77 | 1393 | 113 | 52 | 402 | 40507_at | solute carrier |
| | | | | | | | | | | family 2 |
| | | | | | | | | | | (facilitated |
| | | | | | | | | | | glucose |
| | | | | | | | | | | transporter), |
| | | | | | | | | | | member 1 |
| 90 | 122 | 187 | 77 | 127 | 117 | 75 | 93 | 335 | 34760_at | KIAA0022 |
| | | | | | | | | | | gene product |
| 91 | 133 | 249 | 54 | 86 | 67 | 85 | 129 | 214 | 2092_s_at | secreted |
| | | | | | | | | | | phosphoprotein |
| | | | | | | | | | | 1 (osteopontin, |
| | | | | | | | | | | bone |
| | | | | | | | | | | sialoprotein I, |
| | | | | | | | | | | early T- |
| | | | | | | | | | | lymphocyte |
| | | | | | | | | | | activation 1) |
| 92 | 428 | 609 | 248 | 604 | 598 | 123 | 468 | 859 | 1160_at | cytochrome c-1 |
| 93 | 137 | 267 | 127 | 207 | 256 | 81 | 133 | 262 | 37563_at | KIAA0411 |
| | | | | | | | | | | gene product |
| 94 | 82 | 243 | 118 | 101 | 350 | 79 | 64 | 716 | 36647_at | hypothetical |
| | | | | | | | | | | protein |
| | | | | | | | | | | FLJ10326 |
| 95 | 718 | 568 | 174 | 1053 | 427 | 122 | 851 | 661 | 32841_at | zinc finger |
| | | | | | | | | | | protein 9 (a |
| | | | | | | | | | | cellular |
| | | | | | | | | | | retroviral |
| | | | | | | | | | | nucleic acid |
| | | | | | | | | | | binding |
| | | | | | | | | | | protein) |
| 96 | 237 | 79 | 123 | 284 | 51 | 109 | 266 | 107 | 33469_r_at | complement |
| | | | | | | | | | | factor H |
| | | | | | | | | | | related 3 |
| 97 | 61 | 13 | 24 | 26 | 6 | 47 | 35 | 17 | 1711_at | tumor protein |
| | | | | | | | | | | p53-binding |
| | | | | | | | | | | protein, 1 |
| 98 | 136 | 302 | 46 | 98 | 103 | 89 | 137 | 231 | 32822_at | solute carrier |
| | | | | | | | | | | family 25 |
| | | | | | | | | | | (mitochondrial |
| | | | | | | | | | | carrier; |
| | | | | | | | | | | adenine |
| | | | | | | | | | | nucleotide |
| | | | | | | | | | | translocator), |
| | | | | | | | | | | member 4 |
| 99 | 51 | 19 | 183 | 106 | 78 | 116 | 63 | 31 | 41252_s_at | Homo sapiens |
| | | | | | | | | | | cDNA |
| | | | | | | | | | | FLJ30436 fis, |
| | | | | | | | | | | clone |
| | | | | | | | | | | BRACE2009037 |
| 100 | 71 | 414 | 53 | 42 | 252 | 87 | 58 | 693 | 34965_at | cystatin F |
| | | | | | | | | | | (leukocystatin) |

### Example X

### Threshold Independent Approach to Accessing Significance of OPAL1/G0 and OPAL1/G0-Like Genes

Threshold independent supervised learning algorithms (ROC) and Common Odds Ratio) were used to identify genes associated with outcome in the 167 member pediatric ALL training set described in Example II. Data were normalized using Helman-Veroff algorithm. Nonhuman genes and genes with all call being absent were removed from the data.

The following lists of genes associated with outcome (CCR vs. FAIL) were identified.

TABLE 31
ROC Curve Approach (Threshold Independent Method 1)
Top genes ranked in terms of ROC Accuracy

| Rank | A | Access# | Gene Description |
|---|---|---|---|
| 1 | 0.7131 | 38652_at | hypothetical protein FLJ20154 |
| 2* | 0.6905 | 39418_at | DKFZP564M182 protein |
| 3 | 0.6667 | 41478_at | Homo sapiens cDNA FLJ30991 fis, |
| | | | clone HLUNG1000041 |
| 4* | 0.6653 | 37674_at | aminolevulinate, delta-, synthase 1 |
| 5 | 0.6612 | 38270_at | poly (ADP-ribose) glycohydrolase |
| 6* | 0.6572 | 671_at | secreted protein, acidic, cysteine-rich |
| | | | (osteonectin) |
| 7* | 0.6546 | 1126_s_at | Homo sapiens CD44 isoform |
| | | | RC (CD44) mRNA, |
| | | | complete cds |
| 8* | 0.6529 | 38119_at | glycophorin C (Gerbich blood group) |
| 9 | 0.6527 | 625_at | membrane protein of cholinergic synaptic |
| | | | vesicles |
| 10* | 0.6524 | 31527_at | ribosomal protein S2 |
| 11 | 0.6516 | 587_at | endothelial differentiation, sphingolipid |
| | | | G-protein-coupled receptor, 1 |
| 12* | 0.6513 | 36144_at | KIAA0080 protein |
| 13 | 0.6485 | 41819_at | FYN-binding protein (FYB-120/130) |
| 14 | 0.6454 | 36927_at | hypothetical protein, expressed in |
| | | | osteoblast |
| 15* | 0.6451 | 34760_at | KIAA0022 gene product |
| 16 | 0.6434 | 37748_at | KIAA0232 gene product |
| 17 | 0.6433 | 33188_at | peptidylprolyl isomerase |
| | | | (cyclophilin)-like 2 |
| 18* | 0.6425 | 32336_at | aldolase A, fructose-bisphosphate |
| 19 | 0.6419 | 34349_at | SEC63 protein |
| 20 | 0.6418 | 35796_at | protein tyrosine kinase 9-like |
| | | | (A6-related protein) |

*indicates low expression value predicts CCR

TABLE 32
Common Odds Ratio Approach (Threshold Independent Method 2)
Top genes ranked in terms of common odds ratio

| Rank 1 | Odds Ratio | Rank 2 | A | Access# | Gene Description |
|---|---|---|---|---|---|
| 1 | 3.696 | 1 | 0.7131 | 38652_at | hypothetical protein FLJ20154 |
| 2* | 3.232 | 2 | 0.6905 | 39418_at | DKFZP564M182 protein |
| 3 | 2.725 | 3 | 0.6667 | 41478_at | Homo sapiens cDNA FLJ30991 fis, clone HLUNG1000041 |
| 4* | 2.696 | 4 | 0.6653 | 37674_at | aminolevulinate, delta-, synthase 1 |
| 5 | 2.592 | 5 | 0.6612 | 38270_at | poly (ADP-ribose) glycohydrolase |
| 6* | 2.575 | 6 | 0.6572 | 671_at | secreted protein, acidic, cysteine-rich (osteonectin) |
| 7* | 2.558 | 7 | 0.6546 | 1126_s_at | Homo sapiens CD44 isoform RC (CD44) mRNA, complete cds |
| 8* | 2.541 | 8 | 0.6529 | 38119_at | glycophorin C (Gerbich blood group) |
| 9 | 2.522 | 9 | 0.6527 | 625_at | membrane protein of cholinergic synaptic vesicles |
| 10* | 2.512 | 12 | 0.6513 | 36144_at | KIAA0080 protein |
| 11 | 2.469 | 11 | 0.6516 | 587_at | endothelial differentiation, sphingolipid G-protein-coupled receptor, 1 |
| 12* | 2.449 | 10 | 0.6524 | 31527_at | ribosomal protein S2 |
| 13* | 2.441 | 15 | 0.6451 | 34760_at | KIAA0022 gene product |
| 14 | 2.426 | 16 | 0.6434 | 37748_at | KIAA0232 gene product |
| 15 | 2.413 | 14 | 0.6454 | 36927_at | hypothetical protein, expressed in osteoblast |
| 16 | 2.406 | 13 | 0.6485 | 41819_at | FYN-binding protein (FYB-120/130) |
| 17* | 2.398 | 18 | 0.6425 | 32336_at | aldolase A, fructose-bisphosphate |
| 18* | 2.367 | 24 | 0.6393 | 2062_at | insulin-like growth factor binding protein 7 |
| 19 | 2.363 | 17 | 0.6433 | 33188_at | peptidylprolyl isomerase (cyclophilin)-like 2 |

*indicates low expression value predicts CCR

TABLE 33
Comparison between several gene lists

| | | Rank | Odds | Rank | | | |
|---|---|---|---|---|---|---|---|
| Rank 1 | A | 2 | Ratio | 3 | F | p-value | Access# |
| 1 | 0.7131 | 1 | 3.696 | 1 | 23.327 | 0 | 38652_at |
| 2* | 0.6905 | 2 | 3.232 | 2 | 14.964 | 0.00016 | 39418_at |
| 3 | 0.6667 | 3 | 2.725 | 5 | 13.543 | 0.00032 | 41478_at |
| 4* | 0.6653 | 4 | 2.696 | 14 | 10.31 | 0.00159 | 37674_at |
| 5 | 0.6612 | 5 | 2.592 | 6 | 13.314 | 0.00035 | 38270_at |
| 6* | 0.6572 | 6 | 2.575 | 4 | 13.886 | 0.00027 | 671_at |
| 7* | 0.6546 | 7 | 2.558 | 20 | 10.037 | 0.00183 | 1126_s_at |
| 8* | 0.6529 | 8 | 2.541 | 3 | 14.874 | 0.00016 | 38119_at |
| 9 | 0.6527 | 9 | 2.522 | 22 | 9.958 | 0.0019 | 625_at |
| 10* | 0.6524 | 12 | 2.449 | 7 | 13.178 | 0.00038 | 31527_at |
| 11 | 0.6516 | 11 | 2.469 | 9 | 12.544 | 0.00052 | 587_at |
| 12* | 0.6513 | 10 | 2.512 | 26 | 9.759 | 0.00211 | 36144_at |
| 13 | 0.6485 | 16 | 2.406 | 109 | 7.091 | 0.00851 | 41819_at |
| 14 | 0.6454 | 15 | 2.413 | 18 | 10.16 | 0.00172 | 36927_at |
| 15* | 0.6451 | 13 | 2.441 | 10 | 10.867 | 0.0012 | 34760_at |
| 16 | 0.6434 | 14 | 2.426 | 198 | 5.68 | 0.0183 | 37748_at |
| 17 | 0.6433 | 19 | 2.363 | 161 | 6.039 | 0.01503 | 33188_at |
| 18* | 0.6425 | 17 | 2.398 | 35 | 9.335 | 0.00262 | 32336_at |
| 19 | 0.6419 | 21 | 2.339 | 43 | 8.71 | 0.00363 | 34349_at |
| 20* | 0.6418 | 27 | 2.278 | 8 | 12.545 | 0.00052 | 35796_at |

*indicates low expression value predicts CCR

TABLE 34
Comparison between several gene lists

| Rank 1 | A1 | Rank 2 | A2 | Access # | Gene Description |
|---|---|---|---|---|---|
| 1 | 0.7093 | 1 | 0.713 | 38652_at | hypothetical protein FLJ20154 |
| 2* | 0.6931 | 4* | 0.665 | 37674_at | aminolevulinate, delta-, synthase 1 |
| 3 | 0.6865 | 3 | 0.667 | 41478_at | Homo sapiens cDNA FLJ30991 fis, clone HLUNG1000041 |
| 4* | 0.6776 | 50* | 0.629 | 34433_at | docking protein 1, 62 kD (downstream of tyrosine kinase 1) |
| 5* | 0.6771 | 18* | 0.643 | 32336_at | aldolase A, fructose-bisphosphate |
| 6* | 0.6763 | 15* | 0.645 | 34760_at | KIAA0022 gene product |
| 7 | 0.6723 | 108 | 0.618 | 40027_at | hypothetical protein |
| 8* | 0.6685 | 7* | 0.655 | 1126_s_at | Homo sapiens CD44 isoform RC (CD44) mRNA, complete cds |
| 9 | 0.6666 | 151 | 0.613 | 599_at | H2.0 (Drosophila)-like homeo box 1 |
| 10* | 0.666 | 49* | 0.629 | 40817_at | nucleobindin 1 |
| 11* | 0.6642 | 69* | 0.624 | 1403_s_at | small inducible cytokine A5 (RANTES) |
| 12 | 0.663 | 40 | 0.632 | 1452_at | LIM domain only 4 |
| 13 | 0.6627 | 34 | 0.634 | 39607_at | myotubularin related protein 8 |
| 14* | 0.6623 | 110* | 0.618 | 1062_g_at | interleukin 10 receptor, alpha |
| 15 | 0.6615 | 238 | 0.604 | 35260_at | KIAA0867 protein |
| 16* | 0.6602 | 12* | 0.651 | 36144_at | KIAA0080 protein |
| 17* | 0.6573 | 2* | 0.69 | 39418_at | DKFZP564M182 protein |
| 18 | 0.6562 | 268 | 0.603 | 39931_at | dual-specificity tyrosine-(Y)-phosphorylation regulated kinase 3 |
| 19 | 0.6558 | 22 | 0.64 | 38440_s_at | hypothetical protein |

Rank 1 and A1 are calculated based on the data with T-cell patients removed.
Rank 2 and A2 are calculated based on all 167 training data.
*indicates low expression value predicts CCR

TABLE 35
Comparison between several gene lists

| Rank 1 | A1 | Rank 2 | A2 | Access# | Gene Description |
|---|---|---|---|---|---|
| 1* | 0.9615 | 6956* | 0.512 | 35808_at | [symbol] factor, arginine/serine-rich6 |
| 2 | 0.9231 | 160 | 0.612 | 33469_r_at | complement factor Hrelated3 |
| 3 | 0.9135 | 719 | 0.582 | 31776_at | Human pre-T7NK cell associated protein(1F6) mRNA, 3′ end |
| 4 | 0.9071 | 548 | 0.588 | 38343_at | KIAA0328 protein |
| 5 | 0.9071 | 392 | 0.595 | 33249_at | nuclear receptor subfamily3, groupC, member 2 |
| 6 | 0.9038 | 2720 | 0.549 | 33204_at | forkheed box D1 |
| 7 | 0.9006 | 860 | 0.579 | 32159_at | v-Ki-ras2 Krstenrat sarcoma2viral oncogen homolog |
| 8 | 0.9006 | 7992* | 0.504 | 2021_s_at | cyclin E1 |
| 9 | 0.8974 | 2425 | 0.562 | 32525_r_at | hypothetical protein FLJ14529 |
| 10 | 0.8878 | 144 | 0.614 | 41727_at | KIAA1007 protein |
| 11 | 0.8878 | 5788 | 0.521 | 34484_at | brefeldin Airhibited guerine nuclectide-exchange protein 2 |
| 12 | 0.8878 | 2466 | 0.562 | 34364_at | peptidylpropyl isomerase E(cyclophilin E) |
| 13 | 0.8878 | 1938 | 0.559 | 40606_at | ELL-RELATED RNA POLYMERASE II, ELONGATIONFACTOR |
| 14 | 0.8814 | 842 | 0.579 | 36666_at | CD36 antigen(collagen type I receptor, thrombospondin receptor) |
| 15 | 0.8782 | 7928 | 0.506 | 608_at | apdipoprotein E |
| 16 | 0.875 | 779 | 0.581 | 40332_at | [symbol] growth factor receptor |
| 17 | 0.875 | 2926 | 0.547 | 37238_s_at | membrane-associated tyrosine-and threonine-specific [symbol] 2-inhibitory kinase |
| 18 | 0.875 | 4024 | 0.536 | 39844_at | Homo sapiens, Similar to RKENcDNA 2600001B17 gene, done IMAGE2822298, mRNA, partial cds |
| 19* | 0.8718 | 2* | 0.69 | 39418_at | DKFZP564M182 protein |

Rank 1 and A1 are calculated based on the T-cell data only.
Rank 2 and A2 are calculated based on all 167 training data.

The following tables represent consolidations of a number of different gene lists representing rankings in B-Cell and T-Cell data sets.

TABLE 36
Ranks of Significant Genes Generated in B-Cell, T-Cell and Overall Data Sets
(Genes are ordered on the A ranks in B-Cell Data)

| In B-Cell Data Set | | | In T-Cell Data Set | | | In Overall Data Set | | | | |
|---|---|---|---|---|---|---|---|---|---|---|
| A | F | TNoM | A | F | TNoM | A | F | TNoM | | |
| Rank | Rank | Rank | Rank | Rank | Rank | Rank | Rank | Rank | Accession # | Gene Description |
| 1 | 1 | 1 | 7353 | 5095 | 6931 | 5 | 4 | 1 | 577_at | midkine (neurite growth-promoting factor 2) |
| 2 | 2 | 27 | 7647 | 6799 | 7856 | 3 | 5 | 42 | 37981_at | drebrin 1 |
| 3 | 9 | 63 | 60 | 99 | 98 | 1 | 1 | 7 | 39418_at | DKFZP564M182 protein |
| 4 | 3 | 33 | 7439 | 7001 | 5204 | 7 | 9 | 71 | 32058_at | HNK-1 sulfotransferase |
| 5 | 4 | 17 | 8225 | 6463 | 4257 | 59 | 27 | 82 | 38124_at | midkine (neurite growth-promoting factor 2) |
| 6 | 13 | 11 | 3914 | 2489 | 1617 | 2 | 8 | 2 | 41819_at | FYN-binding protein (FYB-120/130) |
| 7 | 5 | 69 | 3694 | 7740 | 3025 | 16 | 13 | 205 | 824_at | glutathione-S-transferase like; glutathione |
| | | | | | | | | | | transferase omega |
| 8 | 6 | 51 | 2239 | 1452 | 1091 | 67 | 68 | 112 | 34433_at | docking protein 1, 62 kD (downstream of tyrosine |
| | | | | | | | | | | kinase 1) |
| 9 | 8 | 7 | 1528 | 2577 | 824 | 44 | 50 | 95 | 1403_s_at | small inducible cytokine A5 (RANTES) |
| 10 | 12 | 13 | 2701 | 2358 | 3492 | 9 | 10 | 11 | 33412_at | lectin, galactoside-binding, soluble, 1 (galectin 1) |
| 11 | 15 | 9 | 3492 | 4805 | 1951 | 15 | 19 | 10 | 32724_at | phytanoyl-CoA hydroxylase (Refsum disease) |
| 12 | 10 | 21 | 6151 | 7120 | 7344 | 11 | 14 | 35 | 32970_f_at | intracellular hyaluronan-binding protein |
| 13 | 17 | 6 | 7415 | 6374 | 6823 | 14 | 17 | 32 | 41478_at | Homo sapiens cDNA FLJ30991 fis, clone |
| | | | | | | | | | | HLUNG1000041 |
| 14 | 20 | 16 | 1635 | 1359 | 2448 | 4 | 6 | 3 | 37343_at | inositol 1,4,5-triphosphate receptor, type 3 |
| 15 | 7 | 59 | 8019 | 8350 | 7680 | 23 | 16 | 122 | 36524_at | Rho guanine nucleotide exchange factor (GEF) 4 |
| 16 | 26 | 29 | 5415 | 4331 | 1671 | 8 | 12 | 66 | 1126_s_at | Homo sapiens CD44 isoform RC (CD44) mRNA, |
| | | | | | | | | | | complete cds |
| 17 | 14 | 91 | 5628 | 5194 | 4351 | 48 | 28 | 84 | 38684_at | ATPase, Ca++ transporting, type 2C, member 1 |
| 18 | 22 | 56 | 1444 | 1767 | 1145 | 340 | 668 | 117 | 35260_at | KIAA0867 protein |
| 19 | 31 | 65 | 4131 | 4988 | 2772 | 143 | 124 | 194 | 40027_at | hypothetical protein |
| 20 | 18 | 8 | 7175 | 5829 | 5050 | 47 | 35 | 17 | 1711_at | tumor protein p53-binding protein, 1 |
| 21 | 64 | 208 | 1890 | 4989 | 607 | 132 | 253 | 266 | 37674_at | aminolevulinate, delta-, synthase 1 |
| 22 | 52 | 55 | 3432 | 2281 | 2216 | 18 | 33 | 53 | 35145_at | MAX binding protein |
| 23 | 32 | 10 | 5701 | 6669 | 5757 | 86 | 61 | 38 | 35414_s_at | jagged 1 (Alagille syndrome) |
| 24 | 48 | 175 | 7697 | 7982 | 8415 | 41 | 79 | 313 | 32629_f_at | butyrophilin, subfamily 3, member A1 |
| 25 | 19 | 344 | 761 | 865 | 774 | 6 | 3 | 65 | 671_at | secreted protein, acidic, cysteine-rich (osteonectin) |
| 26 | 45 | 174 | 5179 | 4943 | 7299 | 37 | 54 | 149 | 32623_at | gamma-aminobutyric acid (GABA) B receptor, 1 |
| 27 | 21 | 640 | 3961 | 6152 | 4056 | 20 | 21 | 359 | 36927_at | hypothetical protein, expressed in osteoblast |
| 28 | 29 | 30 | 7179 | 6734 | 8385 | 42 | 37 | 94 | 1189_at | cyclin-dependent kinase 8 |
| 29 | 27 | 111 | 1401 | 1436 | 1894 | 171 | 92 | 306 | 32227_at | proteoglycan 1, secretory granule |
| 30 | 77 | 238 | 1583 | 1643 | 795 | 274 | 443 | 1646 | 1062_g_at | interleukin 10 receptor, alpha |
| 31 | 70 | 85 | 8373 | 8005 | 5864 | 30 | 86 | 168 | 36144_at | KIAA0080 protein |
| 32 | 42 | 122 | 8022 | 8223 | 7494 | 75 | 93 | 335 | 34760_at | KIAA0022 gene product |
| 33 | 11 | 40 | 8133 | 8431 | 8188 | 70 | 15 | 44 | 40617_at | hypothetical protein FLJ20274 |
| 34 | 44 | 57 | 7761 | 8070 | 7571 | 63 | 56 | 54 | 35368_at | zinc finger protein 207 |
| 35 | 24 | 39 | 1454 | 1520 | 2607 | 10 | 7 | 6 | 38652_at | hypothetical protein FLJ20154 |
| 36 | 38 | 117 | 5715 | 5390 | 5431 | 105 | 82 | 152 | 33362_at | Cdc42 effector protein 3 |
| 37 | 40 | 19 | 7440 | 5956 | 7128 | 95 | 163 | 18 | 1923_at | cyclin C |
| 38 | 155 | 293 | 6855 | 6239 | 6001 | 200 | 612 | 257 | 37023_at | lymphocyte cytosolic protein 1 (L-plastin) |
| 39 | 74 | 254 | 6737 | 7864 | 5349 | 52 | 84 | 663 | 32878_f_at | Homo sapiens cDNA FLJ32819 fis, clone |
| | | | | | | | | | | TESTI2002937, weakly similar to HISTONE H3.2 |
| 40 | 61 | 171 | 6463 | 6933 | 5257 | 175 | 205 | 460 | 32336_at | aldolase A, fructose-bisphosphate |
| 41 | 54 | 271 | 2220 | 3427 | 2148 | 192 | 190 | 685 | 34481_at | vav 1 oncogene |
| 42 | 72 | 608 | 5332 | 5119 | 3789 | 125 | 181 | 1408 | 35340_at | mel transforming oncogene (derived from cell line |
| | | | | | | | | | | NK14)-RAB8 homolog |
| 43 | 94 | 475 | 3397 | 2541 | 6535 | 430 | 1237 | 1143 | 39931_at | dual-specificity tyrosine-(Y)-phosphorylation |
| | | | | | | | | | | regulated kinase 3 |
| 44 | 103 | 185 | 4222 | 2988 | 5550 | 27 | 71 | 156 | 34171_at | hypothetical protein from EUROIMAGE 2021883 |
| 45 | 35 | 25 | 5963 | 3969 | 7638 | 32 | 18 | 15 | 36129_at | KIAA0397 gene product |
| 46 | 37 | 123 | 5297 | 6905 | 3724 | 162 | 65 | 115 | 34889_at | ATPase, H+ transporting, lysosomal (vacuolar |
| | | | | | | | | | | proton pump), alpha polypeptide, 70 kD, isoform 1 |
| 47 | 75 | 22 | 2740 | 2174 | 2125 | 17 | 29 | 12 | 34332_at | glucosamine-6-phosphate isomerase |
| 48 | 97 | 107 | 7195 | 6468 | 3221 | 83 | 236 | 142 | 31472_s_at | Homo sapiens CD44 isoform RC (CD44) mRNA, |
| | | | | | | | | | | complete cds |
| 49 | 39 | 326 | 7834 | 7858 | 8167 | 118 | 96 | 401 | 40446_at | PHD finger protein 1 |
| 50 | 16 | 210 | 297 | 414 | 624 | 12 | 2 | 81 | 38119_at | glycophorin C (Gerbich blood group) |

TABLE 37
Ranks of Significant Genes Generated in B-Cell, T-Cell and Overall Data Sets
(Genes are ordered on the ranks in T-Cell Data)

| In B-Cell Data Set | | | In T-Cell Data Set | | | In Overall Data Set | | | | |
|---|---|---|---|---|---|---|---|---|---|---|
| A | F | TNoM | A | F | TNoM | A | F | TNoM | | |
| Rank | Rank | Rank | Rank | Rank | Rank | Rank | Rank | Rank | Accession # | Gene Description |
| 4227 | 4648 | 7022 | 1 | 4 | 19 | 872 | 941 | 2400 | 33141_at | hydroxysteroid (17-beta) dehydrogenase 1 |
| 3417 | 2087 | 5974 | 2 | 1 | 2 | 8500 | 7256 | 6418 | 35808_at | splicing factor, arginine/serine-rich 6 |
| 8473 | 8339 | 5826 | 3 | 3 | 10 | 4217 | 3608 | 5137 | 34327_at | SWI/SNF related, matrix associated, actin dependent |
| | | | | | | | | | | regulator of chromatin, subfamily a, member 3 |
| 459 | 3158 | 340 | 4 | 2 | 36 | 19 | 767 | 9 | 41727_at | KIAA1007 protein |
| 7881 | 8248 | 4494 | 5 | 11 | 11 | 2600 | 2695 | 4094 | 34364_at | peptidylprolyl isomerase E (cyclophilin E) |
| 4905 | 2975 | 864 | 6 | 16 | 27 | 7007 | 8506 | 4106 | 34484_at | brefeldin A-inhibited guanine nucleotide-exchange |
| | | | | | | | | | | protein 2 |
| 7078 | 6036 | 1760 | 7 | 6 | 69 | 2709 | 2150 | 2447 | 33878_at | hypothetical protein FLJ13612 |
| 8103 | 8490 | 2366 | 8 | 19 | 20 | 3142 | 4146 | 936 | 33204_at | forkhead box D1 |
| 7007 | 8397 | 6795 | 9 | 21 | 3 | 3279 | 3018 | 7118 | 160022_at | colony stimulating factor 1 receptor, formerly |
| | | | | | | | | | | McDonough feline sarcoma viral (v-fms) oncogene |
| | | | | | | | | | | homolog |
| 3913 | 5807 | 5248 | 10 | 7 | 33 | 651 | 1741 | 590 | 41248_at | likely ortholog of mouse variant polyadenylation |
| | | | | | | | | | | protein CSTF-64 |
| 4933 | 4225 | 1734 | 11 | 5 | 7 | 987 | 1078 | 1820 | 33523_at | alkaline phosphatase, intestinal |
| 1131 | 1246 | 2410 | 12 | 25 | 24 | 6050 | 5789 | 5100 | 33848_r_at | cyclin-dependent kinase inhibitor 1B (p27, Kip1) |
| 702 | 1080 | 180 | 13 | 81 | 6 | 109 | 266 | 107 | 33469_r_at | complement factor H related 3 |
| 1767 | 934 | 2781 | 14 | 9 | 99 | 531 | 265 | 3543 | 39423_f_at | sortilin-related receptor, L(DLR class) A repeats- |
| | | | | | | | | | | containing |
| 7380 | 7385 | 4988 | 15 | 45 | 95 | 3353 | 4297 | 378 | 38981_at | NADH dehydrogenase (ubiquinone) 1 beta |
| | | | | | | | | | | subcomplex, 3 (12 kD, B12) |
| 6933 | 6743 | 8142 | 16 | 18 | 9 | 1958 | 1879 | 2443 | 33841_at | hypothetical protein FLJ11560 |
| 4189 | 4746 | 8069 | 17 | 15 | 17 | 1009 | 1432 | 3069 | 32524_s_at | hypothetical protein FLJ14529 |
| 4835 | 4238 | 4281 | 18 | 13 | 4 | 1236 | 1311 | 4953 | 32159_at | v-Ki-ras2 Kirsten rat sarcoma 2 viral oncogene |
| | | | | | | | | | | homolog |
| 2075 | 2706 | 824 | 19 | 8 | 57 | 252 | 388 | 105 | 32707_at | katanin p60 (ATPase-containg) subunit A 1 |
| 8356 | 5954 | 7079 | 20 | 101 | 8 | 3544 | 2120 | 6238 | 33710_at | putative protein similar to nessy (Drosophila) |
| 5756 | 5167 | 5700 | 21 | 216 | 5 | 5820 | 7418 | 6196 | 33259_at | semenogelin II |
| 8044 | 5787 | 6955 | 22 | 42 | 18 | 3536 | 2270 | 6130 | 32525_r_at | hypothetical protein FLJ14529 |
| 3251 | 2715 | 7856 | 23 | 50 | 312 | 981 | 820 | 2853 | 41276_at | sin3-associated polypeptide, 18 kD |
| 6319 | 7703 | 3893 | 24 | 47 | 13 | 1820 | 3337 | 130 | 40332_at | opioid growth factor receptor |
| 3443 | 4786 | 4018 | 25 | 23 | 35 | 936 | 1573 | 839 | 41650_at | Homo sapiens cDNA FLJ31861 fis, clone |
| | | | | | | | | | | NT2RP7001319 |
| 8248 | 8233 | 7137 | 26 | 30 | 25 | 3962 | 3430 | 7388 | 34340_at | cytochrome b5 outer mitochondrial membrane |
| | | | | | | | | | | precursor |
| 7589 | 6840 | 5732 | 27 | 62 | 64 | 3052 | 2012 | 946 | 33514_at | calcium/calmodulin-dependent protein kinase IV |
| 4330 | 3220 | 4320 | 28 | 31 | 56 | 1286 | 959 | 3067 | 32520_at | nuclear receptor subfamily 1, group D, member 1 |
| 1691 | 1545 | 2690 | 29 | 106 | 12 | 422 | 464 | 756 | 38343_at | KIAA0328 protein |
| 6441 | 6847 | 4723 | 30 | 10 | 234 | 5264 | 5548 | 3346 | 36656_at | CD36 antigen (collagen type I receptor, |
| | | | | | | | | | | thrombospondin receptor) |
| 7508 | 8315 | 5679 | 31 | 29 | 60 | 3200 | 3632 | 5028 | 33056_at | endonuclease G-like 2 |
| 4643 | 2514 | 7830 | 32 | 69 | 14 | 1238 | 584 | 5804 | 41010_at | Homer, neuronal immediate early gene, 1B |
| 599 | 937 | 674 | 33 | 199 | 90 | 692 | 722 | 1107 | 38545_at | inhibin, beta B (activin AB beta polypeptide) |
| 7770 | 4260 | 7989 | 34 | 12 | 15 | 2026 | 933 | 2286 | 1496_at | protein tyrosine phosphatase, receptor type, A |
| 3888 | 3837 | 2088 | 35 | 27 | 32 | 6483 | 7269 | 4626 | 40755_at | MHC class I polypeptide-related sequence A |
| 7021 | 7032 | 3878 | 36 | 55 | 104 | 4386 | 4289 | 5702 | 400_at | insulin promoter factor 1, homeodomain |
| | | | | | | | | | | transcription factor |
| 2560 | 3586 | 6450 | 37 | 46 | 103 | 552 | 1082 | 2127 | 40006_at | sialyltransferase 4B (beta-galactosidase alpha-2,3- |
| | | | | | | | | | | sialytransferase) |
| 520 | 355 | 282 | 38 | 65 | 78 | 77 | 44 | 25 | 35856_r_at | glutamate receptor, ionotropic, kainate 1 |
| 6991 | 5758 | 6881 | 39 | 73 | 16 | 2798 | 2155 | 4910 | 31627_f_at | amine oxidase, copper containing 3 (vascular |
| | | | | | | | | | | adhesion protein 1) |
| 3229 | 1662 | 1989 | 40 | 20 | 266 | 8368 | 7230 | 5560 | 38719_at | N-ethylmaleimide-sensitive factor |
| 6541 | 4081 | 1331 | 41 | 120 | 232 | 3084 | 1584 | 1447 | 36573_at | DEAD/H (Asp-Glu-Ala-Asp/His) box binding |
| | | | | | | | | | | protein 1 |
| 5103 | 6423 | 6115 | 42 | 22 | 83 | 6302 | 5531 | 6548 | 37152_at | peroxisome proliferative activated receptor, delta |
| 4017 | 2364 | 8554 | 43 | 14 | 319 | 1597 | 812 | 7024 | 41840_r_at | Homo sapiens clone IMAGE 25997 |
| 404 | 339 | 1131 | 44 | 64 | 1 | 33 | 41 | 294 | 160030_at | growth hormone receptor |
| 5163 | 4910 | 1442 | 45 | 24 | 272 | 1553 | 1714 | 382 | 39198_s_at | CGI-87 protein |
| 1281 | 946 | 1421 | 46 | 91 | 91 | 296 | 213 | 764 | 38741_at | pleckstrin homology, Sec7 and coiled/coil domains |
| | | | | | | | | | | 2-like |
| 5170 | 2594 | 1027 | 47 | 148 | 101 | 5261 | 8400 | 2776 | 39844_at | Homo sapiens, Similar to RIKEN cDNA |
| | | | | | | | | | | 2600001B17 gene, clone IMAGE: 2822298, mRNA, |
| | | | | | | | | | | partial cds |
| 154 | 223 | 38 | 48 | 108 | 222 | 39 | 53 | 78 | 36069_at | KIAA0456 protein |
| 3290 | 3985 | 4509 | 49 | 39 | 189 | 858 | 1170 | 975 | 34465_at | retinoschisis (X-linked, juvenile) 1 |
| 6433 | 3468 | 4504 | 50 | 122 | 26 | 2185 | 976 | 6308 | 34426_at | major histocompatibility complex, class I-like |
| | | | | | | | | | | sequence |

TABLE 38
Ranks of Significant Genes Generated in B-Cell, T-Cell and Overall Data Sets
(Genes are ordered on the A ranks in Overall Data)

| In B-Cell Data Set | | | In T-Cell Data Set | | | In Overall Data Set | | | | |
|---|---|---|---|---|---|---|---|---|---|---|
| A | F | TNoM | A | F | TNoM | A | F | TNoM | Accession | |
| Rank | Rank | Rank | Rank | Rank | Rank | Rank | Rank | Rank | # | Gene Description |
| 3 | 9 | 63 | 60 | 99 | 98 | 1 | 1 | 7 | 39418_at | DKFZP564M182 protein |
| 6 | 13 | 11 | 3914 | 2489 | 1617 | 2 | 8 | 2 | 41819_at | FYN-binding protein (FYB-120/130) |
| 2 | 2 | 27 | 7647 | 6799 | 7856 | 3 | 5 | 42 | 37981_at | drebrin 1 |
| 14 | 20 | 16 | 1635 | 1359 | 2448 | 4 | 6 | 3 | 37343_at | inositol 1,4,5-triphosphate receptor, type 3 |
| 1 | 1 | 1 | 7353 | 5095 | 6931 | 5 | 4 | 1 | 577_at | midkine (neurite growth-promoting factor 2) |
| 25 | 19 | 344 | 761 | 865 | 774 | 6 | 3 | 65 | 671_at | secreted protein, acidic, cysteine-rich (osteonectin) |
| 4 | 3 | 33 | 7439 | 7001 | 5204 | 7 | 9 | 71 | 32058_at | HNK-1 sulfotransferase |
| 16 | 26 | 29 | 5415 | 4331 | 1671 | 8 | 12 | 66 | 1126_s_at | Homo sapiens CD44 isoform RC (CD44) mRNA, |
| | | | | | | | | | | complete cds |
| 10 | 12 | 13 | 2701 | 2358 | 3492 | 9 | 10 | 11 | 33412_at | lectin, galactoside-binding, soluble, 1 (galectin 1) |
| 35 | 24 | 39 | 1454 | 1520 | 2607 | 10 | 7 | 6 | 38652_at | hypothetical protein FLJ20154 |
| 12 | 10 | 21 | 6151 | 7120 | 7344 | 11 | 14 | 35 | 32970_f_at | intracellular hyaluronan-binding protein |
| 50 | 16 | 210 | 297 | 414 | 624 | 12 | 2 | 81 | 38119_at | glycophorin C (Gerbich blood group) |
| 88 | 184 | 86 | 837 | 444 | 1212 | 13 | 24 | 40 | 36331_at | Homo sapiens mRNA; cDNA DKFZp586C091 |
| | | | | | | | | | | (from clone DKFZp586C091) |
| 13 | 17 | 6 | 7415 | 6374 | 6823 | 14 | 17 | 32 | 41478_at | Homo sapiens cDNA FLJ30991 fis, clone |
| | | | | | | | | | | HLUNG1000041 |
| 11 | 15 | 9 | 3492 | 4805 | 1951 | 15 | 19 | 10 | 32724_at | phytanoyl-CoA hydroxylase (Refsum disease) |
| 7 | 5 | 69 | 3694 | 7740 | 3025 | 16 | 13 | 205 | 824_at | glutathione-S-transferase like; glutathione |
| | | | | | | | | | | transferase omega |
| 47 | 75 | 22 | 2740 | 2174 | 2125 | 17 | 29 | 12 | 34332_at | glucosamine-6-phosphate isomerase |
| 22 | 52 | 55 | 3432 | 2281 | 2216 | 18 | 33 | 53 | 35145_at | MAX binding protein |
| 459 | 3158 | 340 | 4 | 2 | 36 | 19 | 767 | 9 | 41727_at | KIAA1007 protein |
| 27 | 21 | 640 | 3961 | 6152 | 4056 | 20 | 21 | 359 | 36927_at | hypothetical protein, expressed in osteoblast |
| 185 | 318 | 821 | 446 | 491 | 424 | 21 | 59 | 314 | 32650_at | neuronal protein |
| 181 | 414 | 137 | 281 | 313 | 1354 | 22 | 62 | 27 | 38437_at | MLN51 protein |
| 15 | 7 | 59 | 8019 | 8350 | 7680 | 23 | 16 | 122 | 36524_at | Rho guanine nucleotide exchange factor (GEF) 4 |
| 247 | 242 | 150 | 132 | 158 | 301 | 24 | 31 | 62 | 40523_at | hepatocyte nuclear factor 3, beta |
| 112 | 210 | 362 | 1610 | 1034 | 1839 | 25 | 78 | 143 | 31527_at | ribosomal protein S2 |
| 159 | 832 | 262 | 1147 | 990 | 464 | 26 | 310 | 154 | 33637_g_at | cancer/testis antigen |
| 44 | 103 | 185 | 4222 | 2988 | 5550 | 27 | 71 | 156 | 34171_at | hypothetical protein from EUROIMAGE 2021883 |
| 77 | 216 | 1883 | 1706 | 1656 | 3994 | 28 | 120 | 1044 | 36576_at | H2A histone family, member Y |
| 74 | 264 | 54 | 3350 | 2695 | 3750 | 29 | 142 | 37 | 35059_at | Homo sapiens clone FBA1 Cri-du-chat region |
| | | | | | | | | | | mRNA |
| 31 | 70 | 85 | 8373 | 8005 | 5864 | 30 | 86 | 168 | 36144_at | KIAA0080 protein |
| 226 | 116 | 668 | 304 | 181 | 637 | 31 | 23 | 83 | 38270_at | poly (ADP-ribose) glycohydrolase |
| 45 | 35 | 25 | 5963 | 3969 | 7638 | 32 | 18 | 15 | 36129_at | KIAA0397 gene product |
| 404 | 339 | 1131 | 44 | 64 | 1 | 33 | 41 | 294 | 160030_at | growth hormone receptor |
| 94 | 137 | 215 | 749 | 5206 | 653 | 34 | 149 | 28 | 38865_at | GRB2-related adaptor protein 2 |
| 133 | 136 | 286 | 1442 | 957 | 2329 | 35 | 36 | 121 | 36154_at | KIAA0263 gene product |
| 56 | 336 | 90 | 3557 | 3257 | 4183 | 36 | 238 | 80 | 37748_at | KIAA0232 gene product |
| 26 | 45 | 174 | 5179 | 4943 | 7299 | 37 | 54 | 149 | 32623_at | gamma-aminobutyric acid (GABA) B receptor, 1 |
| 54 | 43 | 447 | 3621 | 2573 | 4252 | 38 | 26 | 371 | 38087_s_at | S100 calcium-binding protein A4 (calcium protein, |
| | | | | | | | | | | calvasculin, metastasin, murine placental homolog) |
| 154 | 223 | 38 | 48 | 108 | 222 | 39 | 53 | 78 | 36069_at | KIAA0456 protein |
| 337 | 207 | 2027 | 102 | 87 | 674 | 40 | 32 | 273 | 39518_at | Homo sapiens, clone MGC: 9628 IMAGE: |
| | | | | | | | | | | 3913311, mRNA, complete cds |
| 24 | 48 | 175 | 7697 | 7982 | 8415 | 41 | 79 | 313 | 32629_f_at | butyrophilin, subfamily 3, member A1 |
| 28 | 29 | 30 | 7179 | 6734 | 8385 | 42 | 37 | 94 | 1189_at | cyclin-dependent kinase 8 |
| 106 | 126 | 84 | 425 | 1480 | 1194 | 43 | 77 | 165 | 36081_s_at | chromosome 21 open reading frame 18 |
| 9 | 8 | 7 | 1528 | 2577 | 824 | 44 | 50 | 95 | 1403_s_at | small inducible cytokine A5 (RANTES) |
| 84 | 171 | 245 | 7903 | 5919 | 3193 | 45 | 161 | 139 | 2062_at | insulin-like growth factor binding protein 7 |
| 63 | 98 | 114 | 4077 | 4359 | 979 | 46 | 75 | 150 | 32739_at | N-acetylglucosamine-phosphate mutase |
| 20 | 18 | 8 | 7175 | 5829 | 5050 | 47 | 35 | 17 | 1711_at | tumor protein p53-binding protein, 1 |
| 17 | 14 | 91 | 5628 | 5194 | 4351 | 48 | 28 | 84 | 38684_at | ATPase, Ca++ transporting, type 2C, member 1 |
| 202 | 194 | 526 | 174 | 85 | 43 | 49 | 40 | 442 | 1818_at | NO_.SIF_seq |
| 373 | 415 | 523 | 299 | 310 | 131 | 50 | 69 | 296 | 1676_s_at | eukaryotic translation elongation factor 1 gamma |

TABLE 39
Ranks of Uniformly Significant Genes Generated in Data Sets with T-Cell Data Removed

| In Random | | | | | | | | | | |
|---|---|---|---|---|---|---|---|---|---|---|
| Training Set | | | In Random Test Set | | | In Overall B-Cell Data | | | | |
| A | F | TNoM | A | F | TNoM | A | F | TNoM | | |
| Rank | Rank | Rank | Rank | Rank | Rank | Rank | Rank | Rank | Accession # | Gene Description |
| 1 | 1 | 1 | 1 | 1 | 1 | 1 | 1 | 1 | 577_at | midkine (neurite growth-promoting factor 2) |
| 2 | 2 | 25 | 2 | 5 | 21 | 2 | 2 | 27 | 37981_at | drebrin 1 |
| 3 | 8 | 44 | 6 | 21 | 86 | 3 | 9 | 63 | 39418_at | DKFZP564M182 protein |
| 4 | 15 | 7 | 11 | 19 | 8 | 6 | 13 | 11 | 41819_at | FYN-binding protein (FYB-120/130) |
| 5 | 3 | 19 | 4 | 3 | 20 | 5 | 4 | 17 | 38124_at | midkine (neurite growth-promoting factor 2) |
| 6 | 4 | 26 | 3 | 2 | 6 | 4 | 3 | 33 | 32058_at | HNK-1 sulfotransferase |
| 7 | 7 | 53 | 10 | 9 | 32 | 8 | 6 | 51 | 34433_at | docking protein 1, 62 kD (downstream of tyrosine |
| | | | | | | | | | | kinase 1) |
| 8 | 9 | 12 | 16 | 17 | 13 | 9 | 8 | 7 | 1403_s_at | small inducible cytokine A5 (RANTES) |
| 9 | 5 | 54 | 5 | 4 | 80 | 7 | 5 | 69 | 824_at | glutathione-S-transferase like; glutathione |
| | | | | | | | | | | transferase omega |
| 10 | 6 | 40 | 15 | 8 | 43 | 15 | 7 | 59 | 36524_at | Rho guanine nucleotide exchange factor (GEF) 4 |
| 11 | 12 | 6 | 18 | 24 | 4 | 11 | 15 | 9 | 32724_at | phytanoyl-CoA hydroxylase (Refsum disease) |
| 12 | 17 | 11 | 13 | 14 | 7 | 14 | 20 | 16 | 37343_at | inositol 1,4,5-triphosphate receptor, type 3 |
| 13 | 13 | 18 | 7 | 10 | 16 | 10 | 12 | 13 | 33412_at | lectin, galactoside-binding, soluble, 1 (galectin 1) |
| 14 | 11 | 17 | 9 | 6 | 12 | 12 | 10 | 21 | 32970_f_at | intracellular hyaluronan-binding protein |
| 15 | 20 | 10 | 12 | 12 | 17 | 13 | 17 | 6 | 41478_at | Homo sapiens cDNA FLJ30991 fis, clone |
| | | | | | | | | | | HLUNG1000041 |
| 16 | 26 | 15 | 14 | 25 | 9 | 16 | 26 | 29 | 1126_s_at | Homo sapiens CD44 isoform RC (CD44) mRNA, |
| | | | | | | | | | | complete cds |
| 17 | 22 | 62 | 8 | 11 | 33 | 17 | 14 | 91 | 38684_at | ATPase, Ca++ transporting, type 2C, member 1 |
| 18 | 23 | 63 | 17 | 15 | 45 | 18 | 22 | 56 | 35260_at | KIAA0867 protein |
| 19 | 31 | 85 | 20 | 26 | 100 | 19 | 31 | 65 | 40027_at | hypothetical protein |
| 20 | 18 | 5 | 21 | 16 | 2 | 20 | 18 | 8 | 1711_at | tumor protein p53-binding protein, 1 |
| 21 | 14 | 208 | 32 | 29 | 417 | 25 | 19 | 344 | 671_at | secreted protein, acidic, cysteine-rich (osteonectin) |
| 22 | 69 | 99 | 23 | 75 | 93 | 21 | 64 | 208 | 37674_at | aminolevulinate, delta-, synthase 1 |
| 23 | 49 | 68 | 19 | 48 | 44 | 22 | 52 | 55 | 35145_at | MAX binding protein |
| 24 | 30 | 31 | 44 | 42 | 27 | 28 | 29 | 30 | 1189_at | cyclin-dependent kinase 8 |
| 25 | 39 | 140 | 28 | 51 | 47 | 26 | 45 | 174 | 32623_at | gamma-aminobutyric acid (GABA) B receptor, 1 |
| 26 | 50 | 103 | 27 | 46 | 57 | 24 | 48 | 175 | 32629_f_at | butyrophilin, subfamily 3, member A1 |
| 27 | 56 | 469 | 43 | 88 | 737 | 42 | 72 | 608 | 35340_at | mel transforming oncogene (derived from cell line |
| | | | | | | | | | | NK14)-RAB8 homolog |
| 28 | 74 | 171 | 26 | 70 | 96 | 30 | 77 | 238 | 1062_g_at | interleukin 10 receptor, alpha |
| 29 | 21 | 384 | 29 | 20 | 457 | 27 | 21 | 640 | 36927_at | hypothetical protein, expressed in osteoblast |
| 30 | 34 | 8 | 22 | 23 | 3 | 23 | 32 | 10 | 35414_s_at | jagged 1 (Alagille syndrome) |
| 31 | 27 | 60 | 25 | 28 | 38 | 29 | 27 | 111 | 32227_at | proteoglycan 1, secretory granule |
| 32 | 147 | 159 | 42 | 216 | 277 | 38 | 155 | 293 | 37023_at | lymphocyte cytosolic protein 1 (L-plastin) |
| 33 | 46 | 59 | 41 | 71 | 88 | 32 | 42 | 122 | 34760_at | KIAA0022 gene product |
| 34 | 36 | 65 | 38 | 41 | 90 | 34 | 44 | 57 | 35368_at | zinc finger protein 207 |
| 35 | 10 | 36 | 37 | 7 | 67 | 33 | 11 | 40 | 40617_at | hypothetical protein FLJ20274 |
| 36 | 58 | 123 | 40 | 84 | 152 | 40 | 61 | 171 | 32336_at | aldolase A, fructose-bisphosphate |
| 37 | 24 | 41 | 48 | 27 | 78 | 35 | 24 | 39 | 38652_at | hypothetical protein FLJ20154 |
| 38 | 93 | 95 | 24 | 78 | 79 | 31 | 70 | 85 | 36144_at | KIAA0080 protein |
| 39 | 44 | 27 | 35 | 39 | 35 | 37 | 40 | 19 | 1923_at | cyclin C |
| 40 | 33 | 21 | 54 | 36 | 31 | 45 | 35 | 25 | 36129_at | KIAA0397 gene product |
| 41 | 63 | 296 | 34 | 45 | 221 | 41 | 54 | 271 | 34481_at | vav 1 oncogene |
| 42 | 97 | 657 | 33 | 86 | 404 | 51 | 113 | 772 | 1637_at | mitogen-activated protein kinase-activated protein |
| | | | | | | | | | | kinase 3 |
| 43 | 45 | 184 | 31 | 40 | 170 | 36 | 38 | 117 | 33362_at | Cdc42 effector protein 3 |
| 44 | 72 | 20 | 39 | 76 | 18 | 47 | 75 | 22 | 34332_at | glucosamine-6-phosphate isomerase |
| 45 | 100 | 161 | 52 | 128 | 123 | 44 | 103 | 185 | 34171_at | hypothetical protein from EUROIMAGE 2021883 |
| 46 | 79 | 368 | 45 | 68 | 248 | 39 | 74 | 254 | 32878_f_at | Homo sapiens cDNA FLJ32819 fis, clone |
| | | | | | | | | | | TESTI2002937, weakly similar to HISTONE H3.2 |
| 47 | 102 | 397 | 49 | 98 | 428 | 43 | 94 | 475 | 39931_at | dual-specificity tyrosine-(Y)-phosphorylation |
| | | | | | | | | | | regulated kinase 3 |
| 48 | 16 | 261 | 55 | 18 | 329 | 50 | 16 | 210 | 38119_at | glycophorin C (Gerbich blood group) |
| 49 | 323 | 96 | 83 | 348 | 292 | 56 | 336 | 90 | 37748_at | KIAA0232 gene product |
| 50 | 42 | 401 | 66 | 55 | 623 | 54 | 43 | 447 | 38087_s_at | S100 calcium-binding protein A4 (calcium protein, |
| | | | | | | | | | | calvasculin, metastasin, murine placental homolog) |

### Example XI

### Correlated Gene Lists for Outcome Prediction in Pre-B ALL Cohort

Introduction. This Example summarizes and correlates selected gene lists predictive of outcome (specifically, CCR vs. Failure) obtained for the pre-B ALL cohort described in Example IB. “Task 2” refers to CCR vs. FAIL for B-cell+T-cell patients; “Task 2a” is CCR vs. FAIL for B-cell only patients. Gene lists selected for evaluation were produced by the following methods: (1) a compilation of genes identified using feature selection combined with a supervised learning techniques such as SVM/RFE, Discriminant Analysis/t-test, Fuzzy Inference/rank-ordering statistics, and Bayesian Nets/TNoM; note that SVM/RFE and Bayesian Net/TNoM are both multivariate (MV) gene selection techniques; the others are univariate; (2) TNoM gene selection; (3) supervised classification; (4) empirical CDF/MaxDiff method; (5) threshold independent approach; (6) GA/KNN; (7) uniformly significant genes via resampling; (8) ANOVA “gene contrast” lists derived via VxInsight.

The techniques fall into two broad categories, which we have termed univariate and multivariate.

Group I (univariate). These methods evaluate the significance of a given gene in contributing to outcome discrimination on an individual basis. They include:

two-sample t-test (here equivalent to F-test or one-way ANOVA)
Rank-ordering statistics
ROC curves (“threshold-independent method 1”)
Common odds ratio approach (“threshold-independent method 2”)
“Most uniformly significant genes” via resampling—average rank from 172 train/test resamplings of the dataset, for each of 3 different methods: F-test, ROC accuracy A, and TNoM score;
GA/KNN
Empirical cumulative distribution function (CDF) MaxDiff approach
TNoM method—used to pre-filter genes for use as parent sets in constructing (and scoring) competing Bayesian nets that best explain the training set data.
   Group 2 (multivariate). These methods identify groups of genes that act in concert to discriminate outcome. The optimal gene groups are determined via an iterative (SVM, stepwise DA) or combinatoric exploration (Bayesian) procedure. They include:
SVM/RFE (Support Vector Machines with Recursive Feature Elimination)
Bayesian net evaluation of (via BD metric) of highest-scoring parent sets (gene combinations)
Stepwise discriminant analysis

The top genes in each group are identified and to determine how often the same genes turn up repeatedly within each group. The following two tables correspond to Tasks 2 (Table 40) and 2a (Table 41). The top 20 genes found in Table 40 are listed in Table 42 with more detailed annotations.

TABLE 40
Task 2 (CCR vs. FAIL, full dataset of pre-B and T-cell cases)
Univariate and multivariate (MV) methods, comparative gene rankings:
Bayesian Net-derived G0, G1, G2 (MV) indicated in yellow
All methods used training set only, except for the method of column 1, which used combined train/test set, and gave results comparable to
172 resampled training sets (“uniformly most significant genes”), and column 3, ANOVA (VxInsight “User Contrast”).
Gene descriptions are from Affy Complete Entry (in some cases supplemented by additional/different information
provided by analysts, in parentheses)

| HK ROC- | | | | | HK | | | HK | | | |
|---|---|---|---|---|---|---|---|---|---|---|---|
| accuracy- | | | | HK | Threshold- | XW | | Stepwise | | | |
| selected | SM | GD | | F- | Independent | Rank- | | Discrim- | EA | | |
| genes, | Empirical | ANOVA | | test, | Method 1 | Order- | XW | inant | SVM/ | | |
| overall | CDF | (Vx “User | RV/PH | Table | (ROC | ing | GA/ | Analysis, | RFE | Affy | |
| dataset | MaxDiff | Contrast”) | TNoM | 3 | Curves) | Statistic | KNN | HK (MV) | (MV) | Accession # | Description |
| 1 | 4 | 1 | | 3 | 2 | | 15 | | | 39418_at | DKFZP564M182 protein |
| 2 | | 19 | 12 | | 13 | | 9 | | | 41819_at | FYN-binding protein |
| | | | | | | | | | | | (FYB-120/130) |
| 3 | | 2 | | | | | | | | 37981_at | drebrin 1 |
| 5 | | 6 | | | | | | | | 577_at | midkine (neurite growth- |
| | | | | | | | | | | | promoting factor 2) |
| 4 | 5 | 7 | 21 | | | 26 | | | | 37343_at | inositol 1,4,5-triphosphate |
| | | | | | | | | | | | receptor, type 3 |
| 7 | | 20 | | | | | | | | 32058_at | HNK-1 sulfotransferase |
| 9 | | | | | | | | | 5 | 33412_at | lectin, galactoside-binding, |
| | | | | | | | | | | | soluble, 1 (galectin 1) |
| 8 | 22 | 8 | 13 | 15 | 7 | | | | | 1126_s_at | Homo sapiens CD44 |
| | | | | | | | | | | | isoform RC (CD44) |
| | | | | | | | | | | | mRNA, complete cds |
| 6 | | 5 | | 4 | 6 | 3 | 13 | 2 | 19 | 671_at | secreted protein, acidic, |
| | | | | | | | | | | | cysteine-rich (osteonectin) |
| 11 | | 9 | | | | 20 | | | | 32970_f_at | intracellular hyaluronan- |
| | | | | | | | | | | | binding protein |
| 16 | | 13 | | | | 4 | | | | 824_at | glutathione-S-transferase |
| | | | | | | | | | | | like; glutathione transferase |
| | | | | | | | | | | | omega |
| 15 | | | | | | | | | | 32724_at | phytanoyl-CoA |
| | | | | | | | | | | | hydroxylase (Refsum |
| | | | | | | | | | | | disease) |
| 10 | 1 | 12 | 1 | 1 | 1 | 1 | 1 | 1 | 2 | 38652_at | hypothetical protein |
| | | | | | | | | | | | FLJ20154 (aka |
| | | | | | | | | | | | hypothetical protein |
| | | | | | | | | | | | FLJ20367, NM_017787) |
| | | | | | | | | | | | (G0) |
| 13 | | | | | | | | | | 36331_at | Homo sapiens mRNA; |
| | | | | | | | | | | | cDNA DKFZp586C091 |
| | | | | | | | | | | | (from clone |
| | | | | | | | | | | | DKFZp586C091) |
| 14 | | | 23 | 5 | 3 | 7 | 24 | | 10 | 41478_at | Homo sapiens cDNA |
| | | | | | | | | | | | FLJ30991 fis, clone |
| | | | | | | | | | | | HLUNG1000041 |
| 12 | 11 | 4 | | 2 | 8 | 13 | | | | 38119_at | glycophorin C (Gerbich |
| | | | | | | | | | | | blood group) (NM_002101 |
| | | | | | | | | | | | analysis glycophorin C |
| | | | | | | | | | | | isoform 1 NM_016815 |
| | | | | | | | | | | | analysis glycophorin C |
| | | | | | | | | | | | isoform 2) |
| 20 | | | | 17 | 14 | | | 4 | 6 | 36927_at | hypothetical protein, |
| | | | | | | | | | | | expressed in osteoblast |
| 18 | | | | | | | | | | 35145_at | MAX binding protein |
| 26 | 14 | | | | | | | | | 33637_g_at | cancer/testis antigen |
| | | | | | | | | | | 34610_at | guanine nucleotide binding |
| | | | | | | | | | | | protein (G protein), beta |
| | | | | | | | | | | | polypeptide 2-like 1 (G1) |
| | | | | | | | | | | 35659_at | interleukin 10 receptor, |
| | | | | | | | | | | | alpha (G2) |
| | 2 | | | | | | | | | 38585_at | hemoglobin gamma A |
| | 3 | | | | | | | | | 35965_at | heat shock 70 kD protein 6 |
| | | | | | | | | | | | HSP70B |
| | 6 | | | | | | | | | 32557_at | U2 small nuclear |
| | | | | | | | | | | | ribonucleoprotein auxiliary |
| | | | | | | | | | | | factor 65 kD |
| | 7 | | | | | | | | | 40435_at | solute carrier family 25 |
| | | | | | | | | | | | (mitochondrial carrier; |
| | | | | | | | | | | | adenine nucleotide |
| | | | | | | | | | | | translocator), member 6 |
| | 8 | | 8 | | | 27 | 17 | | | 32624_at | DKFZp566D133 protein |
| | | | | | | | | | | | (likely ortholog of mouse |
| | | | | | | | | | | | tuberin-like protein 1) |
| | 9 | | 2 | | | | | | | 33415_at | non-metastatic cells 2 |
| | | | | | | | | | | | protein NM23B expressed |
| | | | | | | | | | | | in |
| | 10 | | 5 | | | | | | | 41559_at | Homo sapiens, clone |
| | | | | | | | | | | | IMAGE: 3880654, mRNA |
| | 12 | | 29 | | | | | | | 31472_s_at | Homo sapiens CD44 |
| | | | | | | | | | | | isoform RC (CD44) |
| | | | | | | | | | | | mRNA, complete cds |
| | 13 | | | | | | | | | 38750_at | Notch Drosophila homolog 3 |
| | 15 | | 6 | | | | | | | 1980_s_at | non-metastatic cells 2 |
| | | | | | | | | | | | protein NM23B expressed |
| | | | | | | | | | | | in |
| | 16 | | | | | | | | | 32703_at | serine/threonine kinase 18 |
| | 17 | 23 | 25 | | | | 3 | | | 1403_s_at | small inducible cytokine |
| | | | | | | | | | | | A5 RANTES (chemokine |
| | | | | | | | | | | | (C-C motif) ligand 5) |
| | 18 | | | | | | | | | 2091_at | wingless-type MMTV |
| | | | | | | | | | | | integration site family, |
| | | | | | | | | | | | member 4 |
| | 19 | | | | | | | | | 36624_at | IMP inosine |
| | | | | | | | | | | | monophosphate |
| | | | | | | | | | | | dehydrogenase 2 |
| | 20 | | | | | | | | | 176_at | protein phosphatase 2 |
| | | | | | | | | | | | regulatory subunit B B56 |
| | | | | | | | | | | | gamma isoform |
| | 21 | | | | | | | | | 38794_at | upstream binding |
| | | | | | | | | | | | transcription factor RNA |
| | | | | | | | | | | | polymerase I |
| | 23 | | | | | | 5 | | | 37986_at | erythropoietin receptor |
| | | | | | | | | | | | precursor |
| | 24 | | | | | | | | | 36386_at | pyruvate dehydrogenase |
| | | | | | | | | | | | kinase isoenzyme 1 |
| | 25 | | | | | | | | | 38865_at | GRB2-related adaptor |
| | | | | | | | | | | | protein 2 |
| | | 3 | | 9 | | | | | | 38971_r_at | Nef-associated factor 1 |
| | | 10 | | | | | | | | 41185_f_at | SMT3 (suppressor of mif |
| | | | | | | | | | | | two 3, yeast) homolog 2 |
| | | 11 | | | | | | | | 33362_at | Cdc42 effector protein 3 |
| | | 14 | 18 | 6 | 20 | 5 | | | | 35796_at | protein tyrosine kinase 9- |
| | | | | | | | | | | | like (A6-related protein) |
| | | 15 | | | | | | | | 40523_at | hepatocyte nuclear factor 3, |
| | | | | | | | | | | | beta |
| 24 | | 16 | | | | | | | | 37184_at | syntaxin 1A (brain) |
| | | 17 | | | | | | | | 34890_at | ATPase, H+ transporting, |
| | | | | | | | | | | | lysosomal (vacuolar proton |
| | | | | | | | | | | | pump), alpha polypeptide, |
| | | | | | | | | | | | 70 kD, isoform 1 |
| | | 18 | | | | | | | | 41257_at | type 1 tumor necrosis factor |
| | | | | | | | | | | | receptor shedding |
| | | | | | | | | | | | aminopeptidase regulator |
| | | | | | | | | | | | (NM_001750 analysis |
| | | | | | | | | | | | calpastatin) |
| | | 21 | | | | | | | | 38970_s_at | Nef-associated factor 1 |
| | | 22 | | | | | | | | 34809_at | KIAA0999 protein |
| | | | | | | | | | | | (hypothetical protein |
| | | | | | | | | | | | FLJ12240) |
| | | 24 | | | | | | | | 33866_at | tropomyosin 4 |
| 17 | | 25 | | | | | | | | 34332_at | glucosamine-6-phosphate |
| | | | | | | | | | | | isomerase |
| | | | 3 | | | | | | | 36012_at | PIBF1 gene product |
| | | | | | | | | | | | (progesterone-induced |
| | | | | | | | | | | | blocking factor 1) |
| | | | 4 | | | | | | | 38838_at | polymyositis/scleroderma |
| | | | | | | | | | | | autoantigen 1 (75 kD) |
| | | | 7 | | | | | | | 31444_s_at | annexin A2 pseudogene 3 |
| | | | 9 | | | | | | | 36295_at | zinc finger protein 134 |
| | | | | | | | | | | | (clone pHZ-15) |
| | | | 10 | | | | | | | 38134_at | pleiomorphic adenoma |
| | | | | | | | | | | | gene 1 |
| | | | 11 | 7 | 5 | 12 | | | 18 | 38270_at | poly (ADP-ribose) |
| | | | | | | | | | | | glycohydrolase |
| | | | 14 | | | | 19 | | | 32224_at | KIAA0769 gene product |
| | | | 15 | 19 | 18 | | | | | 32336_at | aldolase A, fructose- |
| | | | | | | | | | | | bisphosphate |
| | | | 16 | | | | | | | 32398_s_at | low density lipoprotein |
| | | | | | | | | | | | receptor-related protein 8, |
| | | | | | | | | | | | apolipoprotein e receptor |
| | | | 17 | | | | | | | 35756_at | chromosome 19 open |
| | | | | | | | | | | | reading frame 3 (regulator |
| | | | | | | | | | | | of G-protein signalling 19 |
| | | | | | | | | | | | interacting protein 1) |
| | | | 19 | 14 | | | | 7 | | 36154_at | KIAA0263 gene product |
| | | | 20 | | | 14 | | | | 37147_at | stem cell growth factor; |
| | | | | | | | | | | | lymphocyte secreted C-type |
| | | | | | | | | | | | lectin |
| | | | 22 | | | | | | | 40141_at | cullin 4B |
| 19 | | | 24 | | | | | | | 41727_at | KIAA1007 protein |
| | | | 26 | | | | | | | 1488_at | protein tyrosine |
| | | | | | | | | | | | phosphatase, receptor type, K |
| | | | 27 | | | | | | | 1711_at | tumor protein p53-binding |
| | | | | | | | | | | | protein, 1 |
| | | | 28 | | | | | | | 307_at | arachidonate 5- |
| | | | | | | | | | | | lipoxygenase |
| | | | 30 | | | | | | | 31473_s_at | tankyrase, TRF1- |
| | | | | | | | | | | | interacting ankyrin-related |
| | | | | | | | | | | | ADP-ribose polymerase |
| | | | | 8 | 11 | 2 | | | 11 | 587_at | endothelial differentiation, |
| | | | | | | | | | | | sphingolipid G-protein- |
| | | | | | | | | | | | coupled receptor, 1 |
| | | | | 10 | 15 | | | 3 | 29 | 34760_at | KIAA0022 gene product |
| 25 | | | | 11 | 10 | | | | | 31527_at | ribosomal protein S2 |
| | | | | 12 | 4 | 19 | | | | 37674_at | Aminolevulinate, delta-, |
| | | | | | | | | | | | synthase 1 |
| | | | | 13 | 12 | | 8 | 6 | 21 | 36144_at | KIAA0080 protein |
| | | | | 16 | | | | | | 31695_g_at | regulatory solute carrier |
| | | | | | | | | | | | protein, family 1, member 1 |
| | | | | 18 | | | | | | 34965_at | cystatin F (leukocystatin) |
| | | | | 20 | 9 | 9 | | 5 | 14 | 625_at | membrane protein of |
| | | | | | | | | | | | cholinergic synaptic |
| | | | | | | | | | | | vesicles |
| | | | | | 16 | | | | | 37748_at | KIAA0232 gene product |
| | | | | | 17 | | | | | 33188_at | peptidylprolyl isomerase |
| | | | | | | | | | | | (cyclophilin)-like 2 |
| | | | | | 19 | | | 9 | | 34349_at | SEC63 protein |
| | | | | | | 8 | | 11 | | 40817_at | nucleobindin 1 |
| | | | | | | 24 | | | | 2065_s_at | BCL2-associated X protein |
| | | | | | | 25 | | | | 404_at | interleukin 4 receptor |
| | | | | | | | 2 | 25 | | 35991_at | Sm protein F |
| | | | | | | | 4 | | | 41097_at | telomeric repeat binding |
| | | | | | | | | | | | factor 2 |
| | | | | | | | 6 | | | 40276_at | proteasome (prosome, |
| | | | | | | | | | | | macropain) 26S subunit, |
| | | | | | | | | | | | non-ATPase, 7 (Mov34 |
| | | | | | | | | | | | homolog) |
| | | | | | | | 7 | | | 40272_at | collapsin response mediator |
| | | | | | | | | | | | protein 1 |
| | | | | | | | 10 | | | 40898_at | sequestosome 1 |
| | | | | | | | 11 | | | 33229_at | ribosomal protein S6 |
| | | | | | | | | | | | kinase, 90 kD, polypeptide 3 |
| | | | | | | | 12 | | | 35633_at | engulfment and cell |
| | | | | | | | | | | | motility 1 (ced-12 |
| | | | | | | | | | | | homolog, C. elegans) |
| | | | | | | | 14 | | | 514_at | Cas-Br-M (murine) |
| | | | | | | | | | | | ectropic retroviral |
| | | | | | | | | | | | transforming sequence b |
| | | | | | | | 16 | | | 38155_at | origin recognition complex, |
| | | | | | | | | | | | subunit 5 (yeast homolog)- |
| | | | | | | | | | | | like |
| | | | | | | | 18 | | | 32227_at | proteoglycan 1, secretory |
| | | | | | | | | | | | granule |
| | | | | | | | 20 | | | 40953_at | calponin 3, acidic |
| | | | | | | | 21 | | | 41188_at | putative integral membrane |
| | | | | | | | | | | | transporter |
| | | | | | | | 22 | | | 39552_at | phosphatase and tensin |
| | | | | | | | | | | | homolog (mutated in |
| | | | | | | | | | | | multiple advanced cancers |
| | | | | | | | | | | | 1) |
| | | | | | | | 23 | | | 2062_at | insulin-like growth factor |
| | | | | | | | | | | | binding protein 7 |
| | | | | | | | 25 | | | 746_at | phosphodiesterase 3B, |
| | | | | | | | | | | | cGMP-inhibited |
| | | | | | | | | 8 | | 36783_f_at | Krueppel-related zinc |
| | | | | | | | | | | | finger protein |
| | | | | | | | | 10 | | 36500_at | NAD(P) dependent steroid |
| | | | | | | | | | | | dehydrogenase-like; |
| | | | | | | | | | | | H105e3 |
| | | | | | | | | 12 | 1 | 39932_at | Homo sapiens mRNA; |
| | | | | | | | | | | | cDNA DKFZp586F2224 |
| | | | | | | | | | | | (from clone |
| | | | | | | | | | | | DKFZp586F2224) |
| | | | | | | | | 13 | | 35241_at | KIAA0335 gene product |
| | | | | | | | | 14 | | 38350_f_at | tubulin, alpha 2 |
| | | | | | | | | 15 | | 33595_r_at | recombination activating |
| | | | | | | | | | | | gene 2 |
| | | | | | | | | 16 | | 40446_at | PHD finger protein 1 |
| | | | | | | | | 17 | 24 | 1368_at | interleukin 1 receptor, type I |
| | | | | | | | | 18 | | 1077_at | recombination activating |
| | | | | | | | | | | | gene 1 |
| | | | | | | | | 19 | | 207_at | stress-induced- |
| | | | | | | | | | | | phosphoprotein 1 |
| | | | | | | | | | | | (Hsp70/Hsp90-organizing |
| | | | | | | | | | | | protein) |
| | | | | | | | | 20 | | 32778_at | inositol 1,4,5-triphosphate |
| | | | | | | | | | | | receptor, type 1 |
| | | | | | | | | 21 | | 1479_g_at | IL2-inducible T-cell kinase |
| | | | | | | | | 22 | | 35425_at | BarH-like homeobox 2 |
| | | | | | | | | 23 | | 39430_at | tankyrase, TRF1- |
| | | | | | | | | | | | interacting ankyrin-related |
| | | | | | | | | | | | ADP-ribose polymerase |
| | | | | | | | | 24 | | 40742_at | hemopoietic cell kinase |
| | | | | | | | | | 3 | 33957_at | HCGII-7 protein |
| | | | | | | | | | 4 | 36577_at | mitogen inducible 2 |
| | | | | | | | | | 7 | 39696_at | paternally expressed 10 |
| | | | | | | | | | 8 | 34710_r_at | ESTs |
| | | | | | | | | | 9 | 31407_at | protease, serine, 7 |
| | | | | | | | | | | | (enterokinase) |
| | | | | | | | | | 12 | 35669_at | KIAA0633 protein |
| | | | | | | | | | 13 | 39221_at | leukocyte immunoglobulin- |
| | | | | | | | | | | | like receptor, subfamily B |
| | | | | | | | | | | | (with TM and ITIM |
| | | | | | | | | | | | domains), member 2 |
| | | | | | | | | | 15 | 38840_s_at | profilin 2 |
| | | | | | | | | | 16 | 35961_at | Homo sapiens mRNA; |
| | | | | | | | | | | | cDNA DKFZp586O1318 |
| | | | | | | | | | | | (from clone |
| | | | | | | | | | | | DKFZp586O1318) |
| | | | | | | | | | 17 | 37280_at | MAD (mothers against |
| | | | | | | | | | | | decapentaplegic, |
| | | | | | | | | | | | Drosophila) homolog 1 |
| | | | | | | | | | 20 | 38111_at | chondroitin sulfate |
| | | | | | | | | | | | proteoglycan 2 (versican) |
| | | | | | | | | | 22 | 33914_r_at | ferrochelatase |
| | | | | | | | | | | | (protoporphyria) |
| | | | | | | | | | 23 | 35614_at | transcription factor-like 5 |
| | | | | | | | | | | | (basic helix-loop-helix) |
| | | | | | | | | | 25 | 36342_r_at | H factor (complement)-like 3 |
| | | | | | | | | | 27 | 106_at | runt-related transcription |
| | | | | | | | | | | | factor 3 |
| | | | | | | | | | 28 | 38514_at | immunoglobulin lambda- |
| | | | | | | | | | | | like polypeptide 1 |
| | | | | | | | | | 30 | 38940_at | AD024 protein |

TABLE 41
Task 2a (CCR vs. FAIL, pre-B cases only)
Same notation, etc. as Task 2

| | | | HK Stepwise | | | |
|---|---|---|---|---|---|---|
| SM ANOVA | | | Discriminant | | | |
| (Vx “User | XW Rank- | | Analysis, HK | EA SVM/RFE | | |
| Contrast”) | Ordering Statistic | XW GA/KNN | (MV) | (MV) | Affy Accession # | Description |
| 1 | | | | | 577_at | midkine (neurite growth- |
| | | | | | | promoting factor 2) |
| 2 | | | | | 41819_at | FYN-binding protein (FYB- |
| | | | | | | 120/130) |
| 3 | | | | | 37981_at | drebrin 1 |
| 4 | | | | | 32058_at | HNK-1 sulfotransferase |
| 5 | | | | | 39418_at | DKFZP564M182 protein |
| 6 | | | 16 | 11 | 32970_f_at | intracellular hyaluronan-binding |
| | | | | | | protein |
| 7 | 12 | | 2 | 1 | 34433_at | docking protein 1, 62 kD |
| | | | | | | (downstream of tyrosine kinase |
| | | | | | | 1) |
| 8 | 3 | | | | 38971_r_at | Nef-associated factor 1 |
| 9 | | | | | 38124_at | midkine (neurite growth- |
| | | | | | | promoting factor 2) |
| 10 | | | | | 36524_at | Rho guanine nucleotide |
| | | | | | | exchange factor (GEF) 4 |
| 11 | | | | | 824_at | glutathione-S-transferase like; |
| | | | | | | glutathione transferase omega |
| 12 | | | | | 34809_at | KIAA0999 protein |
| 13 | | | | | 38119_at | glycophorin C (Gerbich blood |
| | | | | | | group) |
| 14 | | | | | 37343_at | inositol 1,4,5-triphosphate |
| | | | | | | receptor, type 3 |
| 15 | 11 | 1 | | | 1403_s_at | small inducible cytokine A5 |
| | | | | | | (RANTES) |
| 16 | | | | | 33362_at | Cdc42 effector protein 3 |
| 17 | 5 | 13 | | | 41478_at | Homo sapiens cDNA FLJ30991 |
| | | | | | | fis, clone HLUNG1000041 |
| 18 | | | | | 671_at | secreted protein, acidic, |
| | | | | | | cysteine-rich (osteonectin) |
| 19 | | | | | 35260_at | KIAA0867 protein |
| 20 | | | | | 37364_at | B-cell associated protein |
| 21 | | | | | 38940_at | AD024 protein |
| 22 | | | | | 1062_g_at | interleukin 10 receptor, alpha |
| 23 | 10 | | | | 37184_at | syntaxin 1A (brain) |
| 24 | | | | | 32724_at | phytanoyl-CoA hydroxylase |
| | | | | | | (Refsum disease) |
| 25 | | | | | 1126_s_at | Homo sapiens CD44 isoform |
| | | | | | | RC (CD44) mRNA, complete |
| | | | | | | cds |
| 26 | | | | | 31538_at | ribosomal protein, large, P0 |
| 27 | | | | | 40617_at | hypothetical protein FLJ20274 |
| 28 | 1 | 6 | 1 | 2 | 38652_at | hypothetical protein FLJ20154 |
| | | | | | | (G0) |
| 29 | | | | | 38203_at | potassium intermediate/small |
| | | | | | | conductance calcium-activated |
| | | | | | | channel, subfamily N, member 1 |
| 30 | 6 | | | | 40027_at | hypothetical protein |
| | 2 | 28 | 3 | | 34760_at | KIAA0022 gene product |
| | 4 | | | | 37674_at | aminolevulinate, delta-, synthase 1 |
| | 7 | | 9 | | 2065_s_at | BCL2-associated X protein |
| | 8 | | | | 33963_at | azurocidin 1 (cationic |
| | | | | | | antimicrobial protein 37) |
| | 9 | | | | 32254_at | vesicle-associated membrane |
| | | | | | | protein 2 (synaptobrevin 2) |
| | 13 | | | | 31888_s_at | tumor suppressing |
| | | | | | | subtransferable candidate 3 |
| | 14 | 26 | 7 | | 35322_at | Kelch-like ECH-associated |
| | | | | | | protein 1 |
| | | 2 | | | 36970_at | KIAA0182 protein |
| | | 3 | | | 41097_at | telomeric repeat binding factor 2 |
| | | 4 | | | 37986_at | erythropoietin receptor |
| | | 5 | | | 40272_at | collapsin response mediator |
| | | | | | | protein 1 |
| | | 7 | | | 35991_at | Sm protein F |
| | | 8 | | | 38155_at | “origin recognition complex, |
| | | | | | | subunit 5 (yeast homolog)-like” |
| | | 9 | | | 32624_at | DKFZp566D133 protein |
| | | 10 | | | 40534_at | “protein tyrosine phosphatase, |
| | | | | | | receptor type, D” |
| | | 11 | | | 39742_at | TRAF family member- |
| | | | | | | associated NFKB activator |
| | | 12 | | | 37218_at | “BTG family, member 3” |
| | | 14 | | | 39552_at | phosphatase and tensin homolog |
| | | | | | | (mutated in multiple advanced |
| | | | | | | cancers 1) |
| | | 15 | 6 | | 36144_at | KIAA0080 protein |
| | | 16 | | | 41667_s_at | “dTDP-D-glucose 4,6- |
| | | | | | | dehydratase” |
| | | 17 | 4 | | 35614_at | transcription factor-like 5 (basic |
| | | | | | | helix-loop-helix) |
| | | 18 | | | 32227_at | “proteoglycan 1, secretory |
| | | | | | | granule” |
| | | 19 | | | 41214_at | “ribosomal protein S4, Y- |
| | | | | | | linked” |
| | | 20 | | | 39212_at | hypothetical protein FLJ11191 |
| | | 21 | | | 39696_at | paternally expressed 10 |
| | | 22 | | | 34194_at | Homo sapiens mRNA; cDNA |
| | | | | | | DKFZp564B076 (from clone |
| | | | | | | DKFZp564B076) |
| | | 23 | | | 40276_at | “proteasome (prosome, |
| | | | | | | macropain) 26S subunit, non- |
| | | | | | | ATPase, 7 (Mov34 homolog)” |
| | | 24 | | | 38278_at | modulator recognition factor I |
| | | 25 | | | 35362_at | myosin X |
| | | | 5 | | 38270_at | poly (ADP-ribose) |
| | | | | | | glycohydrolase |
| | | | 8 | | 39607_at | myotubularin related protein 8 |
| | | | 10 | 13 | 33957_at | HCGII-7 protein |
| | | | 11 | 5 | 39932_at | Homo sapiens mRNA; cDNA |
| | | | | | | DKFZp586F2224 (from clone |
| | | | | | | DKFZp586F2224) |
| | | | 12 | 4 | 1923_at | cyclin C |
| | | | 13 | | 38496_at | ELK4, ETS-domain protein |
| | | | | | | (SRF accessory protein 1) |
| | | | 14 | 9 | 37024_at | LPS-induced TNF-alpha factor |
| | | | 15 | | 404_at | interleukin 4 receptor |
| | | | 17 | | 39116_at | putative membrane protein |
| | | | 18 | | 36207_at | SEC14 (S. cerevisiae)-like 1 |
| | | | 19 | 10 | 40713_at | nuclear factor of activated T- |
| | | | | | | cells 5, tonicity-responsive |
| | | | 20 | | 41795_at | NCK adaptor protein 1 |
| | | | 21 | | 38005_at | nucleotide-sugar transporter |
| | | | | | | similar to C. elegans sqv-7 |
| | | | 22 | | 38779_r_at | hepatoma-derived growth factor |
| | | | | | | (high-mobility group protein 1- |
| | | | | | | like) |
| | | | 23 | | 41509_at | heat shock 70 kD protein 9B |
| | | | | | | (mortalin-2) |
| | | | 24 | | 37231_at | KIAA0008 gene product |
| | | | 25 | | 35414_s_at | jagged 1 (Alagille syndrome) |
| | | | | 3 | 40817_at | nucleobindin 1 |
| | | | | 6 | 37908_at | guanine nucleotide binding |
| | | | | | | protein 11 |
| | | | | 7 | 36342_r_at | H factor (complement)-like 3 |
| | | | | 8 | 38113_at | synaptic nuclei expressed gene |
| | | | | | | 1b |
| | | | | 12 | 40364_at | solute carrier family 31 (copper |
| | | | | | | transporters), member 1 |
| | | | | 14 | 31407_at | protease, serine, 7 (enterokinase) |
| | | | | 15 | 39681_at | zinc finger protein 145 |
| | | | | | | (Kruppel-like, expressed in |
| | | | | | | promyelocytic leukemia) |
| | | | | 16 | AFFX-BioB- | NO_.SIF_seq |
| | | | | | M_at | |
| | | | | 17 | 41620_at | KIAA0716 gene product |
| | | | | 18 | 31862_at | wingless-type MMTV |
| | | | | | | integration site family, member |
| | | | | | | 5A |
| | | | | 19 | 39265_at | type 1 tumor necrosis factor |
| | | | | | | receptor shedding |
| | | | | | | aminopeptidase regulator |
| | | | | 20 | 38866_at | GRB2-related adaptor protein 2 |
| | | | | 21 | 33316_at | KIAA0808 gene product |
| | | | | 22 | 1881_at | NO_.SIF_seq |
| | | | | 23 | 346_s_at | angiotensin receptor 1 |
| | | | | 24 | 39457_r_at | sorting nexin 4 |
| | | | | 25 | 40549_at | cyclin-dependent kinase 5 |

TABLE 42
Annotation Tool for Table 40

| | | | | LOCUS | | | | | Map |
|---|---|---|---|---|---|---|---|---|---|
| | AFFYID | VALUE | SYMBOL | LINK | GENBANK | OMIM | GENE NAME | SUMMARY | Location |
| 39418_at | 39418_at | | DKFZP564M182 | 26156 | AK025446, | | DKFZP564M182 | | 16p13.13 |
| | | | | | AK025446, | | protein | | Bottom of |
| | | | | | AL049999, All | | | | Form |
| | | | | | Genbank | | | | |
| | | | | | Accessions | | | | |
| 41819_at | 41819_at | | FYB | 2533 | AF001862, | 602731 | FYN binding | [Proteome | 5p13.1 Bottom |
| | | | | | AF001862, | | protein (FYB- | FUNCTION:] FYN- | of |
| | | | | | AF116653, | | 120/130) | binding protein; | Form |
| | | | | | AF198052, | | | modulates | |
| | | | | | BC015933, | | | interleukin 2 | |
| | | | | | BC017775, | | | production | |
| | | | | | BX647195, | | | | |
| | | | | | BX647196, | | | | |
| | | | | | NM_001465, | | | | |
| | | | | | All Genbank | | | | |
| | | | | | Accessions | | | | |
| 37981_at | 37981_at | | DBN1 | 1627 | AI683844, | 126660 | drebrin 1 | [SUMMARY:] The | 5q35.3 |
| | | | | | AI683844, | | | protein encoded by | Bottom of |
| | | | | | AK094125, | | | this gene is a | Form |
| | | | | | AL110225, | | | cytoplasmic actin- | |
| | | | | | AW950551, | | | binding protein | |
| | | | | | BC000283, | | | thought to play a | |
| | | | | | BC007281, | | | role in the process | |
| | | | | | BC007567, | | | of neuronal growth. | |
| | | | | | BF205663, | | | It is a member of | |
| | | | | | D17530, | | | the drebrin family of | |
| | | | | | NM_004395, | | | proteins that are | |
| | | | | | NM_080881, | | | developmentally | |
| | | | | | All Genbank | | | regulated in the | |
| | | | | | Accessions | | | brain. A decrease in | |
| | | | | | | | | the amount of this | |
| | | | | | | | | protein in the brain | |
| | | | | | | | | has been implicated | |
| | | | | | | | | as a possible | |
| | | | | | | | | contributing factor | |
| | | | | | | | | in the pathogenesis | |
| | | | | | | | | of memory | |
| | | | | | | | | disturbance in | |
| | | | | | | | | Alzheimer's | |
| | | | | | | | | disease. At least | |
| | | | | | | | | two alternative | |
| | | | | | | | | splice variants | |
| | | | | | | | | encoding different | |
| | | | | | | | | protein isoforms | |
| | | | | | | | | have been | |
| | | | | | | | | described for this | |
| | | | | | | | | gene. | |
| 577_at | 577_at | | MDK | 4192 | BC011704, | 162096 | midkine (neurite | | 11p11.2 |
| | | | | | BC011704, | | growth- | | Bottom of |
| | | | | | D10604, | | promoting factor | | Form |
| | | | | | M69148, | | 2) | | |
| | | | | | M94250, | | | | |
| | | | | | NM_002391, | | | | |
| | | | | | X55110, All | | | | |
| | | | | | Genbank | | | | |
| | | | | | Accessions | | | | |
| 37343_at | 37343_at | | ITPR3 | 3710 | D26351, | 147267 | inositol 1,4,5- | | 6p21 |
| | | | | | D26351, | | triphosphate | | Bottom of |
| | | | | | NM_002224, | | receptor, type 3 | | Form |
| | | | | | U01062, All | | | | |
| | | | | | Genbank | | | | |
| | | | | | Accessions | | | | |
| 32058_at | 32058_at | | CHST10 | 9486 | AF033827, | 606376 | carbohydrate | [SUMMARY:] Cell | 2q12.1 |
| | | | | | AF033827, | | sulfotransferase | surface | Bottom of |
| | | | | | AF070594, | | 10 | carbohydrates | Form |
| | | | | | BC010441, All | | | modulate a variety | |
| | | | | | Genbank | | | of cellular functions | |
| | | | | | Accessions | | | and are typically | |
| | | | | | | | | synthesized in a | |
| | | | | | | | | stepwise manner. | |
| | | | | | | | | HNK1ST plays a | |
| | | | | | | | | role in the | |
| | | | | | | | | biosynthesis of | |
| | | | | | | | | HNK1 (CD57; MIM | |
| | | | | | | | | 151290), a | |
| | | | | | | | | neuronally | |
| | | | | | | | | expressed | |
| | | | | | | | | carbohydrate that | |
| | | | | | | | | contains a | |
| | | | | | | | | sulfoglucuronyl | |
| | | | | | | | | residue [supplied by | |
| | | | | | | | | OMIM] | |
| 33412_at | 33412_at | | LGALS1 | 3956 | AB097036, | 150570 | lectin, | [SUMMARY:] The | 22q13.1 |
| | | | | | AB097036, | | galactoside- | galectins are a | Bottom of |
| | | | | | BC001693, | | binding, soluble, | family of beta- | Form |
| | | | | | BC020675, | | 1 (galectin 1) | galactoside-binding | |
| | | | | | BT006775, | | | proteins implicated | |
| | | | | | J04456, | | | in modulating cell- | |
| | | | | | M57678, | | | cell and cell-matrix | |
| | | | | | NM_002305, | | | interactions. | |
| | | | | | S44881, | | | LGALS1 may act as | |
| | | | | | X14829, | | | an autocrine | |
| | | | | | X15256, All | | | negative growth | |
| | | | | | Genbank | | | factor that regulates | |
| | | | | | Accessions | | | cell proliferation. | |
| 1126_s_at | 1126_s_{—} | | CD44 | 960 | AJ251595, | 107269 | CD44 antigen | | 11p13 |
| | at | | | | AJ251595, | | (homing function | | Bottom of |
| | | | | | AY101192, | | and Indian blood | | Form |
| | | | | | AY101193, | | group system) | | |
| | | | | | BC004372, | | | | |
| | | | | | BC052287, | | | | |
| | | | | | L05424, | | | | |
| | | | | | M24915, | | | | |
| | | | | | M25078, | | | | |
| | | | | | M59040, | | | | |
| | | | | | NM_000610, | | | | |
| | | | | | S66400, | | | | |
| | | | | | U40373, | | | | |
| | | | | | X56794, | | | | |
| | | | | | X62739, | | | | |
| | | | | | X66733, All | | | | |
| | | | | | Genbank | | | | |
| | | | | | Accessions | | | | |
| 671_at | 671_at | | SPARC | 6678 | AK096969, | 182120 | secreted protein, | | 5q31.3-q32 |
| | | | | | AK096969, | | acidic, cysteine- | | Bottom of |
| | | | | | BC004974, | | rich | | Form |
| | | | | | BC008011, | | (osteonectin) | | |
| | | | | | J03040, | | | | |
| | | | | | NM_003118, | | | | |
| | | | | | Y00755, All | | | | |
| | | | | | Genbank | | | | |
| | | | | | Accessions | | | | |
| 32970_f_at | 32970_f_{—} | | HABP4 | 22927 | AF241831, | | hyaluronan | | 9q22.3-q31 |
| | at | | | | AF241831, | | binding protein 4 | | Bottom of |
| | | | | | AK000610, | | | | Form |
| | | | | | AK025144, | | | | |
| | | | | | AK055161, | | | | |
| | | | | | NM_014282, | | | | |
| | | | | | All Genbank | | | | |
| | | | | | Accessions | | | | |
| 824_at | 824_at | | GSTO1 | 9446 | AF212303, | 605482 | glutathione S- | [SUMMARY:] This | 10q25.1 |
| | | | | | AF212303, | | transferase | gene encodes a | Bottom of |
| | | | | | BC000127, | | omega 1 | member of the | Form |
| | | | | | D17168, | | | theta class | |
| | | | | | NM_004832, | | | glutathione S- | |
| | | | | | U90313, All | | | transferase-like | |
| | | | | | Genbank | | | (GSTTL) protein | |
| | | | | | Accessions | | | family. In mouse, | |
| | | | | | | | | the encoded protein | |
| | | | | | | | | acts as a small | |
| | | | | | | | | stress response | |
| | | | | | | | | protein, likely | |
| | | | | | | | | involved in cellular | |
| | | | | | | | | redox homeostasis. | |
| 32724_at | 32724_at | | PHYH | 5264 | AF023462, | 602026 | phytanoyl-CoA | [SUMMARY:] The | 10pter-p11.2 |
| | | | | | AF023462, | | hydroxylase | protein encoded by | Bottom of |
| | | | | | AF112977, | | (Refsum | this gene is a | Form |
| | | | | | AF242379, | | disease) | peroxisomal | |
| | | | | | BC021011, | | | enzyme. It | |
| | | | | | BC029512, | | | catalyzes the initial | |
| | | | | | NM_006214, | | | alpha-oxidation | |
| | | | | | All Genbank | | | step in the | |
| | | | | | Accessions | | | degradation of | |
| | | | | | | | | phytanic acid and | |
| | | | | | | | | converts phytanoyl- | |
| | | | | | | | | CoA to 2- | |
| | | | | | | | | hydroxyphytanoyl- | |
| | | | | | | | | CoA. It interacts | |
| | | | | | | | | specifically with the | |
| | | | | | | | | immunophilin | |
| | | | | | | | | FKBP52. Refsum | |
| | | | | | | | | disease, an | |
| | | | | | | | | autosomal | |
| | | | | | | | | recessive | |
| | | | | | | | | neurologic disorder, | |
| | | | | | | | | is caused by the | |
| | | | | | | | | deficiency of this | |
| | | | | | | | | encoded protein. | |
| 38652_at | 38652_at | | FLJ20154 | 54838 | AF070644, | | hypothetical | | 10q24.33 |
| | | | | | AF070644, | | protein | | Bottom of |
| | | | | | AK000161, | | FLJ20154 | | Form |
| | | | | | AK000374, | | | | |
| | | | | | AK056285, | | | | |
| | | | | | BC010506, | | | | |
| | | | | | NM_017690, | | | | |
| | | | | | All Genbank | | | | |
| | | | | | Accessions | | | | |
| 36331_at | 36331_at | | TMEM1 | 7109 | NM_003274, | 602103 | transmembrane | | 21q22.3 |
| | | | | | NM_003274, | | protein 1 | | Bottom of |
| | | | | | U19252, | | | | Form |
| | | | | | U61500, | | | | |
| | | | | | U61520, All | | | | |
| | | | | | Genbank | | | | |
| | | | | | Accessions | | | | |
| 41478_at | 41478_at | | | | | | Homo sapiens | | |
| | | | | | | | cDNA FLJ30991 | | |
| | | | | | | | fis, clone | | |
| | | | | | | | HLUNG1000041 | | |
| | | | | | | | Bottom of | | |
| | | | | | | | Form | | |
| 38119_at | 38119_at | | GYPC | 2995 | BC016653, | 110750 | glycophorin C | [SUMMARY:] | 2q14-q21 |
| | | | | | BC016653, | | (Gerbich blood | Glycophorin C | Bottom of |
| | | | | | M11802, | | group) | (GYPC) is an | Form |
| | | | | | M28335, | | | integral membrane | |
| | | | | | M29662, | | | glycoprotein. It is a | |
| | | | | | M36284, | | | minor species | |
| | | | | | NM_002101, | | | carried by human | |
| | | | | | NM_016815, | | | erythrocytes, but | |
| | | | | | X12496, | | | plays an important | |
| | | | | | X13890, | | | role in regulating | |
| | | | | | X14242, | | | the mechanical | |
| | | | | | X51973, All | | | stability of red cells. | |
| | | | | | Genbank | | | A number of | |
| | | | | | Accessions | | | glycophorin C | |
| | | | | | | | | mutations have | |
| | | | | | | | | been described. | |
| | | | | | | | | The Gerbich and | |
| | | | | | | | | Yus phenotypes are | |
| | | | | | | | | due to deletion of | |
| | | | | | | | | exon 3 and 2, | |
| | | | | | | | | respectively. The | |
| | | | | | | | | Webb and Duch | |
| | | | | | | | | antigens, also | |
| | | | | | | | | known as | |
| | | | | | | | | glycophorin D, | |
| | | | | | | | | result from single | |
| | | | | | | | | point mutations of | |
| | | | | | | | | the glycophorin C | |
| | | | | | | | | gene. The | |
| | | | | | | | | glycophorin C | |
| | | | | | | | | protein has very | |
| | | | | | | | | little homology with | |
| | | | | | | | | glycophorins A and | |
| | | | | | | | | B. | |
| 36927_at | 36927_at | | C1orf29 | 10964 | AB000115, | | chromosome 1 | [Proteome | 1p31.1 |
| | | | | | AB000115, | | open reading | FUNCTION:] | Bottom of |
| | | | | | AL832618, | | frame 29 | Moderately similar | Form |
| | | | | | BC015932, All | | | to MTAP44 | |
| | | | | | Genbank | | | | |
| | | | | | Accessions | | | | |
| 35145_at | 35145_at | | MNT | 4335 | NM_020310, | 603039 | MAX binding | | 17p13.3 |
| | | | | | NM_020310, | | protein | | Bottom of |
| | | | | | X96401, | | | | Form |
| | | | | | Y13440, | | | | |
| | | | | | Y13444, All | | | | |
| | | | | | Genbank | | | | |
| | | | | | Accessions | | | | |
| 33637_g_at | 33637_g_{—} | | CTAG1 | 1485 | AF038567, | 300156 | cancer/testis | [Proteome | Xq28 |
| | at | | | | AF038567, | | antigen 1 | FUNCTION:] | Bottom of |
| | | | | | AF277315, | | | Cancer-testis | Form |
| | | | | | AJ003149, | | | antigen | |
| | | | | | AJ275977, | | | | |
| | | | | | AJ275978, | | | | |
| | | | | | NM_001327, | | | | |
| | | | | | All Genbank | | | | |
| | | | | | Accessions | | | | |
| 34610_at | 34610_at | | GNB2L1 | 10399 | AK095666, | 176981 | guanine | | 5q35.3 |
| | | | | | AK095666, | | nucleotide | | Bottom of |
| | | | | | BC000214, | | binding protein | | Form |
| | | | | | BC000366, | | (G protein), beta | | |
| | | | | | BC010119, | | polypeptide 2- | | |
| | | | | | BC014256, | | like 1 | | |
| | | | | | BC014788, | | | | |
| | | | | | BC017287, | | | | |
| | | | | | BC019093, | | | | |
| | | | | | BC019362, | | | | |
| | | | | | BC021993, | | | | |
| | | | | | BC029996, | | | | |
| | | | | | BC032006, | | | | |
| | | | | | BC035460, | | | | |
| | | | | | M24194, | | | | |
| | | | | | NM_006098, | | | | |
| | | | | | All Genbank | | | | |
| | | | | | Accessions | | | | |
| 35659_at | 35659_at | | IL10RA | 3587 | BC028082, | 146933 | interleukin 10 | [SUMMARY:] The | 11q23 |
| | | | | | BC028082, | | receptor, alpha | protein encoded by | |
| | | | | | BM193545, | | | this gene is a | |
| | | | | | NM_001558, | | | receptor for | |
| | | | | | U00672, All | | | interleukin 10. This | |
| | | | | | Genbank | | | protein is | |
| | | | | | Accessions | | | structurally related | |
| | | | | | | | | to interferon | |
| | | | | | | | | receptors. It has | |
| | | | | | | | | been shown to | |
| | | | | | | | | mediate the | |
| | | | | | | | | immunosuppressive | |
| | | | | | | | | signal of interleukin | |
| | | | | | | | | 10, and thus inhibits | |
| | | | | | | | | the synthesis of | |
| | | | | | | | | proinflammatory | |
| | | | | | | | | cytokines. This | |
| | | | | | | | | receptor is reported | |
| | | | | | | | | to promote survival | |
| | | | | | | | | of progenitor | |
| | | | | | | | | myeloid cells | |
| | | | | | | | | through the insulin | |
| | | | | | | | | receptor substrate- | |
| | | | | | | | | 2/PI 3-kinase/AKT | |
| | | | | | | | | pathway. Activation | |
| | | | | | | | | of this receptor | |
| | | | | | | | | leads to tyrosine | |
| | | | | | | | | phosphorylation of | |
| | | | | | | | | JAK1 and TYK2 | |
| | | | | | | | | kinases. | |

### Example XII

### Gene Expression Profiling of Pediatric Acute Lymphoblastic Leukemia Reveals Unique Subgroups not Predicted by Current Genetic Risk Stratification

Summary

Current ALL classification schemes mask inherent biologic predictors of outcome. Classification schemes that reflect the underlying biology of this disease could guide patients to more tailored treatments. To develop gene expression-based classification schemes related to the pathogenic basis of pediatric lymphoblastic leukemia, gene expression patterns observed in the statistically designed cohort containing 254 pediatric acute lymphoid leukemia (ALL) cases described in Example IA were examined using Affymetrix U95AV2 oligonucleotide microarrays. Additionally, in order to model remission vs. failure conditioned to predictive cytogenetics, matched patients were selected among all major genetic prognostic groups (MLL/AF4, BCR/ABL, E2A/PBX1, TEL/AML1, hyperdiploidy, and hypodiploidy).

The data were analyzed for class discovery using unsupervised clustering methods (hierarchical clustering and a force directed algorithm) and for class prediction using supervised learning techniques including Bayesian Nets, Fisher's Discriminant, and Support Vector Machines. During initial exploratory data analysis, several distinct clusters were observed using unsupervised clustering methods. Interestingly, no correlation between the currently employed risk classification groups and these clusters was evident. In particular, ALL cases characterized by accepted “good” and “poor” risk genetics were distributed differentially among the identified clusters. This class discovery analysis indicates a more complex intrinsic genetic and biologic background in pediatric ALL than currently appreciated.

Gene expression profiles associated with achievement of remission vs. treatment failure were then sought using supervised learning techniques. Derived predictive algorithms were applied to a training set of the data. Their performance was evaluated with multiple cross validation and bootstrap runs, with an average accuracy of 72% and low variance. These models are being tested on the validation set. The results provide evidence of additional heterogeneity of pediatric ALL, which may relate to novel transformation pathways and clinical outcomes.

Data Analysis

The analysis of the gene expression data was done in a two-step approach. First, in order to identify potential clusters and inherent biologic groups, a large number of clinical co-variables were correlated with the expression data using unsupervised clustering methods such as hierarchical clustering, principal component analysis and a force-directed clustering algorithm coupled with a novel visualization tool (VxInsight). For class prediction, supervised learning methods such as Bayesian Networks, Support Vector Machines with Recursive Feature Elimination (SVM-RFE), Neuro-Fuzzy Logic and Discriminant Analysis were employed to create classification algorithms. The performance of these classification algorithms was evaluated using fold-dependent leave-one-out cross validation (LOOCV) techniques. These methods combined allowed the identification of genes associated with remission or treatment failure and with the different translocations across the dataset.

Results

To explore potential clusters driven by gene expression profiles, the initial analysis of the pediatric ALL cohort was accomplished using a force directed clustering algorithm coupled with a novel visualization tool, VxInsight as described in Example IB. Unexpectedly, we discovered 9 novel biologic clusters of ALL (2 distinct T-cell ALL clusters (S1 and S2) and 7 (2 related clusters are seen in cluster X) distinct B-lineage ALL clusters (A, B, C, X, Y, Z)) each with distinguishing gene expression profiles. Using ANOVA, we identified over 100 statistically significant genes uniquely distinguishing each of these cohorts; a list of the top statistically significant genes distinguishing each cluster is provided in Table 43. Review of these lists of genes reveals many interesting signaling molecules and transcription factors. The X cluster (which contains two highly related clusters) is quite unique in having expression of several genes regulating methylation and folate metabolism.

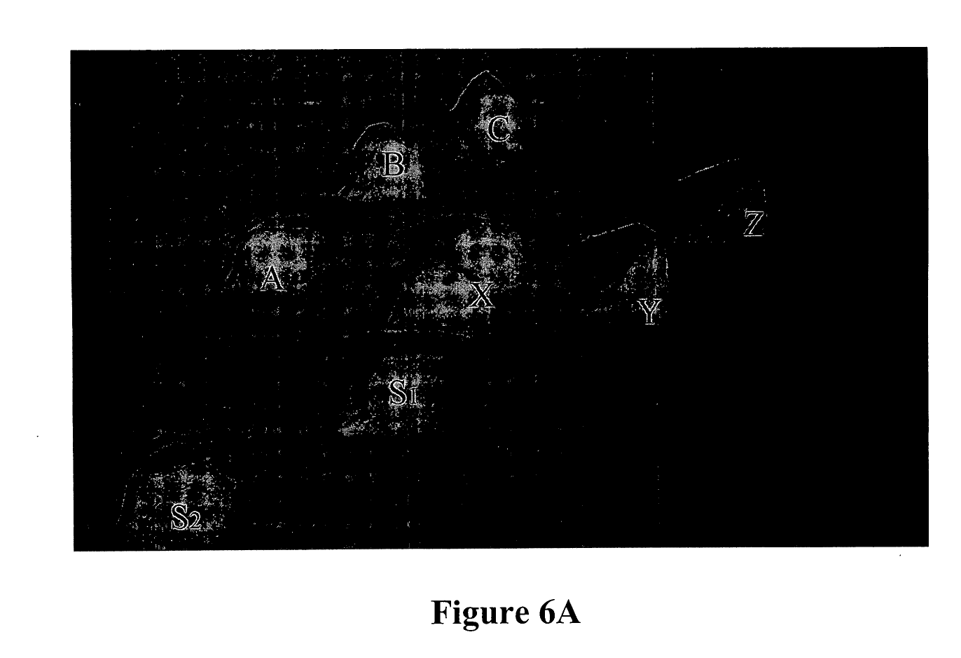

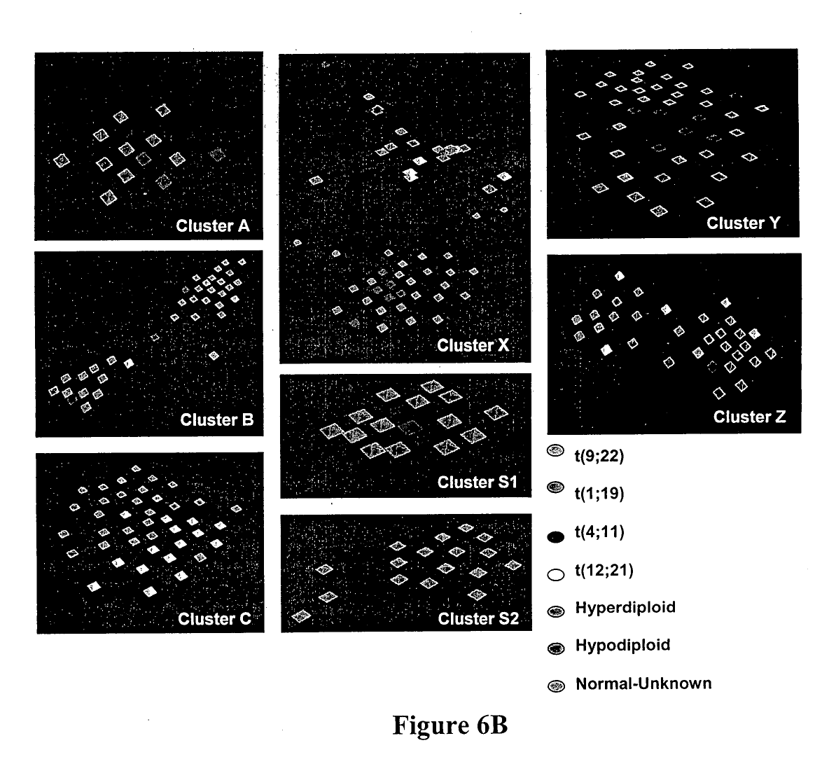

Examination of the cluster data reveals that while there are some trends, no cytogenetic abnormality precisely defines or is correlated with any specific cluster. It is interesting that cases with a t(12;21) or hyperdiploidy, both conferring low risk and good outcomes, tend to cluster together; although combinations of these cases can be seen primarily in clusters C and Z as well as the top component of the X cluster indicating that there is still heterogeneity in gene expression profiles associated with these clusters. On the terrain map from VxInsight (FIG. 6, top) these three cluster regions (C, Z, and X) are actually fairly closely approximated indicating they are more related than for example cluster C to cluster S2. Although our correlations between outcome and clusters are still underway, it is interesting that the hyperdiploid and t(12;21) cases in cluster X had a significantly poorer outcome than those in cluster C or Z, suggesting that these cluster groupings may reflect different biologic propensities that confer differing responses to therapy. Similarly, the t(1;19) cases clustered in Y had a poorer outcome than those in clusters A and B. Finally, it is of interest that ALL cases with t(9;22) simply don't cluster, they appear to be distributed among virtually all B precursor clusters. While we do not understand the significance of this result, it suggests that the t(9;22) is a pre-leukemic or initiating genetic lesion that may not be sufficient for leukemogenesis, or alternatively, that clones with a t(9;22) are quite genetically unstable and transformation and genetic progression may occur along many pathways. Results similar to our own were recently reported by Fine et al. (Blood Abstract, Blood Supplement 2002 (753a, Abstract #2979)). Using hierarchical clustering on a small series of 35 cell lines and ALL cases, these investigators found a limited correlation between intrinsic biologic clusters in ALL and cytogenetic abnormalities; cases with a t(9;22) were found to be particularly heterogeneous in their gene expression profiles.

The stability and structure of the clusters was explored using methods of data perturbation. Because the clusters appeared to be steady, subsequent exploration of the group-characterizing genes was performed using analysis of variance (ANOVA). This method was applied to order all of the genes with respect to differential expressions between the groups. The strongest 0.1% of the genes were tabulated in lists. The strength of these gene lists was studied using statistical bootstrapping as described in Example IB, and suggested that the identified groups represented well-separated patient subclasses.

Surprisingly, with the exception of the T-ALL cases (clusters S₁ and S₂), the clustering of ALL patients was independent of karyotype, suggesting that common tumor genetics, as currently applied to prognostic schema, do not strongly influence or drive innate expression profiling in pediatric ALL. However, fewer “adverse prognosis” genetics were distributed among certain clusters (e.g. C and Z). Remarkably, patients with translocations such as t(9;22)/BCR-ABL, t(1;19)/E2A/PBX1, and t(12;21)/TEL/AML1, were distributed among several clusters, suggesting biologic heterogeneity beyond the present tendency to group these various entities for the purpose of prognosis and outcome prediction. The results of these class discovery methods suggested that, when applied to our patient data set, unsupervised techniques elucidate underlying novel subgroups pediatric ALL. In turn, this reassessment of tumor heterogeneity encourages the design of additional studies to ascertain whether these data can enhance the discriminatory power of currently employed prognostic variables.

Analysis was therefore next focused on class prediction. The process of defining the best set of discriminating genes between known subsets of samples can be accomplished using supervised learning techniques such as Bayesian Networks, linear discriminant analysis and support vector machines (SVM). In contrast to unsupervised methods that generate inherent “classes” for each gene or patient, supervised learning methods are trained to recognize “known classes”, creating classification algorithms that may also uncover interesting and novel therapeutic targets.

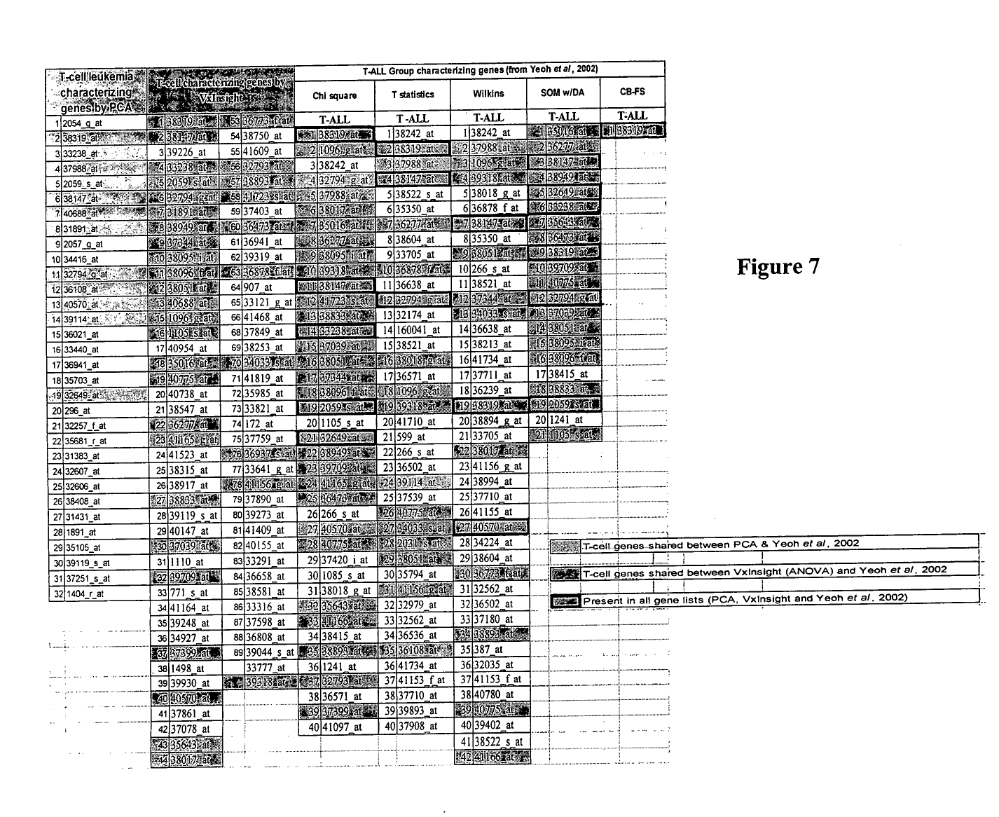

Genes that best discriminated T-lineage ALL from B-lineage ALL were identified using principal component analysis and ANOVA of the cluster-differentiating genes generated from the VxInsight analysis. Significant overlap was observed between the 2 methods used in our analysis of the T-cell ALL gene expression profile, as well as with published data (Yeoh et al., Cancer Cell 1; 133-143, 2002), both in the actual presence of the same genes, as well as in relative rank (FIG. 7). Importantly, this is evident across data sets and regardless of analytic approach for T-cell ALL, suggesting that these genes define important features of T-ALL biology. It also implies that T-ALL gene expression is inherently “less complex” in delineating this leukemic entity, than for B-lineage ALL.

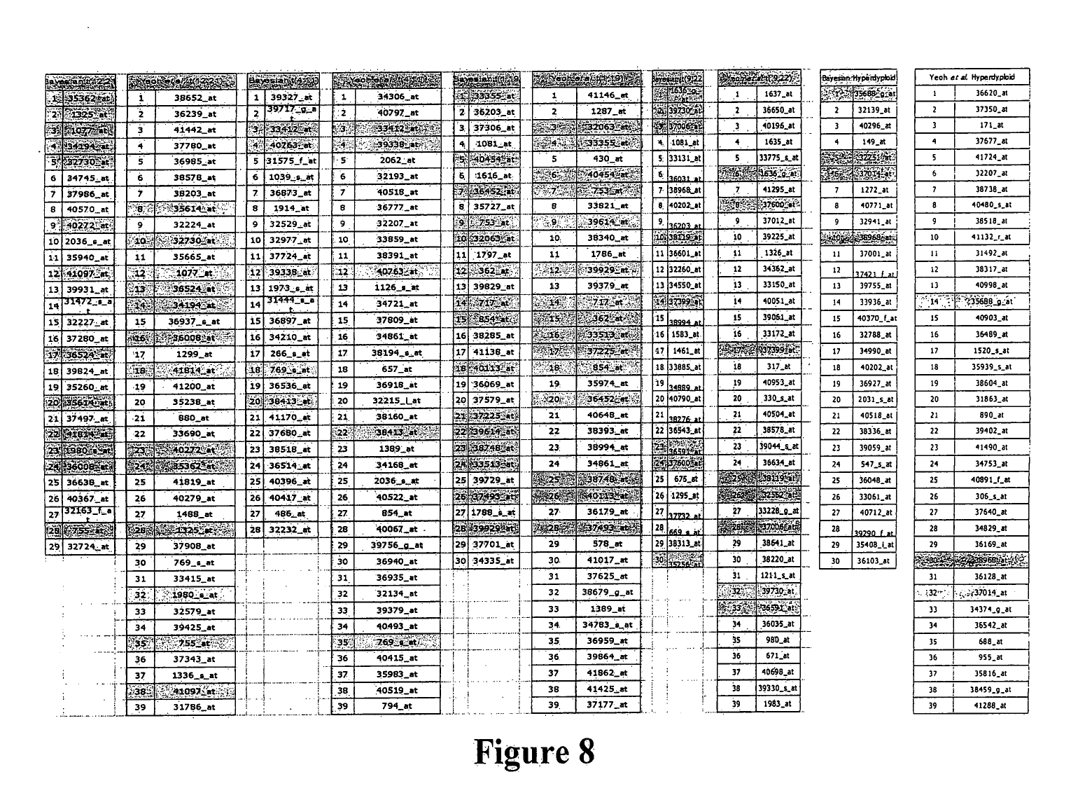

Gene expression profiles characteristic of translocation types were derived using supervised learning techniques. 147 genes derived from Bayesian network analysis that allowed the identification of samples within each of the major translocation groups with accuracy rates higher than 90%, as calculated by fold dependent leave-one-out cross validation. This filtered data analysis of gene expression conditioned on karyotype generated distinct case clustering, confirming that unique gene expression “signatures” identify defined genetic subsets of ALL. This corroborates recently published data (Yeoh et al., Cancer Cell 1; 133-143, 2002) which revealed that karyotypic sub-groups of ALL are characterized by specific gene expression profiles (FIG. 8). Unsupervised methods do not clearly identify clusters of patients by therapeutic outcome. Nonetheless, some clusters (e.g. C, Y, S1) contain a greater number of remission cases. When the clusters are examined for remission versus failure by karyotype, it is evident that there is only minimal correlation between the distribution of prognostically important tumor genetics and outcome. For example, while clusters C and Z have similar distributions of case number and karyotypic sub-types, more C group patients achieved remission. Cluster Y, which harbors a greater proportion of adverse prognosis genetic types, unexpectedly demonstrates a relatively high percentage of remission cases. These findings imply that the biology of clinical outcome in pediatric ALL is more complex than previously appreciated and is not readily determined by the relatively gross examination of tumor cytogenetics. These data thus support the observation that relapse in pediatric ALL occurs regardless of NCI clinical risk category, or current genetic risk modifiers. It is notable that gene expression analysis identifies 2 sub-populations of T-ALL, one of which (S1) demonstrates a favorable therapeutic outcome.

Comparison with Method and Results of Yeoh et al. (Cancer Cell 1; 133-143, 2002)

Yeoh et al., in a study performed on the “Downing” or “St. Jude” data set as described above, reported that pediatric ALL cases clustered according to the recurrent cytogenetic abnormalities associated with ALL, and thus, that cytogenetics could define these intrinsic groups. However, careful reading of this report and the methods of analysis employed reveals that these investigators did not perform and/or report the results of true unsupervised learning methods and class discovery. Rather, these investigators first used supervised learning algorithms (primarily Support Vector Machines) to identify short lists of expressed genes that were associated with each recurrent cytogenetic abnormality in ALL. Using a highly selected set of only 271 genes that resulted from this supervised learning approach, they then performed hierarchical clustering or PCA using the expression data derived from only this set of selected genes. As would be expected from this approach, distinct ALL clusters could be defined based on shared gene expression profiles and each cluster was associated with a specific cytogenetic abnormality. However, this approach did not reveal what the underlying structure was in the gene expression profiles if one took a truly unbiased approach and performed real class discovery.

Furthermore, although Yeoh et al. attempted to use supervised learning methods to identify genes associated with outcome, they were not successful. Potential outcome genes identified in training sets could not be confirmed in independent test sets, indicating that the learning algorithms employed were “over-fitting” the data—a not uncommon problem with supervised learning algorithms. Another potential problem with these studies was that was no statistical design for the cases selected for study in this St. Jude cohort; cases were selected simply based on sample availability. Thus, in contrast to our retrospective POG cohort design in which cases with long term remission were balanced roughly 50:50 with cases that failed, the St. Jude cases were predominantly cases with long term remission (>80%), making the modeling in the St. Jude dataset far more difficult. We have come to appreciate is how important statistical design and case selection is to any array study (indeed for any scientific study) and that for supervised learning algorithms and class prediction, it is very important to have the label that one is trying to predict (such as outcome or the presence of a particular genetic abnormality) balanced 50:50 in the cohort undergoing modeling and within the training and test sets.

TABLE 43
GENES THAT DISTINGUISH BETWEEN THE VxINSIGHT
CLUSTERS (BY ANOVA) IN THE PEDIATRIC
ALL MICROARRAY COHORT

| | | |
|---|---|---|
| PROBE | GENE SYMBOL | LOCATION |

CLUSTER A

| | TITLE - CLUSTER A | | |
|---|---|---|---|
| 37188_at | phosphoenolpyruvate carboxykinase 2 (mitochondrial) | PCK2 | 14q11.2 |
| 33342_at | RNA, U transporter 1 | RNUT1 | 15q22.33 |
| 35701_at | v-Ha-ras Harvey rat sarcoma viral oncogene homolog | HRAS | 11p15.5 |
| 36193_at | partner of RAC1 (arfaptin 2) | POR1 | 11p15 |
| 40084_at | transcription factor CP2 | TFCP2 | 12q13 |
| 38895_i_at | neutrophil cytosolic factor 4 (40 kD) | NCF4 | 22q13.1 |
| 39780_at | protein phosphatase 3 (formerly 2B), catalytic subunit, beta isoform | PPP3CB | 10q21-q22 |
| 33430_at | DKFZP586M1523 protein | DKFZP586M1523 | 18q12.1 |
| 35911_r_at | matrix metalloproteinase-like 1 | MMPL1 | 16p13.3 |
| 34255_at | diacylglycerol O-acyltransferase homolog 1 (mouse) | DGAT1 | 8qter |
| 39009_at | Lsm3 protein | LSM3 | 3p25.1 |
| 1382_at | replication protein A1 (70 kD) | RPA1 | 17p13.3 |
| 35695_at | Chediak-Higashi syndrome 1 | CHS1 | 1q42.1-q42.2 |
| 40676_at | integrin beta 3 binding protein (beta3-endonexin) | ITGB3BP | 1p31.3 |
| 40472_at | Homo sapiens clone 23763 unknown mRNA, partial cds | no gene symbol | no location |
| 37479_at | CD72 antigen | CD72 | 9p11.2 |
| 41198_at | granulin | GRN | 17q21.32 |
| 40486_g_at | DIPB protein | HSA249128 | 11p11.2 |
| 41057_at | uncharacterized hypothalamus protein HT012 | HT012 | 6p21.32 |
| 34359_at | CGI-130 protein | LOC51020 | 6q13-q24.3 |
| 37303_at | ADP-ribosyltransferase (NAD+; poly polymerase)-like 1 | ADPRTL1 | 13q11 |
| 36626_at | hydroxysteroid (17-beta) dehydrogenase 4 | HSD17B4 | 5q21 |
| 36276_at | contactin 2 (axonal) | CNTN2 | 1q32.1 |
| 41308_at | C-terminal binding protein 1 | CTBP1 | 4p16 |
| 39965_at | ras-related C3 botulinum toxin substrate 3 | RAC3 | 17q25.3 |
| 40487_at | DIPB protein | HSA249128 | 11p11.2 |
| 39043_at | actin related protein 2/3 complex, subunit 1B (41 kD) | ARPC1B | 7q11.21 |
| 467_at | osteoclast stimulating factor 1 | OSTF1 | 12q24.1-24.2 |
| 37898_r_at | Homo sapiens, clone MGC: 22588 IMAGE: 4696566, complete cds | no gene symbol | no location |
| 38104_at | 2,4-dienoyl CoA reductase 1, mitochondrial | DECR1 | 8q21.3 |
| 36091_at | src family associated phosphoprotein 2 | SCAP2 | 7p21-p15 |
| 399_at | serine/threonine kinase 25 (STE20 homolog, yeast) | STK25 | 2q37.3 |
| 34970_r_at | 5-oxoprolinase (ATP-hydrolysing) | OPLAH | 8 |
| 39743_at | hypothetical protein FLJ20580 | FLJ20580 | 1p33 |
| 35843_at | NIMA (never in mitosis gene a) - related kinase 9 | NEK9 | 14q24.2 |
| 1250_at | protein kinase, DNA-activated, catalytic polypeptide | PRKDC | 8q11 |
| 33250_at | chromosome 6 open reading frame 11 | C6orf11 | 6p21.3 |
| 32245_at | KIAA0737 gene product | KIAA0737 | 14q11.1 |
| 37845_at | hematopoietic protein 1 | HEM1 | 12q13.1 |
| 1599_at | cyclin-dependent kinase inhibitor 3 | CDKN3 | 14q22 |
| 33727_r_at | tumor necrosis factor receptor superfamily, member 6b, decoy | TNFRSF6B | 20q13.3 |
| 35820_at | GM2 ganglioside activator protein | GM2A | 5q31.3-q33.1 |
| 39896_at | DEAD/H (Asp-Glu-Ala-Asp/His) box polypeptide 16 | DDX16 | 6p21.3 |
| 40509_at | electron-transfer-flavoprotein, alpha polypeptide (aciduria II) | ETFA | 15q23-q25 |
| 35986_at | histone acetyltransferase MYST1 | MYST1 | 16p11.1 |
| 34765_at | KIAA0020 gene product | KIAA0020 | 9p24.2 |
| 40063_at | nuclear domain 10 protein | NDP52 | 17q23.2 |
| 40415_at | acetyl-Coenzyme A acyltransferase 1 | ACAA1 | 3p23-p22 |
| 1553_r_at | no title | no gene symbol | no location |
| 37251_s_at | glycoprotein M6B | GPM6B | Xp22.2 |
| 567_s_at | promyelocytic leukemia | PML | 15q22 |
| 1804_at | kallikrein 3, (prostate specific antigen) | KLK3 | 19q13.41 |
| 1280_i_at | no title | no gene symbol | no location |
| 32701_at | armadillo repeat gene deletes in velocardiofacial syndrome | ARVCF | 22q11.21 |
| 39779_at | TAR (HIV) RNA binding protein 1 | TARBP1 | 1q42.3 |
| 40323_at | CD38 antigen (p45) | CD38 | 4p15 |
| 41058_g_at | uncharacterized hypothalamus protein HT012 | HT012 | 6p21.32 |
| 38990_at | F-box only protein 9 | FBXO9 | 6p12.3-p11.2 |
| 40133_s_at | glyoxylate reductase/hydroxypyruvate reductase | GRHPR | 9q12 |
| 33350_s_at | JM5 protein | JM5 | Xp11.23 |
| 1238_at | mitogen-activated protein kinase 9 | MAPK9 | 5q35 |
| 40982_at | hypothetical protein FLJ10534 | FLJ10534 | 17p13.3 |
| 32866_at | KIAA0605 gene product | KIAA0605 | 9q34.3 |
| 38571_at | FGFR1 oncogene partner | FOP | 6q27 |
| 37955_at | transmembrane protein 4 | TMEM4 | 12q15 |
| 41799_at | DnaJ (Hsp40) homolog, subfamily C, member 7 | DNAJC7 | 17q11.2 |
| 33493_at | erythroid differentiation and denucleation factor 1 | HFL-EDDG1 | 18p11.1 |
| 38242_at | B-cell linker | BLNK | 10q23.2-q23.33 |
| 34894_r_at | protease, serine, 22 | PRSS22 | 16p13.3 |
| 41322_s_at | nucleolar protein family A, member 2 | NOLA2 | 5q35.3 |
| 37885_at | hypothetical protein AF038169 | AF038169 | 2q22.1 |
| 32789_at | nuclear cap binding protein subunit 2, 20 kD | NCBP2 | 3q29 |
| 34294_at | kinesin family member C3 | KIFC3 | 16q13-q21 |
| 1827_s_at | v-myc myelocytomatosis viral oncogene homolog (avian) | MYC | 8q24.12-q24.13 |
| 37905_r_at | no title | no gene symbol | no location |
| 33323_r_at | stratifin | SFN | 1p35.3 |
| 33126_at | glycosyltransferase AD-017 | AD-017 | 3p21.31 |
| 32484_at | chemokine binding protein 2 | CCBP2 | 3p21.3 |
| 37392_at | phosphorylase kinase, beta | PHKB | 16q12-q13 |
| 396_f_at | erythropoietin receptor | EPOR | 19p13.3-p13.2 |
| 40789_at | adenylate kinase 2 | AK2 | 1p34 |
| 34573_at | ephrin-A3 | EFNA3 | 1q21-q22 |
| 1008_f_at | protein kinase, interferon-inducible double stranded RNA dependent | PRKR | 2p22-p21 |
| 721_g_at | heat shock transcription factor 4 | HSF4 | 16q21 |
| 948_s_at | peptidylprolyl isomerase D (cyclophilin D) | PPID | 4q31.3 |
| 38640_at | zinc finger protein | LOC51042 | 1p35.3 |
| 36907_at | mevalonate kinase (mevalonic aciduria) | MVK | 12q24 |
| 32220_at | high-mobility group (nonhistone chromosomal) protein 1 | HMG1 | 13q12 |
| 41184_s_at | proteasome (prosome, macropain) subunit, beta type, 8 | PSMB8 | 6p21.3 |

CLUSTER B

| | | | |
|---|---|---|---|
| | TITLE -CLUSTER B | | |
| 32854_at | F-box and WD-40 domain protein 1B | FBXW1B | 5q35.1 |
| 39224_at | centaurin, delta 1 | CENTD1 | 4p15.1 |
| 41625_at | thyroid hormone receptor-associated protein, 240 kDa subunit | TRAP240 | 17q22-q23 |
| 35289_at | rab6 GTPase activating protein (GAP and centrosome-associated) | GAPCENA | 9q34.11 |
| 38082_at | KIAA0650 protein | KIAA0650 | 18p11.31 |
| 35268_at | axotrophin | AXOT | 2q24.2 |
| 36827_at | golgi phosphoprotein 1 | GOLPH1 | 1q41 |
| 39759_at | homolog of mouse quaking QKI (KH domain RNA binding protein) | QKI | 6q26-27 |
| 34879_at | dolichyl-phosphate mannosyltransferase polypeptide 1, catalytic | DPM1 | 20q13.13 |
| 38462_at | NADH dehydrogenase (ubiquinone) 1 alpha subcomplex, 5 | NDUFA5 | 7q32 |
| 38659_at | soc-2 suppressor of clear homolog (C. elegans) | SHOC2 | 10q25 |
| 38837_at | hypothetical protein DJ971N18.2 | DJ971N18.2 | 20p12 |
| 36144_at | KIAA0080 protein | KIAA0080 | 1 |
| 37731_at | epidermal growth factor receptor pathway substrate 15 | EPS15 | 1p32 |
| 38685_at | syntaxin 12 | STX12 | 1p35-34.1 |
| 38765_at | Dicer1, Dcr-1 homolog (Drosophila) | DICER1 | 14q32.2 |
| 38056_at | KIAA0195 gene product | KIAA0195 | 17 |
| 38764_at | Homo sapiens clone 23938 mRNA sequence | no gene symbol | no location |
| 41651_at | KIAA1033 protein | KIAA1033 | 12q24.11 |
| 38041_at | UDP-N-acetyl-alpha-D-galactosamine: polypeptide N- | GALNT1 | 18q12.1 |
| | acetylgalactosaminyltransferase 1 (GalNAc-T1) | | |
| 34654_at | myotubularin related protein 1 | MTMR1 | Xq28 |
| 1814_at | transforming growth factor, beta receptor II (70-80 kD) | TGFBR2 | 3p22 |
| 34370_at | archain 1 | ARCN1 | 11q23.3 |
| 36474_at | KIAA0776 protein | KIAA0776 | 6q16.3 |
| 33805_at | centrosome-associated protein 350 | CAP350 | 1p36.13-q41 |
| 33418_at | RAB3 GTPase-ACTIVATING PROTEIN | RAB3GAP | 2q14.3 |
| 35279_at | Tax1 human T-cell leukemia virus type I) binding protein 1 | TAX1BP1 | 7p15 |
| 34800_at | ortholog of mouse integral membrane glycoprotein LIG-1 | LIG1 | no location |
| 34825_at | TRAF and TNF receptor-associated protein | AD022 | 6p22.1-22.3 |
| 39389_at | CD9 antigen (p24) | CD9 | 12p13.3 |
| 39964_at | retinitis pigmentosa 2 (X-linked recessive) | RP2 | Xp11.4-p11.21 |
| 40610_at | zinc finger RNA binding protein | ZFR | 5p13.2 |
| 706_at | no title | no gene symbol | no location |
| 33761_s_at | KIAA0493 protein | KIAA0493 | 1q21.3 |
| 35793_at | Ras-GTPase activating protein SH3 domain-binding protein 2 | G3BP2 | 4q21.1 |
| 33893_r_at | KIAA0470 gene product | KIAA0470 | 1q44 |
| 35258_f_at | splicing factor, arginine/serine-rich 2, interacting protein | SFRS2IP | 12p11.21 |
| 40839_at | ubiquitin-like 3 | UBL3 | 13q12-q13 |
| 32857_at | son of sevenless homolog 2 (Drosophila) | SOS2 | 14q21 |
| 40591_at | cell division cycle 27 | CDC27 | 17q12-17q23.2 |
| 33381_at | nuclear receptor coactivator 3 | NCOA3 | 20q12 |
| 35205_at | cofactor of BRCA1 | COBRA1 | no location |
| 32872_at | Homo sapiens mRNA; cDNA DKFZp564I083 | no gene symbol | no location |
| 39695_at | decay accelerating factor for complement (CD55) | DAF | 1q32 |
| 39691_at | SH3-domain GRB2-like endophilin B1 | SH3GLB1 | 1p22 |
| 35153_at | Nijmegen breakage syndrome 1 (nibrin) | NBS1 | 8q21-q24 |
| 38818_at | serine palmitoyltransferase, long chain base subunit 1 | SPTLC1 | 9q21-q22 |
| 34877_at | Janus kinase 1 (a protein tyrosine kinase) | JAK1 | 1p32.3-p31.3 |
| 33879_at | sigma receptor (SR31747 binding protein 1) | SR-BP1 | 9p11.2 |
| 37685_at | phosphatidylinositol binding clathrin assembly protein | PICALM | 11q14 |
| 40865_at | thymine-DNA glycosylase | TDG | 12q24.1 |
| 35847_at | ubiquitin specific protease 24 | USP24 | 1p32.2 |
| 38505_at | Homo sapiens mRNA; cDNA DKFZp586J0720 | no gene symbol | no location |
| 35973_at | Huntingtin interacting protein H | HYPH | 12q21.1 |
| 37683_at | ubiquitin specific protease 10 | USP10 | 16q24.1 |
| 40901_at | nuclear autoantigen | GS2NA | 14q13-q21 |
| 39745_at | optic atrophy 1 (autosomal dominant) | OPA1 | 3q28-q29 |
| 41360_at | CCR4-NOT transcription complex, subunit 8 | CNOT8 | 5q31-q33 |
| 36002_at | KIAA1012 protein | KIAA1012 | 18q11.2 |
| 37537_at | ADP-ribosylation factor domain protein 1, 64 kD | ARFD1 | 5q12.3 |
| 40438_at | protein phosphatase 1, regulatory (inhibitor) subunit 12A | PPP1R12A | 12q15-q21 |
| 34394_at | activity-dependent neuroprotector | ADNP | 20q13.13-q13.2 |
| 34312_at | nuclear receptor coactivator 2 | NCOA2 | 8q13.1 |
| 1827_s_at | v-myc myelocytomatosis viral oncogene homolog (avian) | MYC | 8q24.12-q24.13 |
| 32336_at | aldolase A, fructose-bisphosphate | ALDOA | 16q22-q24 |
| 34349_at | SEC63 protein | SEC63L | 6q21 |
| 37828_at | hypothetical protein FLJ11220 | FLJ11220 | 1p11.2 |
| 36579_at | ubiquitination factor E4A (UFD2 homolog, yeast) | UBE4A | 11q23.3 |
| 39140_at | hypothetical protein | LOC54505 | 5q11.2 |
| 39965_at | ras-related C3 botulinum toxin substrate 3 (rho family) | RAC3 | 17q25.3 |
| 38115_at | lung cancer candidate | FUS1 | 3p21.3 |
| 41457_at | KIAA0423 protein | KIAA0423 | 14q21.1 |
| 41634_at | KIAA0256 gene product | KIAA0256 | 15q15.1 |
| 32172_at | SMART/HDAC1 associated repressor protein | SHARP | 1p36.33-p36.11 |
| 40801_at | DKFZP434C212 protein | DKFZP434C212 | 9q34.11 |
| 40138_at | COP9 subunit 6 (MOV34 homolog, 34 kD) | MOV34-34KD | 7q11.1 |
| 35734_at | ARP2 actin-related protein 2 homolog (yeast) | ACTR2 | 2p14 |
| 33727_r_at | tumor necrosis factor receptor superfamily, member 6b, decoy | TNFRSF6B | 20q13.3 |
| 39099_at | Sec23 homolog A (S. cerevisiae) | SEC23A | 14q13.2 |
| 35747_at | stromal cell derived factor receptor 1 | SDFR1 | 15q22 |
| 37575_at | Homo sapiens mRNA; cDNA DKFZp586C1723 | no gene symbol | no location |
| 38443 at | hypothetical protein MGC14433 | MGC14433 | 12q24.11 |
| 35199_at | KIAA0982 protein | KIAA0982 | 10p15.3 |
| 969_s_at | ubiquitin specific protease 9, X chromosome (Drosophila) | USP9X | Xp11.4 |
| 41601_at | tumor necrosis factor, alpha, converting enzyme | ADAM17 | 2p25 |
| 34329_at | p21 (CDKN1A)-activated kinase 2 | PAK2 | 3 |
| 33831_at | CREB binding protein (Rubinstein-Taybi syndrome) | CREBBP | 16p13.3 |
| 35295_g_at | Sjogren syndrome antigen A2 (60 kD, SS-A/Ro) | SSA2 | 1q31 |
| 40613_at | beta-site APP-cleaving enzyme | BACE | 11q23.2-q23.3 |

CLUSTER C

| | | | |
|---|---|---|---|
| | TITLE - CLUSTER C | | |
| 840_at | zinc finger protein 220 | ZNF220 | 8p11 |
| 1463_at | protein tyrosine phosphatase, non-receptor type 12 | PTPN12 | 7q11.23 |
| 35739_at | myotubularin related protein 3 | MTMR3 | 22q12.2 |
| 39809_at | HMG-box containing protein 1 | HBP1 | 7q31.1 |
| 40140_at | zinc finger protein 103 homolog (mouse) | ZFP103 | 2p11.2 |
| 37497_at | hematopoietically expressed homeobox | HHEX | 10q24.1 |
| 38148_at | cryptochrome 1 (photolyase-like) | CRY1 | 12q23-q24.1 |
| 33861_at | CCR4-NOT transcription complex, subunit 2 | CNOT2 | 12q13.2 |
| 40570_at | forkhead box O1A (rhabdomyosarcoma) | FOXO1A | 13q14.1 |
| 39696_at | paternally expressed 10 | PEG10 | 7q21 |
| 33392_at | DKFZP434J154 protein | DKFZP434J154 | 7p22.3 |
| 40128_at | KIAA0171 gene product | K1AA0171 | 5q23.1-q33.3 |
| 34892_at | tumor necrosis factor receptor superfamily, member 10b | TNFRSF 10B | 8p22-p21 |
| 1039_s_at | hypoxia-inducible factor 1, alpha subunit | HIF1A | 14q21-q24 |
| | (basic helix-loop-helix transcription factor) | | |
| 36949_at | casein kinase 1, delta | CSNK1D | 17q25 |
| 38278_at | modulator recognition factor I | MRF-1 | 2q11.1 |
| 35338_at | paired basic amino acid cleaving enzyme | PACE | 15q26.1 |
| | (furin, membrane associated receptor protein) | | |
| 34740_at | forkhead box O3A | FOXO3A | 6q21 |
| 36942_at | KIAA0174 gene product | KIAA0174 | 16q23.1 |
| 41577_at | protein phosphatase 1, regulatory (inhibitor) subunit 16B | PPP1R16B | 20q11.23 |
| 32025_at | transcription factor 7-like 2 (T-cell specific, HMG-box) | TCF7L2 | 10q25.3 |
| 38666_at | pleckstrin homology, Sec7 and coiled/coil domains 1(cytohesin 1) | PSCD1 | 17q25 |
| 32916_at | protein tyrosine phosphatase, receptor type, E | PTPRE | 10q26 |
| 1556_at | RNA binding motif protein 5 | RBM5 | 3p21.3 |
| 36978_at | KIAA0077 protein | KIAA0077 | 2p16.2 |
| 35321_at | tousled-like kinase 2 | TLK2 | 17q23 |
| 38980_at | mitogen-activated protein kinase kinase kinase 7 interacting protein 2 | MAP3K7IP2 | 6q25.1-q25.3 |
| 1377_at | nuclear factor of kappa light polypeptide gene enhancer in B-cells 1 | NFKB1 | 4q24 |
| 41409_at | basement membrane-induced gene | ICB-1 | 1p35.3 |
| 40841_at | transforming, acidic coiled-coil containing protein 1 | TACC1 | 8p11 |
| 36150_at | KIAA0842 protein | K1AA0842 | 1p36.13 |
| 31895_at | BTB and CNC homology 1, basic leucine zipper transcription factor | BACH1 | 21q22.11 |
| 1150_at | no title | no gene symbol | no location |
| 32160_at | seven in absentia homolog 1 (Drosophila) | SIAH1 | 16q12 |
| 31936_s_at | limkain b1 | LKAP | 16p13.2 |
| 37718_at | KIAA0096 protein | KIAA0096 | 3p24.3-p22.1 |
| 40839_at | ubiquitin-like 3 | UBL3 | 13q12-q13 |
| 493_at | casein kinase 1, delta | CSNK1D | 17q25 |
| 1519_at | v-ets erythroblastosis virus E26 oncogene homolog 2 (avian) | ETS2 | 21q22.2 |
| 36845_at | KIAA0136 protein | KIAA0136 | 21q22.13 |
| 39231_at | chromodomain helicase DNA binding protein 1 | CHD1 | 5q15-q21 |
| 2035_s_at | enolase 1, (alpha) | ENO1 | 1p36.3-p36.2 |
| 39897_at | KIAA1966 protein | KIAA1966 | 4q13.1 |
| 32804_at | RNA binding motif protein 5 | RBM5 | 3p21.3 |
| 34369_at | mitofusin 2 | MFN2 | 1p36.21 |
| 37280_at | MAD, mothers against decapentaplegic homolog 1 (Drosophila) | MADH1 | 4q28 |
| 41836_at | calcium homeostasis endoplasmic reticulum protein | CHERP | 19p13.1 |
| 32544_s_at | Ras suppressor protein 1 | RSU1 | 10p12.31 |
| 33304_at | interferon stimulated gene (20 kD) | ISG20 | 15q26 |
| 37539_at | Ra1GDS-like gene | RGL | 1q24.3 |
| 32069_at | Nedd4 binding protein 1 | N4BP1 | 16q12.1 |
| 38438_at | nuclear factor of kappa light polypeptide gene enhancer in B-cells 1 | NFKB1 | 4q24 |
| 34274_at | KIAA1116 protein | KIAA1116 | 6q25.1-q25.3 |
| 32977_at | chromosome 6 open reading frame 32 | C6orf32 | 6p22.3-p21.32 |
| 40130_at | follistatin-like 1 | FSTL1 | 3q13.33 |
| 954_s_at | no title | no gene symbol | no location |
| 1113_at | bone morphogenetic protein 2 | BMP2 | 20p12 |
| 40215_at | UDP-glucose ceramide glucosyltransferase | UGCG | 9q31 |
| 36115_at | CDC-like kinase 3 | CLK3 | 15q24 |
| 35163_at | KIAA1041 protein | KIAA1041 | 1pter-q31.3 |
| 38810_at | histone deacetylase 5 | HDAC5 | 17q21 |
| 35260_at | MIx interactor | MONDOA | 12q21.31 |
| 39839_at | cold shock domain protein A | CSDA | 12p13.1 |
| 38372_at | Homo sapiens unknown mRNA | no gene symbol | no location |
| 1512_at | dual-specificity tyrosine-(Y)-phosphorylation regulated kinase 1A | DYRK1A | 21q22.13 |
| 38767_at | sprouty homolog 1, antagonist of FGF signaling (Drosophila) | SPRY1 | 4q26 |
| 37970_at | mitogen-activated protein kinase 8 interacting protein 3 | MAPK8IP3 | 16p13.3 |
| 41814_at | fucosidase, alpha-L-1, tissue | FUCA1 | 1p34 |
| 41532_at | zinc finger protein 151 (pHZ-67) | ZNF151 | 1p36.2-p36.1 |
| 37585_at | small nuclear ribonucleoprotein polypeptide A1 | SNRPA1 | 22q |
| 39692_at | hypothetical protein DKFZp586F2423 | DKFZP586F2423 | 7q34 |
| 34745_at | Homo sapiens clone 24473 mRNA sequence | no gene symbol | no location |
| 35760_at | ATP synthase, H+ transporting, mitochondrial F0 complex | ATP5H | 12q13 |
| 32751_at | interleukin enhancer binding factor 3, 90 kD | ILF3 | 19p13 |
| 307_at | arachidonate 5-lipoxygenase | ALOX5 | 10q11.2 |
| 38911_at | nucleoporin 98 kD | NUP98 | 11p15.5 |
| 41464_at | KIAA0339 gene product | KIAA0339 | 16 |
| 34773_at | tubulin-specific chaperone a | TBCA | 5q13.2 |
| 1325_at | MAD, mothers against decapentaplegic homolog 1 (Drosophila) | MADH1 | 4q28 |
| 33873_at | transcription factor-like 1 | TCFL1 | 1q21 |
| 32051_at | hypothetical protein MGC2840 similar to glucosyltransferase | MGC2840 | 11pter-p15.5 |
| 34883_at | ring finger protein 10 | RNF10 | 12q24.23 |
| 37609_at | nucleotide binding protein 1 (MinD homolog, E. coli) | NUBP1 | 16p12.3 |
| 38095_i_at | major histocompatibility complex, class II, DP beta 1 | HLA-DPB1 | 6p21.3 |
| 40437_at | DKFZP564G2022 protein | DKFZP564G2022 | 15q14 |
| 36946_at | dual-specificity tyrosine-(Y)-phosphorylation regulated kinase 1A | DYRK1A | 21q22.13 |
| 38208_at | solute carrier family 35 (UDP-N-acetylglucosamine (UDP-GlcNAc)) | SLC35A3 | 1p21 |
| 755_at | inositol 1,4,5-triphosphate receptor, type 1 | ITPR1 | 3p26-p25 |
| 40898_at | sequestosome 1 | SQSTM1 | 5q35 |

CLUSTER X

| | | | |
|---|---|---|---|
| | TITLE - CLUSTER X | | |
| 36553_at | acetylserotonin O-methyltransferase-like | ASMTL | Xp22.3 |
| 35869_at | MD-1, RP105-associated | MD-1 | 6p24.1 |
| 38287_at | proteasome (prosome, macropain) subunit, beta type, 9 | PSMB9 | 6p21.3 |
| 38413_at | defender against cell death 1 | DAD1 | 14q11-q12 |
| 37311_at | transaldolase 1 | TALDO1 | 11p15.5-p15.4 |
| 41213_at | peroxiredoxin 1 | PRDX1 | 1p34.1 |
| 38780_at | aldo-keto reductase family 1, member A1 (aldehyde reductase) | AKR1A1 | 1p33-p32 |
| 674_g_at | methylenetetrahydrofolate dehydrogenase (NADP+ dependent), | MTHFD1 | 14q24 |
| | methenyltetrahydrofolate cyclohydrolase, formyltetrahydrofolate | | |
| | synthetase | | |
| 38824_at | HIV-1 Tat interactive protein 2, 30 kD | HTATIP2 | 11p14.3 |
| 32715_at | vesicle-associated membrane protein 8 (endobrevin) | VAMP8 | 2p12-p11.2 |
| 35983_at | WD repeat domain 18 | WDR18 | 19p13.3 |
| 36083_at | sarcoma amplified sequence | SAS | 12q13.3 |
| 41597_s_at | SEC22 vesicle trafficking protein-like 1 (S. cerevisiae) | SEC22L1 | 1q21.2-q21.3 |
| 34651_at | catechol-O-methyltransferase | COMT | 22q11.21 |
| 40774_at | chaperonin containing TCP1, subunit 3 (gamma) | CCT3 | 1q23 |
| 38410_at | centrin, EF-hand protein, 2 | CETN2 | Xq28 |
| 2052_g_at | O-6-methylguanine-DNA methyltransferase | MGMT | 10q26 |
| 41171_at | proteasome (prosome, macropain) activator subunit 2 (PA28 beta) | PSME2 | 14q11.2 |
| 37510_at | syntaxin 8 | STX8 | 17p12 |
| 1521_at | non-metastatic cells 1, protein (NM23A) expressed in | NME1 | 17q21.3 |
| 34699_at | CD2-associated protein | CD2AP | 6p12 |
| 1878_g_at | excision repair cross-complementing rodent repair deficiency, | ERCC1 | 19q13.2-q13.3 |
| | complementation group 1 (includes overlapping antisense sequence) | | |
| 32051_at | hypothetical protein MGC2840 similar to a putative | MGC2840 | 11pter-p15.5 |
| | glucosyltransferase | | |
| 37033_s_at | glutathione peroxidase 1 | GPX1 | 3p21.3 |
| 38076_at | ATP synthase, H+ transporting, mitochondrial F0 complex, subunit c | ATP5G1 | 17q23.2 |
| 37955_at | transmembrane protein 4 | TMEM4 | 12q15 |
| 33908_at | calpain 1, (mu/I) large subunit | CAPN1 | 11q13 |
| 39728_at | interferon, gamma-inducible protein 30 | IFI30 | 19p13.1 |
| 32166_at | HLA-B associated transcript 1 | BAT1 | 6p21.3 |
| 34268_at | regulator of G-protein signalling 19 | RGS19 | 20q13.3 |
| 36529_at | hypothetical protein MGC2650 | MGC2650 | 19q13.32 |
| 1184_at | proteasome (prosome, macropain) activator subunit 2 (PA28 beta) | PSME2 | 14q11.2 |
| 38893_at | neutrophil cytosolic factor 4 (40 kD) | NCF4 | 22q13.1 |
| 37246_at | hypothetical protein 24432 | 24432 | 16q22.3 |
| 37390_at | DEAD/H (Asp-Glu-Ala-Asp/His) box polypeptide 38 | DDX38 | 16q21-q22.3 |
| 41400_at | thymidine kinase 1, soluble | TK1 | 17q23.2-q25.3 |
| 36009_at | weakly similar to glutathione peroxidase 2 | CL683 | 1q24-q41 |
| 38720_at | chaperonin containing TCP1, subunit 7 (eta) | CCT7 | 2p12 |
| 41401_at | cysteine and glycine-rich protein 2 | CSRP2 | 12q21.1 |
| 32825_at | HMT1 hnRNP methyltransferase-like 2 (S. cerevisiae) | HRMTIL2 | 19q13.3 |
| 410_s_at | casein kinase 2, beta polypeptide | CSNK2B | 6p21.3 |
| 33447_at | myosin, light polypeptide, regulatory, non-sarcomeric (20 kD) | MLCB | 18p11.31 |
| 384_at | proteasome (prosome, macropain) subunit, beta type, 10 | PSMB10 | 16q22.1 |
| 36673_at | mannose phosphate isomerase | MPI | 15q22-qter |
| 37338_at | phosphoribosyl pyrophosphate synthetase-associated protein 1 | PRPSAP1 | 17q24-q25 |
| 39795_at | adaptor-related protein complex 2, mu 1 subunit | AP2M1 | 3q28 |
| 41749_at | chromosome 21 open reading frame 33 | C21orf33 | 21q22.3 |
| 41691_at | KIAA0794 protein | KIAA0794 | 3q29 |
| 36519_at | excision repair cross-complementing rodent repair deficiency, | ERCC1 | 19q13.2-q13.3 |
| | complementation group 1 (includes overlapping antisense sequence) | | |
| 40505_at | ubiquitin-conjugating enzyme E2L 6 | UBE2L6 | 11q12 |
| 38794_at | upstream binding transcription factor, RNA polymerase I | UBTF | 17q21.3 |
| 33441_at | T-cell leukemia translocation altered gene | TCTA | 3p21 |
| 1695_at | neural precursor cell expressed, developmentally down-regulated 8 | NEDD8 | 14q11.2 |
| 32510_at | aldo-keto reductase family 7, member A2 | AKR7A2 | 1p35.1-p36.23 |
| 39391_at | associated molecule with the SH3 domain of STAM | AMSH | 2p12 |
| 39073_at | non-metastatic cells 1, protein (NM23A) expressed in | NME1 | 17q21.3 |
| 241_g_at | spermidine synthase | SRM | 1p36-p22 |
| 40515_at | eukaryotic translation initiation factor 2B, subunit 2 (beta, 39 kD) | EIF2B2 | 14q24.3 |
| 1942_s_at | cyclin-dependent kinase 4 | CDK4 | 12q14 |
| 36496_at | inositol(myo)-1(or 4)-monophosphatase 2 | IMPA2 | 18p11.2 |
| 41332_at | polymerase (RNA) II (DNA directed) polypeptide E (25 kD) | POLR2E | 19p13.3 |
| 32756_at | enoyl Coenzyme A hydratase 1, peroxisomal | ECH1 | 19q13.1 |
| 1917_at | v-raf-1 murine leukemia viral oncogene homolog 1 | RAF1 | 3p25 |
| 32544_s_at | Ras suppressor protein 1 | RSU1 | 10p12.31 |
| 38242_at | B-cell linker | BLNK | 10q23.2-q23.33 |
| 41696_at | hypothetical protein MGC3077 | MGC3077 | 7p15-p14 |
| 37009_at | catalase | CAT | 11p13 |
| 38213_at | Bruton agammaglobulinemia tyrosine kinase | BTK | Xq21.33-q22 |
| 36600_at | proteasome (prosome, macropain) activator subunit 1 (PA28 alpha) | PSME1 | 14q11.2 |
| 37543_at | Rac/Cdc42 guanine nucleotide exchange factor (GEF) 6 | ARHGEF6 | Xq26 |
| 38894_g_at | neutrophil cytosolic factor 4 (40 kD) | NCF4 | 22q13.1 |
| 41146_at | ADP-ribosyltransferase (NAD+; poly (ADP-ribose) polymerase) | ADPRT | 1q41-q42 |
| 37255_at | N-deacetylase/N-sulfotransferase (heparan glucosaminyl) 2 | NDST2 | 10q22 |
| 37988_at | CD79B antigen (immunoglobulin-associated beta) | CD79B | 17q23 |
| 37181_at | MpV17 transgene, murine homolog, glomerulosclerosis | MPVI7 | 2p23-p21 |
| 34773_at | tubulin-specific chaperone a | TBCA | 5q13.2 |
| 38843_at | high-mobility group protein 2-like 1 | HMG2L1 | 22q13.1 |
| 38981_at | NADH dehydrogenase (ubiquinone) 1 beta subcomplex, 3 | NDUFB3 | 2q31.3 |
| 39088_at | seven transmembrane domain protein | NIFIE14 | 19q13.1 |
| 35132_at | myosin IF | MYO1F | 19p13.3-p13.2 |
| 32824_at | ceroid-lipofuscinosis, neuronal 2, late infantile | 11p15 | (Jansky-Bielschowsky disease) |
| | CLN2 | | |
| 35779_at | vacuolar protein sorting 45A (yeast) | VPS45A | 1q21-q22 |
| 37147_at | stem cell growth factor; lymphocyte secreted C-type lectin | SCGF | 19q13.3 |
| 39061_at | bone marrow stromal cell antigen 2 | BST2 | 19p13.2 |
| 36639_at | adenylosuccinate lyase | ADSL | 22q13.2 |
| 38435_at | peroxiredoxin 4 | PRDX4 | Xp22.13 |
| 36122_at | proteasome (prosome, macropain) subunit, alpha type, 6 | PSMA6 | 14q13 |
| 39897_at | KIAA1966 protein | KIAA1966 | 4q13.1 |
| 2062_at | insulin-like growth factor binding protein 7 | IGFBP7 | 4q12 |

CLUSTER Y

| | | | |
|---|---|---|---|
| | TITLE - CLUSTER Y | | |
| 40281_at | neural precursor cell expressed, developmentally down-regulated 5 | NEDD5 | 2q37 |
| 34167_s_at | no title | no gene symbol | no location |
| 36332_at | arylalkylamine N-acetyltransferase | AANAT | 17q25 |
| 38530_at | hypothetical protein FLJ22709 | FLJ22709 | 19p13.12 |
| 36452_at | synaptopodin | KIAA1029 | 5q33.1 |
| 33947_at | G protein-coupled receptor 3 | GPR3 | 1p36.1-p35 |
| 33493_at | erythroid differentiation and denucleation factor 1 | HFL-EDDG1 | 18p11.1 |
| 39122_at | glucose phosphate isomerase | GPI | 19q13.1 |
| 36780_at | clusterin (complement lysis inhibitor, SP-40, 40, sulfated glycoprotein | CLU | 8p21-p12 |
| | 2, testosterone-repressed prostate message 2, apolipoprotein J) | | |
| 31700_at | no title | no gene symbol | no location |
| 1448_at | proteasome (prosome, macropain) subunit, alpha type, 3 | PSMA3 | 14q23 |
| 39965_at | ras-related C3 botulinum toxin substrate 3 (rho family, small GTP | RAC3 | 17q25.3 |
| | binding protein Rac3) | | |
| 32811_at | myosin IC | MYO1C | 17p13 |
| 31559_at | solute carrier family 13 (sodium-dependent dicarboxylate transporter) | SLC13A2 | 17p11.1-q11.1 |
| 33403_at | DKFZP547E1010 protein | DKFZP547E1010 | 1q21.1 |
| 37475_at | DKFZP434J046 protein | DKFZP434J046 | 19q13.13 |
| 41784_at | SR rich protein | DKFZp564B0769 | 6q16.3 |
| 32474_at | paired box gene 7 | PAX7 | 1p36.2-p36.12 |
| 33683_at | no title | no gene symbol | no location |
| 37317_at | platelet-activating factor acetylhydrolase, isoform Ib, alpha subunit | PAFAH1B1 | 17p13.3 |
| 34903_at | KIAA1218 protein | KIAA1218 | 7q22.1 |
| 36826_at | general transcription factor IIF, polypeptide 1 (74 kD subunit) | GTF2F1 | 19p13.3 |
| 39692_at | hypothetical protein DKFZp586F2423 | DKFZP586F2423 | 7q34 |
| 34753_at | synaptobrevin-like 1 | SYBL1 | Xq28 |
| 32329_at | keratin, hair, basic, 6 (monilethrix) | KRTHB6 | 12q13 |
| 32220_at | high-mobility group (nonhistone chromosomal) protein 1 | HMG1 | 13q12 |
| 1169_at | protocadherin gamma subfamily B, 7 | PCDHGB7 | 5q31 |
| 35670_at | ATPase, Na+/K+ transporting, alpha 3 polypeptide | ATP1A3 | 19q13.2 |
| 31745_at | mucin 3A, intestinal | MUC3A | 7q22 |
| 38011_at | RPB5-mediating protein | RMP | 19q12 |
| 943_at | runt-related transcription factor 1 (acute myeloid leukemia 1; | RUNX1 | 21q22.3 |
| | aml1 oncogene) | | |
| 41799_at | DnaJ (Hsp40) homolog, subfamily C, member 7 | DNAJC7 | 17q11.2 |
| 40539_at | myosin IXB | MYO9B | 19p13.1 |
| 564_at | guanine nucleotide binding protein (G protein), alpha 11 (Gq class) | GNA11 | 19p13.3 |
| 36128_at | transmembrane trafficking protein | TMP21 | 14q24.3 |
| 39486_s_at | KIAA1237 protein | KIAA1237 | 3q21.3 |
| 36218_g_at | serine/threonine kinase 38 | STK38 | 6p21 |
| 41202_s_at | conserved gene amplified in osteosarcoma | OS4 | 12q13-q15 |
| 34575_f_at | no title | no gene symbol | no location |
| 37718_at | KIAA0096 protein | KIAA0096 | 3p24.3-p22.1 |
| 38882_r_at | tripartite motif-containing 16 | TRIM16 | 17p11.2 |
| 561_at | follicle stimulating hormone receptor | FSHR | 2p21-p16 |
| 33506_at | inositol polyphosphate-4-phosphatase, type I, 107 kD | INPP4A | 2q11.2 |
| 40337_at | fucosyltransferase 1 (galactoside 2-alpha-L-fucosyltransferase, | FUT1 | 19q13.3 |
| | Bombay phenotype included) | | |
| 36024_at | proline rich 4 (lacrimal) | PROL4 | 12p13 |
| 31936_s_at | limkain b1 | LKAP | 16p13.2 |
| 34333_at | KIAA0063 gene product | KIAA0063 | 22q13.1 |
| 36845_at | KIAA0136 protein | KIAA0136 | 21q22.13 |
| 35530_f_at | immunoglobulin lambda joining 3 | IGLJ3 | 22q11.1-q11.2 |
| 33879_at | sigma receptor (SR31747 binding protein 1) | SR-BP1 | 9p11.2 |
| 34272_at | regulator of G-protein signalling 4 | RGS4 | 1q23.1 |
| 40771_at | moesin | MSN | Xq11.2-q12 |
| 192_at | TAF7 RNA polymerase II, TATA box binding protein (TBP)- | TAF7 | 5q31 |
| | associated factor, 55 kD | | |
| 933_f_at | zinc finger protein 91 (HPF7, HTF10) | ZNF91 | 19p13.1-p12 |
| 38181_at | matrix metalloproteinase 11 (stromelysin 3) | MMP11 | 22q11.23 |
| 31829_r_at | trans-golgi network protein 2 | TGOLN2 | 2p11.2 |
| 38441_s_at | membrane cofactor protein (CD46, trophoblast-lymphocyte cross- | MCP | 1q32 |
| | reactive antigen) | | |
| 39500_s_at | hypothetical protein dJ465N24.2.1 | DJ465N24.2.1 | 1p36.13-p35.1 |
| 34371_at | protein phosphatase 4, regulatory subunit 1 | PPP4R1 | 18p11.21 |
| 34880_at | hypothetical protein MGC10433 | MGC10433 | 19q13.13 |
| 35805_at | likely ortholog of rat golgi stacking protein homolog GRASP55 | GRASP55 | 2p24.3-q21.3 |
| 41619_at | interferon regulatory factor 6 | IRF6 | 1q32.3-q41 |
| 40468_at | formin-binding protein 17 | FBP17 | 9q34 |
| 35292_at | HLA-B associated transcript 1 | BAT1 | 6p21.3 |
| 38607_at | transmembrane 4 superfamily member 5 | TM4SF5 | 17p13.3 |
| 35275_at | adaptor-related protein complex 1, gamma 1 subunit | AP1G1 | 16q23 |
| 36783_f_at | Krueppel-related zinc finger protein | H-plk | 7p14.1 |
| 33248_at | ESTs | no gene symbol | no location |
| 33470_at | KIAA1719 protein | KIAA1719 | 3p24-p23 |
| 38298_at | potassium large conductance calcium-activated channel, subfamily M | KCNMB1 | 5q34 |
| | beta member 1 | | |
| 32092_at | syndecan 3 (N-syndecan) | SDC3 | 1pter-p22.3 |
| 39421_at | runt-related transcription factor 1 (acute myeloid leukemia 1; | RUNX1 | 21q22.3 |
| | aml1 oncogene) | | |
| 38357_at | Homo sapiens mRNA; cDNA DKFZp564D156 | no gene symbol | no location |
| | (from clone DKFZp564D156) | | |
| 31819_at | Homo sapiens cDNA: FLJ23566 fis, clone LNG10880 | no gene symbol | no location |
| 41690_at | Homo sapiens mRNA; cDNA DKFZp586N012 | no gene symbol | no location |
| | (from clone DKFZp586N012) | | |
| 38964_r_at | Wiskott-Aldrich syndrome (eczema-thrombocytopenia) | WAS | Xp11.4-p11.21 |
| 40839_at | ubiquitin-like 3 | UBL3 | 13q12-q13 |
| 33543_s_at | pinin, desmosome associated protein | PNN | 14q13.2 |
| 32085_at | KIAA0981 protein | KIAA0981 | 2q34 |
| 38752_r_at | ATP synthase, H+ transporting, mitochondrial F0 complex, subunit e | ATP5I | 4p16.3 |
| 34137_at | no title | no gene symbol | no location |
| 41279_f_at | mitogen-activated protein kinase 8 interacting protein 1 | MAPK8IP1 | 11p12-p11.2 |
| 442_at | tumor rejection antigen (gp96) 1 | TRA1 | 12q24.2-q24.3 |
| 32508_at | KIAA1096 protein | KIAA1096 | 1q23.3 |
| 35790_at | vacuolar protein sorting 26 (yeast) | VPS26 | 10q21.1 |
| 40094_r_at | Lutheran blood group (Auberger b antigen included) | LU | 19q13.2 |
| 33520_at | coagulation factor VII (serum prothrombin conversion accelerator) | F7 | 13q34 |
| 33792_at | prostate stem cell antigen | PSCA | 8q24.2 |
| 37678_at | putative transmembrane protein | NMA | 10p12.3-p11.2 |

CLUSTER Z

| | | | |
|---|---|---|---|
| | TITLE - CLUSTER Z | | |
| 34400_at | low molecular mass ubiquinone-binding protein (9.5 kD) | QP-C | 5q31.1 |
| 39921_at | cytochrome c oxidase subunit Vb | COX5B | 2cen-q13 |
| 40546_s_at | NADH dehydrogenase (ubiquinone) 1 alpha subcomplex, 2 (8 kD, B8) | NDUFA2 | 5q31 |
| 38085_at | chromobox homolog 3 (HP1 gamma homolog, Drosophila) | CBX3 | 7p21.1 |
| 39778_at | mannosyl (alpha-1,3-)-glycoprotein beta-1,2-N- | MGAT1 | 5q35 |
| | acetylglucosaminyltransferase | | |
| 36600_at | proteasome (prosome, macropain) activator subunit 1 (PA28 alpha) | PSME1 | 14q11.2 |
| 40433_at | Homo sapiens, clone IMAGE: 4391536, mRNA | no gene symbol | no location |
| 35767_at | GABA(A) receptor-associated protein-like 2 | GABARAPL2 | 16q22.3-q24.1 |
| 1450_g_at | proteasome (prosome, macropain) subunit, alpha type, 4 | PSMA4 | 15q24.2 |
| 33738_r_at | Homo sapiens cervical cancer suppressor-1 mRNA, complete cds | no gene symbol | no location |
| 40134_at | ATP synthase, H+ transporting, mitochondrial F0 complex, subunit f, | ATP5J2 | 7q11.21 |
| | isoform 2 | | |
| 567_s_at | promyelocytic leukemia | PML | 15q22 |
| 40881_at | ATP citrate lyase | ACLY | 17q12-q21 |
| 38974_at | RNA-binding protein regulatory subunit | DJ-1 | 1p36.33-p36.12 |
| 33819_at | lactate dehydrogenase B | LDHB | 12p12.2-p12.1 |
| 40854_at | ubiquinol-cytochrome c reductase core protein II | UQCRC2 | 16p12 |
| 41694_at | BN51 (BHK21) temperature sensitivity complementing | BN51T | 8q21 |
| 38771_at | histone deacetylase 1 | HDAC1 | 1p34 |
| 40792_s_at | triple functional domain (PTPRF interacting) | TRIO | 5p15.1-p14 |
| 970_r_at | ubiquitin specific protease 9, X chromosome (fat facets-like | USP9X | Xp11.4 |
| | Drosophila) | | |
| 34381_at | cytochrome c oxidase subunit VIIc | COX7C | 5q14 |
| 35992_at | musculin (activated B-cell factor-1) | MSC | 8q21 |
| 40774_at | chaperonin containing TCP1, subunit 3 (gamma) | CCT3 | 1q23 |
| 32701_at | armadillo repeat gene deletes in velocardiofacial syndrome | ARVCF | 22q11.21 |
| 33011_at | neurotensin receptor 2 | NTSR2 | no location |
| 36676_at | ribophorin II | RPN2 | 20q12-q13.1 |
| 33510_s_at | glutamate receptor, metabotropic 1 | GRM1 | 6q24 |
| 37866_at | Homo sapiens mRNA full length insert cDNA clone | no gene symbol | no location |
| | EUROIMAGE 29222 | | |
| 41175_at | core-binding factor, beta subunit | CBFB | 16q22.1 |
| 39920_r_at | C1q-related factor | CRF | 17q21 |
| 32550_r_at | CCAAT/enhancer binding protein (C/EBP), alpha | CEBPA | 19q13.1 |
| 32104_i_at | calcium/calmodulin-dependent protein kinase (CaM kinase) II gamma | CAMK2G | 10q22 |
| 39747_at | polymerase (RNA) II (DNA directed) polypeptide G | POLR2G | 11q13.1 |
| 38516_at | sodium channel, voltage-gated, type I, beta polypeptide | SCN1B | 19q13.1 |
| 39131_at | similar to yeast Upf3, variant A | UPF3A | 13q34 |
| 35297_at | NADH dehydrogenase (ubiquinone) 1, alpha/beta subcomplex, 1 | NDUFAB1 | 16p11.2 |
| 40764_at | glutamic-oxaloacetic transaminase 2, mitochondrial (2) | GOT2 | 16q21 |
| 41833_at | jumping translocation breakpoint | JTB | 1q21 |
| 39741_at | hydroxyacyl-Coenzyme A dehydrogenase/3-ketoacyl-Coenzyme A | HADHB | 2p23 |
| | thiolase/enoyl-Coenzyme A hydratase (trifunctional protein) | | |
| 34894_r_at | protease, serine, 22 | PRSS22 | 16p13.3 |
| 37796_at | leucine-rich repeat protein, neuronal 1 | LRRN1 | 7q22 |
| 36355_at | involucrin | IVL | 1q21 |
| 1072_g_at | GATA binding protein 2 | GATA2 | 3q21 |
| 33447_at | myosin, light polypeptide, regulatory, non-sarcomeric (20 kD) | MLCB | 18p11.31 |
| 39448_r_at | B7 protein | B7 | 12p13 |
| 37337_at | small nuclear ribonucleoprotein polypeptide G | SNRPG | 2p12 |
| 37414_at | solute carrier family 22 (organic cation transporter), member 1-like | SLC22A1LS | 11p15.5 |
| 41255_at | Homo sapiens mRNA; cDNA DKFZp434E0528 | no gene symbol | no location |
| 721_g_at | heat shock transcription factor 4 | HSF4 | 16q21 |
| 39184_at | transcription elongation factor B (SIII), polypeptide 2 (elongin B) | TCEB2 | 13 |
| 40189_at | SET translocation (myeloid leukemia-associated) | SET | 9q34 |
| 37677_at | phosphoglycerate kinase 1 | PGK1 | Xq13 |
| 34602_at | ficolin (collagen/fibrinogen domain containing lectin) 2 (hucolin) | FCN2 | 9q34 |
| 41374_at | ribosomal protein S6 kinase, 70 kD, polypeptide 2 | RPS6KB2 | 11q12.2 |
| 40467_at | succinate dehydrogenase complex, subunit D, integral protein | SDHD | 11q23 |
| 33137_at | latent transforming growth factor beta binding protein 4 | LTBP4 | 19q13.1-q13.2 |
| 36826_at | general transcription factor IIF, polypeptide 1 (74 kD subunit) | GTF2F1 | 19p13.3 |
| 37546_r_at | secretory carrier membrane protein 5 | SCAMP5 | no location |
| 33632_g_at | similar to S. pombe dim1+ | DIM1 | 18q23 |
| 41146_at | ADP-ribosyltransferase (NAD+; poly (ADP-ribose) polymerase) | ADPRT | 1q41-q42 |
| 36188_at | general transcription factor IIIA | GTF3A | 13q12.3-q13.1 |
| 32511_at | ESTs | no gene symbol | no location |
| 39795_at | adaptor-related protein complex 2, mu 1 subunit | AP2M1 | 3q28 |
| 396_f_at | erythropoietin receptor | EPOR | 19p13.3-p13.2 |
| 31497_at | G antigen 1 | GAGE1 | Xp11.4-p11.2 |
| 34573_at | ephrin-A3 | EFNA3 | 1q21-q22 |
| 37668_at | complement component 1, q subcomponent binding protein | C1QBP | 17p13.3 |
| 37348_s_at | thyroid hormone receptor interactor 7 | TRIP7 | 6q15 |
| 37766_s_at | proteasome (prosome, macropain) 26S subunit, ATPase, 5 | PSMC5 | 17q23-q25 |
| 34380_at | stomatin (EPB72)-like 2 | STOML2 | 9p13.1 |
| 39174_at | nuclear receptor coactivator 4 | NCOA4 | 10q11.2 |
| 36032_at | HSPCO34 protein | LOC51668 | 1p32.1-p33 |
| 160020_at | matrix metalloproteinase 14 (membrane-inserted) | MMP14 | 14q11-q12 |
| 34783_s_at | BUB3 budding uninhibited by benzimidazoles 3 homolog (yeast) | BUB3 | 10q26 |
| 33027_at | no title | no gene symbol | no location |
| 38368_at | dUTP pyrophosphatase | DUT | 15q15-q21.1 |
| 36688_at | sterol carrier protein 2 | SCP2 | 1p32 |
| 38251_at | myosin light chain 1 slow a | MLC1SA | 12q13.13 |
| 39803_s_at | chromosome 21 open reading frame 2 | C21orf2 | 21q22.3 |
| 35734_at | ARP2 actin-related protein 2 homolog (yeast) | ACTR2 | 2p14 |
| 32004_s_at | cell division cycle 2-like 2 | CDC2L2 | 1p36.3 |
| 1827_s_at | v-myc myelocytomatosis viral oncogene homolog (avian) | MYC | 8q24.12-q24.13 |
| 32530_at | tyrosine 3-monooxygenase/tryptophan 5-monooxygenase activation | YWHAQ | 22q12-qter |
| | protein, theta polypeptide | | |
| 33727_r_at | tumor necrosis factor receptor superfamily, member 6b, decoy | TNFRSF6B | 20q13.3 |
| 34970_r_at | 5-oxoprolinase (ATP-hydrolysing) | OPLAH | 8 |
| 36122_at | proteasome (prosome, macropain) subunit, alpha type, 6 | PSMA6 | 14q13 |
| 32849_at | SMC1 structural maintenance of chromosomes 1-like 1 (yeast) | SMC1L1 | Xp11.22-p11.21 |
| 31812_at | guanosine monophosphate reductase | GMPR | 6p23 |
| 36218_g_at | serine/threonine kinase 38 | STK38 | 6p21 |

CLUSTERS S1 + S2 VERSUS ALL OTHER CLUSTERS

| | | | |
|---|---|---|---|
| | TITLE - S1 + S2 against the rest | | |
| 38319_at | CD3D antigen, delta polypeptide (TiT3 complex) | CD3D | 11q23 |
| 38147_at | SH2 domain protein 1A, Duncan's disease (lymphoproliferative | SH2D1A | Xq25-q26 |
| | syndrome) | | |
| 39226_at | CD3G antigen, gamma polypeptide (TiT3 complex) | CD3G | 11q23 |
| 33238_at | lymphocyte-specific protein tyrosine kinase | LCK | 1p34.3 |
| 2059_s_at | lymphocyte-specific protein tyrosine kinase | LCK | 1p34.3 |
| 32794_g_at | T cell receptor beta locus | TRB@ | 7q34 |
| 31891_at | chitinase 3-like 2 | CHI3L2 | 1p13.3 |
| 38949_at | protein kinase C, theta | PRKCQ | 10p15 |
| 37344_at | major histocompatibility complex, class II, DM alpha | HLA-DMA | 6p21.3 |
| 38095_i_at | major histocompatibility complex, class II, DP beta 1 | HLA-DPB1 | 6p21.3 |
| 38096_f_at | major histocompatibility complex, class II, DP beta 1 | HLA-DPB1 | 6p21.3 |
| 38051_at | mal, T-cell differentiation protein | MAL | 2cen-q13 |
| 40688_at | linker for activation of T cells | LAT | no location |
| 1096_g_at | CD19 antigen | CD19 | 16p11.2 |
| 1105_s_at | T cell receptor beta locus | TRB@ | 7q34 |
| 40954_at | FXYD domain-containing ion transport regulator 2 | FXYD2 | 11q23 |
| 35016_at | CD74 antigen (invariant polypeptide of major histocompatibility | CD74 | 5q32 |
| | complex, class II antigen-associated) | | |
| 40775_at | integral membrane protein 2A | ITM2A | Xq13.3-Xq21.2 |
| 40738_at | CD2 antigen (p50), sheep red blood cell receptor | CD2 | 1p13 |
| 38547_at | integrin, alpha L (antigen CD11A (p180), lymphocyte function- | ITGAL | 16p11.2 |
| | associated antigen 1; alpha polypeptide) | | |
| 36277_at | CD3E antigen, epsilon polypeptide (TiT3 complex) | CD3E | 11q23 |
| 41165_g_at | immunoglobulin heavy constant mu | IGHM | 14q32.33 |
| 41523_at | RAB32, member RAS oncogene family | RAB32 | 6q24.3 |
| 38315_at | aldehyde dehydrogenase 1 family, member A2 | ALDH1A2 | 15q21.1-q21.2 |
| 38917_at | T cell receptor delta locus | TRD@ | 14q11.2 |
| 38833_at | major histocompatibility complex, class II, DP alpha 1 | HLA-DPA1 | 6p21.3 |
| 39119_s_at | natural killer cell transcript 4 | NK4 | 16p13.3 |
| 40147_at | vesicle amine transport protein 1 | VATI | 17q21 |
| 37039_at | major histocompatibility complex, class II, DR alpha | HLA-DRA | 6p21.3 |
| 1110_at | T cell receptor delta locus | TRD@ | 14q11.2 |
| 39709_at | selenoprotein W, 1 | SEPW1 | 19q13.3 |
| 771_s_at | CD7 antigen (p41) | CD7 | 17q25.2-q25.3 |
| 41164_at | immunoglobulin heavy constant mu | IGHM | 14q32.33 |
| 39248_at | aquaporin 3 | AQP3 | 9p13 |
| 34927_at | CD1B antigen, b polypeptide | CD1B | 1q22-q23 |
| 37399_at | aldo-keto reductase family 1, member C3 (3-alpha hydroxysteroid | AKR1C3 | 10p15-p14 |
| | dehydrogenase, type II) | | |
| 1498_at | zeta-chain (TCR) associated protein kinase (70 kD) | ZAP70 | 2q12 |
| 39930_at | EphB6 | EPHB6 | 7q33-q35 |
| 40570_at | forkhead box O1A (rhabdomyosarcoma) | FOXO1A | 13q14.1 |
| 37861_at | CD1E antigen, e polypeptide | CD1E | 1q22-q23 |
| 37078_at | CD3Z antigen, zeta polypeptide (TiT3 complex) | CD3Z | 1q22-q23 |
| 35643_at | nucleobindin 2 | NUCB2 | 11p15.1-p14 |
| 38017_at | CD79A antigen (immunoglobulin-associated alpha) | CD79A | 19q13.2 |
| 38408_at | transmembrane 4 superfamily member 2 | TM4SF2 | Xq11.4 |
| 41166_at | immunoglobulin heavy constant mu | IGHM | 14q32.33 |
| 605_at | vesicle amine transport protein 1 | VATI | 17q21 |
| 245_at | selectin L (lymphocyte adhesion molecule 1) | SELL | 1q23-q25 |
| 2047_s_at | junction plakoglobin | JUP | 17q21 |
| 2031_s_at | cyclin-dependent kinase inhibitor 1A (p21, Cip1) | CDKN1A | 6p21.2 |
| 33236_at | retinoic acid receptor responder (tazarotene induced) 3 | RARRES3 | 11q23 |
| 32649_at | transcription factor 7 (T-cell specific, HMG-box) | TCF7 | 5q31.1 |
| 36773_f_at | major histocompatibility complex, class II, DQ beta 1 | HLA-DQB1 | 6p21.3 |
| 38750_at | Notch homolog 3 (Drosophila) | NOTCH3 | 19p13.2-p13.1 |
| 41609_at | major histocompatibility complex, class II, DM beta | HLA-DMB | 6p21.3 |
| 32793_at | T cell receptor beta locus | TRB@ | 7q34 |
| 38893_at | neutrophil cytosolic factor 4 (40 kD) | NCF4 | 22q13.1 |
| 41723_s_at | major histocompatibility complex, class II, DR beta 1 | HLA-DRB1 | 6p21.3 |
| 37403_at | annexin A1 | ANXA1 | 9q12-q21.2 |
| 36473_at | ubiquitin specific protease 20 | USP20 | 9q34.12-q34.13 |
| 36941_at | ALL1-fused gene from chromosome 1 q | AF1Q | 1q21 |
| 39319_at | lymphocyte cytosolic protein 2 (SH2 domain-containing leukocyte | LCP2 | 5q33.1-qter |
| | protein of 76 kD) | | |
| 36878_f_at | major histocompatibility complex, class II, DQ beta 1 | HLA-DQB1 | 6p21.3 |
| 907_at | adenosine deaminase | ADA | 20q12-q13.11 |
| 33121_g_at | regulator of G-protein signalling 10 | RGS10 | 10q25 |
| 41468_at | T cell receptor gamma locus | TRG@ | 7p15-p14 |
| 37849_at | slit homolog 1 (Drosophila) | SLIT1 | 10q23.3-q24 |
| 38253_at | amylo-1, 6-glucosidase, 4-alpha-glucanotransferase (glycogen | AGL | 1p21 |
| | debranching enzyme, glycogen storage disease type III) | | |
| 34033_s_at | leukocyte immunoglobulin-like receptor, subfamily A (with TM | LILRA2 | 19q13.4 |
| | domain), member 2 | | |
| 41819_at | FYN binding protein (FYB-120/130) | FYB | 5p13.1 |
| 35985_at | A kinase (PRKA) anchor protein 2 | AKAP2 | 9q31-q33 |
| 33821_at | homolog of yeast long chain polyunsaturated fatty acid elongation | HELO1 | 6p21.1-p12.1 |
| | enzyme 2 | | |
| 172_at | inositol polyphosphate-5-phosphatase, 145 kD | INPP5D | 2q36-q37 |
| 37759_at | Lysosomal-associated multispanning membrane protein-5 | LAPTM5 | 1p34 |
| 36937_s_at | PDZ and LIM domain 1 (elfin) | PDLIM1 | 10q22-q26.3 |
| 33641_g_at | allograft inflammatory factor 1 | AIF1 | 6p21.3 |
| 41156_g_at | catenin (cadherin-associated protein), alpha 1 (102 kD) | CTNNA1 | 5q31 |
| 37890_at | CD47 antigen (Rh-related antigen, integrin-associated signal | CD47 | 3q13.1-q13.2 |
| | transducer) | | |
| 39273_at | ESTs | no gene symbol | no location |
| 41409_at | basement membrane-induced gene | ICB-1 | 1p35.3 |
| 40155_at | actin binding LIM protein | ABLIM | 10q25 |
| 33291_at | RAS guanyl releasing protein 1 (calcium and DAG-regulated) | RASGRP1 | 15q15 |
| 36658_at | 24-dehydrocholesterol reductase | DHCR24 | 1p33-p31.1 |
| 38581_at | guanine nucleotide binding protein (G protein), q polypeptide | GNAQ | 9q21 |
| 33316_at | KIAA0808 gene product | TOX | 8q12.2-q12.3 |
| 37598_at | Ras association (RalGDS/AF-6) domain family 2 | RASSF2 | 20pter-p12.1 |
| 36808_at | protein tyrosine phosphatase, non-receptor type 22 (lymphoid) | PTPN22 | 1p13.3-p13.1 |
| 39044_s_at | diacylglycerol kinase, delta (130 kD) | DGKD | 2q37.1 |
| 39318_at | T-cell leukemia/lymphoma 1A | TCL1A | 14q32.1 |
| 33777_at | thromboxane A synthase 1 (platelet, cytochrome P450, subfamily V) | TBXAS1 | 7q34-q35 |

CLUSTER S1 vs. S2

| | | | |
|---|---|---|---|
| | TITLE - S1 vs. S2 | | |
| 32528_at | ClpP caseinolytic protease, ATP-dependent, homolog (E. coli) | CLPP | 19p13.3 |
| 34182_at | N-deacetylase/N-sulfotransferase (heparan glucosaminyl) 1 | NDST1 | 5q32-q33.1 |
| 36158_at | dynactin 1 (p150, glued homolog, Drosophila) | DCTN1 | 2p13 |
| 36276_at | contactin 2 (axonal) | CNTN2 | 1q32.1 |
| 39917_at | gamma-tubulin complex protein 2 | GCP2 | 10q26.3 |
| 1942_s_at | cyclin-dependent kinase 4 | CDK4 | 12q14 |
| 31559_at | solute carrier family 13 (sodium-dependent dicarboxylate transporter) | SLC13A2 | 17p11.1-q11.1 |
| 121_at | paired box gene 8 | PAX8 | 2q12-q14 |
| 36126_at | nucleotide binding protein | NBP | 17q12-q21 |
| 31391_at | huntingtin-associated protein 1 (neuroan 1) | HAP1 | 17q21.2-q21.3 |
| 33448_at | serine protease inhibitor, Kunitz type 1 | SPINT1 | 15q13.3 |
| 37905_r_at | no title | no gene symbol | no location |
| 35727_at | uridine kinase-like 1 | URKL1 | 20q13.33 |
| 38998_g_at | solute carrier family 25 (mitochondrial carrier; citrate transporter) | SLC25A1 | 22q11.21 |
| 40862_i_at | creatine kinase, brain | CKB | 14q32 |
| 2025_s_at | APEX nuclease (multifunctional DNA repair enzyme) | APEX | 14q11.2-q12 |
| 33493_at | erythroid differentiation and denucleation factor 1 | HFL-EDDG1 | 18p11.1 |
| 396_f_at | erythropoietin receptor | EPOR | 19p13.3-p13.2 |
| 40115_at | CCR4-NOT transcription complex, subunit 7 | CNOT7 | 8p22-p21.3 |
| 33640_at | allograft inflammatory factor 1 | AIF1 | 6p21.3 |
| 40094_r_at | Lutheran blood group (Auberger b antigen included) | LU | 19q13.2 |
| 1309_at | proteasome (prosome, macropain) subunit, beta type, 3 | PSMB3 | 2q35 |
| 39920_r_at | C1q-related factor | CRF | 17q21 |
| 40299_at | G-protein coupled receptor | RE2 | 1q23.2 |
| 1280_i_at | no title | no gene symbol | no location |
| 33011_at | neurotensin receptor 2 | NTSR2 | no location |
| 34963_at | no title | no gene symbol | no location |
| 38442_at | microfibrillar-associated protein 2 | MFAP2 | 1p36.1-p35 |
| 1827_s_at | v-myc myelocytomatosis viral oncogene homolog (avian) | MYC | 8q24.12-q24.13 |
| 33706_at | squamous cell carcinoma antigen recognised by T cells | SART1 | 11q12.1 |
| 41184_s_at | proteasome (prosome, macropain) subunit, beta type, 8 (large | PSMB8 | 6p21.3 |
| | multifunctional protease 7) | | |
| 40817_at | nucleobindin 1 | NUCB1 | 19q13.2-q13.4 |
| 32335_r_at | ubiquitin C | UBC | 12q24.3 |
| 38964_r_at | Wiskott-Aldrich syndrome (eczema-thrombocytopenia) | WAS | Xp11.4-p11.21 |
| 34970_r_at | 5-oxoprolinase (ATP-hydrolysing) | OPLAH | 8 |
| 34539_at | olfactory receptor, family 7, subfamily A, member 126 pseudogene | OR7E126P | 11 |
| 36565_at | zinc finger protein 183 (RING finger, C3HC4 type) | ZNF183 | Xq25-q26 |
| 160044_g_at | aconitase 2, mitochondrial | ACO2 | 22q13.2-q13.31 |
| 41034_s_at | sulfotransferase family, cytosolic, 2B, member 1 | SULT2B1 | 19q13.3 |
| 39731_at | RNA binding motif protein, X chromosome | RBMX | Xq26 |
| 567_s_at | promyelocytic leukemia | PML | 15q22 |
| 870_f_at | metallothionein 3 (growth inhibitory factor (neurotrophic)) | MT3 | 16q13 |
| 327_f_at | no title | no gene symbol | no location |
| 33132_at | cleavage and polyadenylation specific factor 1, 160 kD subunit | CPSF1 | 8q24.23 |
| 36600_at | proteasome (prosome, macropain) activator subunit 1 (PA28 alpha) | PSME1 | 14q11.2 |
| 39965_at | ras-related C3 botulinum toxin substrate 3 (rho family, small GTP | RAC3 | 17q25.3 |
| | binding protein Rac3) | | |
| 1053_at | replication factor C (activator 1) 2 (40 kD) | RFC2 | 7q11.23 |
| 32007_at | no title | no gene symbol | no location |
| 36452_at | synaptopodin | KIAA1029 | 5q33.1 |
| 884_at | integrin, alpha 3 (antigen CD49C, alpha 3 subunit of VLA-3 | ITGA3 | 17q23.3 |
| | receptor) | | |
| 36881_at | electron-transfer-flavoprotein, beta polypeptide | ETFB | 19q13.3 |
| 34166_at | solute carrier family 6 (neurotransmitter transporter, L-proline), | SLC6A7 | 5q31-q32 |
| | member 7 | | |
| 33247_at | 26S proteasome-associated pad1 homolog | POH1 | 2q24.3 |
| 32104_i_at | calcium/calmodulin-dependent protein kinase (CaM kinase) II | CAMK2G | 10q22 |
| | gamma | | |
| 35385_at | COQ7 coenzyme Q, 7 homolog ubiquinone (yeast) | COQ7 | 16p13.11-p12.3 |
| 31745_at | mucin 3A, intestinal | MUC3A | 7q22 |
| 35595_at | ESTs, Highly similar to calcitonin gene-related peptide-receptor | no gene symbol | no location |
| | component protein [Homo sapiens] [H. sapiens] | | |
| 41703_r_at | A kinase (PRKA) anchor protein 7 | AKAP7 | 6q23 |
| 39608_at | single-minded homolog 2 (Drosophila) | SIM2 | 21q22.13 |
| 37885_at | hypothetical protein AF038169 | AF038169 | 2q22.1 |
| 1470_at | polymerase (DNA directed), delta 2, regulatory subunit (50 kD) | POLD2 | 7p15.1 |
| 37766_s_at | proteasome (prosome, macropain) 26S subunit, ATPase, 5 | PSMC5 | 17q23-q25 |
| 34302_at | eukaryotic translation initiation factor 3, subunit 4 (delta, 44 kD) | EIF3S4 | 19p13.2 |
| 40441_g_at | PAI-1 mRNA-binding protein | PAI-RBP1 | 1p31-p22 |
| 36218_g_at | serine/threonine kinase 38 | STK38 | 6p21 |
| 33255_at | nuclear autoantigenic sperm protein (histone-binding) | NASP | 8q11.23 |
| 39009_at | Lsm3 protein | LSM3 | 3p25.1 |
| 32540_at | protein phosphatase 3 (formerly 2B), catalytic subunit, gamma | PPP3CC | 8p21.2 |
| | isoform (calcineurin A gamma) | | |
| 35911_r_at | matrix metalloproteinase-like 1 | MMPL1 | 16p13.3 |
| 39937_at | chemokine (C-C motif) receptor 2 | CCR2 | 3p21 |
| 1553_r_at | no title | no gene symbol | no location |
| 31550_at | adrenergic, beta-1-, receptor | ADRB1 | 10q24-q26 |
| 1446_at | proteasome (prosome, macropain) subunit, alpha type, 2 | PSMA2 | 7p15.1 |
| 36004_at | inhibitor of kappa light polypeptide gene enhancer in B-cells, kinase gamma | IKBKG | Xq28 |
| 1494_f_at | cytochrome P450, subfamily IIA (phenobarbital-inducible), polypeptide 6 | CYP2A6 | 19q13.2 |
| 41458_at | KIAA0467 protein | KIAA0467 | 1p34.1 |
| 36125_s_at | RNA binding protein (autoantigenic, hnRNP-associated with lethal yellow) | RALY | 20q11.21-q11.23 |
| 33349_at | Homo sapiens mRNA; cDNA DKFZp586I1518 | no gene symbol | no location |
| 38682_at | BRCA1 associated protein-1 (ubiquitin carboxy-terminal hydrolase) | BAP1 | 3p21.31-p21.2 |
| 34577_at | melanoma antigen, family A, 9 | MAGEA9 | Xq28 |
| 35096_at | solute carrier family 1 (high affinity aspartate/glutamate transporter) | SLC1A6 | 19p13.13 |
| 34573_at | ephrin-A3 | EFNA3 | 1q21-q22 |
| 33071_at | H2B histone family, member N | H2BFN | 6p22-p21.3 |
| 34894_r_at | protease, serine, 22 | PRSS22 | 16p13.3 |
| 39448_r_at | B7 protein | B7 | 12p13 |
| 32190_at | fatty acid desaturase 2 | FADS2 | 11q12-q13.1 |
| 34325_at | polyglutamine binding protein 1 | PQBP1 | Xp11.23 |
| 33168_at | Homo sapiens cDNA: FLJ23067 fis, clone LNG04993 | no gene symbol | no location |
| 32681_at | solute carrier family 9 (sodium/hydrogen exchanger), isoform 1 | SLC9A1 | 1p36.1-p35 |
| | (antiporter, Na+/H+, amiloride sensitive) | | |

### Example XIII

### Gene Expression Profiling for Molecular Classification and Outcome Prediction in Infant Leukemia Reveals Novel Biologic Clusters, Etiologies and Pathways for Treatment Failure

To determine if traditional biologic and clinical subgroups of infant leukemia cases could be identified by gene expression profiles, 126 infant leukemia cases registered to NCI-sponsored Infant Oncology Group/Children's Oncology Group treatment trials were studied using oligonucleotide microarrays containing 12,625 probe sets (Affymetrix U95Av2 array platform). Of the 126 cases, 78 were ALL (62%), 48 were AML (38%) and 53 (42%) cases had translocations involving the MLL gene (chromosome segment 11q23).

The exploratory evaluation of our data set was performed in several steps. The first step of the analysis was the construction of predictive classification algorithms that linked the gene expression data to the traditional clinical variables that define treatment, using supervised learning techniques, and further, the exploration of patterns that could predict patient outcomes. As described in Example IA, the 126 patients were divided into statistically balanced and representative training (82 patients) and test sets (44 patients), according to the clinical labels (leukemia lineage, cytogenetics and outcome). For classification purposes, two primary supervised approaches were used; Bayesian networks and recursive feature elimination in the context of Support Vector Machines (SVM-RFE). Additional classification techniques (Fuzzy inference and Discriminant Analysis) were used for comparison purposes.

All of the classification algorithms were established based on the training data set and then used to predict the class of the samples in the test. Two statistical significance tests were employed to further evaluate the prediction accuracy of those algorithms. The first tested whether the success rate of each classification algorithm was significantly greater than the value that would be expected by chance alone (i.e. whether the success rate was significantly greater than 0.5, where the success rate=# of correct predictions/total predictions). The second prediction accuracy test used the true positive proportion (TP) and false positive proportion (FP) value computed for one of the two classes. For a binary classification problem, TP is the ratio of correctly classified samples in the class to the total number in the class. FP is the proportion of misclassified samples in the other class to the total number in that class. To test whether the true positive proportion was significantly greater than the false positive proportion, we used Fisher's exact test. The p-values of the two tests along with the success rates for each of the classification algorithms with respect to the classification tasks of interest are listed in Table 44. As shown in the table, both evaluation methods confirmed that the classification results for the lineage labels (ALL/AML) and the presence or absence of t(4;11) rearrangements were significant at level α=0.05. In other words, all the supervised learning techniques employed were successful in finding a distinction between ALL and AML samples, and the presence/absence of t(4;11) rearrangements. Detailed gene lists that characterize each one of these leukemia subtypes were obtained from all the classifiers used and can be found in the Supplemental Information.

Class Discovery: Expression Profiles Partition Infant Leukemia Cases in Three Groups

To explore the intrinsic structure of the data independent of known class labels, several unsupervised clustering methods were employed. These unsupervised approaches allowed patient separation into potential clusters based on overall similarity in gene expression, without prior knowledge of clinical labels. As discussed below, although certain degree of correlation of our unsupervised clusters with traditional lineage (ALL/AML) and cytogenetics (MLL or not) could be observed, those labels were not enough to completely explain the results of our unsupervised clustering methods, suggesting that leukemia lineage and cytogenetics are not the only important factors in driving the inherent biology of these gene expression groups.

Initially, the data were investigated using agglomerative hierarchical clustering (Eisen et al., 1998). Hierarchical clustering results from the 126 infant leukemia samples using all genes yielded several groups that seemed to have no relation to the known lineage labels or the partition of the data suggested by the presence or absence of MLL rearrangements (see supplemental information).

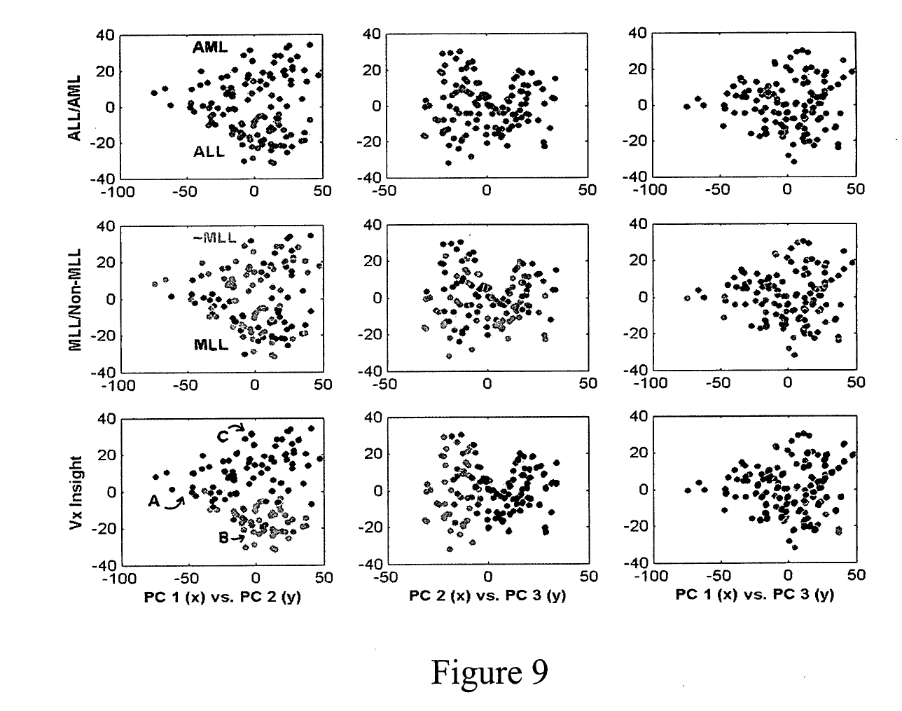

The next technique used was Principal Component Analysis (PCA). PCA, closely related to the Singular Value Decomposition (SVD), is an unsupervised data analysis method whereby the most variance is captured in the least number of coordinates (Joliffe, 1986; Kirby, 2001; Trefethan & Bau, 1997). As shown in FIG. 9, the first three principal components can be seen to partition the infant cohort into two different groups. These groups capture the infant ALL/AML lineage distinction, but only weakly agree with the MLL cytogenetics. Specifically, there is a 92% agreement between the PCA and the ALL/AML labels and only a 65% agreement between the PCA and MLL/non-MLL labels. Unexpectedly, the ALL/AML distinction does not appear until the second principal component, suggesting that morphology is not the most important factor explaining the variance in our data set. However, the first (and most important) principal component does not reveal any obvious clusters. Upon further analysis with a force-directed graph layout algorithm, we found the additional group (discussed later) seen only in the first principal component (colored in blue in FIG. 9).

The force-directed clustering algorithm (Davidson et al., 1998; 2001) places patients into clusters on the two-dimensional plane by minimizing two opposing forces. Briefly, the algorithm forms groups of patients by iteratively moving them toward one another with small steps proportional to the similarity of their gene expression, as measured by Pearson's correlation coefficient. To avoid collecting all of the patients into a single group, a counteracting force pushes nearby patients away from each other. This force increases in proportion to the number of nearby patients and has a strong local effect, thus acting to disperse any concentrated group of patients. This force affects only patients who are near each other, while the attractive force (Pearson's similarity) is independent of distance. The algorithm moves patients into a configuration that balances these two forces, thus grouping patients with similar gene expression. The spatial distribution of patients is then visualized on a three-dimensional plot, similar to a terrain map, where the height of the peaks denotes the local density of patients. This method has been useful in inferring functions of uncharacterized genes clustered near other genes with known functions (Kim, 2001) and for the analysis and mapping of various databases (Davidson, 1998, Werner-Washburne, 2002)

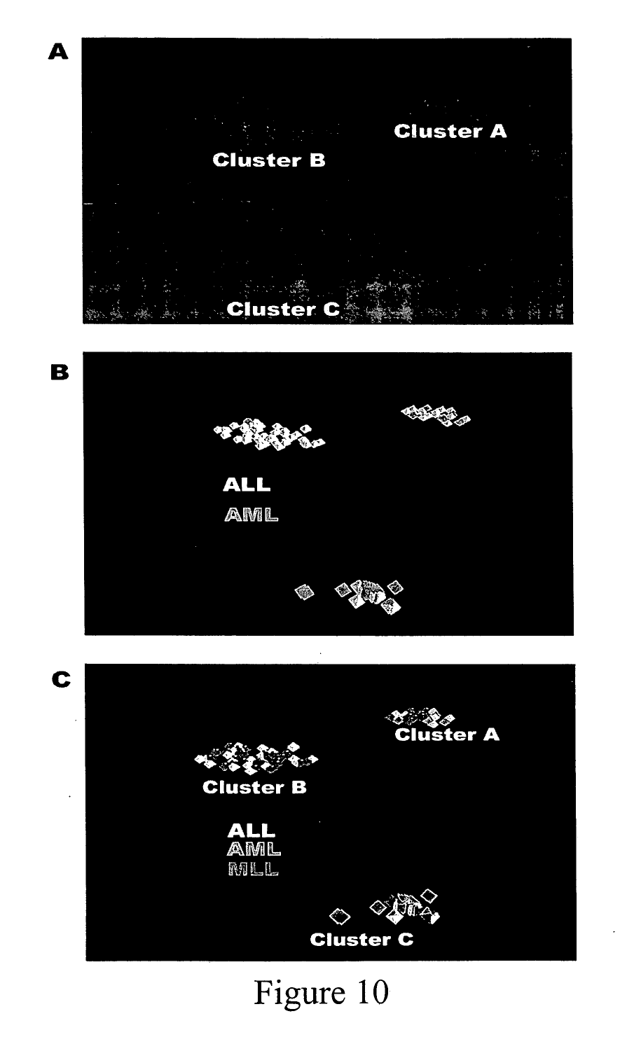

When applied to the infant data, the VxInsight clustering algorithm identifies several pattern of gene expression across the patients, suggesting the existence of three major groups (FIG. 10, and row three in FIG. 9), which hereafter will be denoted clusters A, B, and C. Despite different means of data transformation and different underlying mathematics, a high degree of overlap (92%) was observed between the clusters derived from PCA and the B and C clusters identified through the clustering algorithm native to VxInsight®. In addition, when the A group is displayed in the PCA projections (as seen in row three of FIG. 9), we see that it is distinguished from the B and C clusters in the first principal component. This lends additional support to the existence of and the importance of the A group.

Several further explorations into the VxInsight clusters were pursued. Linear discriminant analysis was used to separate the three clusters. The object of discriminant analysis is to weight and linearly combine information from the feature variables in a manner that clearly distinguishes labeled subclasses of the data. More specifically, the idea is to find a linear function of the feature variables such that the value of this function differs significantly between different classes. This function is the so-called discriminant function. Then, ANOVA was performed to rank cluster-discriminating genes in term of their F-test statistic values. From the top genes, a subset of genes was selected using stepwise discriminant analysis. This subset of genes served as the discriminating variables needed by linear discriminant analysis. The error rate of the derived classification results was 0.03, as estimated using fold-independent leave one out cross-validation (LOOCV). This indicated that the three VxInsight clusters were well separated.

There was also support for the existence of the VxInsight groupings even when only a subset of the data was used. For example, three widely separated groups of patients were observed when using only the patients in the training set. The addition of the rest of the patients in the test set, however, did induce change. In particular, the cores of Groups A and Groups C remained separated while Group B increased to include marginal members of groups A and C. The observation of similar grouping in both the entire set and the training set alone increased our interest in discerning the force driving the clustering for the patients in the VxInsight groups.

Finally, we confirmed our ability to classify patients into the VxInsight groups A, B, and C. Such a demonstration showed that we could categorize new patients into our grouping in the future (e.g. for treatment or diagnosis). To accomplish this, a multi-class Support Vector Machine (SVM) was trained using the actual labels A, B, and C in the patients from the training set. The prediction accuracy of this SVM on the test set was 95%. To verify that this result was improbable by chance alone, a randomization test was also performed. The labels A, B and C were randomly reassigned to the patients in both the training and the test set. Then, another SVM was trained with the re-labeled data in the training set. This SVM achieved a prediction accuracy of only 40% on the test set.

Subsequent exploration of the cluster-characterizing genes was performed using analysis of variance (ANOVA). The F-scores from this method were used to order all of the genes with respect to differential expressions between the groups. The strongest ranking 100 genes were then tabulated. The stability and strength of these gene lists was studied using statistical bootstrapping (Efron, 1979; Hjorth, 1994). This analysis provided a powerful method for determining the likelihood that a gene (high on the gene list determined from the actual data) would remain near the top of any gene list generated from experimental data similar to that which we actually observed. While this method allowed the identification of genes that had a unique pattern in each cluster and defined inter-clusters differences, it is important to make a distinction between these genes and the ones active in each one of the clusters (See supplemental information). Some very surprising findings were uncovered after completing a detailed analysis of the genes responsible for the distinction between clusters. These results, together with the stability of the clusters, suggest that the identified groups represent well-separated patient subclasses.

Approaches to Inherent Biology

Expression profiles identified different clusters of infant leukemia cases, not related to type labels or cytogenetics, but characterized by different genes predominantly expressed in, and probably related to, three independent disease initiation mechanisms. The sets of cluster-discriminating genes can be used to identify each biologic group and hence represent potentially important diagnostic and therapeutic targets (See Table 45). A heat map/dendrogram was produced with the top 30 genes that characterized each one of the three clusters, generated from the ANOVA analysis. Analysis of these genes revealed patterns that imply different features with potential clinical relevance.

The top cluster of cases (FIG. 10, cluster A, n=20, 15 ALL cases and 5 AML cases) has a gene expression profile that would not be recognized as “leukemic” per se. The cases in this cluster are distinguished by high expression of genes such as the novel tumor suppressor gene (ST5), embryonal antigens, adhesion molecules (particularly integrin α3), growth factor receptors for numerous lineages (keratinocytes and epithelial cells, hepatocytes, neuronal cells, and hematopoietic cells) and genes in the TGFB1 signaling pathway. The TGFB cytokines modulate the growth and functions of a wide variety of mammalian cell types. TGFB inhibits the proliferation of most types of cells. Proteins such as the latent transforming growth factor beta binding protein 4 (LTBP4), which is over expressed in this group of patients, are also regulated by TGFB. (Oklu, 2000). For this particular group of patients, cluster-discriminant genes such as CD34 (hematopoietic progenitor cell antigen), ataxin 2 related protein (responsible for specific stages of both cerebellar and vertebral column development), contacting (involved in glial development and tumorigenesis), the ski oncogene (another component of the TGFB 1 signaling pathway) and the erythropoietin receptor, suggest the involvement of an embryonal “common progenitor” primordial cell. Additionally, despite high expression of the above-mentioned characteristic genes, cases in this cluster demonstrated low to moderate expression of most genes. These data supports recent reports of stepwise decrease in transcriptional accessibility for multilineage-affiliated genes may represent progressive restriction of development potentials in early hematopoiesis ((Akashi et al., Blood 2003 Jan. 15; 101(2):383-9)). As suggested by Akashi et al, the size of the “functional genome” may be progressively reduced as hematopoietic stem cells undergo differentiation.

Other genes in this group with an absolutely unique pattern of expression include growth inhibitory factors like methallothionein 3 (MT3), embryonic cell transcription factors (UTF1) and stem cell antigens (prostate stem cell antigen) with remarkable homology to cell surface proteins that characterize the earliest phases of hematopoietic development (Reiter, 1998).

The left cluster of cases (FIG. 10, cluster B, n=52, 51 ALL cases and 1 AML case), is characterized by a high frequency of MLL rearrangements, predominantly t(4;11). This group was also distinguished by expression of lymphoid-characterizing genes (CD19, B lymphoid tyrosine kinase, CD79a) as well as EBV infection-related genes and genes associated with, or induced by, other DNA viruses. It is especially remarkable to find elevated expression of the Epstein-Barr virus-induced gene 2 (EB12) in more than 30% of the cases in this cluster (*82% of this cases have MLL rearrangements). EBI2 has been reported as one of the genes present in EBV infected B-lymphocytes (Birkenbach, 1993). Epstein-Barr virus infection of B lymphocytes, as well as infection of Burkitt lymphoma cells, induces an increase in the expression of this gene, identifiable by subtractive hybridization. We speculate that this group of cases might be initiated by a viral infection and that secondary, but critical MLL translocations stabilize or, alternatively, more fully transform these cells.

Finally, the third rightmost cluster (FIG. 9, cluster C, n=54, 42 AML cases and 12 ALL cases) is more heterogeneous and has a broader spectrum of MLL translocations. The gene expression signature of this group seems to have “myeloid” characteristics, with activation of genes previously reported as “myeloid-specific” such as Cystatin C(CST3), the myeloid cell nuclear differentiation factor (MNDA), and CCAAT/enhancer binding protein delta (C/EBP) (Golub, 1999; Skalnik, 2002). Members of the CCAAT/enhancer binding protein (C/EBP) family of transcription factors are important regulators of myeloid cell development (Skalnik, 2002). Other genes useful for cluster C prediction may also provide new insights into infant leukemia pathogenesis. For example, the mitogen activated protein kinase-activated protein kinase 3 is the first kinase to be activated through all 3 MAPK cascades: extracellular signal-regulated kinase (ERK), MAPKAP kinase-2, and Jun-N-terminal kinases/stress-activated protein kinases (Ludwig, 1996). It has been demonstrated as a determinant integrative element of signaling in both mitogen and stress responses. MAPKAPK3 showed high relative expression in the patients in cluster C. Many of the genes that characterize this cluster encode proteins characteristic of definitive myeloid differentiation (NDUFAB 1, SOD1, GSTTLp28), or which are critical for signal transduction (TYROBP). Interestingly, activation of many DNA repair and GST genes was also evident in this group of cases.

Altogether, the results of our class discovery methods suggested that, when applied to our patient data set, unsupervised techniques elucidate underlying novel subgroups of infant leukemia cases. In turn, this reassessment of tumor heterogeneity encourages the design of additional studies to ascertain whether these data can enhance the discriminatory power of currently employed prognostic variables.

Heterogeneous Distribution of the MLL Cases

The most common mutations in infant leukemia are translocations of the MLL gene at chromosome band 11q23. Interestingly, the MLL cases in cluster A (FIG. 10, lower left panel) are primarily t(4;11) (n=7), as well as two cases with t(10;11) and one with t(11;19). Cluster B, composed of virtually entirely ALL cases, contains a large number of t(4; 11) cases (n=29) as well as four cases with t(11;19), one case of t(10;11), and one case of t(1;11). Finally, the bottom right cluster (n=54), predominantly AML but containing twelve cases with an ALL label that nonetheless have more “myeloid” patterns of gene expression, also comprises five cases with t(9;11), three cases with t(1;11), three cases with t(11;19), one case with t(4;11) and three cases with other MLL translocations.

MLL cases with the same translocation (t(4;11) in clusters A and B) had dramatic differences in their gene expression profiles. The mechanisms that might underlie this striking difference are currently under study. Genes that have common patterns in the MLL cases across all three clusters have been identified; as well as genes that are uniquely expressed and which distinguish each MLL translocation variant. Although MLL cases are not homogeneous, it is interesting that the list of statistically significant genes derived in this study is quite similar to the list of genes derived by previous groups working in infant MLL leukemia (Armstrong, 2002). For reasons not understood, infants are more prone to MLL rearrangements that inhibit apoptosis and cause transformation. (reviewed in Van Limbergen et al, 2002). Our results suggest that the MLL translocation in these patients may not be the “initiating” event in leukemogenesis. It is possible that after a distinct initiating event, the infant patient is more prone to rearrange the MLL gene, and that this rearrangement leads to further cell transformation by preventing apoptosis. Alternatively, an MLL translocation could be a permissive initiating event with leukemogenesis and final gene expression profile determined more strongly by second mutations. Further studies within the MLL group of infant leukemia patients may provide the clues to processes determinant in leukemic transformation.

Pathways to Failure in Infant Leukemia

In general, gene expression data has supported the existence of several categories of acute leukemias related to the traditionally defined leukemia types, ALL and AML (Golub, 1999; Moos, 2002). However, while expression profiling is a robust approach for the accurate identification of known lineage and molecular subtypes across acute leukemia cases, the search for clinically relevant prognosis discriminators based on gene expression patterns has been less successful (Armstrong, 2002; Ferrando, 2002; Yeoh, 2002). As shown in Table 46, only SVM-RFE was able to identify remission vs. failure across the unconditioned data set with a total error rate differing from random prediction (success rate of 64% at a significance level of p<0.1). Interestingly, the performance of our outcome classification algorithms was not increased when conditioned on either of the traditional criterion of lineage (ALL vs. AML) nor cytogenetics (MLL vs. not MLL), providing further support for questioning the predictive value of these traditional clinical labels in explaining outcome in infant patients. However, far greater success in outcome prediction is obtained when conditioning the classifying algorithms on the VxInsight cluster membership. The effect of the three VxInsight clusters on our ability to predict remission vs. failure was then explored. In particular, we attempted to predict remission vs. failure in the entire data set, conditioned on the knowledge of into which VxInsight cluster each case falls. The hope was that, by utilizing knowledge of VxInsight cluster membership, inter-cluster expression profile variability of cases—which is not necessarily relevant to outcome prediction—would be eliminated, allowing intra-cluster variability relevant to outcome prediction to be more easily discovered by our classification algorithms.

Table 46 demonstrates that prediction accuracy is gained by coupling the supervised learning algorithms with VxInsight clustering. In the Bayesian method, accuracy against the test set rises from 0.568 (p=0.256) to 0.703 (p=0.010). Smaller improvements after conditioning are found with the other methods as well. One can look also at the prediction accuracy within the VxInsight clusters individually. There again a general rise in accuracy is observed, though not to a level of statistical significance, possibly due to the small size and/or class balance of the individual clusters.

We note that, from the more abstract perspective of machine learning theory, the construction of the VxInsight clusters is viewed as an external feature creation algorithm that is applied to a data set before the supervised learning algorithms begin their training. In the application at hand, the created feature is 3-valued, indicating membership of a case in VxInsight cluster A, B, or C. This feature creation process is akin to the pre-selection of features, based on measures of information content, that is employed by many supervised learning algorithms when run on problems of high dimensionality. One difference between the VxInsight feature creation step and traditional feature selection is that VxInsight clustering is performed without knowledge of the class label to be predicted (outcome, in this context), and hence it is reasonable to perform the clustering on the entire data set (train and test sets combined) at once.

The relative strength of the gene lists and parent sets can be thought of as being correlated with the prediction accuracy within the corresponding VxInsight cluster. However, it is the application of the lists and parent sets together within the two-step VxInsight/supervised learning conditioning framework described above that achieves statistical significance in its accuracy.

It is rather unlikely that random chance alone would improve such accuracy levels, since a process independent of the best error rate generated the VxInsight clustering. These results are taken as strong evidence that the VxInsight patient clusters reflect biologically important groups and, are clinically exploitable. In contrast, comparable accuracy was not achieved by conditioning on either of the traditional criteria of ALL vs. AML, nor MLL vs. not MLL. This may indicate that, as determined by our molecular analysis, these traditional clinical criteria for segregating treatment cohorts are less defining than has been supposed.

Table 47 illustrates the resulting set of distinguishing genes associated with remission/failure in the overall data set (not partitioning by type, cytogenetics or cluster), which represent potentially important diagnostic and therapeutic targets. Some of these outcome-correlated genes include Smurf1, a new member of the family of E3 ubiquitin ligases. Smurf1 selectively interacts with receptor-regulated MADs (mothers against decapentaplegia-related proteins) specific for the BMP pathway in order to trigger their ubiquitination and degradation, and hence their inactivation. Targeted ubiquitination of SMADs may serve to control both embryonic development and a wide variety of cellular responses to TGF-β signals. (Zhu, 1999). Another interesting gene is the SMA- and MAD-related protein, SMAD5, which plays a critical role in the signaling pathway in the TGF-β inhibition of proliferation of human hematopoietic progenitor cells (Bruno, 1998). The list also included regulators of differentiation and development; bone morphogenetic 2 protein, member of the transforming growth factor-beta (TGF-β) super family and determinant in neural development (White, 2001); DYRK1, a dual-specificity protein kinase involved in brain development (Becker, 1998); a small inducible cytokine A5 (SCYA5), the T cell activation increased late expression (TACTILE), and a myeloid cell nuclear differentiation antigen (MNDA). It is remarkable that this list includes potential diagnostic or therapeutic targets like the ERG oncogene (V-ETS Avian Erythroblastosis virus E26 oncogene related, found in AML patients), the phospholipase C-like protein 1 (PLCL, tumor suppressor gene), a cystein rich angiogenic inducer (CYR61), and the MYC, MYB oncogenes. Other genes in the list are located in critical regions mutated in leukemia, which suggests their connection with the leukemogenic process. Such genes include Selenoprotein P (SPP1, 5q), the protein kinase inhibitor p58 (DNAJC3 in 13q32), and the cyclin C (CCNC).

Discussion

Traditionally, infant leukemia has been classified according to a host of clinical parameters and biological features that tend to correlate with prognosis. This classification system has been used for risk-based classification assignment. However, unexplained variability in clinical courses still exists among some individuals within defined risk-group strata. Differences in the molecular constitution of malignant cells within subgroups may help to explain this variability.

In our initial profiling of 126 infant acute leukemia cases, we have used microarray technology to both segregate patient subgroups and to uncover genetic diversity among patients that fall within the same traditional risk groups. The results reported here identify three previously unrecognized groups of infant leukemia cases, driven by differential gene expression pattern and possibly related to three independent disease initiation mechanisms. Two of these clusters support previous data about leukemic etiology: environmental exposure and viral infections, both of which may occur in utero.

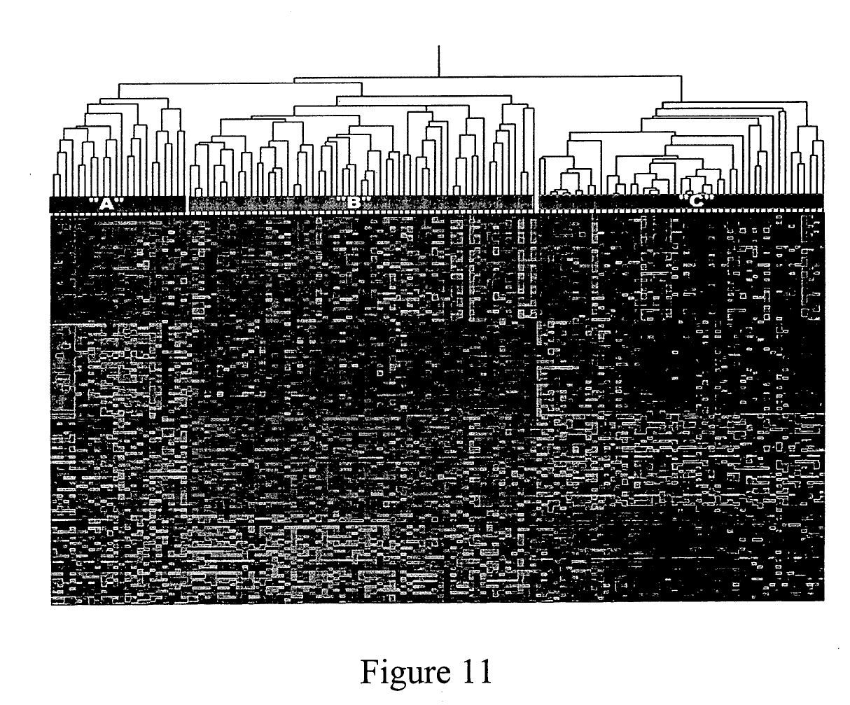

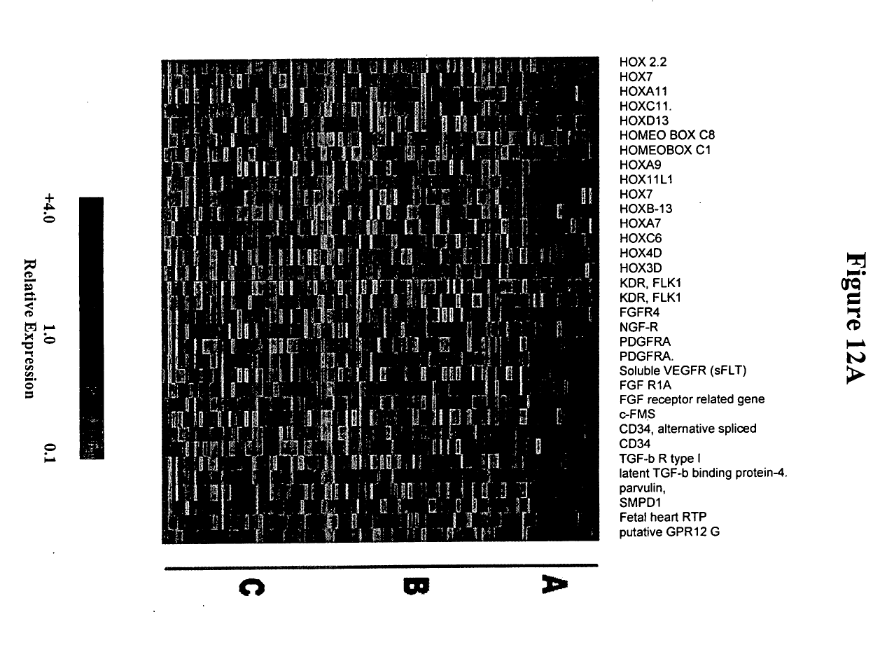

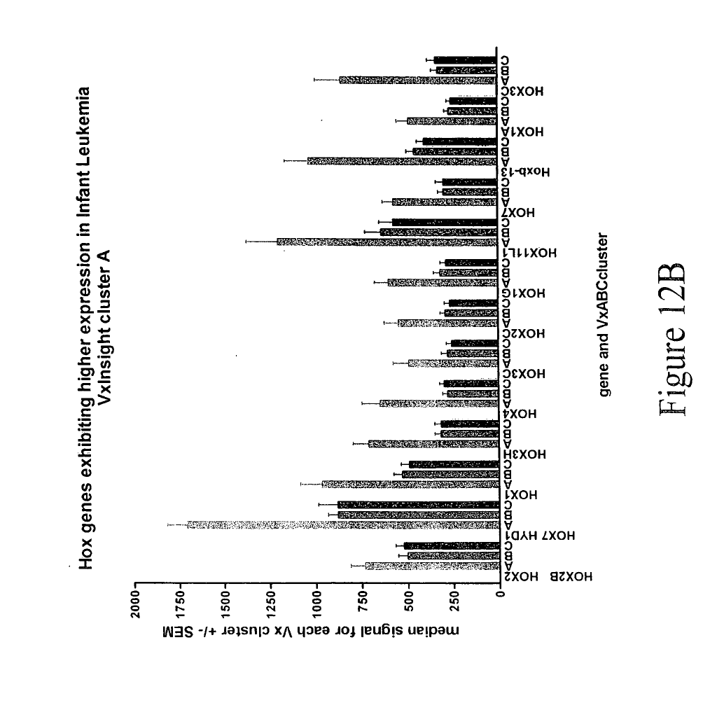

Our data also supports the existence of a third group, with a particular gene expression pattern suggestive of a novel stem cell neoplasia with leukemic behavior. The genes expressed in most of these cases resemble those present in the hematopoietic/angioblastic primordial cell (Young, 1995; Eichman, 1997); see for example, FIGS. 11 and 12. This subgroup may be therapeutically relevant and may also provide additional evidence for the existence of a common progenitor, possibly the primordial hematopoietic/endothelial cell. The gene expression blueprint of this cluster seems to characterize a unique and distinct subclass of infant leukemia that represents transformed, true multi-potent stem cells or “cancer stem cells”. There is an important body of work suggesting that normal hematopoietic stem cells may be target of transforming mutations and that cancer cell proliferation is driven by cancer stem cells (Reya, 2001). Our data provides further evidence in support of the hypothesis that newly arising cancer cells may appropriate the machinery for self-renewing cell divisions, which is normally expressed in stem cells.

Together, these results indicate the occurrence of, at least, three inherent biological subgroups of infant leukemia, not precisely defined by traditional AML vs. ALL or cytogenetics labels; probably driven by characteristics with potential clinical relevance. Consideration of these three categories may enable selection criteria for more powerful clinical trials, and might lead to improved treatments with better success rates.

Methods

To develop gene expression-based classification schemes related to the pathogenic basis underlying the leukemic process in infant acute leukemia, 126 patients registered to NCI-sponsored Infant Oncology Group/Children's Oncology Group treatment trials were examined using Affymetrix U95Av2 oligonucleotide microarrays containing 12,625 probes. Of the 126 cases, 78 were ALL (62%), 48 were AML (38%) and 56 (44%) cases had translocations involving the MLL gene (chromosome segment 11q23). An average of 2×10⁷ cells were used for total RNA extraction with the Qiagen RNeasy mini kit (Valencia, Calif.). The yield and integrity of the purified total RNA were assessed with the RiboGreen assay (Molecular Probes, Eugene, Oreg.) and the RNA 6000 Nano Chip (Agilent Technologies, Palo Alto, Calif.), respectively. Complementary RNA (cRNA) target was prepared from 2.5 μg total RNA using two rounds of Reverse Transcription (RT) and In Vitro Transcription (IVT). Following denaturation for 5 minutes at 70° C., the total RNA was mixed with 100 pmol T7-(dT)₂₄ oligonucleotide primer (Genset Oligos, La Jolla, Calif.) and allowed to anneal at 42° C. The mRNA was reverse transcribed with 200 units Superscript II (Invitrogen, Grand Island, N.Y.) for 1 hour at 42° C. After RT, 0.2 vol. 5× second strand buffer, additional dNTP, 40 units DNA polymerase I, 10 units DNA ligase, 2 units RnaseH (Invitrogen) were added and second strand cDNA synthesis was performed for 2 hours at 16° C. After T4 DNA polymerase (10 units), the mix was incubated an additional 10 minutes at 16° C. An equal volume of phenol:chloroform:isoamyl alcohol (25:24:1) (Sigma, St. Louis, Mo.) was used for enzyme removal. The aqueous phase was transferred to a microconcentrator (Microcon 50. Millipore, Bedford, Mass.) and washed/concentrated with 0.5 ml DEPC water twice the sample was concentrated to 10-2011. The cDNA was then transcribed with T7 RNA polymerase (Megascript, Ambion, Austin, Tex.) for 4 hours at 37° C. Following IVT, the sample was phenol:chloroform:isoamyl alcohol extracted, washed and concentrated to 10-20 μl. The first round product was used for a second round of amplification which utilized random hexamer and T7-(dT)₂₄ oligonucleotide primers, Superscript II, two RNase H additions, DNA polymerase I plus T4 DNA polymerase finally and a biotin-labeling high yield T7 RNA polymerase kit (Enzo Diagnostics, Farmingdale, N.Y.). The biotin-labeled cRNA was purified on Qiagen RNeasy mini kit columns, eluted with 50 μl of 45° C. RNase-free water and quantified using the RiboGreen assay. Following quality check on Agilent Nano 900 Chips, 15 μg cRNA were fragmented following the Affymetrix protocol (Affymetrix, Santa Clara, Calif.). The fragmented RNA was then hybridized for 20 hours at 45° C. to HG_U95Av2 probes. The hybridized probe arrays were washed and stained with the EukGE_WS2 fluidics protocol (Affymetrix), including streptavidin phycoerythrin conjugate (SAPE, Molecular Probes, Eugene, Oreg.) and an antibody amplification step (Anti-streptavidin, biotinylated, Vector Labs, Burlingame, Calif.). HG_U95Av2 chips were scanned at 488 nm, as recommended by Affymetrix. The expression value of each gene was calculated using Affymetrix Microarray Suite 5.0 software.

Data Presentation and Exclusion Criteria

Some of the criteria used as quality controls include: total RNA integrity, cRNA quality, array image inspection, B2 oligo performance, and internal control genes (GAPDH value greater than 1800).

Data Analysis

Affymetrix MAS 5.0 statistical analysis software was used to process the raw microarray image data for a given sample into quantitative signal values and associated present, absent or marginal calls for each probeset. A filter was then applied which excluded from further analysis all Affymetrix “control” genes (probesets labeled with AFFY_prefix), as well as any probeset that did not have a “present” call at least in one of the samples. For this analysis our Bayesian classification and VxInsight clustering analysis omitted this step, choosing instead to assume minimal a priori gene selection (Helman et al, 2003; Davidson et al., 2001). The filtering step reduced the number of probe sets from 12,625 to 8,414, resulting in a matrix of 8,414×N signal values, where N is the number of cases. The first stage of our analysis consisted of a series of binary classification problems defined on the basis of clinical and biologic labels. The nominal class distinctions were ALL/AML, MLL/not-MLL, achieved complete remission CR/not-CR. Additionally, several derived classification problems-based on restrictions of the full cohort to particular subsets of data such as a VxInsight cluster-were considered (see main text). The multivariate unsupervised learning techniques used included Bayesian nets (Helman et al., 2003) and support vector machines (Guyon et al., 2002). The performance of the derived classification algorithms was evaluated using fold-dependent leave-one-out cross validation (LOOCV) techniques. These methods combined allowed the identification of genes associated with remission or treatment failure and with the presence or absence of translocations of the MLL gene across the dataset. In order to identify potential clusters and inherent biologic groups, a large number of clinical co-variables were correlated with the expression data using unsupervised clustering methods such as hierarchical clustering, principal component analysis and a force-directed clustering algorithm coupled with the VxInsight visualization tool. Agglomerative hierarchical clustering with average linkage (similar to Eisen et al., 1998) was performed with respect to both genes and samples, using the MATLAB (The Mathworks, Inc.), the MatArray toolbox and native MATLAB statistics toolbox. The data for a given gene was first normalized by subtracting the mean expression value computed across all patients, and dividing by the standard deviation across all patients for each gene. The distance metric used was one minus Pearson's correlation coefficient; this choice enabled subsequent direct comparison with the VxInsight cluster analysis, which is based on the t-statistic transformation of the correlation coefficient (Davidson et al., 2001). The second clustering method was a particle-based algorithm implemented within the VxInsight knowledge visualization tool (www.sandia.gov/projectsJVxInsight.html). In this approach, a matrix of pair similarities is first computed for all combinations of patient samples. The pair similarities are given by the t-statistic transformation of the correlation coefficient determined from the normalized expression signatures of the samples (Davidson et al., 2001). The program then randomly assigns patient samples to locations (vertices) on a 2D graph, and draws lines (edges), thus linking each sample pair, and assigning each edge a weight corresponding to the pairwise t-statistic of the correlation. The resulting 2D graph constitutes a candidate clustering. To determine the optimal clustering, an iterative annealing procedure is followed, wherein a ‘potential energy’ function that depends on edge distances and weights is minimized, following random moves of the vertices (Davidson et al., 1998, 2001). Once the 2D graph has converged to a minimum energy configuration, the clustering defined by the graph is visualized as a 3D terrain map, where the vertical axis corresponds to the density of samples located in a given 2D region. The resulting clusters are robust with respect to random starting points and to the addition of noise to the similarity matrix, evaluated through its effect on neighbor stability histograms (Davidson et al., 2001).

### REFERENCES

Alizadeh, A. A., Eisen, M. B., Davis, R. E., et al. Distinct types of diffuse large B-cell lymphoma identified by gene expression profiling. Nature 403, 503-511 (2000).
Akashi, K., He, X., Chen, J., Iwasaki, H., Niu, C., Steenhard, B., Zhang, J., Haug, J., Li, L. Transcriptional accessibility for genes of multiple tissues and hematopoietic lineages is hierarchically controlled during early hematopoiesis. Blood 101, 383-90 (2003).
Armstrong, S. A., Staunton, J. E., Silverman, L. B., et al. MLL translocations specify a distinct gene expression profile that distinguishes a unique leukemia. Nature Genetics 30, 41-47 (2002).
Birkenbach, M., Josefsen, K., Yalamanchili, R., Lenoir, G., Kieff, E. Epstein-Barr virus-induced genes: first lymphocyte-specific G protein-coupled peptide receptors. J Virol 67, 2209-20 (1993).
Davidson, G. S., Wylie, B. N., and Boyack, K. W. Cluster stability and the use of noise in interpretation of clustering. Proc. IEEE Information Visualization 2001, 23-30 (2001).
Davidson, G. S., Hendrickson, B., Johnson, D. K., Meyers, C. E., & Wylie, B. N. Knowledge mining with VxInsight: Discovery through interaction. J. Int. Inf. Syst. 11, 259-285 (1998).
Eichmann, A., Corbel, C., Nataf, V., Vaigot, P., Breant, C. and Le Douarin, N. M. Ligand dependent development of the endothelial and hepatopoietic lineages from embryonic mesodermal cells expressing vascular endothelial growth factor receptor 2. Proc. Natl. Acad. Sci. U.S.A. 94, 5141-5146 (1997).
Eisen, M. B., Spellman, P. T., Brown, P. O., and Botstein, D. Cluster analysis and display of genome-wide expression patterns. Proc. Natl. Acad. Sci. USA 95, 14863-14868 (1998).
Efron, B. Bootstrap methods—“another look at the jackknife” Ann. Statist., 7, 1-26 (1979).
Golub T R, Slonim D K, Tarnayo P, Huard C, Gaasenbeek M, Mesirov J P, Coller H, Loh M L, Downing J R, Caligiuri M A, Bloomfield C D, Lander E S. Molecular classification of cancer: class discovery and class prediction by gene expression monitoring. Science 286, 531-7 (1999).
Guyon I, Weston, J, Barnhill S, and Vapnik V. Gene Selection for Cancer Classification Using Support Vector Machines. Machine Learning 46, 389-422 (2002).
Helman P, Veroff R, Atlas S, and Willman C L. A new Bayesian network classification methodology for gene expression data. Journal of Computational Biology, submitted (2002); available on the worldwide web at cs.unm.edu/˜helman/papers/JCB_Total.pdf.
Hjorth, J. S. Urban Computer Intensive Statistical Methods, Validation model selection and bootstrap, ISBN 0412491605, Chapman & Hall, 2-6 Boundary Row, London SE1 8HN, UK. (1994).
Jolliffe, I. T. Principal Component Analysis. Springer-Verlag (1986).
Khan, J., Wei, J. S., Ringnér, M., Saal, L. H., Ladanyi, M., Westermann, F., Berthold, F., Schwab, M., Antonescu, C. R., Peterson, C., and Meltzer, P. S. Classification and diagnostic prediction of cancers using gene expression profiling and artificial neural networks. Nature Medicine 7, 673 (2001).
Kim, S. K., Lund, J., Kiraly, M., Duke, K., Jiang, M., Stuart, J. M., Eizinger, A., Wylie, B. N., and Davidson, G. S. A gene expression map for Caenorhabditis elegans. Science 293, 2087-2092 (2001).
Kirby, M. Geometric Data Analysis. John Wiley & Sons (2001).
Oklu, R., Hesketh, R. The latent transforming growth factor b binding protein (LTBP) family. Biochem. J. 352, 601-610 (2000) Review
Ramaswamy, S., Tamayo, P., Rifkin, R., Mukherjee, S., Yeang, C.-H., Angelo, M., Ladd, C., Reich, M., Latulippe, E., Mesirov, J. P., Poggio, T., Gerald, W., Loda, M., Lander, E. S., and Golub, T. R. Multiclass cancer diagnosis using tumor gene expression signatures. Proc. Natl. Acad. Sci. USA 98, 15149 (2001).
Raychaudhuri, S., Stuart, J., and Altman, R. Principal component analysis to summarize microarray experiments: application to sporulation time series. Pac. Symp. Biocomput., 5, 455-466 (2000).
Rosenwald, A., Wright, G., Chan, W. C., Connors, J. M., Campo, E., Fisher, R. I., Gascoyne, R. D., Muller-Hermelink, H. K., Smeland, E. B., and Staudt, L. M. The use of molecular profiling to predict survival after chemotherapy for diffuse large-B-cell lymphoma. N. Engl. J. Med. 346, 1937 (2002).
Skalnik D G. Transcriptional mechanisms regulating myeloid-specific genes. Gene 284, 1-21 (2002).
Staege, M. S., Lee, S. P., Frisan, T., Maunter, J., Scholz, S., Pajic, A., Rickinson, A. B., Masucci, M. G., Polack, A., Bornkamm, G. W. MYC overexpression imposes a nonimmunogenic phenotype on Epstein-Barr virus-infected B cells. Proc. Natl. Acad. Sci. USA. 99, 4550-4555 (2002).
Tamayo, P., Slonim, D., Merisov, J., Zhu, Q., Kitareewan, S., Dimitrovsky, E., Lander, E., Golub, T. Interpreting patterns of gene expression with self-organizing maps: Methods and application to hematopoietic differentiation. Proc. Natl. Acad. Sci., 96, 2907-2912 (1999).
Trefethen, L. & Bau, D. Numerical Linear Algebra. SIAM, Philadelphia (1997).
van 't Veer, L. J., Dal, H., van de Vijver, M. J., et al. Gene expression profiling predicts clinical outcome of breast cancer. Nature 415, 530-536 (2002).
Werner-Washburne, M., Wylie, B., Boyack, K., Fuge, E. Galbraith, J., Fleharty, M., Weber, J., Davidson, G. S. Concurrent analysis of multiple genome-scale datasets. Genome Research (12), 1564-1573, 2002

Young, P. E., Baumhueter, S. and Laskiy, L. A. The sialomucin CD34 is expressed on hematopoietic cells and blood vessels during murine development. Blood, 85, 96-105 (1995).

TABLE 44
Class Predictor Performance

| | Bayesian Net | | | SVM | | | Fuzzy Inference | | | Discriminant Analysis | | |
|---|---|---|---|---|---|---|---|---|---|---|---|---|
| Description | r | p-value¹ | p-value² | r | p-value¹ | p-value² | r | p-value¹ | p-value² | r | p-value¹ | p-value² |
| ALL vs. AML | .912 | <.001** | <.001** | .971 | <.001** | <.001** | .971 | <.001** | <.001** | .853 | <.001** | <.001** |
| t(4; 11) vs. Not t(4; 11) | .818 | <.001** | .005** | .879 | <.001** | <.001** | .788 | <.001** | .021* | .788 | <.001** | .022* |
| Remission. vs. Fail | .568 | .256 | .507 | .622 | .094 | — | .405 | .906 | .997 | .568 | .256 | .507 |

Table 44. Class Predictor Performance In order to optimize gene selection and determine the success rate of each classifier, fold-dependent leave-one-out cross-validation was used on the training set (n = 82) followed by “single shot” prediction on our validation set (n = 44) using the trained classifiers.
r = Success rate;
p-value¹ = Computed using the first method as described in Supplemental Information;
p-value² = Computed using the second method as described in Supplemental Information.
*means that the predictor is significant at level α = 0.05
**means that the predictor is significant at level α = 0.01.
— indicates that the Fisher's exact test can not be fulfilled because two cells in the contingency table are zero.

TABLE 45

| | | | | |
|---|---|---|---|---|
| | | Affymetrix | | Gene |
| F score | p | number | Gene description | symbol |

Genes with differential expression patterns between the VxInsight clusters A
and the rest of the cases. The gene lists are sorted into decreasing order based
on the resulting F-scores.
Cluster A - Up-regulated genes

| | | | | |
|---|---|---|---|---|
| 167.99 | 0.001 | 37746_r_at | Tumor suppressor gene | TS5 |
| 124.38 | 0.005 | 36276_at | Contactin 2 axonal | CNTN2 |
| 123.10 | 0.006 | 33058_at | Cytokeratin type II | K6HF |
| 122.51 | 0.010 | 33137_at | Transforming growth factor | LTBP4 |
| | | | beta binding protein 4 | |
| 119.66 | 0.004 | 721_g_at | Heat-shock transcription factor 4 | HSF4 |
| 114.94 | 0.019 | 396_f_at | Erythropoietin receptor precursor | EPOR |
| 114.21 | 0.011 | 41565_at | Ataxin 2 related protein | A2LP |
| 113.20 | 0.007 | 40792_s_at | Triple functional domain interacting | PTPRF |
| 109.97 | 0.008 | 884_at | Integrin α3 | ITGA3 |
| 98.55 | 0.010 | 40539_at | Myosin IXB | MYO9B |
| 98.43 | 0.040 | 41694_at | Temperature sensitivity complementing | BHK21 |
| 94.32 | 0.020 | 41347_at | p70 ribosomal S6 kinase beta (iroquois | IRX5 |
| | | | homeobox protein 5) | |
| 92.02 | 0.010 | 38132_at | Serum constituent protein | MSE55 |
| 88.80 | 0.021 | 39448_r_at | B7 protein | B7 |
| 85.44 | 0.035 | 34573_at | Ephrin A3 | EFNA3 |
| 84.99 | 0.020 | 34894_r_at | Protease serine 26 | PRSS22 |
| 82.83 | 0.029 | 39775_at | Complement component inhibitor 1 | SERPING1 |
| 82.51 | 0.031 | 41499_at | v-ski avian sarcoma viral oncogene | SKI |
| 80.85 | 0.010 | 567_s_at | Promyelocitic leukemia | PML |
| 77.97 | 0.020 | 38707_r_at | E2F transcription factor 4 | E2F4 |
| 76.97 | 0.044 | 37061_at | Chitotriosidase | CHIT1 |
| 73.43 | 0.021 | 1804_at | Kallikrein 3 prostate specific antigen | KLK3 |
| 73.74 | 0.041 | 38058_at | Dermatopontin precursor | DPT |
| 72.07 | 0.023 | 39868_at | poly rC binding protein 3 | PCBP3 |
| 72.48 | 0.033 | 35910_f_at | Zinc finger protein 200 | MMPL |
| | | | (matrix metalloproteinase like) | |
| 69.03 | 0.041 | 39920_r_at | C1q-related factor | CRF |
| 68.53 | 0.051 | 37140_s_at | Ectodermal dysplasia 1 anhidrotic | ED1 |
| 68.52 | 0.055 | 39306_at | Protease serine 16 thymus | PRSS16 |
| 68.07 | 0.062 | 1925_at | Cyclin F | CCNF |
| 67.57 | 0.093 | 40501_s_at | Myosin-binding protein C slow-type | MYBPC1 |
| 66.62 | 0.052 | 160020_at | Matrix matelloproteinase 14 preprotein | MMP14 |
| 63.85 | 0.043 | 33448_at | Hepatocyte growth factor activator | SPINT1 |
| | | | inhibitor precursor | |
| 62.14 | 0.035 | 33034_at | Rhomboid veinlet Drosophila like | RHBDL |
| 61.86 | 0.055 | 31393_r_at | Undifferentiated embryonic cell | UTF1 |
| | | | transcription factor 1 | |
| 61.28 | 0.039 | 41359_at | Plakophilin 3 | PKP3 |
| 60.51 | 0.103 | 538_at | CD34 antigen | CD34 |

Genes with differential expression patterns between the VxInsught clusters A
and the rest of the cases.
Cluster A - Down-regulated genes

| | | | | |
|---|---|---|---|---|
| 115.50 | 0.018 | 36991_at | Splicing factor arginine/serine-rich 4 | SFRS4 |
| 114.41 | 0.015 | 1241_at | protein tyrosine phosphatase type | PTP4A |
| | | | IVA member 2 | |
| 108.68 | 0.013 | 41187_at | death-associated protein 6 | DAXX |
| 98.82 | 0.018 | 37675_at | phosphate carrier precursor 1b | PHC |
| 95.63 | 0.026 | 37029_at | ATP synthase H transporting | ATP50 |
| | | | mitochondrial F1 complex O subunit | |
| 95.11 | 0.019 | 41834_g_at | jumping translocation breakpoint | JTB |
| 94.08 | 0.027 | 41295_at | GTT1 protein | GTT1 |
| 92.64 | 0.027 | 1817_at | prefoldin 5 | PFDN5 |
| 90.62 | 0.029 | 35279_at | Tax1 human T-cell leukemia virus | TAX1BP1 |
| | | | type I binding protein 1 | |
| 90.18 | 0.027 | 32832_at | erythroblast macrophage attacher | No symbol |
| 87.74 | 0.028 | 1357_at | ubiquitin specific protease | USP4 |
| | | | proto-oncogene | |
| 87.26 | 0.047 | 1499_at | farnesyltransferase CAAX box alpha | FNTA |
| 84.12 | 0.048 | 37766_s_at | proteasome prosome macropain 26S | PSMC5 |
| | | | subunit ATPase 5 | |
| 83.23 | 0.056 | 1399_at | elongin C | TCEB1 |
| 82.82 | 0.042 | 41241_at | asparaginyl-tRNA synthetase | NARS |
| 78.67 | 0.030 | 36492_at | proteasome prosome macropain 26S | PSMD9 |
| | | | subunit non-ATPase 9 | |
| 78.21 | 0.043 | 37581_at | protein phosphatase 6 catalytic subunit | PPP6C |
| 78.18 | 0.082 | 39360_at | sorting nexin 3 | No symbol |
| 76.07 | 0.054 | 36616_at | DAZ associated protein 2 | No symbol |
| 75.21 | 0.063 | 34330_at | cytochrome c oxidase subunit VIIa | COX7A2L |
| | | | polypeptide 2 like | |
| 74.72 | 0.044 | 31670_s_at | calcium/calmodulin-dependent protein | CAMKG |
| | | | kinase CaM kinase II gamma | |
| 74.30 | 0.045 | 39184_at | elongin B | TCEB2 |
| 73.46 | 0.055 | 34302_at | eukaryotic translation initiation factor 3 | EIF3S4 |
| | | | subunit 4 delta 44 kD | |
| 72.24 | 0.074 | 35298_at | eukaryotic translation initiation factor 3 | EIF3S7 |
| | | | subunit 7 zeta 66/67 kD | |
| 71.36 | 0.055 | 41551_at | similar to S. cerevisiae RER1 | No symbol |
| 71.28 | 0.057 | 35297_at | NADH dehydrogenase ubiquinone | NDUFAB1 |
| | | | 1 alpha/beta subcomplex 1 8 kD SDAP | |
| 71.06 | 0.059 | 40874_at | endothelial differentiation-related 1 | EDF1 |
| 70.73 | 0.045 | 38455_at | small nuclear ribonucleoprotein | SNRPB |
| | | | polypeptides B and B1 | |
| 69.57 | 0.082 | 935_at | adenylyl cyclase-associated protein | No symbol |
| 69.09 | 0.077 | 31492_at | muscle specific gene | No symbol |
| 68.81 | 0.043 | 37672_at | ubiquitin specific protease 7 herpes | USP7 |
| | | | virus-associated | |
| 68.31 | 0.066 | 35319_at | CCCTC-binding factor zinc finger | CTCF |
| | | | protein | |

Genes with differential expression patterns between the
VxInsight cluster B and the rest of the cases.
Cluster B - Up-regulated genes

| | | | | |
|---|---|---|---|---|
| 250.55 | 0.001 | 40103_at | Villin 2 | VIL2 |
| 157.12 | 0.003 | 1096_g_at | CD19 antigen | CD19 |
| 122.41 | 0.005 | 38269_at | Protein kinase D2 | PKD2 |
| 113.79 | 0.005 | 2047_s_at | Junction plakoglobin isoform 1 | JUP |
| 113.35 | 0.006 | 35298_at | Eukariotic translation initiation factor 3 | EIF3 |
| 109.78 | 0.010 | 36991_at | Splicing factor arg/ser rich 4 | SFRS4 |
| 107.87 | 0.011 | 854_at | B lymphoid tyrosine kinase | BLK |
| 105.40 | 0.005 | 41356_at | B-cell CLL/lymphoma 11A | BCL11A |
| 101.07 | 0.006 | 38017_at | CD79A antigen | CD79A |
| 91.63 | 0.010 | 37672_at | Ubiquitin specific protease 7 herpes | USP7 |
| | | | virus associated | |
| 91.08 | 0.020 | 37585_at | Small nuclear ribonucleotide | SNRPA1 |
| | | | polypeptide A | |
| 89.36 | 0.023 | 31492_at | Muscle specific gene | M9 |
| 87.23 | 0.008 | 36111_s_at | Splicing factor arg/ser rich 2 | SFRS2 |
| 85.38 | 0.041 | 1754_at | Death associated protein | DAXX |
| 81.74 | 0.039 | 1357_at | Ubiquitin specific protease protooncogene | USP |
| 74.04 | 0.047 | 41834_g_at | Jumping translocation breakpoint | JTB |
| 73.16 | 0.020 | 39044_s_at | Diacylglycerol kinase delta | DGKD |
| 73.14 | 0.013 | 38604_at | Neuropeptide Y | NPY |
| 71.06 | 0.010 | 32238_at | Binding integrator 1 | BIN1 |
| 70.78 | 0.031 | 38054_at | Hepatitis B virus interacting x-protein | HBXIP |
| 68.13 | 0.050 | 1817_at | Prefoldin 5 | PFDN5 |
| 67.74 | 0.018 | 32842_at | B-cell CLL/lymphoma | BCL2 |
| 63.71 | 0.069 | 40189_at | SET translocation myeloid-leukemia | SET |
| | | | associated | |
| 61.60 | 0.015 | 33304_at | Interferon stimulated gene 20 kD | ISG20 |
| 59.35 | 0.025 | 38989_at | DC 12 protein | DC12 |
| 57.53 | 0.045 | 36630_at | Delta sleep inducing petide | DSIPI |
| 56.43 | 0.035 | 36949_at | Casein kinase 1 delta | CSNK1D |
| 56.22 | 0.027 | 1814_at | Transforming growth factor beta | TGFBR2 |
| | | | receptor | |
| 56.07 | 0.031 | 39318_at | T-cell lymphoma-1 | TCL1A |
| 54.40 | 0.037 | 37028_at | DNA damage inducible | PPP1R15A |
| 53.94 | 0.021 | 1102_s_at | Nuclear receptor subfamily 3 group C | NR3C1 |
| 51.74 | 0.033 | 40828_at | PAK-interacting exchange factor beta | ARHGEF7 |
| 51.32 | 0.025 | 493_at | Casein kinase 1 delta | CSNK1D |
| 50.93 | 0.039 | 40365_at | Guanine nucleotide binding protein G | GNA15 |
| 50.77 | 0.037 | 32070_at | Tyrosin phosphatase receptor type | PTPRCAP |
| 50.59 | 0.054 | 35974_at | Lymphoid-restricted membrane protein | LRMP |
| 50.37 | 0.048 | 34180_at | Rho guanine nucleotide exchange factor | GEF10 |
| 50.06 | 0.031 | 280_g_at | Nuclear receptor subfamily 4 group A1 | NR4A1 |
| 48.15 | 0.017 | 41203_at | Zinc finger protein 162 (splice factor1) | SF1 |
| 47.98 | 0.030 | 40841_at | Transforming acidic coiled-coil | TACC1 |

Genes with differential expression patterns between the
VxInsight cluster B and the rest of the cases.
Cluster B - Down-regulated genes

| | | | | |
|---|---|---|---|---|
| 81.4 | 0.007 | 39689_at | cystatin C amyloid angiopathy | CST3 |
| 78.48 | 0.004 | 36938_at | N-acylsphingosine amidohydrolase | ASAH |
| | | | acid ceramidase | |
| 67 | 0.011 | 1230_g_at | cisplatin resistance associated | No symbol |
| 57.88 | 0.022 | 34885_at | synaptogyrin 2 | SYNGR2 |
| 57.26 | 0.018 | 35367_at | lectin galactoside-binding soluble 3 | LGALS3 |
| | | | galectin 3 | |
| 54.71 | 0.015 | 36766_at | ribonuclease RNase A family 2 liver | RNASE2 |
| | | | eosinophil-derived neurotoxin | |
| 52.66 | 0.029 | 32747_at | aldehyde dehydrogenase 2 family | ALDH2 |
| | | | mitochondrial | |
| 51.51 | 0.022 | 36879_at | endothelial cell growth factor 1 | ECGF1 |
| | | | platelet-derived | |
| 51.32 | 0.021 | 39994_at | chemokine C—C motif receptor 1 | CCR1 |
| 50.88 | 0.014 | 35012_at | myeloid cell nuclear differentiation | MNDA |
| | | | antigen | |
| 50.53 | 0.02 | 36889_at | Fc fragment of IgE high affinity I | FCER1G |
| | | | receptor for gamma polypeptide | |
| | | | precursor | |
| 50.41 | 0.023 | 34789_at | serine or cysteine proteinase inhibitor | PIR6 |
| | | | clade B ovalbumin member 6 | |
| 50.21 | 0.029 | 1052_s_at | CCAAT/enhancer binding protein | CEBPD |
| | | | C/EBP delta | |
| 49.91 | 0.014 | 37398_at | platelet/endothelial cell adhesion | CD31 |
| | | | molecule CD31 antigen | |
| 49.79 | 0.022 | 40580_r_at | parathymosin | PTMS |
| 47.39 | 0.03 | 41096_at | S100 calcium-binding protein A8 | S100A8 |
| 47.26 | 0.031 | 33963_at | azurocidin 1 cationic antimicrobial | No symbol |
| | | | protein 37 | |
| 47.06 | 0.018 | 36465_at | interferon regulatory factor 5 | No symbol |
| 46.95 | 0.03 | 37021_at | cathepsin H | CTSH |
| 46.36 | 0.029 | 35926_s_at | leukocyte immunoglobulin-like receptor | No symbol |
| | | | subfamily B with TM and ITIM domains | |
| 46.02 | 0.02 | 41523_at | RAB32 member RAS oncogene family | RAB32 |
| 45.94 | 0.034 | 38363_at | TYRO protein tyrosine kinase binding | TYROBP |
| | | | protein | |
| 44.74 | 0.032 | 33856_at | CAAX box 1 | CXX1 |
| 44.73 | 0.038 | 40282_s_at | adipsin/complement factor D precursor | DF |
| 44.5 | 0.027 | 32451_at | membrane-spanning 4-domains | No symbol |
| | | | subfamily A member 3 hematopoietic | |
| | | | cell-specific | |
| 44.08 | 0.045 | 38631_at | tumor necrosis factor alpha-induced | TNFAIP2 |
| | | | protein 2 | |
| 44.01 | 0.053 | 40762_g_at | solute carrier family 16 monocarboxylic | SLC16A5 |
| | | | acid transporters member 5 | |

Genes with differential expression patterns between the
VxInsight cluster C and the rest of the cases.
Cluster C - Up-regulated genes

| | | | | |
|---|---|---|---|---|
| 284.97 | 0.001 | 6938_at | N-acylsphingosine aidohydrolase acid | ASAH |
| | | | ceramidase | |
| 132.03 | 0.001 | 9689_at | Cystatin C | CST3 |
| 126.67 | 0.013 | 1637_at | Mitogen-activated protein kinase- | MAPKAPK3 |
| | | | activated protein kinase 3 | |
| 114.85 | 0.010 | 38363_at | Tyro Protein tyrosine kinase binding | TYROBP |
| | | | protein | |
| 104.53 | 0.009 | 35297_at | NADH dehydrogenase ubiquinone 1 | NDUFAB1 |
| 100.84 | 0.008 | 1230_g_at | Cisplatin resistance associated | |
| 93.33 | 0.008 | 36879_at | Endothelial cell growth factor 1 - platelet | ECGF1 |
| | | | derived | |
| 90.92 | 0.009 | 3856_at | Farnesyltransferase CAAX box alpha | FNTA |
| 89.47 | 0.017 | 35279_at | Tax1 human T-cell leukemia virus type I | TAX1BP1 |
| | | | binding protein I | |
| 88.39 | 0.047 | 39160_at | Pyruvate dehydrogenase lipoamide beta | PDHB |
| 84.75 | 0.036 | 41187_at | Death-associated protein 6 | DAP6 |
| 84.18 | 0.029 | 41495_at | GTT1 protein | GTT1 |
| 81.31 | 0.006 | 41523_at | RAB32 member RAS oncogene family | RAB32 |
| 80.08 | 0.048 | 37337_at | Small nuclear ribonucleoprotein G | SNRPG |
| 75.51 | 0.038 | 402_s_at | Intercellular adhesion molecule | ICAM3 |
| 74.82 | 0.014 | 40282_s_at | Adipsin/complement factor D | DF |
| 72.20 | 0.050 | 39360_at | Sortin nexin 3 | SNX3 |
| 70.26 | 0.055 | 37726_at | Mitochondrial ribosomal protein L3 | MRPL3 |
| 69.05 | 0.016 | 39581_at | Cystatin A (stefin A) | CSTA |
| 68.66 | 0.035 | 1817_at | Prefoldin 5 | PFDN5 |
| 67.80 | 0.059 | 36620_at | Superoxide dismutase 1 soluble | SOD1 |
| 66.34 | 0.090 | 37670_at | Annexin VII | ANXA7 |
| 65.36 | 0.065 | 38097_at | Etoposide-induced mRNA | PIG8 |
| 65.07 | 0.092 | 824_at | Glutathione-S-transferase like | GSTTLp28 |
| 64.88 | 0.016 | 39593_at | Similar to fibrinogen-like 2, clone | |
| | | | MGC: 22391, mRNA, complete cds | |
| 63.75 | 0.024 | 35012_at | Myeloid cell nuclear differentiation | MNDA |
| 63.30 | 0.047 | 1399_at | Elongin C | TCEB1 |
| 62.02 | 0.079 | 891_at | YY1 transcription factor | YY1 |
| 61.60 | 0.079 | 38992_at | DEK oncogene DNA binding | DEK |
| 54.78 | 0.036 | 37021_at | Cathepsin H | CTSH |
| 54.28 | 0.029 | 41198_at | Granulin | GRN |
| 54.27 | 0.028 | 38631_at | Tumor necrosis factor alpha-induced | TNFAIP2 |
| | | | protein 2 | |
| 54.26 | 0.032 | 34860_g_at | Melanoma antigen, family D, 2 | MAGED2 |
| 52.80 | 0.037 | 1693_s_at | Tissue inhibitor of metalloprotease 1 | TIMP1 |
| 48.83 | 0.031 | 38533_s_at | Integrin alpha M precursor | ITGAM |
| 48.64 | 0.038 | 36709_at | Integrin alpha X precursor | ITGAX |
| 48.37 | 0.021 | 34885_at | Synaptogyrin 2 | SYNGR2 |

Genes with differential expression patterns between the
VxInsight cluster C and the rest of the cases.
Cluster C - Down-regulated genes

| | | | | |
|---|---|---|---|---|
| 105.94 | 0.006 | 1096_g_at | CD19 antigen | CD19 |
| 103.5 | 0.005 | 40103_at | villin 2 | VIL2 |
| 80.41 | 0.009 | 2047_s_at | junction plakoglobin isoform 1 | JUP |
| 80.14 | 0.013 | 38017_at | CD79A antigen isoform 2 precursor | CD79A |
| 77.12 | 0.025 | 39327_at | p53-responsive gene | PRG2 |
| 72.29 | 0.017 | 38269_at | protein kinase D2 | PKD2 |
| 72.15 | 0.011 | 39318_at | T-cell lymphoma-1 | TCL1A |
| 66.16 | 0.022 | 854_at | B lymphoid tyrosine kinase | BLK |
| 64.49 | 0.019 | 32238_at | bridging integrator 1 | BIN1 |
| 61.79 | 0.028 | 38604_at | neuropeptide Y | NPY |
| 57.28 | 0.049 | 41356_at | hypothetical protein FLJ10173 | FLJ10173 |
| 56.67 | 0.028 | 41165_g_at | Immunoglobulin mu | IGHM |
| 56.67 | 0.028 | 41165_g_at | B-cell CLL/lymphoma 11A zinc finger | BCL11A |
| | | | protein | |
| 55.58 | 0.038 | 32842_at | B-cell CLL/lymphoma 7A | BCL7A |
| 52.05 | 0.025 | 493_at | casein kinase 1 delta | CSNK1D |
| 49.7 | 0.03 | 36933_at | N-myc downstream regulated | NDRG1 |
| 48.04 | 0.025 | 38018_g_at | CD79A antigen isoform 2 precursor | CD79A |
| 47.31 | 0.049 | 41151_at | SKIP for skeletal muscle and kidney | SKIP |
| | | | enriched inositol phosphatase | |

TABLE 46
Overall Success Rates of Class Predictors After Including the A, B, and C Cluster Distinctions

| | Bayesian Net | | | SVM | | | FUZZY Inference | | | Discriminant Analysis | | |
|---|---|---|---|---|---|---|---|---|---|---|---|---|
| Description | r | C.I. | p-value | r | C.I. | p-value | r | C.I. | p-value | r | C.I. | p-value |
| ALL vs. AML | .912 | [.76, .98] | <.001** | .971 | [.85, 1.0] | <.001** | .971 | [.85, 1.0] | <.001** | .853 | [.69, .95] | <.001** |
| Remission. vs. Fail | .568 | [.39, .73] | .256 | .622 | [.45, .78] | .094 | .405 | [.25, .58] | .906 | .568 | [.39, .73] | .256 |
| Remission. vs. Fail in MLL | .471 | [.23, .72] | .685 | .647 | [.38, .86] | .166 | .471 | [.23, .72] | .685 | .353 | [.14, .62] | .928 |
| Remission. vs. Fail in Not MLL | .545 | [.23, .83] | .500 | .636 | [.31, .89] | .274 | .364 | [.11, .69] | .886 | .636 | [.31, .89] | .274 |
| Remission. vs. Fail in ALL | .542 | [.33, .74] | .419 | .625 | [.41, .81] | .153 | .375 | [.19, .59] | .924 | .500 | [.29, .71] | .580 |
| Remission. vs. Fail in AML | .461 | [.19, .75] | .709 | .769 | [.46, .95] | .046* | .461 | [.19, .75] | .709 | .461 | [.19, .75] | .709 |
| Remission. vs. Fail in VX-GA | .714 | [.29, .96] | .226 | .714 | [.29, .96] | .226 | .857 | [.42, .00] | .062 | .714 | [.29, .96] | .226 |
| Remission. vs. Fail in VX-GB | .688 | [.41, .89] | .105 | .563 | [.30, .80] | .401 | .563 | [.30, .80] | .401 | .438 | [.20, .70] | .772 |
| Remission. vs. Fail in VX-GC | .714 | [.42, .92] | .090 | .714 | [.42, .92] | .089 | .500 | [.23, .77] | .604 | .500 | [.23, .77] | .604 |
| R/F Conditioned on VX- | .703 | [.53, .84] | .010** | .649 | [.47, .80] | .049* | .595 | [.42, .75] | .162 | .514 | [.34, .68] | .500 |
| Groups | | | | | | | | | | | | |

Table 46. Overall success rates of class predictors after including the A, B and C cluster predictions.
r = Estimate of the success rate of the class predictor,
C.I. = 95% confidence interval of the success rate of the class predictor,
p-value = p-value of hypothesis test (see Supplemental Information).
*means that r > 0.5 at significance level α = 0.05.
**means that r > 0.5 at significance level α = 0.01.

TABLE 47
Discriminating genes that distinguish between remission and fail
overall derived from SVM analysis.

| | Affymetrix | | |
|---|---|---|---|
| | Locus | | Gene |
| | number | Gene description | symbol |
| 1 | 41165_g_at | immunoglobulin heavy constant mu | IGHM |
| | 14q32.33 | | |
| 1 | 39389_at | CD9 antigen (p24) | CD9 |
| | 12p13 | | |
| 2 | 41058_g_at | uncharacterized hypothalamus protein HT012 | HT012 |
| | 6p22.2 | | |
| 3 | 31459_i_at | immunoglobulin lambda locus | IGL |
| | 22q11.1 | | |
| 4 | 38389_at | 2′,5′-oligoadenylate synthetase 1 (40-46 kD) | OAS1 |
| | 12q24.1 | | |
| 5 | 37504_at | E3 ubiquitin ligase SMURF1 | SMURF1 |
| | 7q21.1 | | |
| 6 | 40367_at | bone morphogenetic protein 2 | BMP2 |
| | 20p12 | | |
| 7 | 32637_r_at | PI-3-kinase-related kinase SMG-1 | SMG1 |
| | 16p12.3 | | |
| 8 | 39931_at | dual-specificity tyrosine-(Y)-phosphorylation regulated | DYRK3 |
| | 1q32 | kinase 3 | |
| 9 | 37054_at | bactericidal/permeability-increasing protein | BPI |
| | 20q11 | | |
| 10 | 1404_r_at | small inducible cytokine A5 (RANTES) | SCYA5 |
| | 17q11.2 | | |
| 11 | 1292_at | dual specificity phosphatase 2 | DUSP2 |
| | 2q11 | | |
| 12 | 37709_at | DNA segment, numerous copies | DXF68 |
| | Xp22.32 | | |
| 13 | 36857_at | RAD1 (S. pombe) homolog | RAD1 |
| | 5p13.2 | | |
| 14 | 41196_at | karyopherin (importin) beta 1 | KPNB1 |
| | 17q21 | | |
| 15 | 1182_at | phospholipase C, epsilon | PLCE |
| | 2q33 | | |
| 16 | 34961_at | T cell activation, increased late expression | TACTILE |
| | 3q13.13 | | |
| 17 | 37862_at | dihydrolipoamide branched chain transacylase | DBT |
| | 1p31 | (E2 component of branched chain keto acid | |
| | | dehydrogenase complex; maple syrup disease) | |
| 18 | 38772_at | cysteine-rich, angiogenic inducer, 61 | CYR61 |
| | 1p31 | | |
| 19 | 33208_at | DnaJ (Hsp40) homolog, subfamily C, member 3 | DNAJC3 |
| | 13q32 | | |
| 20 | 37837_at | KIAA0863 protein | KIAA0863 |
| | 18q23 | | |
| 21 | 34031_i_at | cerebral cavernous malformations 1 | CCM1 |
| | 7q21 | | |
| 22 | 38220_at | dihydropyrimidine dehydrogenase | DPYD |
| | 1p22 | | |
| 23 | 34684_at | RecQ protein-like (DNA helicase Q1-like) | RECQL |
| | 12p12 | | |
| 24 | 39449_at | S-phase kinase-associated protein 2 (p45) | SKP2 |
| | 5p13 | | |
| 25 | 32638_s_at | PI-3-kinase-related kinase SMG-1 | SMG1 |
| | 16p12.3 | | |
| 26 | 35957_at | stannin | SNN |
| | 16p13 | | |
| 27 | 34363_at | selenoprotein P, plasma, 1 | SEPP1 |
| | 5q31 | | |
| 28 | 35431_g_at | RNA polymerase II transcriptional regulation | MED6 |
| | 14q24.1 | mediator (Med6, S. cerevisiae, homolog of) | |
| 29 | 35012_at | myeloid cell nuclear differentiation antigen | MNDA |
| | 1q22 | | |
| 30 | 38432_at | interferon-stimulated protein, 15 kDa | ISG 15 |
| | 1p36.33 | | |
| 31 | 35664_at | multimerin | MMRN |
| | 4q22 | | |
| 32 | 41862_at | KIAA0056 protein | KIAA0056 |
| | 11q25 | | |
| 33 | 33210_at | YY1 transcription factor | YY1 |
| | 14q | | |
| 34 | 35794_at | KIAA0942 protein | KIAA0942 |
| | 8pter | | |
| 35 | 36108_at | HLA, class II, DQ beta 1 | DQB1 |
| | 6p21.3 | | |
| 36 | 35614_at | transcription factor-like 5 (basic helix-loop-helix) | TCFL5 |
| | 20q13.3 | | |
| 37 | 32089_at | sperm associated antigen 6 | SPAG6 |
| | 10p12 | | |
| 38 | 1343_s_at | serine (or cysteine) proteinase inhibitor) | SERPINB |
| | 18q21.3 | | |
| 39 | 665_at | serine/threonine kinase 2 | STK2 |
| | 3p21.1 | | |
| 40 | 40901_at | nuclear autoantigen | GS2NA |
| | 14q13 | | |
| 41 | 39299_at | KIAA0971 protein | KIAA0971 |
| | 2q34 | | |
| 42 | 34446_at | KIAA0471 gene product | KIAA0471 |
| | 1q24 | | |
| 43 | 33956_at | MD-2 protein | MD-2 |
| | 8q13.3 | | |
| 44 | 37184_at | syntaxin 1A (brain) | STX1A |
| | 7q11.23 | | |
| 45 | 1773_at | farnesyltransferase, CAAX box, beta | FNTB |
| | 14q23 | | |
| 46 | 34731_at | KIAA0185 protein | KIAA0185 |
| | 10q24.32 | | |
| 47 | 41700_at | coagulation factor II (thrombin) receptor | F2R |
| | 5q13 | | |
| 48 | 38407_r_at | prostaglandin D2 synthase (21 kD, brain) | GDS |
| | 9q34.2 | | |
| 49 | 40088_at | nuclear receptor interacting protein 1 | NRIP1 |
| | 21q11.2 | | |
| 50 | 33124_at | vaccinia related kinase 2 | VRK2 |
| | 2p16 | | |
| 51 | 32964_at | egf-like module containing, mucin-like, hormone | EMR1 |
| | 19p13.3 | receptor-like sequence 1 | |
| 52 | 39560_at | chromobox homolog 6 | CBX6 |
| | 22q13.1 | | |
| 53 | 39838_at | CLIP-associating protein 1 | CLASP1 |
| | 2q14.2 | | |
| 54 | 40166_at | CS box-containing WD protein | LOC55884 |
| 55 | 36927_at | hypothetical protein, expressed in osteoblast | GS3686 |
| | 1p22.3 | | |
| 56 | 41393_at | zinc finger protein 195 | ZNF195 |
| | 11p15.5 | | |
| 57 | 35041_at | neurotrophin 3 | NTF3 |
| | 12p13 | | |
| 58 | 40238_at | G protein-coupled receptor, family C, group 5, | GPRC5B |
| | 16p12 | | |
| 59 | 39926_at | MAD (mothers against decapentaplegic, Drosoph) | MADH5 |
| | 5q31 | | |
| 60 | 36674_at | small inducible cytokine A4 | SCYA4 |
| | 17q21 | | |
| 61 | 32132_at | KIAA0675 gene product | KIAA0675 |
| | 3q13.13 | | |
| 62 | 38252_s_at | 1,6-glucosidase, 4-alpha-glucanotransferase | AGL |
| | 1p21 | | |
| 63 | 33598_r_at | cold autoinflammatory syndrome 1 | CIAS1 |
| | 1q44 | | |
| 64 | 37409_at | SFRS protein kinase 2 | SRPK2 |
| | 7q22 | | |
| 65 | 41019_at | phosducin-like | PDCL |
| | 9q12 | | |
| 66 | 1113_at | bone morphogenetic protein 2 | BMP2 |
| | 20p12 | | |
| 67 | 37208_at | phosphoserine phosphatase-like | PSPHL |
| | 7q11.2 | | |
| 68 | 32822_at | solute carrier family 25 | SLC25A4 |
| | 4q35 | | |
| 69 | 32249_at | H factor (complement)-like 1 | HFL1 |
| | 1q32 | | |
| 70 | 39600_at | EST | |
| 71 | 32648_at | delta-like homolog (Drosophila) | DLK1 |
| | 14q32 | | |
| 72 | 39269_at | replication factor C (activator 1) 3 (38 kD) | RFC3 |
| | 13q12.3 | | |
| 73 | 37724_at | v-myc avian myelocytomatosis viral oncogene | MYC |
| | 8q24.12 | | |
| 74 | 35606_at | histidine decarboxylase | HDC |
| | 15q21 | | |
| 75 | 31926_at | cytochrome P450, subfamily VIIA | CYP7A1 |
| | 8q11 | | |
| 76 | 32142_at | serine/threonine kinase 3 (Ste20, yeast homolog) | STK3 |
| | 8p22 | | |
| 77 | 32789_at | nuclear cap binding protein subunit 2, 20 kD | NCBP2 |
| | 3q29 | | |
| 78 | 37279_at | GTP-binding protein (skeletal muscle) | GEM |
| | 8q13 | | |
| 79 | 40246_at | discs, large (Drosophila) homolog 1 | DLG1 |
| | 3q29 | | |
| 80 | 37547_at | PTH-responsive osteosarcoma B1 protein | B1 |
| | 7p14 | | |
| 81 | 32298_at | a disintegrin and metalloproteinase domain 2 | ADAM2 |
| | 8p11.2 | | |
| 82 | 40496_at | complement component 1, s subcomponent | C1S |
| | 12p13 | | |
| 83 | 39032_at | transforming growth factor beta-stimulated protein | TSC22 |
| | 13q14 | | |

Supplementary Information
Sample Management

Cell suspensions from diagnostic bone marrow aspirates or peripheral blood samples were handled according to the cryopreservation procedure of the St. Jude's Children's Hospital. Samples were retrieved from cryopreservation at −135° C. and thawed quickly at 37° C. and then washed by centrifugation at 1200 rpm for 5 minutes in warmed 20% (v/v) Fetal Bovine Serum in Dulbecco's Modified Minimum Essential Medium (Invitrogen, Grand Island, N.Y.). Cytospins were prepared from thawed samples, stained with Wright's stain and assessed for percent blasts and cell viability by light microscopy. Decanted cell pellets were used immediately for RNA purification.

RNA Extraction and T7 Amplification

An average of 2×10⁷ cells were used for the total RNA extraction with the Qiagen RNeasy mini kit (VWR International AB, Stockolm, Sweden). The mean of the purified total RNA concentration was 0.5 μg/ul (approximately 25 μg of total RNA yield), as quantified with the RiboGreen assay (Molecular Probes, Eugene, Oreg.). All samples met assay quality standards as recommended by Affymetrix. The A260 nm/A280 nm ratio was determined spectrophotometrically in 10 mM Tris, pH 8.0, 1 mM EDTA, and all samples used for array analysis exceeded values of 1.8. The RNA integrity was analyzed by electrophoresis using the RNA 6000 Nano Assay run in the Lab-on-a Chip (Agilent Technologies, Palo Alto, Calif.). High quality RNA quality criteria included a 28S rRNA/18S rRNA peak area ratio>1.5 and the absence of DNA contamination. To prepare cRNA target, the mRNA was reverse transcribed into cDNA, followed by re-transcription in a method that uses two rounds of amplification devised for small starting RNA samples, kindly provided by Ihor Lemischka (Princeton University), with the following modifications: linear acrylamide (10 ug/ml, Ambion, Austin, Tex.) was used as a co-precipitant in steps that used alcohol precipitation and the starting amount of RNA was 2.5 ug of total RNA. Briefly, a T7-(dT)₂₄ oligonucleotide primer (Genset Oligos, La Jolla, Calif.) was annealed to 2.5 ug of total RNA and reverse transcribed with Superscript II (Invitrogen, Grand Island, N.Y.) at 42° C. for 60 min. Second strand cDNA synthesis by DNA polymerase I (Invitrogen) at 16° C. for 120 min was followed by extraction with phenol:chloroform:isoamyl alcohol (25:24:1)(Sigma, St. Louis, Mo.) and microconcentration (Microcon 50. Millipore, Bedford, Mass.). RNA was then transcribed from the cDNA with a high yield T7 RNA polymerase kit (Megascript, Ambion, Austin, Tex.). The second round of amplification utilized random hexamer and T7-(dT)₂₄ oligonucleotide primers, Superscript II, DNA polymerase I and a biotin labeling high yield T7 RNA polymerase kit (Enzo Diagnostics, Farmingdale, N.Y.). The biotin-labeled cRNA was purified on RNeasy mini kit columns, eluted with 50 ul of 45° C. RNase-free water and quantified using the RiboGreen assay.

Target Labeling and Probe Hybridization

Following quality check on Agilent Lab-on-a-Chip, 15 ug cRNA were fragmented for 35 minutes in 200 mM Tris-acetate pH 8.1, 150 mM MgOAc and 500 mM KOAc following the Affymetrix protocol (Affymetrix, Santa Clara, Calif.). The fragmented RNA was then hybridized for 20 hours at 45° C. to HG_U95Av2 probes. The hybridized probe arrays were washed and stained with the EukGE-WS2 fluidics protocol (Affymetrix), including streptavidin phycoerythrin conjugate (SAPE, Molecular Probes, Eugene, Oreg.) and an antibody amplification step (Anti-streptavidin, biotinylated, Vector Labs, Burlingame, Calif.). HG_U95Av2 chips were scanned at 488 nm, as recommended by Affymetrix. The images were inspected to detect artifacts. The expression value of each gene was calculated using Affymetrix GENECHIP software for the 12,625 Open Reading Frames on the probe set.

Data Presentation and Exclusion Criteria

Criteria used as quality control for exclusion of poor sample arrays included: total RNA integrity, cRNA quality, probe array image inspection, B2 oligo staining (used for Array grid alignment), and internal control genes (GAPDH value greater than 1800). Of the 142 cases initially selected, 126 were ultimately retained in the study; 16 cases were excluded from the final analysis due to poor quality total RNA or cRNA amplification or a poor hybridization (low percentage of expressed genes<10%, poor 3′/5′ amplification ratios).

Data Analysis

1. Data Preprocessing

The preprocessing stage was divided in filtering and transformation. For filtering, the control probesets were removed (i.e. probesets whose accession ID starts with the AFFX prefix), as well as all probesets that had at least one “absent” call (as determined by the Affymetrix MAS 5.0 statistical software) across all training set samples. In the transformation stage, the natural logarithm of the gene expression values (i.e. the signal values) was taken. This is the preprocessing method used for most of the analysis methods; except those in which different preprocessing is mentioned in the detailed information below.

2. Description of the supervised learning methods for class prediction The exploratory evaluation of our data set was performed in several steps. The first step was the construction of predictive classification algorithms that linked gene expression data to patient outcome as well as the traditional clinical variables that define prognosis. With previous knowledge of their sample nature, the 126 patients were divided into statistically balanced and representative training (82 patients) and test sets (44 patients), according to the clinical labels (leukemia lineage, cytogenetics and outcome). For classification purposes, several primary supervised approaches were used, including Bayesian networks, recursive feature elimination in the context of Support Vector Machines (SVM-RFE), linear discriminant analysis and fuzzy logics. Classification tasks were as follows:

| | |
|---|---|
| ALL vs. AML | Remission. vs. Fail |
| t(4; 11) vs. not t(4; 11) | MLL vs. Not MLL |
| Remission. vs. Fail in ALL | Remission. vs. Fail in AML |
| Remission. vs. Fail in VxInsight cluster A | Remission. vs. Fail in |
| | VxInsight cluster B |
| Remission. vs. Fail in VxInsight cluster C | MLL vs. Not MLL in ALL |
| MLL vs. Not MLL in AML | Remission. vs. Fail in MLL |
| Remission. vs. Fail in Not MLL | |

2.1. Bayesian Networks

We employed the Bayesian network framework described in (6), without any data preprocessing. The Bayesian network modeling and learning paradigm was introduced in Pearl (1988) and Heckerman et al. (1995), (7, 8) and has been studied extensively in the statistical machine learning literature. Our work tailors this paradigm to the analysis of gene expression data in general and to the classification problem in particular. A Bayesian net is a graph-based model for representing probabilistic relationships between random variables. The random variables, which may, for example, represent gene expression levels, are modeled as graph nodes; probabilistic relationships are captured by directed edges between the nodes and conditional probability distributions associated with the nodes. A Bayesian net asserts that each node is statistically independent of all its no descendants, once the values of its parents (immediate ancestors) in the graph are known. That is, a node n's parents render n and its no descendants conditionally independent. In our modeling, we consider Bayesian nets in which each gene is a node, and the class label of interest is an additional node C having no children. The conditional independence assertion associated with (leaf) node C implies that the classification of a case q depends only on the expression levels of the genes, which are C's parents in the net. More formally, distribution Pr{q[C]\q[genes]} is identical to distribution Pr{q[C]\q[Par(C)]}, where Par(C) denotes the parent set of C. Note, in particular, that the classification does not depend on other aspects (other than the parent set of C) of the graph structure of the Bayesian net. Thus, while the Bayesian network model ultimately can be a highly appropriate tool for learning global gene regulatory networks, in the context of classification tasks such as those considered in this paper, the Bayesian network learning problem may be reduced to the problem of learning subnetworks consisting only of the class label and its parents. It is important to emphasize how this modeling differs from that of a naïve Bayesian classifier (9, 10) and from the generalization described in (11). A naive Bayesian classifier assumes independence of the attributes (genes), given the value of the class label. Under this assumption, the conditional probability Pr{q[C]\q[genes]} can be computed from the product Πgᵢεgenes Pr{q[gᵢ]\q[C]} of the marginal conditional probabilities. The naive Bayesian model is equivalent to a Bayesian net in which no edges exist between the genes, and in which an edge exists between every gene and the class labels. We make neither assumption. Rather, we ignore the issue of what edges may exist between the genes, and compute Pr{q[C]\q[genes]} as Pr{q[C]\q[Par(C)]}, an equivalence that is valid regardless of what edges exist between the genes, provided only that Par(C) is a set of genes sufficient to render the class label conditionally independent of the remaining genes. Friedman et al. (1997) (11) drops the independence assumption of a naive Bayesian classifier and attempts to learn edges between the attributes (genes, in our context), while maintaining an edge from the class label into each attribute. This approach yields good improvements over naive Bayesian classifiers in the experiments (application domains other than gene expression data) reported in Friedman et al. (1997) (11). Our approach exploits a prior belief (supported by experimental results reported in (6) and in other gene expression analyses) that for the gene expression application domain, only a small number of genes is necessary to render the class label (practically) conditionally independent of the remaining genes. This both makes learning parent sets Par(C) tractable, and generally allows the quantity Pr{q[C]\q[Par(C)]} to be well estimated from a training sample. Even with the focus on restricted subnetworks, the learning problem is enormously difficult. Given a collection of training cases, we must learn one or more “plausible” Bayesian subnetworks, each consisting of class label node C and its parent set Par(C). The main factors contributing to the difficulty of this learning problem are the large number genes, the fact that the expression values of the genes are continuous, and the fact that expression data generally is rather noisy. The approach to Bayesian network learning employed here identifies parent sets which are supported by current evidence by employing an external gene selection algorithm which produces between 20 and 30 genes using a measure of class separation quality similar to the TNoM score described in (12, 13). A binary binning of each selected gene's expression value about a point of maximal class separation also is performed. The set of selected genes then is searched exhaustively for parent sets of size 5 or less, with the induced candidate networks being evaluated by the BD scoring metric (8). This metric, along with a variance factor, is used to blend the predictions made by the 500 best scoring networks (6). Each of these 500 Bayesian networks can be viewed as a competing hypothesis for explaining the current evidence (i.e., training data and simple priors) for the corresponding classification task, and the gene interactions each suggests are potentially of independent interest as well. Another significant aspect of our method involves a distinct normalization of the gene expression data for each classification task. We have found this a necessary follow-up step to the standard Affymetrix scaling algorithm. Our approach to normalization is to consider, for each case, the average expression value over some designated set of genes, and to scale each case so that this average value is the same for all cases. This approach allows the analysis to concentrate on relative gene expression values within a case by standardizing a reference point between cases. The designated reference genes for a given classification task are selected based on poorest class separation quality, which is a heuristic for identifying reference genes likely to be independent of the class label.

2.2 Support Vector Machines

Support vector machines (SVMs) are powerful tools for data classification (14, 15, 16). The development of the SVM was motivated, in the simple case of two linearly separable classes, by the desire to choose an optimal linear classifier out of an infinite number of linear classifiers that can separate the data. This optimal classifier corresponds not only to a hyperplane that separates the classes but also to a hyperplane that attempts to be as far away as possible from all data points. If one imagines inserting the widest possible corridor between data points (with data points belonging to one class on one side of the corridor and data points belonging to the other class on the other side), then the optimal hyperplane would correspond to the imaginary line/plane/hyperplane running through the middle of this corridor.

The SVM has a number of characteristics that make it particularly appealing within the context of gene selection and the classification of gene expression data, namely:
The SVM is a multivariate classification algorithm that takes into account each gene simultaneously in a weighted fashion during training, and
It scales quadratically with the number of training samples, N, and not with the number of features/genes, d.

In order to be computationally feasible, other methods first have to reduce the number of dimensions (features/genes), and then classify the data in the reduced space. A univariate feature selection process or filter ranks genes according to how well each gene individually classifies the data (13,17). The overall SVM classification is then heavily dependent upon how successful the univariate feature selection process is in pruning genes that have little class-distinction information content. In contrast, the SVM provides an effective mechanism for both classification and feature selection via the Recursive Feature Elimination algorithm (18). This is a great advantage in gene expression problems where d is much greater than N because the number of features does not have to be reduced a priori.

Recursive Feature Elimination (RFE) is an SVM-based iterative procedure that generates a nested sequence of gene subsets whereby the subset obtained at iteration k+1 is contained in the subset obtained at iteration k. The genes that are kept per iteration correspond to genes that have the largest weight magnitudes—the rationale being that genes with large weight magnitudes carry more information with respect to class discrimination than those genes with small weight magnitudes.

Implementation of RFE algorithm: The rate of reduction in the number of genes via the RFE algorithm typically been geometric in nature (18,19). For example, in (18), 50% of the genes were removed per RFE iteration. However, as in (19), we have taken a less aggressive pruning approach with respect to the number of genes being removed per RFE iteration. In this work, the number of genes removed was constant within blocks of intervals: from 8000 to 1000 genes, 1000 genes were removed per iteration; from 900 to 200 genes, 100 genes were removed per iteration, etc.

Leave-one-out cross-validation (LOOCV) was used to assess the performance of a linear SVM classifier. The LOOCV procedure divides the training samples into N disjoint sets where the i^{th} set contains samples 1, . . . , i−1, i+1, . . . , N. The SVM classifier is then trained on the i^{th} set and tested on the withheld i^{th} sample. This process is repeated for each set and the LOOCV error is the overall number of misclassifications divided by N. Note that the RFE algorithm was performed separately on each leave-one-out fold—failure to do induces a selection bias that yields LOOCV error rates that are overly optimistic (20). If the benchmark for determining the number of genes to use in training the SVM classifier is based only upon RFE iterations with low LOOCV error, then one finds in practice many sets of gene numbers (e.g. 500, 100 or 50 genes) that satisfy this criterion. Using only the training set LOOCV error, there is no obvious way to choose which number of genes should be used a priori on the test set. Indeed, classifiers using different numbers of genes will often lead to inconsistent predictions on the test set.

Instead of choosing one subset of genes out of many as the definitive gene subset to be used on the test set, we instead use many subsets in a weighted voting scheme fashion. The gene subsets used corresponds to those sets with low LOOCV error. To determine the weight attributed to each subset of genes, metrics of classifier assessment other than LOOCV error were used. Once LOOCV has been performed, the SVM classifier is then retrained on the entire training set.
Let G={G₁, . . . , Gᵣ} denote the collection of gene subsets with low LOOCV error, where r is the number of gene subsets. The number of gene subsets, r, used in this study was determined by inspection. However, one can easily use LOOCV as a mechanism for determining r. Let fᵢ(pⱼ) denote the prediction of the i^{th} set, Gᵢ, for the j^{th} patient, pⱼ, in the test set. The final prediction for the j^{th} patient, f(pⱼ), consists of a linear combination of the predictions made by each set:

$$f ( {p}_{j} ) = {\sum }_{i = 1}^{r} {α}_{i} {f}_{i} ( {p}_{j} )$$

where αᵢ is the weight attributed to each gene subset. In this work, αᵢ is determined solely from the training set and consists of two components:
A margin measure,

$${median}_{i , k} {g}_{i} ( {p}_{k} ) {y}_{k} ,$$

where gᵢ(pₖ) is the prediction made by the i^{th} set, Gᵢ, for the k^{th} patient, pₖ, in the training set; this margin measure, which is typically positive, is similar in spirit to the median margin metric used in (18).
The median number of support vectors across r gene subsets.
The mathematical expression for α is a heuristic one: αᵢ=aᵢ₁+αᵢ₂ where

$${α}_{i1} = \frac{{m}_{i}}{{\sum }_{i = 1}^{p} {m}_{i}} , \qquad and \qquad {α}_{i2} = \frac{1 / {NSV}_{i}}{{\sum }_{i = 1}^{p} ( 1 / {NSV}_{i} )} ,$$

such that mᵢ is the median margin measure, αᵢ₁ is the normalized margin measure, NSVᵢ is the median number of support vectors obtained using Gᵢ as the feature set in the SVM classifier and αᵢ₂ is the normalized reciprocal of the number of support vector patients. The larger mᵢ is, the greater the influence Gᵢ has on the overall vote since larger margins correspond to better separation between classes and presumably better separation in the test set. In contrast, the larger NSVᵢ is, the lesser the influence Gᵢ has on the overall vote since separating hyperplanes determined by fewer support vectors tend to have better generalization.

The SVM and RFE algorithms were written in MATLAB (21). The particular SVM algorithm used was based upon the Lagrangian SVM formulation of Mangasarian and Musicant (22). The RFE approach with the voting scheme extension achieved the highest test set accuracy on the majority of the tasks examined in this work. The best test accuracy was achieved for the AML/ALL classification task while the performance on the other tasks were slightly better than the “majority-class” results—the results obtained if one were to always vote with the majority class. This is not surprising since the AML/ALL class distinctions tend to “dominate” the gene expression behavior. Since SVMs are not dependent upon an a priori and external feature/gene reduction procedure and can efficiently fold feature selection into the classification process, they will continue to perform well on tasks where the class distinctions dominate the gene expression behavior. Non-linear SVMs were trained on several of the classification tasks, but their generalization performance on the test set, as expected, was far worse than the linear SVM classifiers. Since the patients already sparsely populate a very high-dimensional gene space, mapping to even higher-dimensional feature space via a nonlinear kernel will only exacerbate the dilemma of over fitting, a condition already made worse due to the disturbingly small size of the training set relative to the number of genes and the large amount of experimental noise associated with microarray-generated data in general.

2.3 Class Prediction by Linear Discriminant Analysis

Discriminant analysis is a widely used statistical analysis tool (23). It can be applied to classification problems where a training set of samples, depending on some set of feature variables, is available. The idea is to find a linear or non-linear function of the feature variables such that the value of the function differs significantly between different classes. The function is the so-called discriminant function. Once the discriminant function has been determined using the training set, we can predict the class that a new sample most likely belongs to.

Preprocessing: Not all of the original data ware used in our analysis of the infant leukemia dataset. We eliminated all control genes (those with accession ID starting with the AFFX prefix) and those genes with all calls ‘Absent’ for all 142 samples. With these genes removed from the original 12625, we were left with 8414 genes. In addition, a natural log transformation was performed on 8414×142 matrix of the gene expression values prior to further analysis.

Selection of Significant Discriminating Genes for Binary Classifications: We assumed that the discriminating genes will be those with the most statistically significant difference between the two classes in a given binary classification task. We evaluated each gene by checking if its expression value differed significantly between the two classes. This was done using the two-sample t-test. The larger the absolute value of the t-test statistic T, the greater the confidence that there is a difference between the expression values of the two classes. The significance of the difference can be measured via the corresponding p-value, which provides a straightforward means of ranking the genes in order of importance.

Class Prediction: Once the genes have been ranked using the p-value, we need to select a subset as our discriminant variables. The expression values of these genes in the training set are used to determine a linear discriminant function, which discriminates between the two classes and also defines a trained classifier for making the class predictions for each sample in the test set. The question is how to determine the optimal value for n. n must be less than the sample size of the training set, otherwise the covariance matrix of the samples in the training set will be singular and the discriminant function cannot be determined. Also, if n is too large the discriminant function may be over fitted to the data in the training set, which may lead to more misclassifications when it is used to make predictions in test set. On the other hand, if n is too small, then the information contained in the feature set may be not sufficient for making accurate predictions. In practice, different prediction outcomes result when different numbers n of prediction genes are used in the classifier. To determine the class of a given sample from the test set, we have therefore we have chosen to use a simple voting scheme. We make a series of predictions with the number n of prediction genes varying from ⅓ to ⅔ of the sample size of the training set. (For example, if the number of samples in the training set was 85, we computed predictions for the given sample from the test set using n=28, 29, 30, . . . , 56.) The dominant class predicted is then taken as the final prediction result for the sample. Overall, the results of our discriminant analysis for classification tasks were not as good as those of the other multivariate methods (fuzzy logic, Bayesian, SVM) applied to these problems.

2.4 Fuzzy Interference Classification Methodology

Traditional classification methods are based on the theory of crisp sets, where an element is either a member of a particular set or not. However many objects encountered in the real world do not fall into precisely defined membership criteria. Alternative forms of data classification, which allows for continuous membership gradations, have been investigated and introduced fuzzy logic theory (24).

In many applications, it is easier to produce a linguistic description of a system than a complex mathematical model. The advantage of fuzzy logic in these situations is its ability to describe systems linguistically through rule statements (25). Expert human knowledge can then be formulated in a systematic manner. For example, for a gene regulatory model, one rule statement might be: “If the activator A is high and the repressor B is low, then the target C would be high” (26).

A Fuzzy Inference System (FIS) contains four components: fuzzy rules, a fuzzifier, an inference engine, and a “defuzzifier” (27). The fuzzy rules, consisting of a collection of IF-THEN rules, define the behavior of the inference engine. The membership functions μ_{F}(x) provide measure of the degree of similarity of elements to the fuzzy subset.

In fuzzy classification, the training algorithm adapts the fuzzy rules and membership functions so that the behavior of the inference engine represents the sample data sets. The most widely used adaptive fuzzy approach is the neuro-fuzzy technique, in which learning algorithms developed for neural nets are modified so that they can also train a fuzzy logic system (28).

Preprocessing. The infant dataset we used consists of gene expression level for 12625 probesets on the Affymetrix U95Av2 chip, including 67 control genes, measured for 142 patients. The Affymetrix Microarray Suite (MAS) 5.0 assigns a “Present”, “Marginal”, or “Absent” call to the computed signal reported for each probeset [Affymetrix 2001]. Because of strong observed variations in the range of gene expression values across different experiments, it is necessary to preprocess the data prior to further analysis.

In the infant dataset, 17% of all the labels are “Present”, 81% are “Marginal”, and 2% are “Absent”. We prefer not to eliminate too many probesets at the outset. So we choose a loose rule to filter the probesets. We assume that “reliable probesets” satisfy the following criteria:
1. They are not control genes;
2. For a given probeset, at least one label (across all patients) should be “Present”.
   Under these criteria, 8446 probesets survive.
For a given patient, the distribution of gene expression values is not uniform. It grows exponentially. After filtering, we therefore perform a base-10 logarithmic transformation of the gene expression data. This logarithmic transformation scales the data to assist in visualizations, remedies right-skewed distributions and makes error components additive (29). It also removes systematic variations in experiments. Previously, in our analysis of the MIT leukemia dataset (30), we have found that logarithmic transformation of the gene expression data improves fuzzy and neuro-fuzzy classification accuracies compared to untransformed data.
   Feature Selection: Even after filtering, the dimension of our dataset, 8446, is still too large for a classification problem. It is well known that increasing the number of features beyond a value of the order of the number of samples can actually degrade classification performance rather than improving it (31). In addition, reducing the dimensionality of the feature space is necessary to decrease the cost and time of classification (32). Here we use rank ordering statistics for feature selection.
   Our method is as follows. For a given classification task, we rank the genes according to the average signal intensity across the patients in each class. We then calculate the difference in rank position between the two classes for each gene and order these genes with increasing value of the rank difference. The larger the absolute difference in rank for a gene, the more important that gene is. Rank ordering identifies the genes with the most “discriminating power” for distinguishing the two classes. Finally, we select the top 100 genes, corresponding to the 100 largest rank ordering differences, as our discriminating genes, for input to the fuzzy classifier.
   Classification Approach: The 100 “top” genes determined in the feature selection step are in reality an upper bound for the optimal number, k^{*}, of discriminating genes. We note, too, that k^{*} will vary according to classification task because the training model will be different for each task. Here, we have used Leave One Out Cross Validation (LOOCV) to determine k^{*} for each task (33).
We followed standard LOOCV methodology to compute the prediction error of our classification method. This procedure iterated k from 1 to 100 in the dataset, where k is the number of top discriminating genes training our model. Within each iteration, we iteratively removed a single patient from the data set and trained the classification procedure using k discriminating genes on the rest of the patients. We then applied the trained classifier to the held-out patient and compared the predicted class to the true class. The number of prediction errors is f^{k} and the LOOCV error is e^{k}. The optimal solution, k*, corresponds to

$${min}_{k} ( {e}^{k} \times {f}^{k} ) .$$

With the number of genes now fixed at k^{*}, we used the labeled training dataset to generated a Sugeno-type fuzzy inference system using the Fuzzy Logic Toolbox in Matlab (34). This uses the fuzzy c-means technique to partition each data point to a degree specified by a membership grade, and subtractive clustering to initialize the iterative optimization. For comparison, we also implemented an adaptive neuro-fuzzy inference system (ANFIS) to tune the parameters of the fuzzy membership functions based on knowledge learned from the modeling data. Training an ANFIS is an optimization task with the goal of finding a set of weights that minimizes an error measure. In our tests, we found that this procedure increased the computational burden significantly, but provided only marginal performance improvement. Once the classifier was trained, we can use it to predict the class type of the test dataset. For a given new patient, the inputs to the FIS are signal intensities of the top k^{*} genes. The output of the FIS is the classification result for this patient. The ideal output for the ALL class is 1 and the ideal output for the AML class is −1. The larger the distance between the actual prediction and 1/−1 is, the less strong the prediction. Fuzzy methods share a number of features in common with neural networks and with probabilistic methods (such as Bayesian approaches), however they have several unique advantages, which suggest interesting avenues for future research. In particular, their ability to naturally incorporate non-numeric data expert into a model, opens the possibility of the use of expert data priors such as clinical assessments within the classification system. Similarly, incomplete knowledge about gene interrelationships may be incorporated into gene-expression-based models of regulatory networks.
3. Methods for Evaluating the Performance of Class Predictors
Four class predictors—based on the techniques of Bayesian Networks, Support Vector Machines (SVM), Fuzzy Inference and Discriminant Analysis, as described in the previous section—have been applied to thirteen supervised binary classification tasks using gene expression microarray data for the cohort of infant leukemia patients studied in the present work. In this section we describe the statistical methods we have used for evaluating the performance of the four class predictors based on their prediction results with respect to the thirteen tasks.

In any binary classification task, there are four possible prediction outcomes characterized as true-positive (TP), false-positive (FP), true-negative (TN) and false-negative (FN). In the former two instances, a sample is, respectively, correctly or incorrectly classified into Class A, while the latter two instances correspond to classification into Not-Class A. Consequently, the performance of a class predictor can always be completely summarized in terms of a 2×2 matrix as shown in Table 48.

TABLE 48
Prediction Outcome Probabilities of a Class Predictor

| Original | Predicted Classes | | Row |
|---|---|---|---|
| Classes | Class A | Not-Class A | Sum |
| Class A | TP = true-positive probability | FN = false-negative | 1 |
| | | probability | |
| Not-Class A | FP = false-positive | TN = true-negative | 1 |
| | probability | probability | |

Note that because each row sums to 1 only one quantity is required from each row in order to determine the entire matrix. In other words, there are only two independent quantities in Table 48. These can be regarded as evaluating the different aspects of the class predictor's performance. Improving a class predictor's performance in TP may lower its TN, while its TN may be improved at the cost of reducing of its TP. In order to evaluate the overall performance of a class predictor, therefore, a measure that combines the two independent quantities is needed.
We considered two such overall measures: the success rate r, and the odds ratio OR. The success rate is defined as the probability of correct prediction. This is just a weighted average of TP and TN:
r=w₁TP+w₂TN, [1]
where w₁=actual proportion of Class A in the test set, and w₂=1−w₁. TP and TN are intrinsic values associated with a given predictor, and are unknown; therefore r is also unknown and must be estimated. A commonly used point estimate of r, which we have utilized here, is the ratio of the number of correct predictions to the total number of predictions. We have also computed the 95% confidence intervals of r (35). Finally, we have performed a significance test to evaluate the extent to which the performance of a predictor differs from what would have been obtained by chance alone. This is equivalent to testing the statistical hypotheses
H₀:r=0.5 verses H_{A}:r>0.5. [2]
If the p-value (35) of the test is no larger than a given significance level α (here, we have set α=0.05 and α=0.01), then we reject the null hypothesis H₀ and conclude that the difference is significant at level α. The p-value is closely related to the success rate: the larger the success rate, the smaller the expected p-value. Thus, either success rate or the p-value can be used to measure the performance of a predictor. For each of four class predictors, and with respect to each of thirteen tasks, we have computed the point estimate and confidence interval of r. These are presented in Table 48, along with the p-value corresponding to the statistical test of hypotheses [2].
The second overall measure that we utilized is the odds ratio (OR). Since a good class predictor should simultaneously satisfy
TP>FN and FP<TN, [3]
or equivalently,
TP/FN>1 and FP/TN<1, [4]
this implies that the ratio of the right hand sides of the inequalities in [4], i.e.,

$$OR = \frac{TP / FN}{FP / TN} , \qquad [ 5 ]$$

should be large (at least larger than 1). Hence this ratio—known as the odds ratio (29)—can be utilized as an overall measure for evaluating the class predictor's performance. For each of the four class predictors and each of the thirteen tasks, the estimated value of OR and its 95% exact confidence interval (36) have been calculated through the use of SAS package (37), and the results are listed in Table 49.
Above, we observed that the expected values for the TP and FP of a good class predictor should satisfy TP>FP or TP/FP>1, which is mathematically equivalent to OR>1. This suggests that the performance of a classifier can alternatively be evaluated by testing the following hypotheses:
H₀:TP<FP vs. H_{A}:TP>FP, [6]
or equivalently
H₀: OR<1 vs. H_{A}:OR>1. [7]
Hence the p-value of the test also serves as a good measure for evaluating the performance of the class predictor. An uniformly most powerful unbiased test—known as Fisher's exact test (38)—has been used to test the hypotheses [7] and the p-values of the test are given in Table 49.

From Tables 48 and 49 it is evident that all of the four class predictors performed well on Tasks 1 and 3. The statistical test for hypotheses [2] rejects the null hypothesis H₀ and we may conclude that the predictions made by the four class predictors on these tasks are significantly better than those made by chance, at level α=0.01. Fisher's exact test yields the similar results, except that for two of the predictors (fuzzy inference and discriminant analysis), the significance level for Task 3 predictions is α=0.05.

TABLE 49
Overall Success Rates of Class Predictors

| | | Bayesian Net | | | SVM | | | Fuzzy Inference | | | Discriminant Analysis | | |
|---|---|---|---|---|---|---|---|---|---|---|---|---|---|
| Task # | Description | r | C.I. | p-value | r | C.I. | p-value | r | C.I. | p-value | r | C.I. | p-value |
| 1 | ALL vs. AML | .886 | [.73, .97] | .000** | .943 | [.81, .99] | .000** | .943 | [81, .99] | .000** | .829 | [.66, .93] | .000** |
| 2 | Remission. vs. Fail | .514 | [.34, .69] | .500 | .629 | [.45, .79] | .087 | .514 | [.34, .69] | .500 | .514 | [.34, .69] | .500 |
| 3 | t(4; 11) vs. Not t(4; 11) | .818 | [.65, .93] | .000** | .879 | [.72, .97] | .000** | .788 | [.61, .91] | .000** | .788 | [.61, .91] | .000** |
| 4 | MLL vs. Not MLL | .643 | [.44, .81] | .092 | .607 | [.41, .78] | .172 | .679 | [.48, .84] | .043* | .679 | [.48, .84] | .043* |
| 5 | Remission. vs. | .542 | [.33, .74] | .419 | .625 | [.41, .81] | .153 | .375 | [.19, .59] | .924 | .500 | [.29, .71] | .580 |
| | Fail in ALL | | | | | | | | | | | | |
| 6 | Remission. vs. | .429 | [.18, .71] | .788 | .714 | [.42, .92] | .089 | .429 | [.18, .71] | .788 | .500 | [.23, .77] | .604 |
| | Fail in AML | | | | | | | | | | | | |
| 7 | Remission. vs. | .714 | [.29, .96] | .226 | .714 | [.29, .96] | .226 | .857 | [.42, .00] | .062 | .714 | [.29, .96] | .226 |
| | Fail in VX-GA | | | | | | | | | | | | |
| 8 | Remission. vs. | .625 | [.35, .85] | .227 | .563 | [.30, .80] | .401 | .563 | [.30, .80] | .401 | .438 | [.20, .70] | .772 |
| | Fail in VX-GB | | | | | | | | | | | | |
| 9 | Remission. vs. | .786 | [.49, .95] | .028* | .714 | [.42, .92] | .089 | .500 | [.23, .77] | .604 | .500 | [.23, .77] | .604 |
| | Fail in VX-GC | | | | | | | | | | | | |
| 10 | MLL vs. Not | .650 | [.41, .85] | .131 | .600 | [.36, .81] | .251 | .700 | [.46, .88] | .057 | .550 | [.32, .77] | .411 |
| | MLL in ALL | | | | | | | | | | | | |
| 11 | MLL vs. Not | .750 | [.35, .97] | .144 | .375 | [.09, .76] | .855 | .625 | [.24, .91] | .363 | .500 | [.16, .84] | .636 |
| | MLL in AML | | | | | | | | | | | | |
| 12 | Remission. vs. | .471 | [.23, .72] | .685 | .647 | [.38, .86] | .166 | .471 | [.23, .72] | .685 | .353 | [.14, .62] | .928 |
| | Fail in MLL | | | | | | | | | | | | |
| 13 | Remission. vs. | .545 | [.23, .83] | .500 | .636 | [.31, .89] | .274 | .364 | [.11, .69] | .886 | .636 | [.31, .89] | .274 |
| | Fail in Not MLL | | | | | | | | | | | | |

KEY:
r = Estimate of the success rate of the class predictor.
C.I. = 95% confidence interval of the success rate of the class predictor.
p-value = p-value of hypothesis test [2] (see text).
*means that r > 0.5 at significance level α = 0.05.
**means that r > 0.5 at significance level α = 0.01.

TABLE 50
Estimates of Odds Ratios and Fisher's Exact Test

| | Bayesian Net | | | SVM | | | Fuzzy Inference | | | Discriminant Analysis | | |
|---|---|---|---|---|---|---|---|---|---|---|---|---|
| Task # | OR | C.I. | p-value | OR | C.I. | p-value | OR | C.I. | p-value | OR | C.I. | p-value |
| 1 | 76.0 | [5.950, 3408] | 0.000** | 252.00 | [11.3, 11216] | 0.000** | ∞ | [12.84, ∞] | 0.000** | 21.11 | [2.84, 180] | 0.000** |
| 2 | 0.80 | [.134, 4.27] | 0.746 | 2.40 | [.324, 19.3] | 0.270 | 0.68 | [.091, 4.15] | 0.806 | 1.00 | [.204, 4.78] | 0.635 |
| 3 | ∞ | [1.867, ∞] | 0.005** | ∞ | [4.324, ∞] | 0.000** | 14.67 | [1.06, 754] | 0.021* | ∞ | [1.064, ∞] | 0.022* |
| 4 | 2.88 | [.459, 18.5] | 0.175 | 2.50 | [.414, 16.2] | 0.220 | 4.89 | [.739, 37.7] | 0.060 | 3.89 | [.521, 32.1] | 0.123 |
| 5 | 0.79 | [.057, 7.45] | 0.762 | 1.86 | [.109, 30.2] | 0.486 | 0.14 | [.003, 1.678] | 0.991 | 0.91 | [.126, 6.39] | 0.700 |
| 6 | 0.00 | [0.0, 7.081] | 1.000 | ∞ | [.264, ∞] | 0.165 | 0.00 | [0.00, 7.08] | 1.000 | 1.00 | [.077, 13.0] | 0.704 |
| 7 | ∞ | [.142, ∞] | 0.286 | 4.00 | [.026, 391] | 0.524 | ∞ | [.283, ∞] | 0.143 | ∞ | [.142, ∞] | 0.286 |
| 8 | — | — | 1.000 | 0.00 | [0.00, 65] | 1.000 | 0.00 | [0.00, 65] | 1.000 | 0.30 | [.005, 4.884] | 0.942 |
| 9 | ∞ | [.653, ∞] | 0.055 | ∞ | [.264, ∞] | 0.165 | 0.60 | [.009, 15.5] | 0.846 | 0.60 | [.009, 15.5] | 0.846 |
| 10 | 3.00 | [.240, 44.7] | 0.296 | 4.57 | [.316, 253] | 0.221 | 8.00 | [.526, 432] | 0.098 | 1.00 | [.065, 11.8] | 0.693 |
| 11 | 5.00 | [.032, 469.3] | 0.464 | ∞ | [0.009, ∞] | 0.750 | 0.00 | [0.00, 117] | 1.000 | ∞ | [.053, ∞] | 0.536 |
| 12 | 0.00 | [0.00, 4.429] | 1.000 | 2.25 | [.116, 40.2] | 0.445 | 0.60 | [.040, 6.80] | 0.840 | 0.29 | [.019, 3.355] | 0.957 |
| 13 | 0.83 | [.011, 24.1] | 0.788 | 1.50 | [.017, 46.9] | 0.661 | 0.00 | [0.00, 4.16] | 1.000 | 1.50 | [.017, 46.9] | 0.661 |

KEY:
OR = Estimate of the odds ratio.
C.I. = 95% confidence interval of the odds ratio.
p-value = p-value of Fisher's exact test.
*means that OR > 1 at significance level α = 0.05.
**means that OR > 1 at significance level α = 0.01.

4. Unsupervised Methods—Clustering Methodology

Three types of methodologies were used in the clustering analysis, namely agglomerative hierarchical clustering, Principal Component Analysis and a force-directed clustering algorithm coupled with the VxInsight visualization tool.

4.1 Agglomerative Hierarchical Clustering

The grouping together, or clustering, of genes with similar patterns of expression is based on the mathematical measure of their similarity, e.g. the Euclidian distance, angle or dot products of the two n-dimensional vectors of a series of n measurements. Biological interpretation of DNA microarray hybridization gene expression data has utilized clustering to re-order genes, and conversely samples into groups which reflect inherent biological similarity. Clustering methods can be divided into two classes, supervised and unsupervised. In supervised clustering vectors are classified with respect to known reference vectors. Unsupervised clustering uses no defined vectors. With a diverse dataset of 126 infant leukemia patients and our intent to discover unique patterns within, we chose to use an unsupervised clustering approach. In addition, combining the ordered list of genes and patients with a graphical presentation of each data point using relative value-color, termed a “heat map”, aids the viewer in an intuitive manner. Several computer software programs allow one to cluster significant samples and genes and create graphical output (Cluster, Genespring, GeneCluster).

We have applied the Eisen (39) Cluster algorithm utilizing pair wise average-linkage cluster analysis to gene expression data from Affymetrix U95Av2 arrays. Genes were selected for this analysis if the Affymetrix Microarray Analysis Software v. 5.0 predicted at least 1 of 126 patient data were “Present”. The resulting 8,358 genes were z-scored across patients and the standard deviation determined. The clustering algorithm of genes is as follows: the distance between two genes is defined as 1−r where r is the correlation coefficient between the 252 values of the two genes across samples. Two genes with the closest distance are first merged into a super-gene and connected by branches with length representing their distance, and are deleted from future merging. The expression level of the newly formed super-gene is the average of standardized expression levels of the two genes (average-linked) across samples. Then the next super-gene with the smallest distance is chosen to merge and the process repeated 8,352 times to merge all 8,353 genes.

4.2 Principal Component Analysis

Principal component analysis (PCA) is a well-known and convenient method for performing unsupervised clustering of high-dimensional data. Closely related to the Singular Value Decomposition (SVD), PCA is an unsupervised data analysis technique whereby the most variance is captured in the least number of coordinates (40-42). It can serve to reduce the dimensionality of the data while also providing significant noise reduction. PCA can also be applied to gene-expression data obtained from microarray experiments. When gene expressions are available from a large number of genes and from numerous samples, then the noise suppression and dimension reduction properties of PCA can greatly facilitate and simplify the examination and interpretation of the data. In any microarray experiment, the expression profiles of many genes are monitored simultaneously. Because many genes are often up or down regulated in similar patterns in the cells, these responses are correlated. PCA can identify the uncorrelated or independent sources of variation in the gene expression data from multiple samples. Since random noise tends to be uncorrelated with the signal, PCA does an effective job at separating the signal from the noise in the data.

If the gene expression values from each microarray are written as row vectors, then the entire data set from multiple microarray samples can be represented by a data matrix whose rows represent the gene expressions from each microarray chip. PCA can greatly reduce the complexity and dimensionality of the data by factor analyzing the data matrix into the product of two much smaller matrices. The two smaller matrices are known as scores and loading vectors (or eigenvectors). The decomposition is often achieved with a method known as singular value decomposition (SVD). PCA has the unique property that the decomposition is performed such that the rows of the score matrix are orthogonal and the columns of the eigenvector matrix are also orthogonal. Although there is a strict mathematical definition of orthogonal, orthogonal vectors are simply independent and uncorrelated with one another. Therefore, these vectors represent unique sources of variation in the microarray data. Another property of the eigenvectors is that they are calculated such that the first eigenvector represents the largest source of variance in the data, the second represents the next largest unique source of variance in the data, and so on. Since we generally expect the signal in the data to be larger than the noise and since random noise is approximately orthogonal to the signal, PCA has the ability to separate the noise from signal that we are interested in. By ignoring the eigenvectors with low variance, we can observe the portion of the data that contains primarily signal.

The scores matrix represents the amounts of each eigenvector in each sample that are required to reproduce the data matrix. When we eliminate the noisier eigenvectors we also eliminate their associated scores. The scores represent a compressed form of the data matrix in the new coordinate system of the eigenvectors. Since scores are derived from the expression of many genes and many samples, they have much higher signal-to-noise ratios than the individual gene expressions upon which they are based. A plot of the scores for each microarray for each eigenvector then is a new compressed form of the gene expression data for all samples. 2D plots of one set of scores vs. another for two selected eigenvectors allow us an examination of the microarray data in the compressed PCA space so that we can readily observe clusters in expression data. 3D plots are also possible when the scores from three selected eigenvectors are displayed. Statistical metrics can be used to identify groupings or clusters in the data in 2, 3, or higher dimensions that cannot be readily viewed graphically. All the statistical supervised and unsupervised clustering methods that are based on individual genes or groups of genes can be applied to the scores representation of the data.

The first three Principal Components partition the infant cohort into two different groups. Interestingly, these groups display a weak correlation with the infant ALL/AML lineage membership (and none with the MLL cytogenetics), although the correlation is not seen until the second PC. This indicates, according to the theory behind PCA, that the ALL/AML distinction is not the driving force behind the representation of the patient cohort. The first (and most important) Principal Component, on the other hand, does not reveal any obvious clusters. Upon further analysis, however, we did find an additional interesting group correlated with the first Principal Component. This group was discovered by a force-directed graph layout algorithm and the VxInsight® visualization program (43, 44).

4.3 VxInsight and the Force Directed Clustering Algorithm

This clustering algorithm places genes into clusters such that the sum of two opposing forces is minimized. One of these forces is repulsive and pushes pairs of genes away from each other as a function of the density of genes in the local area. The other force pulls pairs of similar genes together based on their degree of similarity. The clustering algorithm stops when these forces are in equilibrium. Every gene has some correlation with every other gene; however, most of these are not strong correlations and may only reflect random fluctuations. By using only the top few genes most similar to a particular gene as it is placed into a cluster we obtain two benefits. First, the algorithm runs much faster. Second, as the number of similar genes is reduced, the average influence of the other, mostly uncorrelated genes diminishes. This change allows the formation of clusters even when the signals are quite weak. However, when too few genes are used in the process, the clusters break up into tiny random islands, so selecting this parameter is an iterative process. One trades off confidence in the reliability of the cluster against refinement into sub-clusters that may suggest biologically important hypotheses. These clusters are only interpreted as suggestions, and require further laboratory and literature work before we assign them any biological importance. However, without accepting this trade off, it may be impossible to uncover any suggestive structure in the collected data. For example, we clustered using the twenty other genes most strongly similar to each gene. When we re-cluster using only the top ten most strongly similar genes, the observed clusters have broken up into smaller groups. We carefully analyzed these for biological support and believe that they may be suggestive of weak, but important groupings in our experimental data. VxInsight was employed to identify clusters of patients with similar gene expression patterns, and then to identify which genes strongly contributed to the separations. That process created lists of genes, which when combined with public databases and research experience, suggest possible biological significances for those clusters. The array expression data were clustered by rows (similar genes clustered together), and by columns (patients with similar gene expression clustered together). In both cases Pearson's R was used to estimate the similarities. These similarities were used together with a force-directed, two-dimensional clustering algorithm (43, 44) to produce maps showing clusters of genes and patients. Different maps were generated by using the top twenty, top ten and top five strongest correlations for each gene (using more similarity links between genes generates more stable clusters, while using fewer links leads to finer, if less stable, divisions). This methodology has been useful in inferring functions of uncharacterized genes clustered near other genes with known functions (45, 46), and did contribute to our analysis here, too. However, patients were the main focus of this study and most of the analysis revolved around the map of patient clusters. Analysis of variance (ANOVA) was used to determine which genes had the strongest differences between pairs of patient clusters. These gene lists were sorted into decreasing order based on the resulting F-scores, and were presented in an HTML format with links to the associated OMIM pages, which were manually examined to hypothesize biological differences between the clusters.

We also investigated the stability of those gene lists using statistical bootstraps (47, 48). For each pair of clusters we computed 1000 random bootstrap cases (resampling with replacement from the observed expressions) and computed the resulting ordered lists of genes using the same ANOVA method as before. The average order in the set of bootstrapped gene lists was computed for all genes, and reported as an indication of rank order stability (the percentile from the bootstraps estimates a p-value for observing a gene at or above the list order observed using the original experimental values). Because the force directed placement algorithm used by VxInsight has a stochastic element (random initial starting conditions), we used massively parallel computers to calculate hundreds of reclustering with different seeds for the random number generator. We compared pairs of ordinations by counting, for every gene, the number of common neighbors found in each ordination. Typically, we looked in a region containing the 20 nearest neighbors around each gene, in which case one could find (around each gene) a minimum of 0 common neighbors in the two ordinations, or a maximum of 20 common neighbors. By summing across every one of the genes an overall comparison of similarity of the two ordinations can be computed. We computed all pair wise comparisons between the randomly restarted ordinations and found the ordination that had the largest count of similar neighbors across the totality of all the comparisons. Note that this corresponds to finding the ordination whose comparison with all the others has minimal entropy, and in a general sense represents the most central ordination (MCO) of the entire set. It is possible to use these comparison counts (or entropies) as similarity measures to compute another round of ordinations. The clusters from this recursive use of the ordination algorithm are generally smaller, much tighter, and are generally more stable with respect to random starting conditions than any single ordination. We used all of these methods during exploratory data analysis to develop intuition about the data.

5. Lists of Informative Genes

TABLE 51
Discriminating genes that distinguish between ALL and AML types,
derived from Bayesian networks analysis.

| | | |
|---|---|---|
| Affymetrix | | |
| Locus | | Gene |
| number | Gene description | symbol |

A. Bayesian Networks

| | | | |
|---|---|---|---|
| 1 | 38269_at | protein kinase D2 | PKD2 |
| | 19q13.2 | | |
| 2 | 40103_at | villin 2 (ezrin) | VIL2 |
| | 6q25-q26 | | |
| 3 | 41165_g_at | immunoglobulin heavy constant mu | IGHM |
| | 14q32.33 | | |
| 4 | 40310_at | toll-like receptor 2 | TLR2 |
| | 4q32 | | |
| 5 | 38604_at | neuropeptide Y | NPY |
| | 7p15.1 | | |
| 6 | 39689_at | cystatin C | CST3 |
| | 20p11.2 | | |
| 7 | 41356_at | B-cell CLL/lymphoma 11A | BCL11A |
| | 2p15 | | |
| 8 | 461_at | N-acylsphingosine amidohydrolase | ASAH |
| | 8p22-p21.3 | | |
| 9 | 1096_g_at | CD19 antigen | CD19 |
| | 16p11.2 | | |
| 10 | 36938_at | N-acylsphingosine amidohydrolase | ASAH |
| | 8p22-p21.3 | | |
| 11 | 41401_at | cysteine and glycine-rich protein 2 | CSRP2 |
| | 12q21.1 | | |
| 12 | 41523_at | RAB32, member RAS oncogene family | RAB32 |
| | 6q24.2 | | |
| 13 | 40432_at | Homo sapiens, clone IMAGE: 4391536 | |
| 14 | 41164_at | immunoglobulin heavy constant mu | IGHM |
| | 14q32.33 | | |
| 15 | 36766_at | ribonuclease, RNase A family, 2 | RNASE2 |
| | 14q24-q31 | | |
| 16 | 39827_at | hypothetical protein | FLJ20500 |
| | 10pterq26 | | |
| 17 | 37001_at | calpain 2, (m/ll) large subunit | CAPN2 |
| | 1q41-q42 | | |
| 18 | 279_at | nuclear receptor subfamily 4 | NR4A1 |
| | 12q13 | | |
| 19 | 39593_at | Similar to fibrinogen-like 2, clone | |
| 20 | 41038_at | neutrophil cytosolic factor 2 | NCF2 |
| | 1q25 | | |
| 21 | 40936_at | cysteine-rich motor neuron 1 | CRIM1 |
| | 2p21 | | |
| 22 | 32227_at | proteoglycan 1, secretory granule | PRG1 |
| | 10q22.1 | | |
| 23 | 478_g_at | interferon regulatory factor 5 | IRF5 |
| | 7q32 | | |
| 24 | 1230_g_at | cisplatin resistance associated | CRA |
| | 1q12-q21 | | |
| 25 | 35367_at | lectin, galactoside-binding, soluble | LGALS3 |
| | 14q21-q22 | | |

TABLE 52

| | | |
|---|---|---|
| Affymetrix | | |
| Locus | | Gene |
| number | Gene description | symbol |

Discriminating genes that distinguish between ALL and AML types,
derived from SVM analysis.
B. SVM

| | | | |
|---|---|---|---|
| 1 | 41165_g_at | immunoglobulin heavy constant mu | IGHM |
| | 14q32.33 | | |
| 2 | 36766_at | ribonuclease, RNase A family, 2 | RNASE2 |
| | 14q24 | | |
| 3 | 38604_at | neuropeptide Y | NPY |
| | 7p15.1 | | |
| 4 | 36879_at | endothelial cell growth factor 1 | ECGF1 |
| | 22q13.33 | (platelet-derived) | |
| 5 | 41401_at | cysteine and glycine-rich protein 2 | CSRP2 |
| | 12q21.1 | | |
| 6 | 36638_at | connective tissue growth factor | CTGF |
| | 6q23.1 | | |
| 7 | 33856_at | CAAX box 1 | CXX1 |
| | Xq26 | | |

Discriminating genes (between ALL and AML types)
derived from SVM analysis.

| | | | |
|---|---|---|---|
| 8 | 35926_s_at | leukocyte immunoglobulin-like receptor, B | LILRB1 |
| | 19q13.4 | | |
| 9 | 40659_at | nuclear receptor subfamily 4, group A, member 3 | NR4A3 |
| | 9q22 | | |
| 10 | 266_s_at | CD24 antigen (small cell lung carcinoma cluster 4) | CD24 |
| | 6q21 | | |
| 11 | 34180_at | Rho guanine nucleotide exchange factor (GEF) 10 | ARHGEF |
| | 8p23 | | |
| 12 | 279_at | nuclear receptor subfamily 4, group A, member 1 | NR4A1 |
| | 12q13 | | |
| 13 | 38661_at | seb4D | HSRNA |
| | 20q13.31 | | |
| 14 | 38363_at | TYRO protein tyrosine kinase binding protein | TYROBP |
| | 19q13.1 | | |
| 15 | 36657_at | apolipoprotein C-II | APOC2 |
| | 19q13.2 | | |
| 16 | 37050_r_at | translocase of outer mitochondrial membrane 34 | TOM34 |
| 17 | 41523_at | RAB32, member RAS oncogene family | RAB32 |
| | 6q24.2 | | |
| 18 | 39878_at | protocadherin 9 | PCDH9 |
| | 13q14.3 | | |
| 19 | 41577_at | protein phosphatase 1, regulatory (inhibitor) | PPP1R1 |
| | 20q11.23 | | |
| 20 | 854_at | B lymphoid tyrosine kinase | BLK |
| | 8p23-p22 | | |
| 21 | 38403_at | lysosomal-associated membrane protein 2 | LAMP2 |
| | Xq24 | | |
| 22 | 39994_at | chemokine (C—C motif) receptor 1 | CCR1 |
| | 3p21 | | |
| 23 | 33186_i_at | ESTs | |
| 24 | 32227_at | proteoglycan 1, secretory granule | PRG1 |
| | 10q22.1 | | |
| 25 | 39827_at | hypothetical protein | FLJ20500 |
| | 10pterq26 | | |
| 26 | 40103_at | villin 2 (ezrin) | VIL2 |
| | 6q25-q26 | | |
| 27 | 34168_at | deoxynucleotidyltransferase, terminal | DNTT |
| | 10q23 | | |
| 28 | 36465_at | interferon regulatory factor 5 | IRF5 |
| | 7q32 | | |
| 29 | 34433_at | docking protein 1 | DOK1 |
| | 2p13 | | |
| 30 | 41239_r_at | cathepsin S | CTSS |
| | 1q21 | | |
| 31 | 40457_at | splicing factor, arginine/serine-rich 3 | SFRS3 |
| | 11 | | |
| 32 | 32827_at | related RAS viral (r-ras) oncogene homolog 2 | RRAS2 |
| | 11pter-p15.5 | | |
| 33 | 33678_i_at | tubulin, beta, 2 | TUBB2 |
| 34 | 40936_at | cysteine-rich motor neuron 1 | CRIM1 |
| | 2p21 | | |
| 35 | 38242_at | B-cell linker | BLNK |
| | 10q23.2-q23.33 | | |
| 36 | 41164_at | immunoglobulin heavy constant mu | IGHM |
| | 14q32.33 | | |
| 37 | 40220_at | HMBA-inducible | HIS1 |
| | 17q21.32 | | |
| 38 | 40310_at | toll-like receptor 2 | TLR2 |
| | 4q32 | | |
| 39 | 39593_at | Similar to fibrinogen-like 2, IMAGE: 4616866 | |
| 40 | 37844_at | class I cytokine receptor | WSX-1 |
| | 19p13.11 | | |
| 41 | 478_g_at | interferon regulatory factor 5 | IRF5 |
| | 7q32 | | |
| 42 | 38138_at | S100 calcium-binding protein A11 (calgizzarin) | S100A11 |
| | 1q21 | | |
| 43 | 40282_s_at | D component of complement (adipsin) | DF |
| | 19p13.3 | | |
| 44 | 36928_at | zinc finger protein 146 | ZNF146 |
| | 19q13.1 | | |
| 45 | 34800_at | ortholog of mouse integral membrane glycoprotein | LIG1 |
| 46 | 33462_at | G protein-coupled receptor 105 | GPR105 |
| | 3q21-q25 | | |
| 47 | 34950_at | OLF-1/EBF associated zinc finger gene | OAZ |
| | 16q12 | | |
| 48 | 34335_at | ephrin-B2 | EFNB2 |
| | 13q33 | | |
| 49 | 37190_at | WAS protein family, member 1 | WASF1 |
| | 6q21-q22 | | |
| 50 | 40195_at | H2A histone family, member X | H2AFX |
| | 11q23.2-q23.3 | | |
| 51 | 38037_at | diphtheria toxin receptor | DTR |
| | 5q23 | | |
| 52 | 38994_at | STAT induced STAT inhibitor-2 | STATI2 |
| | 12q | | |
| 53 | 38096_f_at | MHC class II, DP beta 1 | HLA-DPB |
| | 6p21.3 | | |
| 54 | 2063_at | excision repair cross-complementing rodent repair | ERCC5 |
| | 13q22 | deficiency, complementation group 5 (xeroderma | |
| | | pigmentosum, complementation group G) | |
| 55 | 461_at | N-acylsphingosine amidohydrolase | ASAH |
| | 8p22-p21.3 | | |
| 56 | 35449_at | killer cell lectin-like receptor subfamily B - 1 | KLRB1 |
| | 12p13 | | |
| 57 | 41198_at | granulin | GRN |
| | 17q21.32 | | |
| 58 | 38993_r_at | Homo sapiens cDNA: clone HEP03585 | |
| 59 | 34677_f_at | Homo sapiens mRNA for TL132 | |
| 60 | 33899_at | aldehyde dehydrogenase 9 family, member A1 | ALDH9A1 |
| | 1q22-q23 | | |
| 61 | 40814_at | iduronate 2-sulfatase (Hunter syndrome) | IDS |
| | Xq28 | | |
| 62 | 33228_g_at | interleukin 10 receptor, beta | IL10RB |
| | 21q22.11 | | |
| 63 | 33458_r_at | H2B histone family, member L | H2BFL |
| | 6p21.3 | | |
| 64 | 41356_at | B-cell CLL/lymphoma 11A (zinc finger protein) | BCL11A |
| | 2p15 | | |
| 65 | 40638_at | splicing factor proline/glutamine rich | SFPQ |
| | 1p34.2 | (polypyrimidine tract-binding protein-associated) | |
| 66 | 40570_at | forkhead box O1A (rhabdomyosarcoma) | FOXO1A |
| | 13q14.1 | | |
| 67 | 40432_at | Homo sapiens, clone IMAGE: 4391536, mRNA | |
| 68 | 39398_s_at | tubulin-specific chaperone d | TBCD |
| | 17q25.3 | | |
| 69 | 2003_s_at | mutS (E. coli) homolog 6 | MSH6 |
| | 2p16 | | |
| 70 | 37561_at | Human DNA sequence from clone 34B21 on | |
| | 6p12.1 | chromosome | |
| 71 | 41038_at | neutrophil cytosolic factor 2 | NCF2 |
| | 1q25 | | |
| 72 | 38402_at | lysosomal-associated membrane protein 2 | LAMP2 |
| | Xq24 | | |
| 73 | 37203_at | carboxylesterase 1 (monocyte/macrophage serine | CES1 |
| | 16q13-q22.1 | esterase 1) | |
| 74 | 34749_at | solute carrier family 31 (copper transporters) | SLC31A2 |
| | 9q31-q32 | | |
| 75 | 40601_at | beta-amyloid binding protein precursor | BBP |
| | 1p31.2 | | |
| 76 | 40194_at | Human chromosome 5q13.1 clone 5G8 mRNA | |
| 77 | 39566_at | cholinergic receptor, nicotinic, alpha polypeptide 7 | CHRNA7 |
| | 15q14 | | |
| 78 | 32706_at | HIR (histone cell cycle regulation defective) | HIRA |
| | 22q11.21 | | |

TABLE 53

| | | |
|---|---|---|
| Affymetrix | | |
| Locus | | Gene |
| number | Gene description | symbol |

Discriminating genes that distinguish between remission and fail
overall derived from SVM analysis.

| | | | |
|---|---|---|---|
| 1 | 41165_g_at | immunoglobulin heavy constant mu | IGHM |
| | 14q32.33 | | |
| 2 | 39389_at | CD9 antigen (p24) | CD9 |
| | 12p13 | | |
| 3 | 41058_g_at | uncharacterized hypothalamus protein HT012 | HT012 |
| | 6p22.2 | | |
| 4 | 31459_i_at | immunoglobulin lambda locus | IGL |
| | 22q11.1-q11.2 | | |
| 5 | 38389_at | 2′,5′-oligoadenylate synthetase 1 (40-46 kD) | OAS1 |
| | 12q24.1 | | |
| 6 | 37504_at | E3 ubiquitin ligase SMURF1 | SMURF1 |
| | 7q21.1-q31.1 | | |
| 7 | 40367_at | bone morphogenetic protein 2 | BMP2 |
| | 20p12 | | |
| 8 | 32637_r_at | PI-3-kinase-related kinase SMG-1 | SMG1 |
| | 16p12.3 | | |
| 9 | 39931_at | dual-specificity tyrosine-(Y)-phosphorylation | DYRK3 |
| | 1q32 | regulated kinase 3 | |
| 10 | 37054_at | bactericidal/permeability-increasing protein | BPI |
| | 20q11 | | |
| 11 | 1404_r_at | small inducible cytokine A5 (RANTES) | SCYA5 |
| | 17q11.2-q12 | | |
| 12 | 1292_at | dual specificity phosphatase 2 | DUSP2 |
| | 2q11 | | |
| 13 | 37709_at | DNA segment, numerous copies | DXF68 |
| | Xp22.32 | | |
| 14 | 36857_at | RAD1 (S. pombe) homolog | RAD1 |
| | 5p13.2 | | |
| 15 | 41196_at | karyopherin (importin) beta 1 | KPNB1 |
| | 17q21 | | |
| 16 | 1182_at | phospholipase C, epsilon | PLCE |
| | 2q33 | | |
| 17 | 34961_at | T cell activation, increased late expression | TACTILE |
| | 3q13.13 | | |
| 18 | 37862_at | dihydrolipoamide branched chain transacylase | DBT |
| | 1p31 | (E2 component of branched chain keto acid | |
| | | dehydrogenase complex; maple syrup disease) | |
| 19 | 38772_at | cysteine-rich, angiogenic inducer, 61 | CYR61 |
| | 1p31-p22 | | |
| 20 | 33208_at | DnaJ (Hsp40) homolog, subfamily C, member 3 | DNAJC3 |
| | 13q32 | | |
| 21 | 37837_at | KIAA0863 protein | KIAA0863 |
| | 18q23 | | |
| 22 | 34031_i_at | cerebral cavernous malformations 1 | CCM1 |
| | 7q21 | | |
| 23 | 38220_at | dihydropyrimidine dehydrogenase | DPYD |
| | 1p22 | | |
| 24 | 34684_at | RecQ protein-like (DNA helicase Q1-like) | RECQL |
| | 12p12 | | |
| 25 | 39449_at | S-phase kinase-associated protein 2 (p45) | SKP2 |
| | 5p13 | | |
| 26 | 32638_s_at | PI-3-kinase-related kinase SMG-1 | SMG1 |
| | 16p12.3 | | |
| 27 | 35957_at | stannin | SNN |
| | 16p13 | | |
| 28 | 34363_at | selenoprotein P, plasma, 1 | SEPP1 |
| | 5q31 | | |
| 29 | 35431_g_at | RNA polymerase II transcriptional regulation | MED6 |
| | 14q24.1 | mediator (Med6, S. cerevisiae, homolog of) | |
| 30 | 35012_at | myeloid cell nuclear differentiation antigen | MNDA |
| | 1q22 | | |
| 31 | 38432_at | interferon-stimulated protein, 15 kDa | ISG15 |
| | 1p36.33 | | |
| 32 | 35664_at | multimerin | MMRN |
| | 4q22 | | |
| 33 | 41862_at | KIAA0056 protein | KIAA0056 |
| | 11q25 | | |
| 34 | 33210_at | YY1 transcription factor | YY1 |
| | 14q | | |
| 35 | 35794_at | KIAA0942 protein | KIAA0942 |
| | 8pter | | |
| 36 | 36108_at | HLA, class II, DQ beta 1 | DQB1 |
| | 6p21.3 | | |
| 37 | 35614_at | transcription factor-like 5 (basic helix-loop-helix) | TCFL5 |
| | 20q13.3 | | |
| 38 | 32089_at | sperm associated antigen 6 | SPAG6 |
| | 10p12 | | |

Discriminating genes that distinguish between remissions and fails
overall derived from SVM analysis.

| | | | |
|---|---|---|---|
| 39 | 1343_s_at | serine (or cysteine) proteinase inhibitor) | SERPINB |
| | 18q21.3 | | |
| 40 | 665_at | serine/threonine kinase 2 | STK2 |
| | 3p21.1 | | |
| 41 | 40901_at | nuclear autoantigen | GS2NA |
| | 14q13 | | |
| 42 | 39299_at | KIAA0971 protein | KIAA0971 |
| | 2q34 | | |
| 43 | 34446_at | KIAA0471 gene product | KIAA0471 |
| | 1q24 | | |
| 44 | 33956_at | MD-2 protein | MD-2 |
| | 8q13.3 | | |
| 45 | 37184_at | syntaxin 1A (brain) | STX1A |
| | 7q11.23 | | |
| 46 | 1773_at | farnesyltransferase, CAAX box, beta | FNTB |
| | 14q23 | | |
| 47 | 34731_at | KIAA0185 protein | KIAA0185 |
| | 10q24.32 | | |
| 48 | 41700_at | coagulation factor II (thrombin) receptor | F2R |
| | 5q13 | | |
| 49 | 38407_r_at | prostaglandin D2 synthase (21 kD, brain) | GDS |
| | 9q34.2 | | |
| 50 | 40088_at | nuclear receptor interacting protein 1 | NRIP1 |
| | 21q11.2 | | |
| 51 | 33124_at | vaccinia related kinase 2 | VRK2 |
| | 2p16 | | |
| 52 | 32964_at | egf-like module containing, mucin-like, hormone | EMR1 |
| | 19p13.3 | receptor-like sequence 1 | |
| 53 | 39560_at | chromobox homolog 6 | CBX6 |
| | 22q13.1 | | |
| 54 | 39838_at | CLIP-associating protein 1 | CLASP1 |
| | 2q14.2 | | |
| 55 | 40166_at | CS box-containing WD protein | LOC55884 |
| 56 | 36927_at | hypothetical protein, expressed in osteoblast | GS3686 |
| | 1p22.3 | | |
| 57 | 41393_at | zinc finger protein 195 | ZNF195 |
| | 11p15.5 | | |
| 58 | 35041_at | neurotrophin 3 | NTF3 |
| | 12p13 | | |
| 59 | 40238_at | G protein-coupled receptor, family C, group 5, | GPRC5B |
| | 16p12 | | |
| 60 | 39926_at | MAD (mothers against decapentaplegic, Drosoph) | MADH5 |
| | 5q31 | | |
| 61 | 36674_at | small inducible cytokine A4 | SCYA4 |
| | 17q21 | | |
| 62 | 32132_at | KIAA0675 gene product | KIAA0675 |
| | 3q13.13 | | |
| 63 | 38252_s_at | 1,6-glucosidase, 4-alpha-glucanotransferase | AGL |
| | 1p21 | | |
| 64 | 33598_r_at | cold autoinflammatory syndrome 1 | CIAS1 |
| | 1q44 | | |
| 65 | 37409_at | SFRS protein kinase 2 | SRPK2 |
| | 7q22 | | |
| 66 | 41019_at | phosducin-like | PDCL |
| | 9q12 | | |
| 67 | 1113_at | bone morphogenetic protein 2 | BMP2 |
| | 20p12 | | |
| 68 | 37208_at | phosphoserine phosphatase-like | PSPHL |
| | 7q11.2 | | |
| 69 | 32822_at | solute carrier family 25 | SLC25A4 |
| | 4q35 | | |
| 70 | 32249_at | H factor (complement)-like 1 | HFL1 |
| | 1q32 | | |
| 71 | 39600_at | EST | |
| 72 | 32648_at | delta-like homolog (Drosophila) | DLK1 |
| | 14q32 | | |
| 73 | 39269_at | replication factor C (activator 1) 3 (38 kD) | RFC3 |
| | 13q12.3 | | |
| 74 | 37724_at | v-myc avian myelocytomatosis viral oncogene | MYC |
| | 8q24.12 | | |
| 75 | 35606_at | histidine decarboxylase | HDC |
| | 15q21 | | |
| 76 | 31926_at | cytochrome P450, subfamily VIIA | CYP7A1 |
| | 8q11 | | |
| 77 | 32142_at | serine/threonine kinase 3 (Ste20, yeast homolog) | STK3 |
| | 8p22 | | |
| 78 | 32789_at | nuclear cap binding protein subunit 2, 20 kD | NCBP2 |
| | 3q29 | | |
| 79 | 37279_at | GTP-binding protein (skeletal muscle) | GEM |
| | 8q13 | | |
| 80 | 40246_at | discs, large (Drosophila) homolog 1 | DLG1 |
| | 3q29 | | |
| 81 | 37547_at | PTH-responsive osteosarcoma B1 protein | B1 |
| | 7p14 | | |
| 82 | 32298_at | a disintegrin and metalloproteinase domain 2 | ADAM2 |
| | 8p11.2 | | |
| 83 | 40496_at | complement component 1, s subcomponent | C1S |
| | 12p13 | | |
| 84 | 39032_at | transforming growth factor beta-stimulated protein | TSC22 |
| | 13q14 | | |

TABLE 54
Discriminating genes that distinguish between remission and fail,
inside the ALL type, derived from SVM.

| | Affymetrix | | |
|---|---|---|---|
| | Locus | | Gene |
| | number | Gene description | symbol |
| 1 | 39389_at | CD9 antigen (p24) | CD9 |
| | 12p13 | | |
| 2 | 1292_at | dual specificity phosphatase 2 | DUSP2 |
| | 2q11 | | |
| 3 | 31459_i_at | immunoglobulin lambda locus | IGL |
| | 22q11.1 | | |
| 4 | 36674_at | small inducible cytokine A4 | SCYA4 |
| | 17q21 | | |
| 5 | 32637_r_at | PI-3-kinase-related kinase SMG-1 | SMG1 |
| | 16p12.3 | | |
| 6 | 35756_at | chromosome 19 open reading frame 3 | C19orf3 |
| | 19p13.1 | | |
| 7 | 41700_at | coagulation factor II (thrombin) receptor | F2R |
| | 5q13 | | |
| 8 | 31853_at | embryonic ectoderm development | EED |
| | 11q14.2 | | |
| 9 | 31329_at | putative opioid receptor, neuromedin K | TAC3RL |
| | | (neurokinin B) receptor-like | |
| 10 | 34491_at | 2′-5′-oligoadenylate synthetase-like | OASL |
| | 12q24.2 | | |
| 11 | 34961_at | T cell activation, increased late expression | TACTILE |
| | 3q13.13 | | |
| 12 | 160021_r_at | progesterone receptor | PGR |
| | 11q22 | | |
| 13 | 37773_at | KIAA1005 protein | KIAA1005 |
| | 16 | | |
| 14 | 38367_s_at | complement component 4-binding protein, beta | C4BPB |
| | 1q32 | | |
| 15 | 32279_at | glutamate decarboxylase 2 | GAD2 |
| | 10p11 | | |
| 16 | 36108_at | MHC complex, class II, DQ beta 1 | DQB1 |
| | 6p21.3 | | |
| 17 | 34378_at | adipose differentiation-related protein | ADFP |
| | 9p21.3 | | |
| 18 | 777_at | GDP dissociation inhibitor 2 | GDI2 |
| | 10p15 | | |
| 19 | 35140_at | cyclin-dependent kinase 8 | CDK8 |
| | 13q12 | | |
| 20 | 33208_at | DnaJ (Hsp40) homolog, subfamily C, member 3 | DNAJC3 |
| | 13q32 | | |
| 21 | 33405_at | adenylyl cyclase-associated protein 2 | CAP2 |
| | 6p22.3 | | |
| 22 | 39580_at | KIAA0649 gene product | KIAA0649 |
| | 9q34.3 | | |
| 23 | 32469_at | carcinoembryonic antigen-cell adhesion 3 | CEACAM |
| | 19q13.2 | | |
| 24 | 38539_at | solute carrier family 24, member 1 | SLC24A1 |
| | 15q22 | | |
| 25 | 1454_at | MAD (mothers against decapentaplegic) 3 | MADH3 |
| | 15q21 | | |
| 26 | 35289_at | rab6 GTPase activating protein | GPCENA |
| | 9q34.11 | | |
| 27 | 37724_at | v-myc avian myelocytomatosis viral oncogene | MYC |
| | 8q24.12-q24.13 | | |
| 28 | 32521_at | secreted frizzled-related protein 1 | SFRP1 |
| | 8p12 | | |
| 29 | 1375_s_at | tissue inhibitor of metalloproteinase 2 | TIMP2 |
| | 17q25 | | |
| 30 | 555_at | GTP-binding protein homologous | SEC4 |
| | 17q25.3 | | |
| 31 | 224_at | TGFB inducible early growth response | TIEG |
| | 8q22.2 | | |
| 32 | 40367_at | bone morphogenetic protein 2 | BMP2 |
| | 20p12 | | |
| 33 | 41504_s_at | v-maf aponeurotic fibrosarcoma oncogene | MAF |
| | 16q22 | | |
| 34 | 40166_at | CS box-containing WD protein | LOC55884 |
| 35 | 35228_at | carnitine palmitoyltransferase I, muscle | CPT1B |
| | 22q13 | | |
| 36 | 33491_at | sucrase-isomaltase | SI |
| | 3q25.2 | | |
| 37 | 1182_at | phospholipase C, epsilon | PLCE |
| | 2q33 | | |
| 38 | 38869_at | KIAA1069 protein | KIAA1069 |
| | 3q25.31 | | |
| 39 | 35811_at | ring finger protein 13 | RNF13 |
| | 3q25.1 | | |
| 40 | 37504_at | E3 ubiquitin ligase SMURF1 | SMURF1 |
| | 7q21.1-q31.1 | | |
| 41 | 160025_at | transforming growth factor, alpha | TGFA |
| | 2p13 | | |
| 42 | 35233_r_at | centrin, EF-hand protein, 3 (CDC31 yeast) | CETN3 |
| | 5q14.3 | | |
| 43 | 40399_r_at | mesenchyme homeo box 2 (growth arrest) | MEOX2 |
| | 7p22.1-p21.3 | | |
| 44 | 31810_g_at | contactin 1 | CNTN1 |
| | 12q11 | | |
| 45 | 40789_at | adenylate kinase 2 | AK2 |
| | 1p34 | | |
| 46 | 35614_at | transcription factor-like 5 (basic helix-loop-helix) | TCFL5 |
| | 20q13.3 | | |
| 47 | 34482_at | hypothetical protein MGC4701 | MGC4701 |
| | 4p16.3 | | |
| 48 | 34252_at | hypothetical protein FLJ10342 | FLJ10342 |
| | 6q16.1 | | |
| 49 | 32638_s_at | PI-3-kinase-related kinase SMG-1 | SMG1 |
| | 16p12.3 | | |
| 50 | 39440_f_at | mRNA (from clone DKFZp566H0124) | |
| 51 | 1467_at | epidermal growth factor receptor substrate | EPS8 |
| | 12q23 | | |
| 52 | 37500_at | zinc finger protein 175 | ZNF175 |
| | 19q13.4 | | |
| 53 | 1307_at | xeroderma pigmentosum, complement group A | XPA |
| | 9q22.3 | | |
| 54 | 1530_g_at | ESP | |
| 55 | 37641_at | ESP | |
| 56 | 36849_at | PTPL1-associated RhoGAP 1 | PARG11 |
| 57 | 38797_at | KIAA0062 protein | KIAA0062 |
| | 8p21.2 | | |
| 58 | 40510_at | heparan sulfate 2-O-sulfotransferase | HS2ST1 |
| | 1p31.1 | | |
| 59 | 34168_at | deoxynucleotidyltransferase, terminal | DNTT |
| | 10q23-q24 | | |
| 60 | 36682_at | pericentriolar material 1 | PCM1 |
| | 8p22-p21.3 | | |
| 61 | 34335_at | ephrin-B2 | EFNB2 |
| | 13q33 | | |
| 62 | 41028_at | ryanodine receptor 3 | RYR3 |
| | 15q14-q15 | | |
| 63 | 31434_at | Homo sapiens aconitase precursor (ACON) mRNA, | |
| | | nuclear gene encoding mitochondrial, partial cds | |
| 64 | 35293_at | Sjogren syndrome antigen A2 | SSA2 |
| | 1q31 | | |
| 65 | 32987_at | FSH primary response (LRPR1, rat) homolog 1 | FSHPRH1 |
| | Xq22 | | |
| 66 | 34731_at | KIAA0185 protein | KIAA0185 |
| | 10q24 | | |
| 67 | 35102_at | zinc finger protein | ZFP |
| | 3p22.3 | | |
| 68 | 35664_at | multimerin | MMRN |
| | 4q22 | | |
| 69 | 32461_f_at | zinc finger protein 81 (HFZ20) | ZNF81 |
| | Xp22.1 | | |
| 70 | 37864_s_at | immunoglobulin heavy constant gamma 3 | IGHG3 |
| | 14q32 | | |
| 71 | 37282_at | MAD2 (mitotic arrest deficient, yeast)-like 1 | MAD2L1 |
| | 4q27 | | |
| 72 | 38407_r_at | prostaglandin D2 synthase (21 kD, brain) | PTGDS |
| | 9q34.2-q34.3 | | |
| 73 | 873_at | homeo box A5 | HOXA5 |
| | 7p15-p14 | | |
| 74 | 36539_at | Homo sapiens cDNA FLJ32313 fis, clone PROST | |
| | | 2003232, weakly similar to BETA- | |
| | | GLUCURONIDASE PRECURSOR (EC 3.2.1.31) | |
| 75 | 37602_at | guanidinoacetate N-methyltransferase | GAMT |
| | 19p13.3 | | |
| 76 | 38821_at | progesterone receptor membrane component 2 | PGRMC2 |
| | 4q26 | | |
| 77 | 36248_at | NAG-5 protein | NAG5 |
| | 9p12 | | |
| 78 | 33796_at | ADP-ribosylation factor-like 4 | ARL4 |
| | 7p21 | | |
| 79 | 37760_at | BAI1-associated protein 2 | BAIAP2 |
| | 17q25 | | |
| 80 | 35299_at | MAP kinase-interacting serine/threonine kinase 1 | MKNK1 |
| | 1p33 | | |

TABLE 55
Discriminating genes that distinguish between remission and fail, inside the AML type,
derived from SVM analysis.

| | Affymetrix Locus | | Gene |
|---|---|---|---|
| | number | Gene description | symbol |
| 1 | 32789_at | nuclear cap binding protein subunit 2, 20 kD | NCBP2 |
| | 3q29 | | |
| 2 | 39175_at | phosphofructokinase, platelet | PFKP |
| | 10p15.3 | | |
| 3 | 41058_g_at | uncharacterized hypothalamus protein HT012 | HT012 |
| | 6p22.2 | | |
| 4 | 38299_at | interleukin 6 (interferon, beta 2) | IL6 |
| | 7p21 | | |
| 5 | 41475_at | ninjurin 1 | NINJ1 |
| | 9q22 | | |
| 6 | 38389_at | 2′,5′-oligoadenylate synthetase 1 (40-46 kD) | OAS1 |
| | 12q24.1 | | |
| 7 | 35803_at | ras homolog gene family, member E | ARHE |
| | 2q23.3 | | |
| 8 | 36419_at | phospholipase C, beta 3 | PLCB3 |
| | 11q13 | | |
| 9 | 32067_at | cAMP responsive element modulator | CREM |
| | 10p12.1 | | |
| 10 | 39924_at | KIAA0853 protein | KIAA0853 |
| | 13q14 | | |
| 11 | 39246_at | stromal antigen 1 | STAG1 |
| | 3q22.3 | | |
| 12 | 38252_s_at | glycogen debranching enzyme (disease type III) | AGL |
| | 1p21 | | |
| 13 | 35127_at | H2A histone family, member A | H2AFA |
| | 6p22.2 | | |
| 14 | 35486_at | Vertebrate LIN7, Tax interaction protein 33 | VELI1 |
| | 12q21 | | |
| 15 | 1368_at | interleukin 1 receptor, type I | IL1R1 |
| | 2q12 | | |
| 16 | 40635_at | flotillin 1 | FLOT1 |
| | 6p21.3 | | |
| 17 | 1679_at | postmeiotic segregation increased 2-like 6 | PMS2L6 |
| | 7q11 | | |
| 18 | 37354_at | nuclear antigen Sp100 | SP100 |
| | 2q37.1 | | |
| 19 | 1065_at | fms-related tyrosine kinase 3 | FLT3 |
| | 13q12 | | |
| 20 | 41470_at | prominin (mouse)-like 1 | PROML1 |
| | 4p15.33 | | |
| 21 | 37483_at | histone deacetylase 9 | HDAC9- |
| | 7p21p15 | | |
| 22 | 34363_at | selenoprotein P, plasma, 1 | SEPP1 |
| | 5q31 | | |
| 23 | 34631_at | eyes absent (Drosophila) homolog 4 | EYA4 |
| | 6q23 | | |
| 24 | 33124_at | vaccinia related kinase 2 | VRK2 |
| | 2p16 | | |
| 25 | 39931_at | dual-specificity tyrosine-(Y)-kinase 3 | DYRK3 |
| | 1q32 | | |
| 26 | 37185_at | serine (or cysteine) proteinase inhibitor | SERPINB |
| | 18q21.3 | | |
| 27 | 717_at | GS3955 protein | GS3955 |
| | 2p25.1 | | |
| 28 | 40305_r_at | phosphatidylinositol glycan, class K | PIGK |
| | 1p31.1 | | |
| 29 | 32636_f_at | PI-3-kinase-related kinase SMG-1 | SMG1 |
| | 16p12.3 | | |
| 30 | 38052_at | coagulation factor XIII, A1 polypeptide | F13A1 |
| | 6p25.3-p24.3 | | |
| 31 | 772_at | v-crk avian sarcoma virus oncogene homolog | CRK |
| | 17p13.3 | | |
| 32 | 41362_at | ATP-binding cassette, sub-family G (WHITE) | ABCG1 |
| | 21q22.3 | | |
| 33 | 36849_at | PTPL1-associated RhoGAP 1 | PARG1 1 |
| 34 | 1451_s_at | osteoblast specific factor 2 (fasciclin I-like) | OSF-2 |
| | 13q13.2 | | |
| 35 | 37547_at | PTH-responsive osteosarcoma B1 protein | B1 |
| | 7p14 | | |
| 36 | 37504_at | E3 ubiquitin ligase SMURF1 | SMURF1 |
| | 7q21.1 | | |
| 37 | 33881_at | fatty-acid-Coenzyme A ligase, long-chain 3 | FACL3 |
| | 2q34 | | |
| 38 | 40439_at | arsA (bacterial) arsenite transporter, ATP-binding | ASNA1 |
| | 19q13.3 | | |
| 39 | 1914_at | cyclin A1 | CCNA1 |
| | 13q12.3 | | |
| 40 | 40928_at | DKFZP564A122 protein | DKFZP |
| | 17q11.2 | | |
| 41 | 36014_at | hypothetical protein DKFZp564D0462 | DKFZP |
| | 6q23.1 | | |
| 42 | 34355_at | methyl CpG binding protein 2 (Rett syndrome) | MECP2 |
| | Xq28 | | |
| 43 | 38096_f_at | MHC, class II, DP beta 1 | DPB1 |
| | 6p21.3 | | |
| 44 | 32298_at | a disintegrin and metalloproteinase domain 2 | ADAM2 |
| | 8p11.2 | | |
| 45 | 35699_at | budding uninhibited by benzimidazoles 1 | BUB1B |
| | 15q15 | | |
| 46 | 41165_g_at | immunoglobulin heavy constant mu | IGHM |
| | 14q32 | | |
| 47 | 35422_at | microtubule-associated protein 2 | MAP2 |
| | 2q34 | | |
| 48 | 41471_at | S100 calcium-binding protein A9 (calgranulin B) | S100A9 |
| | 1q21 | | |
| 49 | 34761_r_at | a disintegrin and metalloproteinase domain 9 | ADAM9 |
| 50 | 31786_at | Sam68-like phosphotyrosine protein, T-STAR | T-STAR |
| | 8q24.2 | | |
| 51 | 40318_at | dynein, cytoplasmic, intermediate polypeptide 1 | DNCI1 |
| | 7q21.3 | | |
| 52 | 40497_at | homologous to yeast nitrogen permease | NPR2L |
| | 3p21.3 | | |
| 53 | 34728_g_at | S-adenosylhomocysteine hydrolase-like 1 | AHCYL1 1 |
| 54 | 36857_at | RAD1 (S. pombe) homolog | RAD1 |
| | 5p13.2 | | |
| 55 | 39449_at | bleomycin hydrolase | BLMH |
| | 17q11.2 | | |
| 56 | 40498_g_at | homologous to yeast nitrogen permease | NPR2L |
| | 3p21.3 | | |
| 57 | 37936_at | PRP4/STK/WD splicing factor | HPRP4P |
| | 9q31 | | |
| 58 | 34891_at | dynein, cytoplasmic, light polypeptide | PIN |
| | 14q24 | | |
| 59 | 39061_at | bone marrow stromal cell antigen 2 | BST2 |
| | 19p13.2 | | |
| 60 | 34446_at | KIAA0471 gene product | KIAA0471 |
| | 1q24 | | |
| 61 | 37456_at | serum constituent protein | MSE55 |
| | 22q13.1 | | |
| 62 | 41385_at | erythrocyte membrane protein band 4.1-like 3 | EPB41L3 |
| | 18p11 | | |
| 63 | 990_at | fms-related tyrosine kinase 1 (vascular endothelial | FLT1 |
| | 13q12 | growth factor/vascular permeability factor receptor) | |
| 64 | 37203_at | carboxylesterase 1 | CES1 |
| | 16q13 | | |
| 65 | 40071_at | cytochrome P450, subfamily I | CYP1B1 |
| | 2p21 | | |
| 66 | 1491_at | pentaxin-related gene, induced by IL-1 beta | PTX3 |
| | 3q25 | | |
| 67 | 31558_at | Hr44 antigen | HR44 |
| 68 | 761_g_at | dual-specificity tyrosine-(Y)-phosphorylation | DYRK2 |
| | 12q14.3 | regulated kinase 2 | |
| 69 | 32607_at | brain abundant, membrane signal protein 1 | BASP1 |
| | 5p15.1 | | |
| 70 | 32305_at | collagen, type I, alpha 2 | COL1A2 |
| | 7q22.1 | | |
| 71 | 531_at | glioma pathogenesis-related protein | RTVP1 |
| | 12q15 | | |
| 72 | 40901_at | nuclear autoantigen | GS2NA |
| | 14q13 | | |
| 73 | 35609_at | protocadherin gamma subfamily A, 8 | PCDHGA8 |
| | 5q31 | | |
| 74 | 40851_r_at | Sec23 (S. cerevisiae) homolog B | SEC23B |
| | 20p11 | | |
| 75 | 41022_r_at | glycerol-3-phosphate dehydrogenase 2 | GPD2 |
| | 2q24.1 | | |
| 76 | 40853_at | ATPase, Class V, type 10D | ATP10D |
| | 4p12 | | |
| 77 | 38555_at | dual specificity phosphatase 10 | DUSP10 |
| | 1q41 | | |
| 78 | 41393_at | zinc finger protein 195 | ZNF195 |
| | 11p15.5 | | |
| 79 | 32089_at | sperm associated antigen 6 | SPAG6 |
| | 10p12 | | |
| 80 | 32072_at | mesothelin | MSLN |
| | 16p13.3 | | |
| 81 | 394_at | S-phase kinase-associated protein 2 (p45) | SKP2 |
| | 5p13 | | |
| 82 | 32605_r_at | RAB1, member RAS oncogene family | RAB1 |
| | 2p14 | | |
| 83 | 31665_s_at | CDA02 protein | CDA02 |
| | 3q24 | | |
| 84 | 35940_at | POU domain, class 4, transcription factor 1 | POU4F1 |
| | 13q21.1 | | |
| 85 | 37469_at | Rough Deal (Drosophila) homolog | KIAA0166 |
| | 12q24 | | |
| 86 | 32599_at | tuberous sclerosis 1 | TSC1 |
| | 9q34 | | |
| 87 | 33894_at | neuroepithelial cell transforming gene 1 | NET1 |
| | 10p15 | | |

TABLE 56

| | | |
|---|---|---|
| Affymetrix Locus | | Gene |
| number | Gene description | symbol |

Discriminating genes that distinguish between remission and fail, inside the VxInsight
cluster A, derived from Bayesian Networks and SVM analysis.
A. Bayesian Networks

| | | | |
|---|---|---|---|
| 1 | 1247_g_at | protein tyrosine phosphatase, receptor type, S | PTPRS |
| | 19p13.3 | | |
| 2 | 128_at | cathepsin K (pycnodysostosis) | CTSK |
| | 1q21 | | |
| 3 | 1445_at | chemokine (C—C motif) receptor-like 2 | CCRL2 |
| | 3p21 | | |
| 4 | 1509_at | matrix metalloproteinase 16 (membrane-inserted) | MMP16 |
| | 8q21 | | |
| 5 | 1523_g_at | tyrosine kinase, non-receptor, 1 | TNK1 |
| | 17p13.1 | | |
| 6 | 1578_g_at | androgen receptor (dihydrotestosterone receptor; | AR |
| | Xq11.2-q12 | testicular feminization; spinal and bulbar muscular | |
| | | atrophy; Kennedy disease) | |
| 7 | 158_at | DnaJ (Hsp40) homolog, subfamily B, member 4 | DNAJB4 |
| | 1p22.3 | | |
| 8 | 1777_at | ras inhibitor | RIN1 |
| | 11q13.1 | | |
| 9 | 31375_at | ADP-ribosylation factor-like 3 | ARL3 |
| | 10q23.3 | | |
| 10 | 31440_at | transcription factor 7 (T-cell specific, HMG-box) | TCF7 |
| | 5q31.1 | | |
| 11 | 31552_at | Homo sapiens low density lipoprotein receptor | |
| 12 | 31713_s_at | large (Drosophila) homolog-associated protein 2 | DLGAP2 |
| | 8p23 | | |
| 13 | 31996_at | brefeldin A-inhibited guanine nucleotide-exchange 2 | BIG2 |
| | 20q13 | | |
| 14 | 32029_at | 3-phosphoinositide dependent protein kinase-1 | PDPK1 |
| | 16p13.3 | | |
| 15 | 32823_at | vacuolar protein sorting 11 (yeast homolog) | VPS11 |
| | 11q23 | | |
| 16 | 32903_at | transforming growth factor, beta receptor I | TGFBR1 |
| | 9q22 | | |
| 17 | 33019_at | Parkinson disease (autosomal recessive, juvenile) | PARK2 |
| | 6q25.2 | | |
| 18 | 33280_r_at | SA (rat hypertension-associated) homolog | SAH |
| | 16p13.11 | | |
| 19 | 34110_g_at | proline oxidase homolog | PIG6 |
| 20 | 34124_at | similar to prokaryotic-type class I peptide chain | LOC16 |
| | 6q25 | release factors | |
| 21 | 34181_at | aspartylglucosaminidase | AGA |
| | 4q32 | | |
| 22 | 35044_i_at | bone morphogenetic protein 8 (osteogenic 2) | BMP8 |
| | 1p35 | | |
| 23 | 35375_at | apurinic/apyrimidinic endonuclease(nuclease) | APEXL2 |
| | Xp11.23 | | |
| 24 | 35942_at | GA-binding protein transcription factor, beta 1 | GABPB1 |
| | 7q11.2 | | |

Discriminating genes that distinguish between remission and fail, inside the VxInsight
cluster A, derived from SVM analysis.
B. SVM

| | | | |
|---|---|---|---|
| 1 | 39389_at | CD9 antigen (p24) | CD9 |
| | 12p13.3 | | |
| 2 | 1292_at | dual specificity phosphatase 2 | DUSP2 |
| | 2q11 | | |
| 3 | 36674_at | small inducible cytokine A4 | SCYA4 |
| | 17q12 | | |
| 4 | 32637_r_at | PI-3-kinase-related kinase SMG-1 | SMG1 |
| | 16p13.2 | | |
| 5 | 35756_at | regulator of G-protein signalling 19 interacting | RGS19IP1 |
| | 19p13.1 | | |
| 6 | 41700_at | coagulation factor II (thrombin) receptor | F2R |
| | 5q13 | | |
| 7 | 31853_at | embryonic ectoderm development | EED |
| | 11q14 | | |
| 8 | 31329_at | Human putative opioid receptor mRNA, complete | |
| 9 | 34491_at | 2′-5′-oligoadenylate synthetase-like | OASL |
| | 12q24.2 | | |
| 10 | 34961_at | T cell activation, increased late expression | TACTILE |
| | 3q13.2 | | |
| 11 | 160021_r_at | progesterone receptor | PGR |
| | 11q22-q23 | | |
| 12 | 38367_s_at | complement component 4 binding protein, beta | C4BPB |
| | 1q32 | | |
| 13 | 32279_at | glutamate decarboxylase 2 (pancreas and brain) | GAD2 |
| | 10p11.23 | | |
| 14 | 36108_at | MHC, class II, DQ beta 1 | DQB1 |
| | 6p21.3 | | |
| 15 | 34378_at | adipose differentiation-related protein | ADFP |
| | 9p21.2 | | |
| 16 | 777_at | GOP dissociation inhibitor 2 | GDI2 |
| | 10p15 | | |
| 17 | 35140_at | cyclin-dependent kinase 8 | CDK8 |
| | 13q12 | | |
| 18 | 33208_at | DnaJ (Hsp40) homolog, subfamily C, member 3 | DNAJC3 |
| | 13q32 | | |
| 19 | 33405_at | adenylyl cyclase-associated protein 2 | CAP2 |
| | 6p22.2 | | |
| 20 | 39580_at | KIAA0649 gene product | KIAA0649 |
| | 9q34.3 | | |
| 21 | 32469_at | carcinoembryonic antigen-related cell adhesion | CEACAM |
| | 19q13.2 | | |
| 22 | 38539_at | solute carrier family 24 | SLC24A1 |
| | 15q22 | | |
| 23 | 33739_at | Homo sapiens mRNA full length insert cDNA | |
| 24 | 1454_at | MAD, mothers against decapentaplegic 3 | MADH3 |
| | 15q21-q22 | | |
| 25 | 35289_at | rab6 GTPase activating protein | CENA |
| | 9q34.11 | | |
| 26 | 37724_at | v-myc myelocytomatosis viral oncogene homolog | MYC |
| | 8q24.12 | | |
| 27 | 32521_at | secreted frizzled-related protein 1 | SFRP1 |
| | 8p12-p11.1 | | |
| 28 | 1375_s_at | tissue inhibitor of metalloproteinase 2 | TIMP2 |
| | 17q25 | | |
| 29 | 615_s_at | parathyroid hormone-like hormone | PTHLH |
| | 12p12.1 | | |
| 30 | 555_at | RAB40B, member RAS oncogene family | RAB40B |
| | 17q25.3 | | |
| 31 | 224_at | TGFB inducible early growth response | TIEG |
| | 8q22.2 | | |
| 32 | 40367_at | bone morphogenetic protein 2 | BMP2 |
| | 20p12 | | |
| 33 | 37380_at | general transcription factor IIB | GTF2B |
| | 1p22-p21 | | |
| 34 | 41504_s_at | v-maf aponeurotic fibrosarcoma oncogene | MAF |
| | 16q22-q23 | | |
| 35 | 40166_at | CS box-containing WD protein | LOC55 |
| 36 | 35228_at | carnitine palmitoyltransferase I, muscle | CPT1B |
| | 22q13.33 | | |
| 37 | 36113_s_at | troponin T1, skeletal, slow | TNNT1 |
| | 19q13.4 | | |
| 38 | 33491_at | sucrase-isomaltase | SI |
| | 3q25.2 | | |
| 39 | 1182_at | phospholipase C-like 1 | PLCL1 |
| | 2q33 | | |
| 40 | 38869_at | KIAA1069 protein | KIAA1069 |
| | 3q26.1 | | |
| 41 | 35811_at | ring finger protein 13 | RNF13 |
| | 3q25.1 | | |
| 42 | 33186_i_at | ESTs | |
| 43 | 37504_at | E3 ubiquitin ligase SMURF1 | SMURF1 |
| | 7q21.1 | | |
| 44 | 160025_at | transforming growth factor, alpha | TGFA |
| | 2p13 | | |

Discriminating genes that distinguish between remission and fail, inside the VxInsight
cluster A, derived from SVM analysis.

| | | | |
|---|---|---|---|
| 45 | 32684_at | Homo sapiens clone 23579 mRNA sequence | |
| 46 | 35233_r_at | centrin, EF-hand protein, 3 (CDC31 homolog) | CETN3 |
| | 5q14.3 | | |
| 47 | 40399_r_at | mesenchyme homeo box 2 (growth arrest) | MEOX2 |
| | 7p22.1 | | |
| 48 | 36777_at | DNA segment on chromosome 12 (unique) 2489 | D12S |
| | 12p13.2 | | |
| 49 | 31810_g_at | contactin 1 | CNTN1 |
| | 12q11-q12 | | |
| 50 | 33747_s_at | RNA, U17D small nucleolar | RNU17D |
| | 1p36.1 | | |
| 51 | 37577_at | hypothetical protein MGC14258 | MGC |
| | 10q24.2 | | |
| 52 | 40789_at | adenylate kinase 2 | AK2 |
| | 1p34 | | |
| 53 | 34855_at | hypothetical protein MGC5378 | MGC5378 |
| | 14q32.31 | | |
| 54 | 35614_at | transcription factor-like 5 (basic helix-loop-helix) | TCFL5 |
| | 20q13.3 | | |
| 55 | 34482_at | hypothetical protein MGC4701 | MGC4701 |
| | 4p16.3 | | |
| 56 | 37220_at | Fc fragment of lgG, receptor for - CD64 | FCGR1A |
| | 1q21.2 | | |
| 57 | 36444_s_at | small inducible cytokine subfamily A | SCYA23 |
| | 17q21.1 | | |
| 58 | 34252_at | hypothetical protein FLJ10342 | FLJ10342 |
| | 6q16.1 | | |
| 59 | 32638_s_at | PI-3-kinase-related kinase SMG-1 | SMG1 |
| | 16p13.2 | | |
| 60 | 1467_at | epidermal growth factor receptor 8 | EPS8 |
| | 12q23-q24 | | |
| 61 | 37500_at | zinc finger protein 175 | ZNF175 |
| | 19q13.4 | | |
| 62 | 1307_at | xeroderma pigmentosum, complement group A | XPA |
| | 9q22.3 | | |
| 63 | 1530_g_at | hypothetical protein CG003 | 13CDNA |
| | 13q12.3 | | |
| 64 | 37641_at | interferon-induced protein 44 | IFI44 |
| | 1p31.1 | | |
| 65 | 36849_at | PTPL1-associated RhoGAP 1 | PARG1 |
| | 1p22.1 | | |
| 66 | 38797_at | KIAA0062 protein | KIAA0062 |
| | 8p21.2 | | |
| 67 | 40510_at | heparan sulfate 2-O-sulfotransferase 1 | HS2ST1 |
| | 1p31.1 | | |
| 68 | 34168_at | deoxynucleotidyltransferase, terminal | DNTT |
| | 10q23-q24 | | |
| 69 | 36682_at | pericentriolar material 1 | PCM1 |
| | 8p22-p21.3 | | |
| 70 | 34335_at | ephrin-B2 | EFNB2 |
| | 13q33 | | |
| 71 | 40549_at | cyclin-dependent kinase 5 | CDK5 |
| | 7q36 | | |
| 72 | 41028_at | ryanodine receptor 3 | RYR3 |
| | 15q14-q15 | | |
| 73 | 31434_at | Homo sapiens aconitase precursor (ACON) | |
| 74 | 33031_at | Homo sapiens mRNA full length insert cDNA clone | |
| 75 | 35293_at | Sjogren syndrome antigen A2 (60 kD) | SSA2 |
| | 1q31 | | |
| 76 | 32987_at | FSH primary response (LRPR1 homolog, rat) 1 | FSHPRH1 |
| | Xq22 | | |
| 77 | 34731_at | KIAA0185 protein | KIAA0185 |
| | 10q25.1 | | |
| 78 | 35102_at | zinc finger protein | ZFP |
| | 3p22.3 | | |
| 79 | 35664_at | multimerin | MMRN |
| | 4q22 | | |
| 80 | 34208_at | solute carrier family 12, member 5 | SLC12A5 |
| | 20q13.12 | | |
| 81 | 37864_s_at | immunoglobulin heavy constant gamma 3 | IGHG3 |
| | 14q32.33 | | |
| 82 | 37282_at | MAD2 mitotic arrest deficient-like 1 (yeast) | MAD2L1 |
| | 4q27 | | |
| 83 | 38407_r_at | prostaglandin D2 synthase (21 kD, brain) | PTGDS |
| | 9q34.2 | | |
| 84 | 37602_at | guanidinoacetate N-methyltransferase | GAMT |
| | 19p13.3 | | |
| 85 | 38821_at | progesterone receptor membrane component 2 | PGRMC2 |
| | 4q26 | | |
| 86 | 36248_at | NAG-5 protein | NAG5 |
| | 9p11.2 | | |
| 87 | 33796_at | epithelial protein lost in neoplasm beta | EPLIN |
| | 12q13 | | |
| 88 | 37760_at | BAI1-associated protein 2 | BAIAP2 |
| | 17q25 | | |
| 89 | 35299_at | MAP kinase-interacting serine/threonine kinase 1 | MKNK1 |
| | 1p34.1 | | |

TABLE 57

| | | |
|---|---|---|
| Affymetrix | | |
| Locus | | Gene |
| number | Gene description | symbol |

Discriminating genes that distinguish between remission and fail,
inside the VxInsight cluster C, derived from Bayesian Networks and SVM
analysis.
A. Bayesian Networks

| | | | |
|---|---|---|---|
| 1 | 111_at | Rab geranylgeranyltransferase, alpha subunit | RAB |
| | 14q11.2 | | |
| 3 | 1274_s_at | cell division cycle 34 | CDC34 |
| | 19p13.3 | | |
| 4 | 1561_at | dual specificity phosphatase 8 | DUSP8 |
| | 11p15.5 | | |
| 6 | 31405_at | melatonin receptor 1B | MTNR1B |
| | 11q21-q22 | | |
| 7 | 31803_at | KIAA0653 protein, B7-like protein | KIAA0653 |
| | 21q22.3 | | |
| 8 | 32334_f_at | ubiquitin C | UBC |
| | 12q24.3 | | |
| 9 | 32892_at | ribosomal protein S6 kinase, 90 kD | RPS6KA2 |
| | 6q27 | | |
| 10 | 33095_i_at | beaded filament structural protein 2, phakinin | BFSP2 |
| | 3q21-q25 | | |
| 11 | 33293_at | lifeguard | KIAA0950 |
| | 12q13 | | |
| 12 | 34913_at | calcium channel, voltage-dependent, L type | CACNA1S |
| | 1q32 | | |
| 13 | 35957_at | stannin | SNN |
| | 16p13 | | |
| 14 | 36038_r_at | spectrin, beta, erythrocytic | SPTB |
| | 14q23 | | |
| 15 | 36342_r_at | H factor (complement)-like 3 | HFL3 |
| | 1q31-q32.1 | | |
| 16 | 37596_at | phospholipase C, delta 1 | PLCD1 |
| | 3p22-p21.3 | | |
| 17 | 38299_at | interleukin 6 (interferon, beta 2) | IL6 |
| | 7p21 | | |
| 18 | 41520_at | hypothetical protein | LOC56148 |
| 19 | 772_at | v-crk avian sarcoma virus CT10 oncogene | CRK |
| | 17p13.3 | | |
| 20 | 1001_at | tyrosine kinase with immunoglobulin and | TIE |
| | 1p34-p33 | epidermal growth factor homology domains | |
| 21 | 1707_g_at | v-raf murine sarcoma viral oncogene homolog | ARAF1 |
| | Xp11.4-p11.2 | | |
| 22 | 1719_at | mutS (E. coli) homolog 3 | MSH3 |
| | 5q11-q12 | | |
| 23 | 1962_at | arginase, liver | ARG1 |
| | 6q23 | | |
| 24 | 2034_s_at | cyclin-dependent kinase inhibitor 1B | CDKN1B |
| | 12p13.1 | | |
| 25 | 31505_at | ribosomal protein L8 | RPL8 |
| | 8q24.3 | | |

Discriminating genes that distinguish between
remission and fail, inside the VxInsight cluter C, derived from SVM analysis.
B. SVM

| | | | |
|---|---|---|---|
| 1 | 914_g_at | v-ets erythroblastosis virus E26 oncogene like | ERG |
| | 21q22.3 | | |
| 2 | 32789_at | nuclear cap binding protein subunit 2, 20 kD | NCBP2 |
| | 3q29 | | |
| 3 | 38299_at | interleukin 6 (interferon, beta 2) | IL6 |
| | 7p21 | | |
| 4 | 39175_at | phosphofructokinase, platelet | PFKP |
| | 10p15.3 | | |
| 5 | 1368_at | interleukin 1 receptor, type I | IL1R1 |
| | 2q12 | | |
| 6 | 41219_at | Homo sapiens mRNA; cDNA DKFZp586J101 | |
| 7 | 38389_at | 2′,5′-oligoadenylate synthetase 1 (40-46 kD) | OAS1 |
| | 12q24.1 | | |
| 8 | 32067_at | cAMP responsive element modulator | CREM |
| | 10p12.1 | | |
| 9 | 41058_g_at | uncharacterized hypothalamus protein HT012 | HT012 |
| | 6p21.32 | | |
| 10 | 41425_at | Friend leukemia virus integration 1 | FLI1 |
| | 11q24.1 | | |
| 11 | 33124_at | vaccinia related kinase 2 | VRK2 |
| | 2p16-p15 | | |
| 12 | 41475_at | ninjurin 1 | NINJ1 |
| | 9q22 | | |
| 13 | 38866_at | EST | |
| 14 | 35803_at | ras homolog gene family, member E | ARHE |
| | 2q23.3 | | |
| 15 | 41096_at | S100 calcium binding protein A8 (calgranulin A) | S100A8 |
| | 1q21 | | |
| 16 | 33800_at | adenylate cyclase 9 | ADCY9 |
| | 16p13.3 | | |
| 17 | 37143_s_at | phosphoribosylformylglycinamidine synthase | PFAS |
| | 17p13 | | |
| 18 | 37535_at | cAMP responsive element binding protein 1 | CREB1 |
| | 2q32.3-q34 | | |
| 19 | 38253_at | amylo-1, 6-glucosidase, 4-alpha- | AGL |
| | 1p21 | | |
| 20 | 36857_at | RAD1 homolog (S. pombe) | RAD1 |
| | 5p13.2 | | |
| 21 | 39931_at | dual-specificity tyrosine-(Y)-phosphorylation | DYRK3 |
| | 1q32 | regulated kinase 3 | |
| 22 | 772_at | v-crk sarcoma virus CT10 oncogene homolog | CRK |
| | 17p13.3 | | |
| 23 | 35957_at | stannin | SNN |
| | 16p13 | | |
| 24 | 41755_at | KIAA0977 protein | KIAA0977 |
| | 2q24.3 | | |
| 25 | 31786_at | RNA binding, signal transduction associated 3 | KHDRBS3 |
| | 8q24.2 | | |
| 26 | 35127_at | H2A histone family, member A | H2AFA |
| | 6p22. | | |
| 27 | 40928_at | SOCS box-containing WD protein SWiP-1 | WSB1 |
| | 17q11.1 | | |
| 28 | 32636_f_at | PI-3-kinase-related kinase SMG-1 | SMG1 |
| | 16p13.2 | | |
| 29 | 531_at | glioma pathogenesis-related protein | RTVP1 |
| | 12q14.1 | | |
| 30 | 35860_r_at | ESTs | |
| 31 | 41471_at | S100 calcium binding protein A9 (calgranulin B) | S100A9 |
| | 1q21 | | |
| 32 | 35582_at | ESTs | |
| 33 | 39878_at | protocadherin 9 | PCDH9 |
| | 13q14.3 | | |
| 34 | 37504_at | E3 ubiquitin ligase SMURF1 | SMURF1 |
| | 7q21.1 | | |
| 33 | 34965_at | cystatin F (leukocystatin) | CST7 |
| | 20p11.21 | | |
| 34 | 37050_r_at | translocase of outer mitochondrial membrane 34 | TOMM34 |
| 35 | 32034_at | zinc finger protein 217 | ZNF217 |
| | 20q13.2 | | |
| 36 | 33104_at | PH domain containing protein in retina 1 | PHRET1 |
| | 11q13.5 | | |
| 37 | 40318_at | dynein, cytoplasmic, intermediate polypeptide 1 | DNCI1 |
| | 7q21.3 | | |
| 38 | 34387_at | KIAA0205 gene product | KIAA0205 |
| | 1p36.13 | | |
| 39 | 37208_at | phosphoserine phosphatase-like | PSPHL |
| | 7q11.2 | | |
| 40 | 38139_at | fucose-1-phosphate guanylyltransferase | FPGT |
| | 1p31.1 | | |
| 41 | 1914_at | cyclin A1 | CCNA1 |
| | 13q12.3 | | |
| 42 | 717_at | GS3955 protein | GS3955 |
| | 2p25.1 | | |
| 43 | 36123_at | thiosulfate sulfurtransferase (rhodanese) | TST |
| | 22q13.1 | | |
| 44 | 33881_at | fatty-acid-Coenzyme A ligase, long-chain 3 | FACL3 |
| | 2q34-q35 | | |
| 45 | 35606_at | histidine decarboxylase | HDC |
| | 15q21-q22 | | |
| 46 | 36478_at | transcription termination factor, RNA polymerase I | TTF1 |
| | 9q34.3 | | |
| 47 | 34363_at | selenoprotein P, plasma, 1 | SEPP1 |
| | 5q31 | | |
| 48 | 34631_at | eyes absent homolog 4 (Drosophila) | EYA4 |
| | 6q23 | | |
| 49 | 37773_at | KIAA1005 protein | KIAA1005 |
| | 16q12.2 | | |
| 50 | 1451_s_at | osteoblast specific factor 2 (fasciclin I-like) | OSF-2 |
| | 13q13.2 | | |
| 51 | 40635_at | flotillin 1 | FLOT1 |
| | 6p21.3 | | |
| 52 | 34961_at | T cell activation, increased late expression | TACTILE |
| | 3q13.2 | | |
| 53 | 32637_r_at | PI-3-kinase-related kinase SMG-1 | SMG1 |
| | 16p13.2 | | |
| 54 | 1808_s_at | tumor necrosis factor receptor superfamily, 6 | TNFRSF6 |
| | 10q24.1 | | |
| 55 | 1369_s_at | interleukin 8 | IL8 |
| | 4q13-q21 | | |
| 56 | 35614_at | transcription factor-like 5 (basic helix-loop-helix) | TCFL5 |
| | 20q13.3 | | |
| 57 | 40511_at | GATA binding protein 3 | GATA3 |
| | 10p15 | | |
| 58 | 1229_at | cisplatin resistance associated | CRA |
| | 1q12-q21 | | |
| 59 | 34247_at | protease, serine, 12 (neurotrypsin, motopsin) | PRSS12 |
| | 4q25-q26 | | |
| 60 | 35980_at | phospholipase C, beta 1 | PLCB1 |
| | 20p12 | | |
| 61 | 33715_r_at | general transcription factor IIH, polypeptide 2 | GTF2H2 |
| | 5q12.2 | | |
| 62 | 852_at | integrin, beta 3 | ITGB3 |
| | 17q21.32 | | |
| 63 | 1913_at | cyclin G2 | CCNG2 |
| | 4q13.3 | | |
| 64 | 36569_at | tetranectin (plasminogen binding protein) | TNA |
| | 3p22-p21.3 | | |
| 65 | 41708_at | KIAA1034 protein | KIAA1034 |
| | 2q33 | | |
| 66 | 41348_at | iroquois homeobox protein 5 | IRX5 |
| | 16q11.2 | | |
| 67 | 38952_s_at | collagen, type XIII, alpha 1 | COL13A1 |
| | 10q22 | | |
| 68 | 33553_r_at | chemokine (C-C motif) receptor 6 | CCR6 |
| | 6q27 | | |
| 69 | 41165_g_at | immunoglobulin heavy constant mu | IGHM |
| | 14q32.33 | | |
| 70 | 34435_at | aquaporin 9 | AQP9 |
| | 15q22.1 | | |
| 71 | 1679_at | postmeiotic segregation increased 2-like 6 | PMS2L6 |
| | 7q11-q22 | | |
| 72 | 41742_s_at | optineurin | OPTN |
| | 10p12.33 | | |
| 73 | 36998_s_at | spinocerebellar ataxia 2 | SCA2 |
| | 12q24 | | |
| 74 | 39032_at | transforming growth factor beta-stimulated protein | TSC22 |
| | 13q14 | | |
| 75 | 1065_at | fms-related tyrosine kinase 3 | FLT3 |
| | 13q12 | | |
| 76 | 40584_at | nucleoporin 88 kD | NUP88 |
| | 17p13 | | |
| 77 | 41470_at | prominin-like 1 (mouse) | PROML1 |
| | 4p15.33 | | |
| 78 | 38470_i_at | amyloid beta precursor protein | APPBP2 |
| | 17q21-q23 | | |
| 79 | 37676_at | phosphodiesterase 8A | PDE8A |
| | 15q25.1 | | |
| 80 | 35449_at | killer cell lectin-like receptor B, member 1 | KLRB1 |
| | 12p13 | | |
| 81 | 36474_at | KIAA0776 protein | KIAA0776 |
| | 6q16.3 | | |
| 82 | 32142_at | serine/threonine kinase 3 (STE20 homolog, yeast) | STK3 |
| | 8q22.1 | | |
| 83 | 39299_at | KIAA0971 protein | KIAA0971 |
| | 2q33.3 | | |
| 84 | 38252_s_at | 1,6-glucosidase, 4-alpha-glucanotransferase | AGL |
| | 1p21 | | |
| 85 | 39246_at | stromal antigen 1 | STAG1 |
| | 3q22.3 | | |
| 86 | 160030_at | growth hormone receptor | GHR |
| | 5p13-p12 | | |
| 87 | 33736_at | stomatin (EBP72)-like 1 | STOML1 |
| | 15q24-q25 | | |
| 88 | 36014_at | hypothetical protein DKFZp564D0462 | DKFZP56 |
| | 6q23.1 | | |
| 89 | 32072_at | mesothelin | MSLN |
| | 16p13.12 | | |

6. Additional Explorations on VxInsight Clustering Results with the Genetic Algorithm K-Nearest Neighbor Method (GA/KNN).

As it was previously mentioned, the VxInsight clustering algorithm identified three major groups, A, B, and C, in the infant leukemia dataset. We hypothesized these groups correspond to distinct biologic clusters, correlated with unique disease etiologies. Several approaches were used to evaluate cluster stability and to determine genes that discriminate between the clusters. In order to test how well these three clusters can be distinguished using supervised classification and cross-validation methods (49) we used a genetic algorithm training methodology to perform feature selection using a simple K-nearest neighbor classifier (50, 51). This approach was applied using VxInsight cluster train/test class labels, creating three implied one-vs.-all classification problems (A vs. B+C, etc.) The “top 50” discriminating gene lists are reported for each problem, and compared with previously obtained ANOVA gene lists.

To compare this “top 50” gene lists with the gene lists generated using ANOVA, we used a one-vs-all-others (OVA) approach to form three binary classification problems: a) A vs. BC; b) B vs. CA; c) C vs. AB. Based on our subsequent numerical results (time to solution for the genetic algorithm), Task (a) appears to have been the easiest and Task (b) the hardest. We also did three-way classification for VxInsight groups. It is Task (d).

6.1. GA/KNN Procedure and Parallel Program Parameters

The Genetic Algorithm (GA) K Nearest Neighbor (KNN) method (50, 51) is a supervised feature selection method based on the non-parametric k-nearest neighbor classification approach (52). GA uses a direct analogy of natural behavior and works with a “population” of “chromosomes.” Each chromosome represents a possible solution to a given problem. A chromosome is assigned a fitness score according to how good a solution to the problem it is. Highly fit individuals are given opportunities to “reproduce,” by “cross breeding” with other individuals in the population. This produces new individuals (offspring), which share some features taken from each parent. The least fit members of the population are less likely to get selected for reproduction, and so die out. Selecting the best individuals from the current “generation” and mating them to produce a new set of individuals produce an entirely new population of possible solutions. This new generation contains a higher proportion of the characteristics possessed by the good members of the previous generation. In this way, over many generations, good characteristics are spread throughout the population, being mixed and exchanged with other good characteristics. The fitness of each chromosome is determined by its ability to classify the training set samples according to the KNN procedure. In KNN, each sample was classified according to its k nearest neighbors, using the Euclidean distance metric in d-dimensional space (d is the number of probesets in the expression profile for a given patient sample). In our initial experiments, we have chosen k=3. In consensus rule, if all of the k nearest neighbors of a sample belong to the same class, the sample is classified as that class; otherwise, the sample is considered unclassifiable. In majority rule, if more than half of the k nearest neighbors of a sample belong to the same class, the sample is classified as that class; otherwise, the sample is considered unclassifiable.

The GA/KNN methodology was implemented as a C/MPI parallel program on the LosLobos Linux supercluster. The program terminates when 2000 good solutions have been obtained. Following this initial processing, the frequency with which each probeset was selected was analyzed.

The parameters used were as follows:

Number of independent GA runs: 2000
Number of generations/run: 1000
Number of chromosomes in population: 100
Number of genes in each chromosome: 20
Number of neighbors (k) in KNN: 3
KNN rules: consensus in training; majority in test
Number of parallel compute nodes (2 processors/node): 26
Number of master nodes: 1
Number of slave processes: 50
   6.2. Methods
   1) Select Predictor Probesets
   Using the VxInsight cluster labels, we applied the GA/KNN methodology to select the top 50 discriminating probesets from the initial list of 8446 probesets for each task. Here we used consensus rule.
   2) Compare with VxInsight Cluster-Characterizing Genes
   The VxInsight clustering algorithm identified 126 cluster-characterizing genes for each task according to the F values in ANOVA. The lists include top up-regulated and down-regulated genes. Here we compared them with our predictor probesets.
   3) Evaluate Classifier Performance
   Both leave-one-out cross validation (LOOCV) and evaluation on an independent test set were used to evaluate classifier performance for the VxInsight clusters. Note that we have made no attempt at this stage to optimize—using the training set only, and blinded to the test set the number of features selected for the final out-of-sample test set evaluation. Here LOOCV based on consensus rule and prediction for test dataset based on majority rule.
   4) Statistical Significance Analysis
   The statistical significance of the predictions was calculated. We tested whether the Success Rate (SR) was larger than 0.5 and whether the Odds Ratio (OR=TP/FP) was larger than 1.
   6.3. Results
1) Top gene selections—-Z-score plots were computed from gene selection frequencies in the GA (see (50, 51) for details). A very high Z-score gene “40103_at” was found for cluster B vs. CA and C vs. AB.
2) Top gene lists—Tables 58 (A vs. BC), 59 (B vs. CA) and 60 (C vs. AB) show the overlap with ‘up’- and ‘down’-regulated gene lists in the infant cohort as indicated. The numbers of overlapping genes between the cluster-characterizing genes and our top 50 genes are 20, 17, and 17 for A vs. BC, B vs. CA, and C vs. AB tasks respectively.
   3) Evaluating the Performance of a Classifier

See Table 61. Here pVal1 is p-value of testing whether the SR is larger than 0.5 and pVal2 is p-value of testing whether the OR is larger than 1. Both pVal1s and pVal2s are very small (<<0.05) for our predictions. So they are significant.

4) Classification Results with DIFF Genes

The numbers of DIFF calls are 46, 32, and 36 in top 50 discriminating genes, for A vs. BC, B vs. CA, and C vs. AB respectively. We did classification only based on DIFF genes, for A vs. BC, B vs. CA, and C vs. AB respectively. Unfortunately, no improvement of SRs was observed for test dataset (Table 62).

TABLE 58
Top gene list for Cluster A vs. BC

| | | | | Paper | | 126 | | | |
|---|---|---|---|---|---|---|---|---|---|
| Rank | Affx Num | Gene description | Z-score | List | Rank | Rank | H/L | High % | Low % |
| 1 | 31497_at | G antigen 2 | 180.92 | | | 101 | L | 20.2 | 79.8 |
| 2 | 40539_at | myosin IXB | 134.92 | up | 10 | 30 | L | 14.6 | 85.4 |
| 3 | 31829_r_at | trans-golgi network protein 2 | 86.15 | | | | L | 20.2 | 79.8 |
| 4 | 34573_at | ephrin-A3 | 65.45 | up | 15 | 28 | L | 18.0 | 82.0 |
| 5 | 34415_at | activin A receptor, type IB | 65.45 | | | | L | 18.0 | 82.0 |
| 6 | 34970_r_at | 5-oxoprolinase (ATP-hydrolysing) | 58.09 | | | 85 | L | 18.0 | 82.0 |
| 7 | 1280_i_at | NO_.SIF_seq | 55.33 | | | 35 | L | 19.1 | 80.9 |
| 8 | 39306_at | protease, serine, 16 (thymus) | 52.57 | up | 28 | 25 | L | 16.9 | 83.2 |
| 9 | 41374_at | ribosomal protein S6 kinase, 70 kD, polypeptide 2 | 51.65 | | | 10 | L | 16.9 | 83.2 |
| 10 | 39775_at | serine (or cysteine) proteinase inhibitor, clade G | 45.67 | up | 17 | | L | 24.7 | 75.3 |
| | | (C1 inhibitor), member 1 | | | | | | | |
| 11 | 36276_at | contactin 2 (axonal) | 37.85 | up | 2 | 6 | L | 23.6 | 76.4 |
| 12 | 32104_i_at | calcium/calmodulin-dependent protein kinase (CaM kinase) II gamma | 34.17 | | | 3 | L | 23.6 | 76.4 |
| 13 | 36991_at | splicing factor, arginine/serine-rich 4 | 32.33 | down | 1 | 9 | H | 73.0 | 27.0 |
| 14 | 1925_at | cyclin F | 30.95 | up | 29 | 72 | L | 18.0 | 82.0 |
| 15 | 35571_at | coagulation factor II (thrombin) receptor-like 3 | 28.64 | | | | L | 19.1 | 80.9 |
| 16 | 538_at | CD34 antigen | 28.18 | up | 36 | | L | 36.0 | 64.0 |
| 17 | 34755_at | ADP-ribosyltransferase (NAD+; poly(ADP-ribose) polymerase)-like 2 | 26.34 | | | | L | 20.2 | 79.8 |
| 18 | 33034_at | rhomboid (veinlet, Drosophila)-like | 25.88 | up | 33 | 60 | L | 13.5 | 86.5 |
| 19 | 33338_at | signal transducer and activator of transcription 1, 91 kD | 23.58 | | | | H | 74.2 | 25.8 |
| 20 | 396_f_at | erythropoietin receptor | 21.28 | up | 6 | 12 | L | 23.6 | 76.4 |
| 21 | 34949_at | KIAA1048 protein | 20.36 | | | | L | 25.8 | 74.2 |
| 22 | 31508_at | thioredoxin interacting protein | 19.90 | | | | H | 68.5 | 31.5 |
| 23 | 41101_at | KIAA0274 gene product | 19.44 | | | 99 | L | 27.0 | 73.0 |
| 24 | 884_at | integrin, alpha 3 (antigen CD49C, alpha 3 subunit of VLA-3 receptor) | 17.60 | up | 9 | 16 | L | 29.2 | 70.8 |
| 25 | 838_s_at | ubiquitin-conjugating enzyme E2I (homologous to yeast UBC9) | 17.60 | | | | H | 71.9 | 28.1 |
| 26 | 41749_at | ES1 (zebrafish) protein, human homolog of | 17.14 | | | | H | 69.7 | 30.3 |
| 27 | 33516_at | hemoglobin, delta | 17.14 | | | | L | 31.5 | 68.5 |
| 28 | 41206_r_at | cytochrome c oxidase subunit Vla polypeptide 1 | 16.68 | | | | H | 58.4 | 41.6 |
| 29 | 1357_at | ubiquitin specific protease 4 (proto-oncogene) | 16.68 | down | 11 | 51 | H | 84.3 | 15.7 |
| 30 | 41734_at | KIAA0870 protein | 16.22 | | | | H | 79.8 | 20.2 |
| 31 | 39196_i_at | ortholog of mouse integral membrane glycoprotein LIG-1 | 16.22 | | | | L | 23.6 | 76.4 |
| 32 | 37341_at | glutamate dehydrogenase 1 | 16.22 | | | | L | 24.7 | 75.3 |
| 33 | 41264_at | Homo sapiens mRNA; cDNA DKFZp586F1322 | 15.76 | | | | L | 20.2 | 79.8 |
| | | (from clone DKFZp586F1322) | | | | | | | |
| 34 | 35503_at | 5-hydroxytryptamine (serotonin) receptor 1B | 15.76 | | | | L | 25.8 | 74.2 |
| 35 | 33470_at | KIAA1719 protein | 15.76 | | | 11 | L | 25.8 | 74.2 |
| 36 | 459_s_at | bridging integrator 1 | 15.30 | | | | H | 67.4 | 32.6 |
| 37 | 37203_at | carboxylesterase 1 (monocyte/macrophage serine esterase 1) | 15.30 | | | | H | 61.8 | 38.2 |
| 38 | 1653_at | ribosomal protein S3A | 15.30 | | | | L | 44.9 | 55.1 |
| 39 | 1052_s_at | CCAAT/enhancer binding protein (C/EBP), delta | 15.30 | | | | L | 29.2 | 70.8 |
| 40 | 40830_at | DnaJ (Hsp40) homolog, subfamily C, member 4 | 14.84 | | | | L | 25.8 | 74.2 |
| 41 | 38648_at | trinucleotide repeat containing 1 | 14.84 | | | | H | 67.4 | 32.6 |
| 42 | 32878_f_at | Homo sapiens cDNA FLJ32819 fis, clone TESTI2002937, | 14.38 | | | | H | 84.3 | 15.7 |
| | | weakly similar to HISTONE H3.2 | | | | | | | |
| 43 | 40941_at | VAMP (vesicle-associated membrane protein)-associated | 13.92 | | | | L | 11.2 | 88.8 |
| | | protein B and C | | | | | | | |
| 44 | 38530_at | hypothetical protein FLJ22709 | 13.46 | | | | L | 19.1 | 80.9 |
| 45 | 35355_at | Homo sapiens cDNA FLJ11214 fis, clone PLACE1007990 | 13.46 | | | | H | 64.0 | 36.0 |
| 46 | 40501_s_at | myosin-binding protein C, slow-type | 13.00 | up | 30 | 68 | L | 19.1 | 80.9 |
| 47 | 36242_at | small proline-rich protein 2C | 12.08 | | | 82 | L | 27.0 | 73.0 |
| 48 | 36616_at | DAZ associated protein 2 | 11.62 | down | 19 | 84 | H | 70.8 | 29.2 |
| 49 | 33792_at | prostate stem cell antigen | 11.62 | | | | L | 16.9 | 83.2 |
| 50 | 39500_s_at | hypothetical protein dJ465N24.2.1 | 11.16 | | | 24 | | | |

TABLE 59
Top gene list for Cluster B vs. CA

| | | | | Paper | | 126 | | | |
|---|---|---|---|---|---|---|---|---|---|
| Rank | Affx Num | Gene description | Z-score | List | Rank | Rank | H/L | High % | Low % |
| 1 | 40103_at | villin 2 (ezrin) | 605.55 | up | 1 | 2 | L | 42.7 | 57.3 |
| 2 | 31497_at | G antigen 2 | 136.76 | | | | L | 21.4 | 78.7 |
| 3 | 32104_i_at | calcium/calmodulin-dependent protein kinase (CaM kinase) II gamma | 128.48 | | | | L | 38.2 | 61.8 |
| 4 | 41264_at | Homo sapiens mRNA; cDNA DKFZp586F1322 | 99.03 | | | | H | 61.8 | 38.2 |
| | | (from clone DKFZp586F1322) | | | | | | | |
| 5 | 37348_s_at | thyroid hormone receptor interactor 7 | 92.13 | | | | L | 28.1 | 71.9 |
| 6 | 39775_at | serine (or cysteine) proteinase inhibitor, clade G | 89.83 | | | | L | 40.5 | 59.6 |
| | | (C1 inhibitor), member 1 | | | | | | | |
| 7 | 1096_g_at | CD19 antigen | 67.29 | up | 2 | 1 | L | 46.1 | 53.9 |
| 8 | 36938_at | N-acyisphingosine amidohydrolase (acid ceramidase) | 64.99 | down | 2 | 11 | H | 50.6 | 49.4 |
| 9 | 39184_at | transcription elongation factor B (SIII), polypeptide 2 | 47.97 | | | | H | 73.0 | 27.0 |
| | | (18 kD, elongin B) | | | | | | | |
| 10 | 33390_at | CD68 antigen | 47.51 | | | 15 | H | 50.6 | 49.4 |
| 11 | 1637_at | mitogen-activated protein kinase-activated protein kinase 3 | 40.61 | | | | L | 37.1 | 62.9 |
| 12 | 41577_at | protein phosphatase 1, regulatory (inhibitor) subunit 16B | 32.33 | | | 28 | L | 42.7 | 57.3 |
| 13 | 40828_at | Rho guanine nucleotide exchange factor (GEF) 7 | 32.33 | up | 32 | 19 | L | 39.3 | 60.7 |
| 14 | 37672_at | ubiquitin specific protease 7 (herpes virus-associated) | 30.03 | up | 10 | 50 | L | 41.6 | 58.4 |
| 15 | 32774_at | NADH dehydrogenase (ubiquinone) 1 beta | 28.18 | | | | L | 21.4 | 78.7 |
| | | subcomplex, 8 (19 kD, ASHI) | | | | | | | |
| 16 | 38363_at | TYRO protein tyrosine kinase binding protein | 25.42 | down | 22 | 91 | L | 48.3 | 51.7 |
| 17 | 34573_at | ephrin-A3 | 25.42 | | | | L | 25.8 | 74.2 |
| 18 | 39044_s_at | diacylglycerol kinase, delta (130 kD) | 24.96 | up | 17 | 27 | L | 49.4 | 50.8 |
| 19 | 1519_at | v-ets avian erythroblastosis virus E26 oncogene homolog 2 | 23.12 | | | 46 | L | 43.8 | 56.2 |
| 20 | 1389_at | membrane metallo-endopeptidase (neutral | 22.20 | | | | L | 37.1 | 62.9 |
| | | endopeptidase, enkephalinase, CALLA, CD10) | | | | | | | |
| 21 | 39866_at | ubiquitin specific protease 22 | 19.90 | | | | L | 38.2 | 61.8 |
| 22 | 33137_at | latent transforming growth factor beta binding protein 4 | 19.90 | | | | L | 31.5 | 68.5 |
| 23 | 35367_at | lectin, galactoside-binding, soluble, 3 (galectin 3) | 19.44 | down | 5 | 33 | L | 46.1 | 53.9 |
| 24 | 33470_at | KIAA1719 protein | 19.44 | | | | L | 28.1 | 71.9 |
| 25 | 37325_at | farnesyl diphosphate synthase (farnesyl | 18.98 | | | | L | 18.0 | 82.0 |
| | | pyrophosphate synthetase, dimethylallyltranstransferase, | | | | | | | |
| | | geranyltranstransferase) | | | | | | | |
| 26 | 32174_at | solute carrier family 9 (sodium/hydrogen exchanger), | 18.52 | | | | L | 36.0 | 64.0 |
| | | isoform 3 regulatory factor 1 | | | | | | | |
| 27 | 40281_at | neural precursor cell expressed, developmentally down-regulated 5 | 18.06 | | | | L | 41.6 | 58.4 |
| 28 | 38269_at | protein kinase D2 | 18.06 | up | 3 | 7 | L | 42.7 | 57.3 |
| 29 | 41187_at | myosin regulatory light chain | 17.14 | | | | H | 82.0 | 18.0 |
| 30 | 40877_s_at | D15F37 (pseudogene) | 17.14 | | | | H | 51.7 | 48.3 |
| 31 | 39139_at | signal peptidase complex (18 kD) | 17.14 | | | | L | 41.6 | 58.4 |
| 32 | 210_at | phospholipase C, beta 2 | 17.14 | | | | H | 65.2 | 34.8 |
| 33 | 1179_at | NO_.SIF_seq | 17.14 | | | | H | 50.6 | 49.4 |
| 34 | 36952_at | hydroxyacyl-Coenzyme A dehydrogenase/3- | 16.22 | | | | H | 83.2 | 16.9 |
| | | ketoacyl-Coenzyme A thiolase/enoyl-Coenzyme A | | | | | | | |
| | | hydratase (trifunctional protein), alpha subunit | | | | | | | |
| 35 | 35974_at | lymphoid-restricted membrane protein | 16.22 | up | 36 | 23 | H | 52.8 | 47.2 |
| 36 | 40134_at | ATP synthase, H+ transporting, mitochondrial F0 complex, | 15.76 | | | | L | 21.4 | 78.7 |
| | | subunit f, isoform 2 | | | | | | | |
| 37 | 37759_at | Lysosomal-associated multispanning membrane protein-5 | 15.76 | | | 58 | L | 48.3 | 51.7 |
| 38 | 34508_r_at | KIAA1079 protein | 15.76 | | | | L | 44.9 | 55.1 |
| 39 | 31955_at | Finkel-Biskis-Reilly murine sarcoma virus (FBR-MuSV) | 14.84 | | | | L | 41.6 | 58.4 |
| | | ubiquitously expressed (fox derived); | | | | | | | |
| | | ribosomal protein S30 | | | | | | | |
| 40 | 1827_s_at | v-myc avian myelocytomatosis viral oncogene homolog | 14.84 | | | | L | 23.6 | 76.4 |
| 41 | 37347_at | ESTs, Highly similar to A36670 cell division control | 14.38 | | | | L | 31.5 | 68.5 |
| | | protein CKS1 [H. sapiens] | | | | | | | |
| 42 | 36111_s_at | splicing factor, arginine/serine-rich 2 | 14.38 | up | 13 | 12 | L | 44.9 | 55.1 |
| 43 | 35835_at | Homo sapiens cDNA FLJ30217 fis, clone BRACE2001709, | 14.38 | | | | H | 68.5 | 31.5 |
| | | highly similar to Homo sapiens anaphase- | | | | | | | |
| | | promoting complex subunit 5 (APC5) mRNA | | | | | | | |
| 44 | 32510_at | aldo-keto reductase family 7, member A2 (aflatoxin aldehyde reductase) | 13.92 | | | | L | 38.2 | 61.8 |
| 45 | 34415_at | activin A receptor, type IB | 13.46 | | | | L | 23.6 | 76.4 |
| 46 | 32051_at | hypothetical protein MGC2840 similar to a putative glucosyltransferase | 13.00 | | | | L | 44.9 | 55.1 |
| 47 | 40570_at | forkhead box O1A (rhabdomyosarcoma) | 12.54 | | | | L | 27.0 | 73.0 |
| 48 | 34965_at | cystatin F (leukocystatin) | 12.54 | | | | L | 30.3 | 69.7 |
| 49 | 37376_at | ORF | 12.08 | | | 20 | L | 44.9 | 55.1 |
| 50 | 39994_at | chemokine (C—C motif) receptor 1 | 11.62 | down | 9 | 57 | H | 50.6 | 49.4 |

TABLE 60
Top gene list for Cluster C vs. AB

| | | | | Paper | | 126 | | | |
|---|---|---|---|---|---|---|---|---|---|
| Rank | Affx Num | Gene description | Z-score | List | Rank | Rank | H/L | High % | Low % |
| 1 | 40103_at | villin 2 (ezrin) | 650.18 | down | 2 | 8 | H | 50.6 | 49.4 |
| 2 | 36938_at | N-acylsphingosine amidohydrolase (acid ceramidase) | 140.44 | | | 1 | H | 50.6 | 49.4 |
| 3 | 35755_at | inositol 1,3,4-triphosphate 5/6 kinase | 96.27 | | | 99 | L | 38.2 | 61.8 |
| 4 | 37348_s_at | thyroid hormone receptor interactor 7 | 94.89 | | | | L | 30.3 | 69.7 |
| 5 | 39184_at | transcription elongation factor B (SIII), polypeptide | 81.55 | | | | L | 18.0 | 82.0 |
| | | 2 (18 kD, elongin B) | | | | | | | |
| 6 | 35367_at | lectin, galactoside-binding, soluble, 3 (galectin 3) | 81.55 | | | 2 | L | 38.2 | 61.8 |
| 7 | 35841_at | polymerase (RNA) II (DNA directed) polypeptide L (7.6 kD) | 74.19 | | | 112 | H | 55.1 | 44.9 |
| 8 | 1637_at | mitogen-activated protein kinase-activated protein kinase 3 | 67.29 | up | 3 | 3 | L | 37.1 | 62.9 |
| 9 | 40539_at | myosin IXB | 62.23 | | | | L | 15.7 | 84.3 |
| 10 | 38485_at | NADH dehydrogenase (ubiquinone) 1, subcomplex | 57.63 | | | 75 | H | 64.0 | 36.0 |
| | | unknown, 1 (6 kD, KFYI) | | | | | | | |
| 11 | 33768_at | dystrophia myotonica-containing WD repeat motif | 52.11 | | | | H | 80.9 | 19.1 |
| 12 | 31626_i_at | amine oxidase, copper containing 3 (vascular adhesion protein 1) | 50.73 | | | | L | 24.7 | 75.3 |
| 13 | 40819_at | RNA binding motif protein 8A | 39.69 | | | | L | 12.4 | 87.6 |
| 14 | 34573_at | ephrin-A3 | 39.23 | | | | L | 31.5 | 68.5 |
| 15 | 40094_r_at | Lutheran blood group (Auberger b antigen included) | 37.85 | | | | L | 18.0 | 82.0 |
| 16 | 36517_at | U2(RNU2) small nuclear RNA auxillary factor 1 (non-standard symbol) | 36.47 | | | | H | 57.3 | 42.7 |
| 17 | 40109_at | serum response factor (c-fos serum response | 33.25 | | | | H | 73.0 | 27.0 |
| | | element-binding transcription factor) | | | | | | | |
| 18 | 39689_at | cystatin C (amyloid angiopathy and cerebral hemorrhage) | 32.33 | | | 13 | L | 38.2 | 61.8 |
| 19 | 37672_at | ubiquitin specific protease 7 (herpes virus-associated) | 32.33 | | | | L | 31.5 | 68.5 |
| 20 | 40522_at | glutamate-ammonia ligase (glutamine synthase) | 31.87 | | | | L | 49.4 | 50.6 |
| 21 | 32166_at | Homo sapiens clone 24775 mRNA sequence | 31.41 | | | 125 | L | 47.2 | 52.8 |
| 22 | 39994_at | chemokine (C—C motif) receptor 1 | 29.10 | | | 9 | L | 37.1 | 62.9 |
| 23 | 1096_g_at | CD19 antigen | 26.80 | down | 1 | 7 | H | 55.1 | 44.9 |
| 24 | 36952_at | hydroxyacyl-Coenzyme A dehydrogenase/3-ketoacyl-Coenzyme | 22.66 | | | | H | 61.8 | 38.2 |
| | | A thiolase/enoyl-Coenzyme A hydratase | | | | | | | |
| | | (trifunctional protein), alpha subunit | | | | | | | |
| 25 | 39866_at | ubiquitin specific protease 22 | 21.74 | | | | L | 38.2 | 61.8 |
| 26 | 38368_at | dUTP pyrophosphatase | 21.74 | | | | L | 23.6 | 76.4 |
| 27 | 1450_g_at | proteasome (prosome, macropain) subunit, alpha type, 4 | 21.74 | | | 54 | L | 37.1 | 62.9 |
| 28 | 39827_at | hypothetical protein | 21.28 | | | | L | 49.4 | 50.6 |
| 29 | 33308_at | glucuronidase, beta | 19.90 | | | 86 | L | 39.3 | 60.7 |
| 30 | 32774_at | NADH dehydrogenase (ubiquinone) 1 beta | 19.90 | | | | H | 55.1 | 44.9 |
| | | subcomplex, 8 (19 kD, ASHI) | | | | | | | |
| 31 | 1034_at | tissue inhibitor of metalloproteinase 3 (Sorsby | 19.90 | | | | L | 36.0 | 64.0 |
| | | fundus dystrophy, pseudoinflammatory) | | | | | | | |
| 32 | 39139_at | signal peptidase complex (18 kD) | 19.44 | | | 30 | L | 41.6 | 58.4 |
| 33 | 35337_at | F-box only protein 7 | 18.52 | | | | H | 67.4 | 32.6 |
| 34 | 38363_at | TYRO protein tyrosine kinase binding protein | 18.06 | up | 4 | 14 | L | 43.8 | 56.2 |
| 35 | 37341_at | glutamate dehydrogenase 1 | 18.06 | | | | L | 29.2 | 70.8 |
| 36 | 32174_at | solute carrier family 9 (sodium/hydrogen | 18.06 | | | | L | 36.0 | 64.0 |
| | | exchanger), isoform 3 regulatory factor 1 | | | | | | | |
| 37 | 40774_at | chaperonin containing TCP1, subunit 3 (gamma) | 17.60 | | | | H | 68.5 | 31.5 |
| 38 | 39803_s_at | chromosome 21 open reading frame 2 | 17.60 | | | | H | 55.1 | 44.9 |
| 39 | 36630_at | delta sleep inducing peptide, immunoreactor | 17.60 | | | | L | 42.7 | 57.3 |
| 40 | 40792_s_at | triple functional domain (PTPRF interacting) | 16.68 | | | | L | 36.0 | 64.0 |
| 41 | 40134_at | ATP synthase, H+ transporting, mitochondrial | 16.68 | | | 118 | L | 34.8 | 65.2 |
| | | F0 complex, subunit f, isoform 2 | | | | | | | |
| 42 | 37028_at | protein phosphatase 1, regulatory (inhibitor) subunit 15A | 16.68 | | | | L | 46.1 | 53.9 |
| 43 | 34161_at | lactoperoxidase | 16.68 | | | | L | 49.4 | 50.6 |
| 44 | 39044_s_at | diacylglycerol kinase, delta (130 kD) | 15.76 | | | | H | 67.4 | 32.6 |
| 45 | 37351_at | uridine phosphorylase | 15.30 | | | 69 | H | 51.7 | 48.3 |
| 46 | 39795_at | adaptor-related protein complex 2, mu 1 subunit | 14.84 | | | | L | 36.0 | 64.0 |
| 47 | 37294_at | B-cell translocation gene 1, anti-proliferative | 14.84 | | | | H | 57.3 | 42.7 |
| 48 | 33821_at | homolog of yeast long chain polyunsaturated fatty | 14.84 | | | | L | 43.8 | 56.2 |
| | | acid elongation enzyme 2 | | | | | | | |
| 49 | 41374_at | ribosomal protein S6 kinase, 70 kD, polypeptide 2 | 14.38 | | | | H | 51.7 | 48.3 |
| 50 | 37026_at | core promoter element binding protein | 14.38 | | | | H | 52.8 | 47.2 |

TABLE 61
Statistical significance of the prediction for VxInsight clusters

| | A vs. not-A | | B vs. not-B | | C vs. not-C | |
|---|---|---|---|---|---|---|
| # of genes | pVal1 | pVal2 | pVal1 | pVal2 | pVal1 | pVal2 |
| 1 | 0.000096 | 0.346847 | 0.000004 | 0.000010 | 0.000021 | 0.000065 |
| 2 | 0.000004 | 0.016428 | 0.000000 | 0.000000 | 0.000000 | 0.000000 |
| 3 | 0.000021 | 0.085586 | 0.000000 | 0.000000 | 0.000000 | 0.000000 |
| 4 | 0.000021 | 0.085586 | 0.000000 | 0.000000 | 0.000000 | 0.000000 |
| 5 | 0.000021 | 0.085586 | 0.000000 | 0.000000 | 0.000000 | 0.000000 |
| 6 | 0.000021 | 0.085586 | 0.000000 | 0.000000 | 0.000000 | 0.000000 |
| 7 | 0.000004 | 0.031532 | 0.000001 | 0.000002 | 0.000000 | 0.000000 |
| 8 | 0.000004 | 0.031532 | 0.000000 | 0.000000 | 0.000000 | 0.000000 |
| 9 | 0.000004 | 0.031532 | 0.000000 | 0.000000 | 0.000000 | 0.000000 |
| 10 | 0.000004 | 0.031532 | 0.000000 | 0.000000 | 0.000000 | 0.000000 |
| 11 | 0.000004 | 0.031532 | 0.000000 | 0.000000 | 0.000000 | 0.000000 |
| 12 | 0.000004 | 0.031532 | 0.000000 | 0.000000 | 0.000000 | 0.000000 |
| 13 | 0.000004 | 0.031532 | 0.000000 | 0.000000 | 0.000000 | 0.000000 |
| 14 | 0.000004 | 0.031532 | 0.000000 | 0.000000 | 0.000000 | 0.000000 |
| 15 | 0.000004 | 0.031532 | 0.000000 | 0.000000 | 0.000000 | 0.000000 |
| 16 | 0.000004 | 0.031532 | 0.000000 | 0.000000 | 0.000000 | 0.000000 |
| 17 | 0.000004 | 0.031532 | 0.000000 | 0.000000 | 0.000000 | 0.000000 |
| 18 | 0.000004 | 0.031532 | 0.000000 | 0.000000 | 0.000000 | 0.000000 |
| 19 | 0.000004 | 0.031532 | 0.000000 | 0.000000 | 0.000000 | 0.000000 |
| 20 | 0.000004 | 0.031532 | 0.000000 | 0.000000 | 0.000000 | 0.000000 |
| 21 | 0.000004 | 0.031532 | 0.000000 | 0.000000 | 0.000000 | 0.000000 |
| 22 | 0.000004 | 0.031532 | 0.000000 | 0.000000 | 0.000000 | 0.000000 |
| 23 | 0.000021 | 0.085586 | 0.000000 | 0.000000 | 0.000000 | 0.000000 |
| 24 | 0.000021 | 0.085586 | 0.000000 | 0.000000 | 0.000000 | 0.000000 |
| 25 | 0.000021 | 0.085586 | 0.000000 | 0.000000 | 0.000000 | 0.000000 |
| 26 | 0.000021 | 0.085586 | 0.000000 | 0.000000 | 0.000000 | 0.000000 |
| 27 | 0.000021 | 0.085586 | 0.000000 | 0.000000 | 0.000000 | 0.000000 |
| 28 | 0.000021 | 0.037385 | 0.000000 | 0.000000 | 0.000000 | 0.000000 |
| 29 | 0.000004 | 0.006823 | 0.000000 | 0.000000 | 0.000000 | 0.000000 |
| 30 | 0.000004 | 0.006823 | 0.000000 | 0.000000 | 0.000000 | 0.000000 |
| 31 | 0.000004 | 0.006823 | 0.000000 | 0.000000 | 0.000000 | 0.000000 |
| 32 | 0.000004 | 0.006823 | 0.000000 | 0.000000 | 0.000000 | 0.000000 |
| 33 | 0.000004 | 0.006823 | 0.000000 | 0.000000 | 0.000000 | 0.000000 |
| 34 | 0.000004 | 0.006823 | 0.000000 | 0.000000 | 0.000000 | 0.000000 |
| 35 | 0.000004 | 0.006823 | 0.000000 | 0.000000 | 0.000000 | 0.000000 |
| 36 | 0.000004 | 0.006823 | 0.000000 | 0.000000 | 0.000000 | 0.000000 |
| 37 | 0.000004 | 0.006823 | 0.000000 | 0.000000 | 0.000000 | 0.000000 |
| 38 | 0.000004 | 0.006823 | 0.000000 | 0.000000 | 0.000000 | 0.000000 |
| 39 | 0.000001 | 0.000908 | 0.000000 | 0.000000 | 0.000000 | 0.000000 |
| 40 | 0.000004 | 0.002288 | 0.000000 | 0.000000 | 0.000000 | 0.000000 |
| 41 | 0.000004 | 0.002288 | 0.000000 | 0.000000 | 0.000000 | 0.000000 |
| 42 | 0.000004 | 0.002288 | 0.000000 | 0.000000 | 0.000000 | 0.000000 |
| 43 | 0.000004 | 0.002288 | 0.000000 | 0.000000 | 0.000000 | 0.000000 |
| 44 | 0.000004 | 0.002288 | 0.000000 | 0.000000 | 0.000000 | 0.000000 |
| 45 | 0.000004 | 0.002288 | 0.000000 | 0.000000 | 0.000000 | 0.000000 |
| 46 | 0.000004 | 0.002288 | 0.000000 | 0.000000 | 0.000000 | 0.000000 |
| 47 | 0.000004 | 0.002288 | 0.000000 | 0.000000 | 0.000000 | 0.000000 |
| 48 | 0.000004 | 0.002288 | 0.000000 | 0.000000 | 0.000000 | 0.000000 |
| 49 | 0.000001 | 0.000908 | 0.000000 | 0.000000 | 0.000000 | 0.000000 |
| 50 | 0.000001 | 0.000908 | 0.000000 | 0.000000 | 0.000000 | 0.000000 |

TABLE 62
OVA classification results for VxInsight clusters (only with DIFF genes)

| | A vs B C | | | | B vs CA | | | | C vs A B | | | |
|---|---|---|---|---|---|---|---|---|---|---|---|---|
| | Training | | Test | | Training | | Test | | Training | | Test | |
| # of genes | Correct | SR | Correct | SR | Correct | SR | Correct | SR | Correct | SR | Correct | SR |
| 1 | 79 | 0.89 | 30 | 0.81 | 54 | 0.61 | 32 | 0.86 | 54 | 0.61 | 31 | 0.84 |
| 2 | 82 | 0.92 | 32 | 0.86 | 72 | 0.81 | 35 | 0.95 | 77 | 0.87 | 36 | 0.97 |
| 3 | 84 | 0.94 | 31 | 0.84 | 76 | 0.85 | 35 | 0.95 | 79 | 0.89 | 35 | 0.95 |
| 4 | 87 | 0.98 | 31 | 0.84 | 73 | 0.82 | 34 | 0.92 | 80 | 0.90 | 35 | 0.95 |
| 5 | 87 | 0.98 | 31 | 0.84 | 70 | 0.79 | 34 | 0.92 | 77 | 0.87 | 36 | 0.97 |
| 6 | 88 | 0.99 | 31 | 0.84 | 76 | 0.85 | 35 | 0.95 | 77 | 0.87 | 36 | 0.97 |
| 7 | 84 | 0.94 | 32 | 0.86 | 74 | 0.83 | 35 | 0.95 | 77 | 0.87 | 36 | 0.97 |
| 8 | 84 | 0.94 | 32 | 0.86 | 77 | 0.87 | 35 | 0.95 | 80 | 0.90 | 36 | 0.97 |
| 9 | 84 | 0.94 | 32 | 0.86 | 77 | 0.87 | 35 | 0.95 | 80 | 0.90 | 36 | 0.97 |
| 10 | 83 | 0.93 | 32 | 0.86 | 77 | 0.87 | 36 | 0.97 | 80 | 0.90 | 36 | 0.97 |
| 11 | 82 | 0.92 | 32 | 0.86 | 76 | 0.85 | 36 | 0.97 | 82 | 0.92 | 36 | 0.97 |
| 12 | 83 | 0.93 | 32 | 0.86 | 78 | 0.88 | 36 | 0.97 | 82 | 0.92 | 36 | 0.97 |
| 13 | 83 | 0.93 | 32 | 0.86 | 76 | 0.85 | 36 | 0.97 | 81 | 0.91 | 36 | 0.97 |
| 14 | 84 | 0.94 | 32 | 0.86 | 76 | 0.85 | 36 | 0.97 | 82 | 0.92 | 35 | 0.95 |
| 15 | 84 | 0.94 | 32 | 0.86 | 75 | 0.84 | 36 | 0.97 | 82 | 0.92 | 36 | 0.97 |
| 16 | 83 | 0.93 | 32 | 0.86 | 77 | 0.87 | 36 | 0.97 | 82 | 0.92 | 36 | 0.97 |
| 17 | 84 | 0.94 | 32 | 0.86 | 78 | 0.88 | 36 | 0.97 | 82 | 0.92 | 36 | 0.97 |
| 18 | 84 | 0.94 | 32 | 0.86 | 78 | 0.88 | 36 | 0.97 | 82 | 0.92 | 36 | 0.97 |
| 19 | 84 | 0.94 | 32 | 0.86 | 76 | 0.85 | 36 | 0.97 | 81 | 0.91 | 36 | 0.97 |
| 20 | 84 | 0.94 | 32 | 0.86 | 75 | 0.84 | 36 | 0.97 | 81 | 0.91 | 36 | 0.97 |
| 21 | 83 | 0.93 | 32 | 0.86 | 76 | 0.85 | 36 | 0.97 | 82 | 0.92 | 36 | 0.97 |
| 22 | 83 | 0.93 | 32 | 0.86 | 75 | 0.84 | 36 | 0.97 | 83 | 0.93 | 35 | 0.95 |
| 23 | 85 | 0.96 | 31 | 0.84 | 76 | 0.85 | 35 | 0.95 | 79 | 0.89 | 36 | 0.97 |
| 24 | 85 | 0.96 | 31 | 0.84 | 78 | 0.88 | 36 | 0.97 | 79 | 0.89 | 36 | 0.97 |
| 25 | 85 | 0.96 | 31 | 0.84 | 73 | 0.82 | 35 | 0.95 | 79 | 0.89 | 36 | 0.97 |
| 26 | 85 | 0.96 | 31 | 0.84 | 72 | 0.81 | 36 | 0.97 | 80 | 0.90 | 36 | 0.97 |
| 27 | 85 | 0.96 | 31 | 0.84 | 75 | 0.84 | 35 | 0.95 | 81 | 0.91 | 36 | 0.97 |
| 28 | 85 | 0.96 | 31 | 0.84 | 76 | 0.85 | 34 | 0.92 | 80 | 0.90 | 35 | 0.95 |
| 29 | 85 | 0.96 | 31 | 0.84 | 76 | 0.85 | 34 | 0.92 | 82 | 0.92 | 34 | 0.92 |
| 30 | 85 | 0.96 | 31 | 0.84 | 76 | 0.85 | 34 | 0.92 | 81 | 0.91 | 34 | 0.92 |
| 31 | 85 | 0.96 | 31 | 0.84 | 76 | 0.85 | 34 | 0.92 | 80 | 0.90 | 33 | 0.89 |
| 32 | 85 | 0.96 | 31 | 0.84 | 76 | 0.85 | 34 | 0.92 | 77 | 0.87 | 34 | 0.92 |
| 33 | 85 | 0.96 | 31 | 0.84 | | | | | 79 | 0.89 | 35 | 0.95 |
| 34 | 85 | 0.96 | 32 | 0.86 | | | | | 79 | 0.89 | 35 | 0.95 |
| 35 | 85 | 0.96 | 32 | 0.86 | | | | | 78 | 0.88 | 35 | 0.95 |
| 36 | 84 | 0.94 | 33 | 0.89 | | | | | 81 | 0.91 | 35 | 0.95 |
| 37 | 84 | 0.94 | 33 | 0.89 | | | | | | | | |
| 38 | 84 | 0.94 | 34 | 0.92 | | | | | | | | |
| 39 | 84 | 0.94 | 34 | 0.92 | | | | | | | | |
| 40 | 84 | 0.94 | 34 | 0.92 | | | | | | | | |
| 41 | 84 | 0.94 | 35 | 0.95 | | | | | | | | |
| 42 | 84 | 0.94 | 34 | 0.92 | | | | | | | | |
| 43 | 84 | 0.94 | 35 | 0.95 | | | | | | | | |
| 44 | 84 | 0.94 | 35 | 0.95 | | | | | | | | |
| 45 | 85 | 0.96 | 34 | 0.92 | | | | | | | | |
| 46 | 85 | 0.96 | 34 | 0.92 | | | | | | | | |

### REFERENCES FOR SUPPLEMENTARY INFORMATION

1. Becton D, Ravindrinath Y, Dahl G V, Berkow R L, Chang M, Stine K, Behm F G, Raimondi S C, Massey G, Weinstein H J: A Phase III study of intensive cytarabine (Ara-C) induction followed by cyclosporine (CSA) modulation of drug resistance in de novo pediatric AML; POG 9421. Blood. 98, 461a (2001).
2. Dreyer Z E, Steuber C P, Bowman W P, Murray J C, Coppes M J, Dinndorf P, Camitta B: High risk infant ALL-improved survival with intensive chemotherapy (POG9407). Proc Am. Soc. Clin. Oncol. 17, 529a (1998).
3. Frankel L S, Ochs J, Shuster J J, Dubowy R, Bowman W P, Hockenberry-Eaton M, Borowitz M, Carroll A J, Steuber C P, Pullen D J: Therapeutic trial for infant acute lymphoblastic leukemia: the Pediatric Oncology Group experience (POG 8493). J. Pediatr. Hematol. Oncol. 19, 35-42 (1997).
4. Lauer S J, Camitta B M, Leventhal B G, Mahoney D, Shuster J, Keifer G, Pullen J, Steuber C P, Carroll A J, Kamen B: Intensive alternating drug pairs after remission induction for treatment of infants with acute lymphoblastic leukemia: a Pediatric Oncology Group study (POG8398). J. Pediatr. Hematol. Oncol. 20, 229-33 (1998).
5. Ravindrinath Y, Yeager A M, Chang M, Steuber C P, Krischer J, Graham-Pole J, Carroll A, Inoue S, Camitta B, Weinstein H J: Autologous bone marrow transplantation versus intensive consolidation chemotherapy for acute myeloid leukemia in childhood (POG8821). N. Engl. J. Med. 334, 1428-34 (1996).
6. Helman, P., Veroff, R., Atlas, S., Willman, C. A Bayesian network classification methodology for gene expression data. (submitted 2003; available on the worldwide web at cs.unm.edu/˜helman/papers/JCB_Total.pdf).
7. Pearl, J. Probabilistic reasoning for intelligent systems. Morgan Kaufmann, San Francisco (1988).
8. Heckerman, D., Geiger, D., Chickering, D. Learning Bayesian networks: The combination of knowledge and statistical data. Machine Learning. 20, 197-243 (1995).
9. Duda, R., Hart, P. Pattern classification and scene analysis. John Wiley and Sons, New York. (1973).
10. Langley, P., Iba, W., Thompson, K. An analysis of Bayesian classifiers. In Proc. 10th National Conference on Artificial Intelligence 223-228, AAAI Press. (1992).
11. Friedman, N., Geiger, D., Goldszmidt, M. Bayesian network classifiers. Machine Learning. 29, 131-163 (1997).
12. Ben-Dor, A., Bruhn, L., Friedman, N., Nachman, I., Schummer, M., & Yakhini, Z. Tissue Classification with Gene Expression Profiles. J. Comput. Biol. 7, 559-584 (2000).
13. Ben-Dor A., Friedman N. and Yakhini Z. Class discovery in gene expression data, In Proc. Fifth Annual Conference of Computational Biology, 31-38, ACM Press, New York (2001)
14. Cristianini N. and Shawe-Taylor, J. An Introduction to Support Vector Machines and other kernel-based learning methods, Cambridge University Press, Cambridge (2000).
15. Mangasarian O. Generalized Support Vector Machines, Smola A., Barlett P., Scholköpf B. and Schuurmans C., editors, Advances in Large Margin Classifiers, MIT Press, Cambridge, Mass. (1999).
16. Vapnik V. Statistical Learning Theory, John Wiley & Sons, New York (1999).
17. Golub T., Slonim D., Tamayo P., Huard C., Caasenbeek J., Coller H., Loh M., Downing J., Caligiuri M., Bloomfield M., and Lander E. Molecular classification of cancer: class discovery and prediction by gene expression monitoring, Science 286, 531-537 (1999).
18. Guyon I., Weston J., Barnhill S. and Vapnik V. 2002, Gene Selection for Cancer Classification using Support Vector Machines, Machine Learning 46, 389-422.
19. Ramaswamy S., Tamayo P., Rifkin R., Mukherjee S., Yeang C., Angelo M., Ladd C., Reich M., Latulippe E., Mesirov J., Poggio T., Gerald W., Loda M., Lander E. and Golub T. Multiclass cancer diagnosis using tumor gene expression signatures, Proc. Natl. Acad. Sci. 98, 15149-15154 (2002).
20. Ambriose S. and McLachlan G. Selection Bias in gene extraction on the basis on microarray gene expression data. Proc. Natl. Aca. Sci. 99, 6562-6566 (2002).
21. The MathWorks, Inc. MATLAB User's Guide, Natick, Mass. 01760 (1992).
22. Mangasarian O. and Musicant D. Lagrangian Support Vector Machines, Journal of Machine Learning Research. 1, 161-177 (2001).
23. Michael T. Brown and Lori R. Wricker, Discriminant Analysis. In: Handbook of Applied Multivariate Statistics and Mathematical Modeling. Academic, New York. Affymetrix Statistical Algorithms Reference Guide. Affymetrix Inc. (2001).
24. Zadeh L. A. Fuzzy logic and its application to approximate reasoning. Information Processing. 74, 591-594 (1974).
25. Nguyen, H. T. and Walker, E. A. A First Course in Fuzzy Logic. CRC press (1997).
26. Woolf, P. J. and Wang, Y. A fuzzy logic approach to analyzing gene expression data. Physiol Genomics. 3, 9-15. (2000).
27. Mendel, J. M. Fuzzy logic systems for engineering: a tutorial. Proceedings of the IEEE, 83, 345-377 (1995).
28. Wang, L. Adaptive Fuzzy Systems and Control. Prentice-Hall (1994).
29. Moore, D. S. The Basic Practice of Statistics. W.H. Freeman and Co. (2000).
30. Wang, X., Atlas, S., Willman, C. L., and Li, B. L. Adaptive Neuro-Fuzzy Clustering Analysis of Gene Microarray Data. Preprint. Univ. of New Mexico. (2002).
31. Liu, H., Motoda, H., and Dash, M. A monotonic measure for optimal feature selection. In Proceedings of European Conference on Machine Learning, pp 101-106. (1998).
32. Siedlecki, W. and Sklansky, L. A not on genetic algorithms for large-scale feature selection. Pattern Recognition Letters. 10, 335-347 (1989).
33. Moore, A. and Lee, M. Efficient algorithms for minimizing cross validation error. In Proceedings of 11th International Machine Learning Conference. Morgan Kaufmann. (1994).
34. Mathworks User's Guide of Fuzzy Logic Toolbox. The Mathworks Inc. (2000).
35. Casella, G. & Berger, R. L. Statistical Inference. Belmont, Calif.:Duxbury Press. (2002).
36. Agresti, A, Categorical Data Analysis, 2^{nd} Ed., Hoboken:John Wiley & Sons. (2002).
37. The SAS System for Windows, Release 8.02, SAS Institute, Inc. (2001).
38. Lehmann, E. L. Testing Statistical Hypotheses, Belmont, Calif.: Wadsworth & Brooks. (1991).
39. Eisen, M. B., Spellman, P. T., Brown, P. O., and Botstein, D. Cluster analysis and display of genome-wide expression patterns. Proc. Natl. Acad. Sci. USA 95, 14863-14868 (1998).
40. Jolliffe, I. T. Principal Component Analysis. Springer-Verlag (1986).
41. Kirby, M. Geometric Data Analysis. John Wiley & Sons (2001).
42. Trefethen, L. & Bau, D. Numerical Linear Algebra. SIAM, Philadelphia (1997).
43. Davidson, G. S., Wylie, B. N., & Boyack, K. W. Cluster Stability and the Use of Noise in Interpretation of Clustering. Proc. IEEE Information Visualization 2001, 23-30 (2001).
44. Davidson, G. S., Hendrickson, B., Johnson, D. K., Meyers, C. E., & Wylie, B. N. Knowledge mining with VxInsight: Discovery Through Interaction. Journal of Intelligent Information Systems. 11, 259-285 (1998).
45. Kim, S. K., Lund, J., Kiraly, M., Duke, K., Jiang, M., Stuart, J. M., Eizinger, A., Wylie, B. N., and Davidson, G. S. A gene expression map for Caenorhabditis elegans. Science 293, 2087-2092 (2001).
46. Werner-Washburne, M., Wylie, B., Boyack, K., Fuge, E., Galbraith, J., Fleharty, M., Weber, J., Davidson, G. S. Concurrent analysis of multiple genome-scale datasets. Genome Research. 12, 1564-1573 (2002).
47. Efron, B. Bootstrap methods—“another look at the jackknife” Ann. Statist. 7, 1-26 (1979).
48. Hjorth, J. S. Urban Computer Intensive Statistical Methods, Validation model selection and bootstrap, ISBN 0412491605, Chapman & Hall, 2-6 Boundary Row, London SE1 8HN, UK. (1994).
49. Slonim, D. K., Tamayo, P., Mesirov, J. P., Golub, T. P., and Lander, E. S. Class prediction and discovery using gene expression data. In: Proc. 4th Annual International Conf. on Computational Molecular Biology (RECOMB) pp. 263-272, Universal Academy Press, Tokyo, Japan. (1999).
50. Li, L., Weinberg, C. R., Darden, T. A., and Pedersen, L. G. Gene selection for sample classification based on gene expression data: study of sensitivity to choice of parameters of the GA/KNN method. Bioinformatics, 17, 1131-1142 (2001).
51. Li, L., Darden, T. A., Weinberg, C. R., Levine, A. J., and Pedersen, L. G. Gene assessment and sample classification for gene expression data using a genetic algorithm/k-nearest neighbor method. Combinatorial Chemistry and High Throughput Screening, 4, 727-739 (2001).
52. Hastie, T., Tibshirani, R., and Friedman, J. The Elements of Statistical Learning. Springer:New York. (2001).

### Example XIV

### Heterogeneity of Gene Expression Profiles in MLL-Associated Infant Leukemia: Identification of Distinct Expression Profiles and Novel Therapeutics Targets

Summary

Translocations involving the MLL (ALL-1, HRX, Htrx-1) gene at chromosome band 11q23 are the most common cytogenetic abnormality seen in infant leukemia. While there is evidence that MLL-associated chromosomal rearrangements carry a poorer prognosis, the pathogenesis and unique gene expression for each MLL rearrangement remain largely undefined. Using oligonucleotide arrays (Affymetrix U95Av2) and both unsupervised and supervised analysis methods we derived comprehensive gene expression profiles from a retrospective cohort of 126 infant cases registered to NCI-sponsored clinical trials. Fifty-three of those cases had MLL rearrangements with several partner genes (AF4, ENL, AF10, AF9 and AF1Q). We used class identification methods (Bayesian networks, Support Vector Machines and Discriminant Analysis) to determine genes with common patterns of expression across all the MLL cases as well as genes that were uniquely expressed and distinguishing of each MLL translocation variant. However, class discovery tools suggested that the MLL-associated profiles were quite heterogeneous among different translocation variants and were dominated by three differential expression patterns. Interpretation of our data indicated that infant MLL is an entity comprising several intrinsic biologic classes not precisely predicted by current standards of morphology, immunophenotyping, or cytogenetics. Consideration of such class-membership could improve classification schemes and reveal potential therapeutic targets for MLL-associated leukemias.

Introduction

In Example XIII, we analyzed the gene expression profiles in samples of 126 infant acute leukemia patients. Three inherent biologic subgroups were identified. These groups were not well defined by traditional cell types (AML vs. ALL) or cytogenetic (MLL vs. not) labels. Instead, they reflected different etiologic events with biological and clinical relevance. The distribution of the MLL infant cases between those “etiology-driven” clusters is the focus of this study.

Materials and Methods

For this study we analyzed 126 diagnostic bone marrow samples from patients with acute leukemia who were aged <1 year at diagnosis. In each case, the percentage of blast was >80%. The cohort was designed from cases registered to NCI-sponsored Infant Oncology Group/Children's Oncology Group treatment trials number 8398, 8493, 8821, 9107, 9407 and 9421. Of the 126 cases, 78 (62%) were acute lymphocytic leukemia (ALL) and 48 (38%) were acute myeloid leukemia (AML) by standard morphological and immunophenotypic criteria. Fifty-three (42%) cases had translocations involving the MLL gene (chromosome segment 11q23). An average of 2×10⁷ cells were used for total RNA extraction with the Qiagen RNeasy mini kit (Valencia, Calif.). The yield and integrity of the purified total RNA were assessed using the RiboGreen assay (Molecular Probes, Eugene, Oreg.) and the RNA 6000 Nano Chip (Agilent Technologies, Palo Alto, Calif.), respectively. Complementary RNA (cRNA) target was prepared from 2.5 μg total RNA using two rounds of Reverse Transcription (RT) and In Vitro Transcription (IVT). Following denaturation for 5 min at 70° C., the total RNA was mixed with 100 pmol T7-(dT)₂₄ oligonucleotide primer (Genset Oligos, La Jolla, Calif.) and allowed to anneal at 42° C. The mRNA was reverse transcribed with 200 units Superscript II (Invitrogen, Grand Island, N.Y.) for 1 hr at 42° C. After RT, 0.2 vol 5× second strand buffer, additional dNTP, 40 units DNA polymerase I, 10 units DNA ligase, 2 units RnaseH (Invitrogen) were added and second strand cDNA synthesis was performed for 2 hr at 16° C. After T4 DNA polymerase (10 units), the mix was incubated an additional 10 min at 16° C. An equal volume of phenol:chloroform:isoamyl alcohol (25:24:1) (Sigma, St. Louis, Mo.) was used for enzyme removal. The aqueous phase was transferred to a microconcentrator (Microcon 50, Millipore, Bedford, Mass.) and washed/concentrated with 0.5 ml DEPC water until the sample was concentrated to 10-20 ul. The cDNA was then transcribed with T7 RNA polymerase (Megascript, Ambion, Austin, Tex.) for 4 hr at 37° C. Following IVT, the sample was phenol:chloroform:isoamyl alcohol extracted, washed and concentrated to 10-20 ul. The first round product was used for a second round of amplification which utilized random hexamer and T7-(dT)₂₄ oligonucleotide primers, Superscript II, two RNase H additions, DNA polymerase I plus T4 DNA polymerase finally and a biotin-labeling high yield T7 RNA polymerase kit (Enzo Diagnostics, Farmingdale, N.Y.). The biotin-labeled cRNA was purified on Qiagen RNeasy mini kit columns, eluted with 50 ul of 45° C. RNase-free water and quantified using the RiboGreen assay. Following quality check on Agilent Nano 900 Chips, 15 ug cRNA were fragmented following the Affymetrix protocol (Affymetrix, Santa Clara, Calif.). The fragmented RNA was then hybridized for 20 hours at 45° C. to HG_U95Av2 probes. The hybridized probe arrays were washed and stained with the EukGE_WS2 fluidics protocol (Affymetrix), including streptavidin phycoerythrin conjugate (SAPE, Molecular Probes, Eugene, Oreg.) and an antibody amplification step (Anti-streptavidin, biotinylated, Vector Labs, Burlingame, Calif.). HG_U95Av2 chips were scanned at 488 nm, as recommended by Affymetrix. The expression value of each gene was calculated using Affymetrix Microarray Suite 5.0 software.

Data Presentation and Exclusion Criteria

Criteria used as quality controls included: total RNA integrity, cRNA quality, array image inspection, B2 oligo performance, and internal control genes (GAPDH value greater than 1800). Of the initial cohort of 142 infant acute leukemia cases, 126 were finally part of this study.

Data Analysis

Affymetrix MAS 5.0 statistical analysis software was used to process the raw microarray image data for a given sample into quantitative signal values and associated present, absent or marginal calls for each probe set. A filter was then applied which excluded from further analysis all Affymetrix “control” genes (probe sets labelled with AFFX—prefix), as well as any probe set that did not have a “present” call at least in one of the samples. This filtering step reduced the number of probe sets from 12625 to 8414, resulting in a matrix of 8,414×126 signal values. Our Bayesian classification and VxInsight clustering analyses omitted this step; choosing instead to assume minimal a priori gene selection, as described in Helman et al., 2002 and Davidson et al., 2001. The first stage of our analysis consisted of a series of binary classification problems defined on the basis of clinical and biologic labels. The nominal class distinctions were ALL/AML, MLL/not-MLL, and achieved complete remission CR/not-CR. Additionally, several derived classification problems were considered based on restrictions of the full cohort to particular subsets of the data (such as the VxInsight clusters). The multivariate supervised learning techniques used included Bayesian nets (Helman et al., 2002) and support vector machines (Guyon et al., 2002). The performance of the derived classification algorithms was evaluated using fold-dependent leave-one-out cross validation (LOOCV) techniques. These methods allowed the identification of genes associated with remission or treatment failure and with the presence or absence of translocations of the MLL gene across the dataset.

In order to identify potential clusters and inherent biologic groups, a large number of clinical co-variables were correlated with the expression data using unsupervised clustering methods such as hierarchical clustering, principal component analysis and a force-directed clustering algorithm coupled with the VxInsight visualization tool. Agglomerative hierarchical clustering with average linkage (similar to Eisen et al., 1998) was performed with respect to both genes and samples, using the MATLAB (The Mathworks, Inc.), MatArray toolbox, as well as the native MATLAB statistics toolbox. The data for a given gene was first normalized by subtracting the mean expression value computed across all patients, and dividing by the standard deviation. The distance metric used for the hierarchical clustering was one minus Pearson's correlation coefficient. This metric was chosen to enable subsequent direct comparison with the VxInsight cluster analysis, which is based on the t-statistic transformation of the correlation coefficient (Davidson et al., 2001).

The second clustering method was a particle-based algorithm implemented within the VxInsight knowledge visualization tool. In this approach, a matrix of pair similarities is first computed for all combinations of patient samples. The pair similarities are given by the t-statistic transformation of the correlation coefficient determined from the normalized expression signatures of the samples (Davidson et al., 2001). The program then randomly assigns patient samples to locations (vertices) on a two dimensions graph, and draws lines (edges) linking each sample pair, assigning each edge a weight corresponding to the pairwise t-statistic of the correlation. The resulting two-dimensional graph constitutes a candidate clustering. To determine the optimal clustering, an iterative annealing procedure is followed. In this procedure a ‘potential energy’ function that depends on edge distances and weights is minimized by following random moves of the vertices (Davidson et al., 1998, 2001). Once the 2D graph has converged to a minimum energy configuration, the clustering defined by the graph is visualized as a 3D terrain map, where the vertical axis corresponds to the density of samples located in a given 2D region. The resulting clusters are robust with respect to random starting points and to the addition of noise to the similarity matrix, evaluated through effects on neighbour stability histograms (Davidson et al., 2001).

Results

Expression Profiling Demonstrates Heterogeneity Across Infant MLL Cases

The determine the variations in gene expression profiles of infant leukemia cases involving different MLL rearrangements, 126 infant leukemia cases registered to NCI-sponsored Infant Oncology Group/Children's Oncology Group treatment trials were studied using oligonucleotide microarrays containing 12,625 probe sets (Affymetrix U95Av2 array platform). Of the 126 cases, fifty-three (42%) cases had translocations involving the MLL gene (chromosome segment 11q23). The distribution of the MLL cytogenetic abnormalities across this data set is provided in Table 63.

TABLE 63
Distribution of MLL Cytogenetic Abnormalities in the Infant Cohort

| | Total # of Cases | | |
|---|---|---|---|
| MLL Translocation | in Infant Cohort | ALL | AML |
| t(4; 11) | 29 | 28 | 1 |
| t(11; 19) | 9 | 7 | 2 |
| t(10; 11) | 4 | 2 | 2 |
| t(1; 11) | 4 | 2 | 2 |
| t(9; 11) | 4 | 1 | 3 |
| Other MLL | 3 | 1 | 2 |
| Not MLL | 42 | 26 | 16 |
| Unknown | 31 | 11 | 20 |

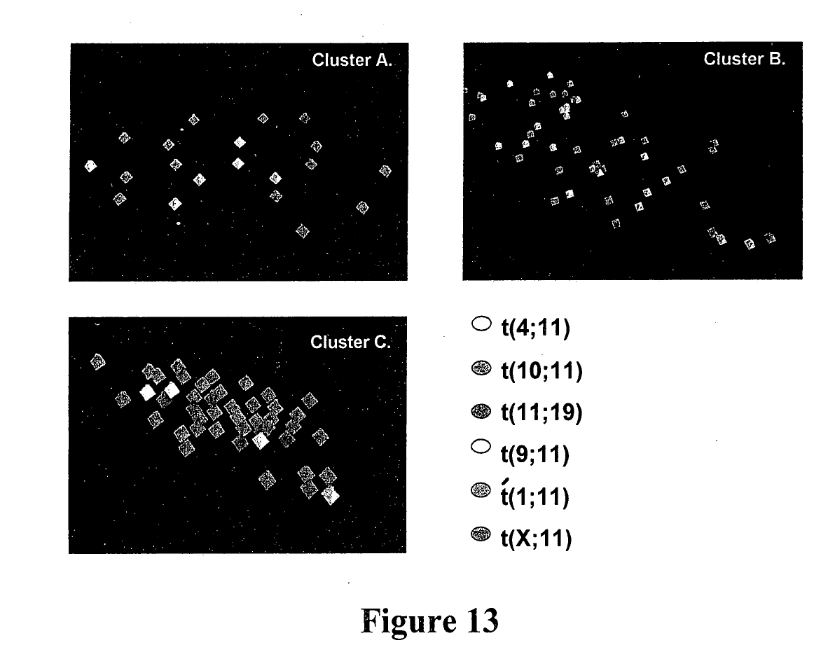

The initial examination of the data was accomplished using the force directed clustering algorithm coupled with the visualization tool, (Davidson et al., 1998; 2001). When applied to the infant cohort, this particle-based clustering algorithm demonstrated the existence of three well-separated groups of patients that displayed similar patterns of gene expression (FIG. 10) These major clusters were statistically robust and internally consistent as demonstrated by linear discrimination analysis with fold-dependent leave one out cross-validation (LOOCV). Further analysis demonstrated that the clusters could not be completely explained by the traditional diagnostic parameters (morphology: ALL vs. AML, or cytogenetics: MLL rearrangement vs. not), implying that the intrinsic biology may not be driven by these variables. Further analysis suggested an association between the three clusters and different leukemogenic mechanisms (previously submitted data), called hereafter “stem cell-like”, “lymphoid” and “myeloid”/“environmental”. MLL cases were seen in each of the mentioned patient clusters (FIG. 13). The MLL cases in the “stem cell-like” cluster (Cluster A, n=20) were primarily t(4;11) (n=7), as well as two cases with t(10;11) and one with t(11;19). The “lymphoid” cluster (Cluster B, n=52) included only one AML case and contained a large number of t(4;11) (n=21) cases as well as four cases with t(11;19), one case with t(10;11), and one case with t(1;11). Finally, the “myeloid” cluster (Cluster C, n=54) was predominantly AML but contained twelve cases with an ALL label that nonetheless had a more “myeloid” pattern of gene expression. This cluster included some MLL cases with t(4;11), all the t(9;11), some t(11;19), and t(X;11). It has been suggested that in contrast to ALL, AML patients with MLL rearrangements do not tend to co-express lymphoid- and myeloid-associated antigens simultaneously on leukemic blasts and have outcomes similar to those without the gene rearrangements (Tien, 2000). Our data supports this view, since roughly the same frequencies of long-term remission (30%) and failures (70%) were observed in the “myeloid” cluster in patients irrespective of MLL translocations.

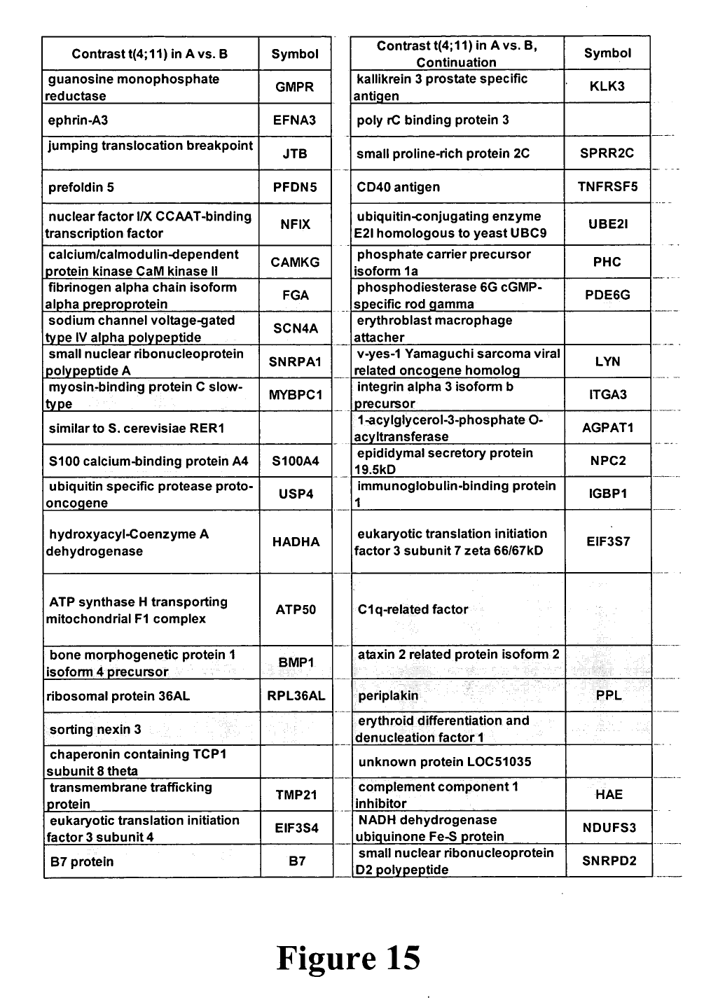

An important finding of the present study is that two very distinct groups of gene expression profiles could be identified across cases with the same t(4;11) rearrangement (VxInsight clusters A and B). Using ANOVA, a gene list that characterizes the t(4;11) groups within the infant clusters A and B was derived (FIG. 15). There is a considerable degree of overlap between the cluster A-characterizing genes and those that distinguish the t(4;11) cases in this group (previously submitted data). Cluster A was typified by genes of particular interest in signal transduction (EFNA3, B7 protein, Cytokeratin type II, latent transforming growth factor beta binding protein 4, Contactin 2 axonal, and Erythropoietin receptor precursor), transcription regulation (Integrin α3 (ITGA3), Ataxin 2 related protein (A2LP) and Heat-shock transcription factor 4, (HSF4)) and cell-to-cell signaling (Myosin-binding protein C slow-type). Although most useful in the separation of the cluster A cases, these genes seem to be separating the t(4;11) cases in this group as well.

Gene Expression Patterns of Different MLL Translocations

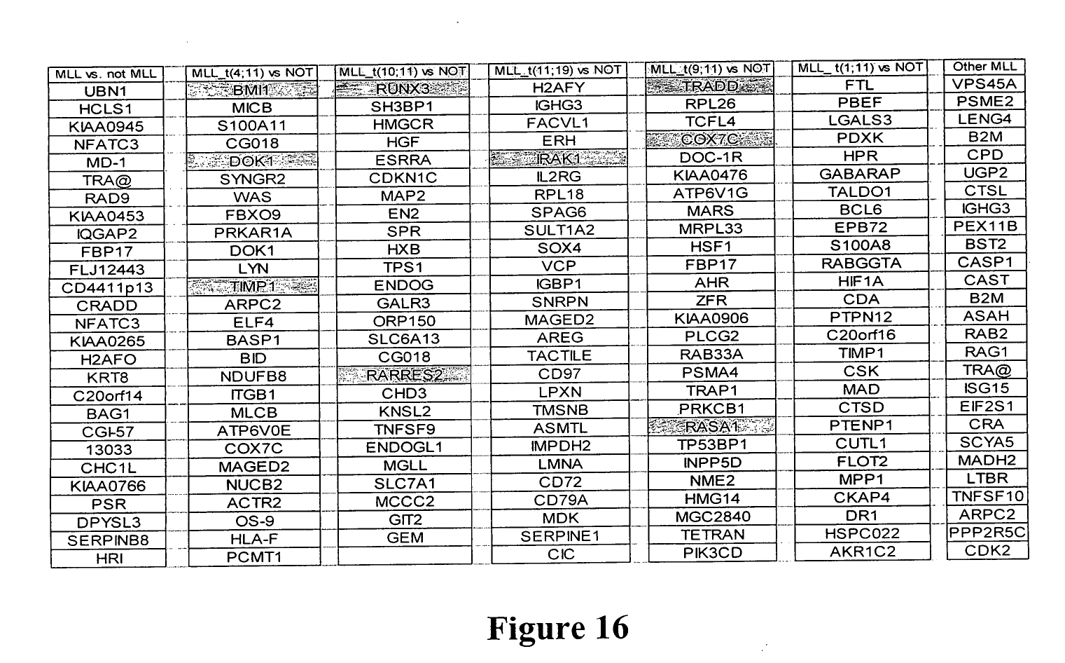

The second method used in our analysis was aimed at uncovering sets of genes that characterized each one of the MLL translocations. The process of defining the best set of discriminating genes was accomplished using supervised learning techniques such as Bayesian Networks, Linear Discriminant Analysis and Support Vector Machines (SVM) (Reviewed in Orr, 2002). In contrast with unsupervised methods, supervised learning methods learn “known classes”, creating classification algorithms that may undercover interesting and novel therapeutic targets. Our characterization of the gene expression profiles per MLL variant and the genes involved in these translocations accomplished using supervised learning techniques is shown in FIG. 16. These genes represent novel diagnostic and therapeutic targets for MLL-associated leukemias.

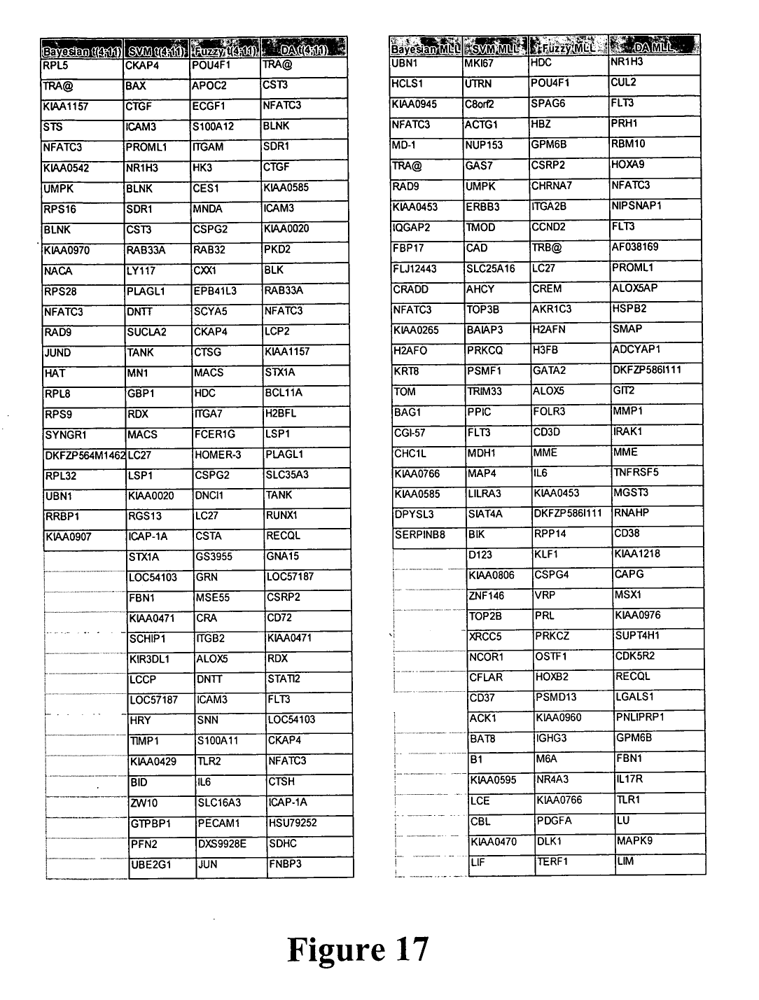

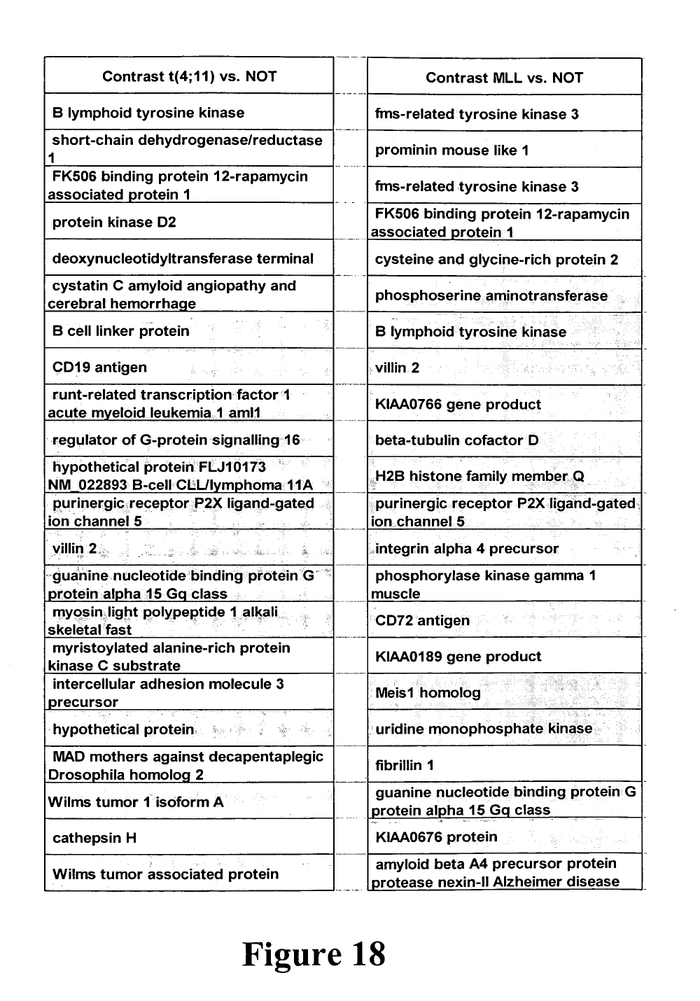

Gene expression profiles characteristic of the t(4;11) and other MLL translocations are shown in FIGS. 17 and 18 (FIG. 17: Bayesian Network analysis, Support Vector Machines analysis, Fuzzy Logics and Discriminant Analysis; FIG. 18: ANOVA from the VxInsight program). The different methods allowed the classification of unknown samples within each of the groups with accuracy rates higher than 90%, as calculated by fold dependent leave-one-out cross validation. This data analysis of gene expression conditioned on karyotype generated distinct case clustering, supporting that unique gene expression “signatures” identify defined genetic subsets of infant leukemia. This confirms recently published data (Armstrong et al, 2002), which revealed that the MLL infant leukemia cases are characterized by specific gene expression profiles. However, while groups of genes uniquely associated with the MLL cases can be identified using supervised learning techniques, infant MLL leukemia seems to be an entity comprised of several intrinsic biologic clusters not precisely predicted by current standards of morphology, immunophenotyping, or cytogenetics.

Expression Levels of FLT3 Across Various MLL Translocations

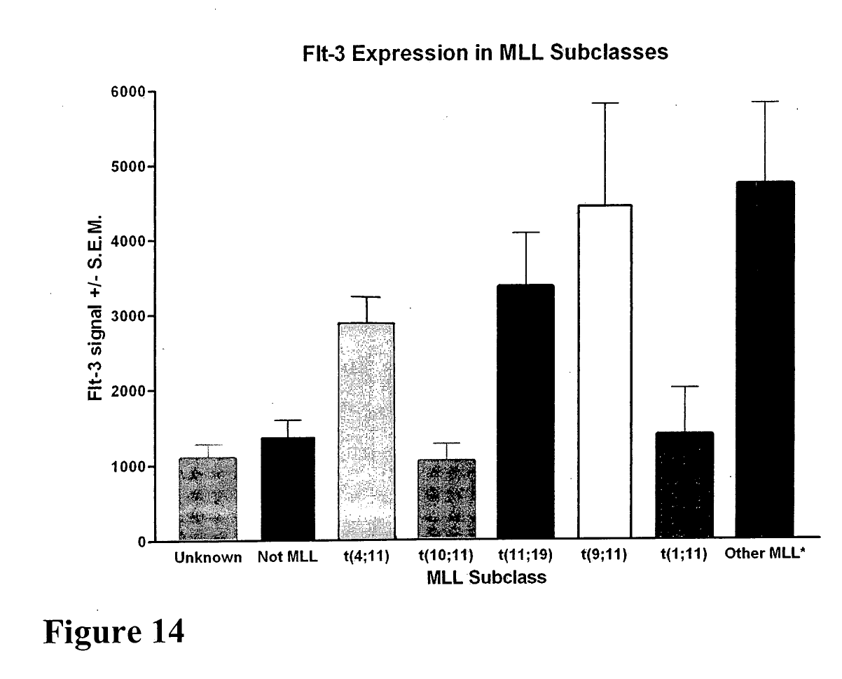

Expression levels of the FMS-related tyrosine kinase 3 (FLT3) gene were analyzed across different MLL translocations. FLT3, a member of the receptor tyrosine kinase (RTK) class III, is preferentially expressed on the surface of a high proportion of acute myeloid leukemia (AML) and B-lineage acute lymphocytic leukemia (ALL) cells in addition to hematopoietic stem cells, brain, placenta and liver (Kiyoe, 2002). Within MLL subgroups FLT3 is variable. The expression levels for this gene were differentially higher in t(4;11), t(11;19), t(9;11) and other MLL translocations (FIG. 14)). However, MLL subgroups such as t(1;11) and t(10;11) had similar expression of FLT3 compared to not MLL cases, suggesting that the various MLL translocations may exert differential influence on the FLT3 expression levels. This may add arguments to the previously proposed potential problems in the clinical use of FLT3 inhibitors for leukemia treatment (Gilliland et al, 2002).

Discussion

Gene expression profiling of our infant MLL leukemia cases revealed new insights into infant leukemia classification that may increase our understanding of the pathogenesis and hence, treatment options for this disease.

While groups of genes uniquely associated with each MLL translocation variant can be identified using supervised learning techniques (as previously shown by others), infant acute MLL leukemia seems to be an entity comprised of several intrinsic biologic clusters not precisely predicted by current standards of morphology, immunophenotyping, or cytogenetics. Unsupervised analysis demonstrated that gene expression in specific MLL rearrangements varied significantly amongst the three infant groups. As these intrinsic clusters appeared to relate to distinct subtypes of infant leukemia, the various MLL translocations may represent a critical secondary transforming event for each biological group, conferring more defined tumor phenotypes. Alternatively, MLL translocations may be permissive for further genetic rearrangements that will strongly influence and define differential gene expression patterns. Our findings of heterogeneity of gene expression within and between MLL subtypes differ from previous reports suggesting more homogeneous gene expression (Armstrong, 2002). This probably reflects mainly the larger number of cases available to us for analysis. However, rigorous exclusion of unsatisfactory samples was also critical for the successful interpretation of the data.

Particular genes that can be selected by supervised methods as characterizing cases with MLL translocations, in the current study the presence or absence of MLL rearrangements did not define a distinct leukemia class during unsupervised learning analysis of the gene expression patterns of these infant patients. Despite the fact that supervised analysis of the microarray data can successfully segregate patients defined by traditional methods such as immunophenotyping and cytogenetics, results from these techniques are most useful in the identification of unanticipated similarities and diversities in individual patients and thus may be useful in augmenting risk-group stratification in the future. Further studies to enhance the ability to classify infant MLL subtypes according to shared pathways of leukemic transformation will have important implications for the development of new therapeutic approaches.

### REFERENCES

Armstrong, S. A., Staunton, J. E., Silverman, L. B., Pieters, R., den Boer, M. L., Minden, M. D., Sallan, S. E., Lander, E. S., Golub, T. R., Korsmeyer, S. J. MLL translocations specify a distinct gene expression profile that distinguishes a unique leukemia. Nat Genet 2002 January; 30(1):41-7
Chen C. S., Sorensen P. H. B., Domer P. H., Reaman G. H., Korsmeyer S. J., Heerema N. A., Hammond G. D., Kersey J. H. Molecular-rearrangements on chromosome-11q23 predominate in infant acute lymphoblastic-leukemia and are associated with specific biologic variables and poor outcome. Blood. 81, 2386-2393 (1993).
Davidson, G. S., Wylie, B. N., and Boyack, K. W. Cluster stability and the use of noise in interpretation of clustering. Proc. IEEE Information Visualization 2001, 23-30 (2001).
Davidson, G. S., Hendrickson, B., Johnson, D. K., Meyers, C. E., & Wylie, B. N. Knowledge mining with VxInsight: Discovery through interaction. J. Int. Inf. Syst. 11, 259-285 (1998).
Efron, B. Bootstrap methods—“another look at the jackknife” Ann. Statist., 7, 1-26 (1979).
Ernst P., Wang J., Korsmeyer S. J. The role of MLL in hematopoiesis and leukemia. Curr. Opin. Hematol. 9, 282-287 (2002).
Felix, C., Lange, B. Leukemia in infants. The Oncologist. 4, 225-240 (1999).
Gilliland, D. G., Griffin, J. D. Role of FLT3 in leukemia. Curr Opin Hematol. 9, 274-81. (2002)
Gu, Y.; Nakamura, T.; Alder, H.; Prasad, R.; Canaani, O.; Cimino, G.; Croce, C. M.; Canaani, E. The t(4;11) chromosome translocation of human acute leukemias fuses the ALL-1 gene, related to Drosophila trithorax, to the AF-4 gene. Cell 71, 701-708 (1992).
Hjorth, J. S. Urban Computer Intensive Statistical Methods, Validation model selection and bootstrap, ISBN 0412491605, Chapman & Hall, 2-6 Boundary Row, London SE1 8HN, UK. (1994).
Kiyoi, H., Naoe, T. FLT3 in human hematologic malignancies. Leuk Lymphoma. 43, 1541-7 (2002).
Orr, M. S., Scherf, U. Large-scale gene expression analysis in molecular target discovery. Leukemia. 16:473-7 (2002). Review.
Parry, P.; Djabali, M.; Bower, M.; Khristich, J.; Waterman, M.; Gibbons, B.; Young, B. D.; Evans, G. Structure and expression of the human trithorax-like gene 1 involved in acute leukemias. Proc. Nat. Acad. Sci. 90, 4738-4742 (1993).
Rowley, J. D. The critical role of chromosome translocation sin human genetics. Annu. Rev. Genet. 32, 495-519, (1998).
Sorensen P. H. B., Chen C. S., Smith F. O., Arthur D. C., Domer P. H., Bernstein I. D., Korsmeyer S. J., Hammond G. D., Kersey J. H. Molecular-rearrangements of the MLL gene are present in most cases of infant acute myeloid-leukemia and are strongly correlated with monocytic or myelomonocytic phenotypes. J. Clin. Investig., 93, 429-437 (1994).
Strick, R., Strissel, P., Borgers, S., Smith, S., Rowley, S. Dietary bioflavonoids induce cleavage in the MLL gene and may contribute to infant leukemia Proc. Natl. Acad. Sci. USA. 97, 4790-4795 (2000).
Tien, H. F., Hsiao, C. H., Tang, J. L., Tsay, W., Hu, C. H., Kuo, Y. Y., Wang, C. H., Chen, Y. C., Shen, M. C., Lin, D. T., Lin, H. K., Lin, K. S. Characterization of acute myeloid leukemia with MLL rearrangement: no increase in the incidence of coexpression of lymphoid-associated antigens on leukemic blasts. Leukemia. 14, 1025-1030 (2000).
   The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An isolated OPAL1 polynucleotide comprising a nucleotide sequence selected from the group consisting of:
(a) SEQ ID NO:1 or 3;
(b) a complement of SEQ ID NO:1 or 3;
(c) a subunit of SEQ ID NO:1 or 3 consisting of at least 60 contiguous nucleotides;
(d) a nucleotide sequence that hybridizes to SEQ ID NO:1 or 3;
(e) a nucleotide sequence having at least 95% identity to SEQ ID NO:1 or 3
(f) a nucleotide sequence having at least 98% identity to SEQ ID NO:1 or 3
(g) a nucleotide sequence encoding a polypeptide encoded by SEQ ID NO:2 or 4.

2. An isolated OPAL1 polynucleotide comprising the nucleotide sequence SEQ ID NO:1 or 3.

3. An isolated OPAL1 polynucleotide comprising a nucleotide sequence encoding the amino sequence SEQ ID NO:2 or 4.

4. An isolated OPAL1 polypeptide comprising an amino acid sequence selected from the group consisting of:
(a) SEQ ID NO:2 or 4;
(b) a subunit of SEQ ID NOs:2 or 4 having at least 20 contiguous amino acids;
(c) an amino acid sequence having at least 90% identity to SEQ ID NOs:2 or 4
(c) an amino acid sequence having at least 95% identity to SEQ ID NOs:2 or 4.

5. An isolated OPAL1 polypeptide comprising the amino acid sequence SEQ ID NO:2 or 4.

6. An isolated OPAL1 polypeptide comprising an amino acid sequence having at least about 90% identity to SEQ ID NO:2 or 4, wherein the polypeptide retains at least a portion of the biological activity of SEQ ID NO:2 or 4.

7. An expression vector comprising a polynucleotide of claim 1 operably linked to an expression control sequence.

8. A host cell transformed or transfected with an expression vector according to claim 3.

9. An isolated antibody, or antigen-binding fragment thereof, that specifically binds to the polypeptide of claim 4.

10. A method for predicting therapeutic outcome in a leukemia patient comprising:
(a) obtaining a biological sample from a patient;
(b) determining the expression level for an OPAL1 gene product to yield an observed OPAL1 gene expression level; and
(c) comparing the observed OPAL1 gene expression level for the OPAL1 gene product to a control OPAL1 gene expression level selected from the group consisting of:
(i) the OPAL1 gene expression level for the OPAL1 gene product observed in a control sample; and
(ii) a predetermined OPAL1 gene expression level for the OPAL1 gene product;
wherein an observed OPAL1 expression level that is higher than the control OPAL1 gene expression level is indicative of predicted remission.

11. The method of claim 10 further comprising determining the expression level for a G1 or G2 gene product to yield an observed G1 or G2 gene expression level; and comparing the observed G1 or G2 gene expression level for the G1 or G2 gene product to a control G1 or G2 gene expression level selected from the group consisting of: (i) the G1 or G2 gene expression level for the G1 or G2 gene product observed in a control sample; and (ii) a predetermined G1 or G2 gene expression level for the G1 or G2 gene product; wherein an observed G1 or G2 expression level that is different from the control G1 or G2 gene expression level is further indicative of predicted remission.

12. A method for detecting an OPAL1 polynucleotide in a biological sample comprising:
(a) contacting the sample with the polynucleotide of claim 1 under conditions in which the polynucleotide selectively hybridizes to an OPAL1 gene; and
(b) detecting hybridization of the nucleic acid molecule to the OPAL1 gene in the sample.

13. A method for detecting an OPAL1 protein in a biological sample comprising:
(a) contacting the sample with the antibody according to claim 9 under conditions in which the antibody selectively binds to an OPAL1 protein; and
(b) detecting the binding of the antibody to the OPAL1 protein in the sample.

14. A pharmaceutical composition comprising:
(a) a therapeutic agent selected from the group consisting of:
(i) a polynucleotide of claim 1;
(ii) a polypeptide of claim 4; and
(iii) a compound that enhances the activity of the polypeptide of claim 4; and
(b) a pharmaceutically acceptable carrier.

15. The pharmaceutical composition of claim 14 further comprising:
(a) a second therapeutic agent selected from the group consisting of:
(i) a polynucleotide encoding G1 or G2;
(ii) a G1 or G2 polypeptide; and
(iii) a compound that alters the activity of a G1 or G2 polypeptide.

16. A method for treating leukemia comprising administering to a leukemia patient a therapeutic agent that increases the amount or activity of the polypeptide of claim 4 in the patient.

17. The method of claim 16 further comprising administering to a leukemia patient a therapeutic agent that alters the amount or activity of a G1 or G2 polypeptide.

18. A method for screening compounds useful for treating leukemia comprising:
(a) determining the expression level for an OPAL1 gene product in a cell culture to yield an observed OPAL1 gene expression level prior to contact with a candidate compound;
(b) contacting the cell culture with a candidate compound;
(c) determining the expression level for the OPAL1 gene product in the cell culture to yield an observed OPAL1 gene expression level after contact with the candidate compound; and
(d) comparing the observed OPAL1 gene expression level before and after contact with the candidate compound wherein an increase in OPAL1 gene expression level after contact with the compound is indicative of therapeutic utility.

19. A method for screening compounds useful for treating leukemia comprising:
(a) contacting an experimental cell culture with a candidate compound;
(b) determining the expression level for an OPAL1 gene product in the cell culture to yield an experimental OPAL1 gene expression level; and
(b) comparing the experimental OPAL1 expression level to the expression level of the OPAL1 gene product in a control cell culture, wherein a relative difference in the gene expression levels between the experimental and control cultures is indicative of therapeutic utility.

20. A method for evaluating a compound for use in treating leukemia, comprising:
(a) obtaining a first biological sample from a patient;
(b) determining the expression level for an OPAL1 gene product in the first biological sample to yield an observed OPAL1 gene expression level prior to administration of a candidate compound;
(c) administering a candidate compound to the patient;
(d) obtaining a second biological sample from the patient;
(e) determining the expression level for an OPAL1 gene product in the second biological sample to yield an observed OPAL gene expression level after administration of the candidate compound; and
(f) comparing the observed OPAL1 gene expression levels before and after administration of the candidate compound to determine whether the compound has therapeutic utility.

21. A method for classifying leukemia in a patient comprising:
(a) obtaining a biological sample from a patient;
(b) determining the expression level for a selected gene product to yield an observed gene expression level; and
(c) comparing the observed gene expression level for the selected gene product to a control gene expression level selected from the group consisting of:
(i) the expression level observed for the gene product in a control sample; and
(ii) a predetermined expression level for the gene product;
wherein an observed expression level that differs from the control gene expression level is indicative of a disease classification.

22. The method of claim 21 wherein the disease classification comprises predicted remission or therapeutic failure.

23. The method of claim 22 wherein the gene product is produced by a gene selected from the group consisting of OPAL1, G1, G2, FYN binding protein, PBK1 and any of the genes listed in Table 42.

24. The method of claim 21 wherein the disease classification comprises a classification based on karyotype.

25. The method of claim 21 wherein the disease classification comprises leukemia subtype.

26. The method of claim 21 wherein the disease classification comprises a classification based on disease etiology.

27. A method for classifying leukemia in a patient comprising:
(a) obtaining a biological sample from a patient;
(b) determining a gene expression profile for selected gene products to yield an observed gene expression profile; and
(c) comparing the observed gene expression profile for the selected gene products to a control gene expression profile for the selected gene products that correlates with a disease classification;
wherein a similarity between the observed gene expression profile and the control gene expression profile is indicative of the disease classification.

28. The method of claim 27 wherein the disease classification comprises predicted remission or therapeutic failure.

29. The method of claim 28 wherein at least one of the gene products is produced by a gene selected from the group consisting of OPAL1, G1, G2, FYN binding protein, PBK1 and any of the genes listed in Table 42.

30. The method of claim 27 wherein the disease classification comprises a classification based on karyotype.

31. The method of claim 27 wherein the disease classification comprises leukemia subtype.

32. The method of claim 27 wherein the disease classification comprises a classification based on disease etiology.

33. A method for screening compounds useful for treating acute leukemia comprising:
(a) determining the expression level for a selected gene product in a cell culture to yield an observed expression level for the gene product prior to contact with a candidate compound, wherein the selected gene product is correlated with therapeutic outcome;
(b) contacting the cell culture with a candidate compound;
(c) determining the expression level for the selected gene product in a cell culture to yield an observed gene expression level after contact with the candidate compound; and
(d) comparing the observed expression levels of the selected gene product before and after contact with the candidate compound wherein a modulation of gene expression level after contact with the compound is indicative of therapeutic utility.

34. The method of claim 33 wherein the gene product is produced by a gene selected from the group consisting of OPAL1, G1, G2, FYN binding protein, PBK1 and any of the genes listed in Table 42.

35. A method for screening compounds useful for treating acute leukemia comprising:
(a) determining a gene expression profile for selected gene products in a cell culture to yield an observed gene expression profile prior to contact with a candidate compound, wherein the selected gene products are correlated with therapeutic outcome;
(b) contacting the cell culture with a candidate compound;
(c) determining a gene expression profile for the selected gene products in the cell culture to yield an observed gene expression profile after contact with the candidate compound; and
(d) comparing the observed expression profiles before and after contact with the candidate compound to determine whether the compound has therapeutic utility.

36. The method of claim 35 wherein at least one of the gene products is produced by a gene selected from the group consisting of OPAL1, G1, G2, FYN binding protein, PBK1 and any of the genes listed in Table 42.

37. A method for screening compounds useful for acute treating leukemia comprising:
(a) contacting an experimental cell culture with a candidate compound;
(b) determining the expression level for a selected gene product in the cell culture to yield an experimental gene expression level for the gene product, wherein the selected gene product is correlated with therapeutic outcome; and
(c) comparing the experimental gene expression level to the expression level of the selected gene product in a control cell culture, wherein a relative difference in the gene expression levels between the experimental and control cultures is indicative of therapeutic utility.

38. The method of claim 37 wherein the gene product is produced by a gene selected from the group consisting of OPAL1, G1, G2, FYN binding protein, PBK1 and any of the genes listed in Table 42.

39. A method for screening compounds useful for acute treating leukemia comprising:
(a) contacting an experimental cell culture with a candidate compound;
(b) determining a gene expression profile for selected gene products in the cell culture to yield an experimental gene expression profile, wherein the selected gene products are correlated with therapeutic outcome; and
(c) comparing the experimental gene expression profile to the gene expression profile for the selected gene products in a control cell culture to determine whether the compound has therapeutic utility.

40. The method of claim 39 wherein at least one of the gene products is produced by a gene selected from the group consisting of OPAL1, G1, G2, FYN binding protein, PBK1 and any of the genes listed in Table 42.

41. A method for evaluating a compound for use in treating leukemia, comprising:
(a) obtaining a first biological sample from a patient;
(b) determining a gene expression profile for selected gene products in the first biological sample to yield an observed gene expression profile prior to administration of a candidate compound, wherein the selected gene products are correlated with therapeutic outcome;
(c) administering a candidate compound to the patient;
(d) obtaining a second biological sample from the patient;
(e) determining a gene expression profile for the selected gene products in the second biological sample to yield an observed gene expression profile after administration of the candidate compound; and
(f) comparing the observed gene expression profiles before and after administration of the candidate compound to determine whether the compound has therapeutic utility.

42. The method of claim 41 wherein at least one of the gene products is produced by a gene selected from the group consisting of OPAL1, G1, G2, FYN binding protein, PBK1 and any of the genes listed in Table 42.
